(19) United States
(12) Patent Application Publication
(10) Pub. No.: US 2025/0017929 A1
(43) Pub. Date: Jan. 16, 2025

(54) Methods Of Treating Skin Cancer With Carboxypeptidase Vitellogenic Like (CPVL) Inhibitors

(71) Applicant: Regeneron Pharmaceuticals, Inc., Tarrytown, NY (US)

(72) Inventors: Xinyuan Zhang, Tarrytown, NY (US); Julie Horowitz, Tarrytown, NY (US); Stefan Semrau, Tarrytown, NY (US); Goncalo Abecasis, Tarrytown, NY (US); Gavin Thurston, Tarrytown, NY (US); Eric Jorgenson, Tarrytown, NY (US); Aris Baras, Tarrytown, NY (US)

(21) Appl. No.: 18/775,603

(22) Filed: Jul. 17, 2024

Related U.S. Application Data
(62) Division of application No. 17/857,625, filed on Jul. 5, 2022, now Pat. No. 12,070,460.
(60) Provisional application No. 63/219,464, filed on Jul. 8, 2021.

(51) Int. Cl. A61K 31/506; A61K 31/713; C07K 16/28

(52) U.S. Cl. CPC A61K 31/506; A61K 31/713; C07K 16/2896

(57) ABSTRACT

The present disclosure provides methods of treating a subject having skin cancer or preventing a subject from developing skin cancer, and methods of identifying subjects having an increased risk of developing skin cancer.

### REFERENCE TO SEQUENCE LISTING

This application includes a Sequence Listing filed electronically as an XML file named 381204287SEQ, created on Jul. 17, 2024 with a size of 402,077 bytes. The Sequence Listing is incorporated herein by reference.

### FIELD

The present disclosure relates generally to the treatment of subjects having skin cancer with Carboxypeptidase Vitellogenic Like (CPVL) inhibitors, and methods of identifying subjects having an increased risk of developing skin cancer.

### BACKGROUND

Skin cancer refers to all cancers that occur in the skin. These relatively common cancers are often mistaken by patients for non-malignant skin abnormalities, which can result in late detection that leads to difficulties in treating the disease and fatal outcomes. The most common of skin cancers is basal cell carcinoma (BCC), which accounts for about 80% of all skin cancers. Other types of skin cancers are squamous cell carcinoma (SCC), which accounts for approximately 16%, of all skin cancers, and melanoma, which accounts for about 4%. BCC and SCC are collectively referred to as non-melanoma skin cancer (NMSC). Melanoma occurs from melanocytes in the epidermis, many of which are metastatic cancers or carcinomas that lead to death. In 2000, 47,000 people were identified as having new melanomas, of which 7,700 were reported to have died (Greenlee et al., Cancer J. Clin., 2000, 50, 7-33. It is estimated that melanoma caused by ultraviolet rays is caused by intermittent exposure, such as intense tanning rather than chronic exposure to ultraviolet rays (Gilchrest et al., New Engl. J. Med., 1999, 340, 1341-1348). Another rare form of aggressive skin cancer is Merkel cell carcinoma (MCC), which is similar to melanoma.

Carboxypeptidase Vitellogenic Like (CPVL) is a carboxypeptidase and bears strong sequence similarity to serine carboxypeptidases. Carboxypeptidases are a large class of proteases that act to cleave a single amino acid from the carboxy termini of proteins or peptides. The exact function of this protein, however, has not been determined. CPVL may be involved in the digestion of phagocytosed particles in the lysosome, participation in an inflammatory protease cascade, and trimming of peptides for antigen presentation.

### SUMMARY

The present disclosure provides methods of treating a subject having skin cancer or preventing a subject from developing skin cancer, the methods comprising administering a CPVL inhibitor to the subject.

The present disclosure also provides methods of treating a subject having non-melanoma skin cancer or preventing a subject from developing non-melanoma skin cancer, the methods comprising administering a CPVL inhibitor to the subject.

The present disclosure also provides methods of treating a subject having basal cell carcinoma or preventing a subject from developing basal cell carcinoma, the methods comprising administering a CPVL inhibitor to the subject.

The present disclosure also provides methods of treating a subject having squamous cell carcinoma or preventing a subject from developing squamous cell carcinoma, the methods comprising administering a CPVL to the subject.

The present disclosure also provides methods of treating a subject having melanoma or preventing a subject from developing melanoma, the methods comprising administering a CPVL inhibitor to the subject.

The present disclosure also provides methods of treating a subject having Merkel cell carcinoma or preventing a subject from developing Merkel cell carcinoma, the methods comprising administering a CPVL inhibitor to the subject.

The present disclosure also provides methods of treating a subject having dermatofibrosarcoma protuberans or preventing a subject from developing dermatofibrosarcoma protuberans, the methods comprising administering a CPVL inhibitor to the subject.

The present disclosure also provides methods of treating a subject having sebaceous carcinoma or preventing a subject from developing sebaceous carcinoma, the methods comprising administering a CPVL inhibitor to the subject.

The present disclosure also provides methods of treating a subject with a therapeutic agent that treats or inhibits skin cancer, wherein the subject has skin cancer, the methods comprising the steps of: determining whether the subject has a CPVL missense variant nucleic acid molecule encoding a CPVL predicted loss-of-function polypeptide by: obtaining or having obtained a biological sample from the subject; and performing or having performed a sequence analysis on the biological sample to determine if the subject has a genotype comprising the CPVL missense variant nucleic acid molecule encoding a CPVL predicted loss-of-function polypeptide; and: i) administering or continuing to administer the therapeutic agent that treats or inhibits skin cancer in a standard dosage amount to a subject that is CPVL reference, and/or administering a CPVL inhibitor to the subject; ii) administering or continuing to administer the therapeutic agent that treats or inhibits skin cancer in an amount that is the same as or less than a standard dosage amount to a subject that is heterozygous for the CPVL missense variant nucleic acid molecule, and/or administering a CPVL inhibitor to the subject; or iii) administering or continuing to administer the therapeutic agent that treats or inhibits skin cancer in an amount that is the same as or less than a standard dosage amount to a subject that is homozygous for the CPVL missense variant nucleic acid molecule; wherein the presence of a genotype having the CPVL missense variant nucleic acid molecule encoding the CPVL predicted loss-of-function polypeptide indicates the subject has a decreased risk of developing skin cancer.

The present disclosure also provides methods of treating a subject with a therapeutic agent that prevents skin cancer, the methods comprising the steps of: determining whether the subject has a CPVL missense variant nucleic acid molecule encoding a CPVL predicted loss-of-function polypeptide by: obtaining or having obtained a biological sample from the subject; and performing or having performed a sequence analysis on the biological sample to determine if the subject has a genotype comprising the CPVL missense variant nucleic acid molecule encoding a CPVL predicted loss-of-function polypeptide; and: i) administering or continuing to administer the therapeutic agent that prevents skin cancer in a standard dosage amount to a subject that is CPVL reference, and/or administering a CPVL inhibitor to the subject; ii) administering or continuing to administer the therapeutic agent that prevents skin cancer in an amount that is the same as or less than a standard dosage amount to a subject that is heterozygous for the CPVL missense variant nucleic acid molecule, and/or administering a CPVL inhibitor to the subject; or iii) administering or continuing to administer the therapeutic agent that prevents skin cancer in an amount that is the same as or less than a standard dosage amount to a subject that is homozygous for the CPVL missense variant nucleic acid molecule; wherein the presence of a genotype having the CPVL missense variant nucleic acid molecule encoding the CPVL predicted loss-of-function polypeptide indicates the subject has a decreased risk of developing skin cancer.

The present disclosure also provides methods of identifying a subject having an increased risk of developing skin cancer, the methods comprising: determining or having determined the presence or absence of a CPVL missense variant nucleic acid molecule encoding a CPVL predicted loss-of-function polypeptide in a biological sample obtained from the subject; when the subject is CPVL reference, then the subject has an increased risk of developing skin cancer; and when the subject is heterozygous or homozygous for the CPVL missense variant nucleic acid molecule encoding the CPVL predicted loss-of-function polypeptide, then the subject has a decreased risk of developing skin cancer.

The present disclosure also provides therapeutic agents that treat or inhibit or prevent skin cancer for use in the treatment or prevention of skin cancer in a subject that: a) is reference for a CPVL genomic nucleic acid molecule, a CPVL mRNA molecule, or a CPVL cDNA molecule; or b) is heterozygous for: i) a CPVL missense variant genomic nucleic acid molecule encoding a CPVL predicted loss-of-function polypeptide; ii) a CPVL missense variant mRNA molecule encoding a CPVL predicted loss-of-function polypeptide; or iii) a CPVL missense variant cDNA molecule encoding a CPVL predicted loss-of-function polypeptide.

The present disclosure also provides CPVL inhibitors for use in the treatment or prevention of skin cancer in a subject that: a) is reference for a CPVL genomic nucleic acid molecule, a CPVL mRNA molecule, or a CPVL cDNA molecule; or b) is heterozygous for: i) a CPVL missense variant genomic nucleic acid molecule encoding a CPVL predicted loss-of-function polypeptide; ii) a CPVL missense variant mRNA molecule encoding a CPVL predicted loss-of-function polypeptide; or iii) a CPVL missense variant cDNA molecule encoding a CPVL predicted loss-of-function polypeptide.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows CPVL expression (recovered molecules/cell) of melanoma tumor.

FIG. 2 shows CPVL expression (recovered molecules/cell) of basal cell carcinoma.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by +10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or Alternately phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human. In some embodiments, the human is a patient under the care of a physician.

It has been observed in accordance with the present disclosure that CPVL missense variant nucleic acid molecules encoding a CPVL predicted loss-of-function polypeptide (whether these variations are homozygous or heterozygous in a particular subject) associate with a decreased risk of developing skin cancer. It is believed that CPVL missense variant nucleic acid molecules encoding a CPVL predicted loss-of-function polypeptide have not been associated with melanoma. Moreover, the identification by the present disclosure of the association between additional variants and gene burden masks indicates that CPVL itself (rather than linkage disequilibrium with variants in another gene) is responsible for a protective effect in non-melanoma skin cancer and melanoma.

Therefore, subjects that are CPVL reference or heterozygous for CPVL missense variant nucleic acid molecules encoding a CPVL predicted loss-of-function polypeptide may be treated with a CPVL inhibitor such that skin cancer is inhibited or prevented, the symptoms thereof are reduced or prevented, and/or development of symptoms is repressed or prevented. It is also believed that such subjects having skin cancer may further be treated with therapeutic agents that treat or inhibit skin cancer.

For purposes of the present disclosure, any particular subject, such as a human, can be categorized as having one of three CPVL genotypes: i) CPVL reference; ii) heterozygous for a CPVL missense variant nucleic acid molecule encoding a CPVL predicted loss-of-function polypeptide; or iii) homozygous for a CPVL missense variant nucleic acid molecules encoding a CPVL predicted loss-of-function polypeptide. A subject is CPVL reference when the subject does not have a copy of a CPVL missense variant nucleic acid molecules encoding a CPVL predicted loss-of-function polypeptide. A subject is heterozygous for a CPVL missense variant nucleic acid molecules encoding a CPVL predicted loss-of-function polypeptide when the subject has a single copy of a CPVL missense variant nucleic acid molecules encoding a CPVL predicted loss-of-function polypeptide. A CPVL missense variant nucleic acid molecules encoding a CPVL predicted loss-of-function polypeptide is any nucleic acid molecule (such as, a genomic nucleic acid molecule, an mRNA molecule, or a cDNA molecule) encoding a variant CPVL polypeptide having a partial loss-of-function, a complete loss-of-function, a predicted partial loss-of-function, or a predicted complete loss-of-function. A subject who has a CPVL polypeptide having a partial loss-of-function (or predicted partial loss-of-function) is hypomorphic for CPVL. A subject is homozygous for a CPVL missense variant nucleic acid molecules encoding a CPVL predicted loss-of-function polypeptide when the subject has two copies (same or different) of a CPVL missense variant nucleic acid molecules encoding a CPVL predicted loss-of-function polypeptide.

For subjects that are genotyped or determined to be CPVL reference, such subjects have an increased risk of developing skin cancer, such as non-melanoma skin cancer, basal cell carcinoma, squamous cell carcinoma, melanoma, Merkel cell carcinoma, dermatofibrosarcoma protuberans, and/or sebaceous carcinoma. For subjects that are genotyped or determined to be either CPVL reference or heterozygous for a CPVL missense variant nucleic acid molecules encoding a CPVL predicted loss-of-function polypeptide, such subjects or subjects can be treated with a CPVL inhibitor.

In any of the embodiments described herein, the subject in whom skin cancer is prevented by administering the CPVL inhibitor can be anyone at risk for developing skin cancer including, but not limited to, subjects with a familial or genetic risk, older subjects, subjects having European descent, and subjects having lighter skin pigmentation. In addition, in some embodiments, any subject can be at risk of developing skin cancer. In some embodiments, administering a CPVL inhibitor may be carried out to prevent development of an additional skin cancer(s) in a subject who has already had one or more skin cancers.

In any of the embodiments described herein, the CPVL missense variant nucleic acid molecules encoding a CPVL predicted loss-of-function polypeptide can be any nucleic acid molecule (such as, for example, genomic nucleic acid molecule, mRNA molecule, or cDNA molecule) encoding a CPVL variant polypeptide having a partial loss-of-function, a complete loss-of-function, a predicted partial loss-of-function, or a predicted complete loss-of-function. In some embodiments, the CPVL missense variant nucleic acid molecules encoding a CPVL predicted loss-of-function polypeptide is associated with a reduced in vitro response to CPVL ligands compared with reference CPVL. In some embodiments, the CPVL missense variant nucleic acid molecules encoding a CPVL predicted loss-of-function polypeptide is a CPVL variant that results or is predicted to result in a premature truncation of a CPVL polypeptide compared to the human reference genome sequence. In some embodiments, the CPVL missense variant nucleic acid molecules encoding a CPVL predicted loss-of-function polypeptide is a variant that is predicted to be damaging by in vitro prediction algorithms such as Polyphen, SIFT, or similar algorithms. In some embodiments, the CPVL missense variant nucleic acid molecules encoding a CPVL predicted loss-of-function polypeptide is a variant that causes or is predicted to cause a nonsynonymous amino-acid substitution in CPVL and whose allele frequency is less than 1/100 alleles in the population from which the subject is selected. In some embodiments, the CPVL missense variant nucleic acid molecules encoding a CPVL predicted loss-of-function polypeptide is any rare missense variant (allele frequency 21 0.1%; or 1 in 1,000 alleles), or any splice-site, stop-gain, start-loss, stop-loss, frameshift, or in-frame indel, or other frameshift CPVL variant.

In any of the embodiments described herein, the CPVL predicted loss-of-function polypeptide can be any CPVL polypeptide having a partial loss-of-function, a complete loss-of-function, a predicted partial loss-of-function, or a predicted complete loss-of-function.

In any of the embodiments described herein, the CPVL missense variant nucleic acid molecules encoding a CPVL predicted loss-of-function polypeptide can include variations at positions of chromosome 7 using the nucleotide sequence of the CPVL reference genomic nucleic acid molecule (SEQ ID NO:1; ENSG00000106066.15 chr7: 28,995,637-29,195,276 in the GRCh38/hg38 human genome assembly) as a reference sequence.

Numerous genetic variants in CPVL exist which cause subsequent changes in the CPVL polypeptide sequence including, but not limited to: rs117744081 (Tyr168His), rs147771477 (Arg464Gln), and rs138216401 (Ser61Asn).

Any one or more (i.e., any combination) of the CPVL missense variant nucleic acid molecules encoding a CPVL predicted loss-of-function polypeptide can be used within any of the methods described herein to determine whether a subject has an increased risk of developing skin cancer. The combinations of particular variants can form a mask used for statistical analysis of the particular correlation of CPVL and increased risk of developing skin cancer.

In any of the embodiments described herein, the skin cancer is non-melanoma skin cancer, basal cell carcinoma, squamous cell carcinoma (including cutaneous squamous cell carcinoma), melanoma, Merkel cell carcinoma, dermatofibrosarcoma protuberans, and/or sebaceous carcinoma. In some embodiments, the skin cancer is non-melanoma skin cancer. In some embodiments, the skin cancer is basal cell carcinoma. In some embodiments, the skin cancer is squamous cell carcinoma. In some embodiments, the skin cancer is melanoma. In some embodiments, the skin cancer is Merkel cell carcinoma. In some embodiments, the skin cancer is dermatofibrosarcoma protuberans. In some embodiments, the skin cancer is sebaceous carcinoma.

Symptoms of basal cell carcinoma include, but are not limited to, a raised, smooth, pearly bump on the sun-exposed skin of an individual's head, neck or shoulders. Often small blood vessels can be seen within the tumor. Crusting of the tumor, as well as bleeding can occur. Individuals sometimes mistake basal cell carcinoma as a sore that will not heal. Basal cell carcinoma is the least deadly form of skin cancer and often times with proper treatment can be completely eliminated.

Symptoms of squamous cell carcinoma include, but are not limited to a red, scaling, thickened patch on the sun exposed skin of an individual. Some forms of squamous cell carcinoma appear as firm hard nodules and as dome shapes. Breaks and bleeding of the nodules may occur. If left untreated, the squamous cell carcinoma could develop into a large mass. Squamous cell carcinoma is the second most common form of skin cancer.

Symptoms of melanoma include, but are not limited to, shades or brown to black lesions. There are also some melanomas which appear pink, red or flesh color, these are called amelanotic melanomas. The amelanotic melanomas are a more aggressive form of melanoma. Some of the warning signs of malignant melanoma could include changes in size, shape, color, elevation of a mole, the development of a new mole in the transitional period from puberty to adulthood, itching, ulceration or bleeding. Melanoma is the most deadly form of skin cancer.

Symptoms of Merkel cell carcinoma include, but are not limited to rapid growing, non-tender flesh colored to red/violet bumps that are usually not painful or itchy. These bumps appear on the highly sun exposed skin of the head, neck and arms. Individuals often mistake Merkel cell carcinoma for a cyst or other type of cancer.

Symptoms of dermatofibrosarcoma protuberans include, but are not limited to small, slightly-raised, red or purple patch of skin 1 to 5 centimeters wide that can become a raised nodule and in some cases may cause redness, open up or bleed.

Symptoms of sebaceous carcinoma include, but are not limited to slow-growing sometimes yellow painless lump at an eyelid. The bump may bleed or ooze and may also have a thickening or yellow or reddish crust, where the eyelid meets the lash

The present disclosure provides methods of treating a subject having skin cancer, the methods comprising administering a CPVL inhibitor to the subject.

The present disclosure also provides methods of treating a subject having non-melanoma skin cancer, the methods comprising administering a CPVL inhibitor to the subject.

The present disclosure also provides methods of treating a subject having basal cell carcinoma, the methods comprising administering a CPVL inhibitor to the subject.

The present disclosure also provides methods of treating a subject having squamous cell carcinoma, the methods comprising administering a CPVL inhibitor to the subject.

The present disclosure also provides methods of treating a subject having melanoma, the methods comprising administering a CPVL inhibitor to the subject.

The present disclosure also provides methods of treating a subject Merkel cell carcinoma, the methods comprising administering a CPVL inhibitor to the subject.

The present disclosure also provides methods of treating a subject having dermatofibrosarcoma protuberans, the methods comprising administering a CPVL inhibitor to the subject.

The present disclosure also provides methods of treating a subject having sebaceous carcinoma, the methods comprising administering a CPVL inhibitor to the subject.

The present disclosure also provides methods of preventing a subject from developing skin cancer, the methods comprising administering a CPVL inhibitor to the subject.

The present disclosure also provides methods of preventing a subject from developing non-melanoma skin cancer, the methods comprising administering a CPVL inhibitor to the subject.

The present disclosure also provides methods of preventing a subject from developing basal cell carcinoma, the methods comprising administering a CPVL inhibitor to the subject.

The present disclosure also provides methods of preventing a subject from developing squamous cell carcinoma, the methods comprising administering a CPVL to the subject.

The present disclosure also provides methods of preventing a subject from developing melanoma, the methods comprising administering a CPVL inhibitor to the subject.

The present disclosure also provides methods of preventing a subject from developing Merkel cell carcinoma, the methods comprising administering a CPVL inhibitor to the subject.

The present disclosure also provides methods of preventing a subject from developing dermatofibrosarcoma protuberans, the methods comprising administering a CPVL inhibitor to the subject.

The present disclosure also provides methods of preventing a subject from developing sebaceous carcinoma, the methods comprising administering a CPVL inhibitor to the subject.

In some embodiments, the CPVL inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of a CPVL nucleic acid molecule. In some embodiments, the antisense RNA, siRNA, or shRNA hybridizes to a sequence within a CPVL genomic nucleic acid molecule or mRNA molecule and decreases expression of the CPVL polypeptide in a cell in the subject. In some embodiments, the CPVL inhibitor comprises an antisense molecule that hybridizes to a CPVL genomic nucleic acid molecule or mRNA molecule and decreases expression of the CPVL polypeptide in a cell in the subject. In some embodiments, the CPVL inhibitor comprises an siRNA that hybridizes to a CPVL genomic nucleic acid molecule or mRNA molecule and decreases expression of the CPVL polypeptide in a cell in the subject. In some embodiments, the CPVL inhibitor comprises an shRNA that hybridizes to a CPVL genomic nucleic acid molecule or mRNA molecule and decreases expression of the CPVL polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6XHis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (l), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S-or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂), OCH₃, —O(CH₂), NH₂, —O(CH₂), CH₃, —O(CH₂)ₙ—ONH₂, and —O(CH₂), ON[(CH₂), CH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo-and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense: mN*mN*/i2FN/mN/i2FN/mN/i2FN/mN/i2FN/mN/i2FN/mN/i2FN/mN/i2FN/mN/i2FN/*mN*/32FN/

### Antisense: /52FN/*/i2FN/*mN/i2FN/mN/i2FN/mN/i2FN/mN/i2FN/mN/i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly (lactic acid) (PLA) microspheres, poly (D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the CPVL inhibitor comprises a nuclease agent that induces one or more nicks or double-strand breaks at a recognition sequence(s) or a DNA-binding protein that binds to a recognition sequence within a CPVL genomic nucleic acid molecule. The recognition sequence can be located within a coding region of the CPVL gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the CPVL gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify a CPVL genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of CPVL nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in a CPVL genomic nucleic acid molecule or it can be a nickase that creates a single-strand break in a CPVL genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3,Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of CPVL genomic nucleic acid molecules can be generated by contacting a cell with a Cas protein and one or more gRNAs that hybridize to one or more gRNA recognition sequences within a target genomic locus in the CPVL genomic nucleic acid molecule. For example, a gRNA recognition sequence can be located within a region of SEQ ID NO:1. The gRNA recognition sequence can include or be proximate to the start codon of a CPVL genomic nucleic acid molecule or the stop codon of a CPVL genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in a CPVL genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where Nis any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within a CPVL genomic nucleic acid molecule. An exemplary gRNA is a gRNA effective to direct a Cas enzyme to bind to or cleave a CPVL genomic nucleic acid molecule, wherein the gRNA comprises a DNA-targeting segment that hybridizes to a gRNA recognition sequence within the CPVL genomic nucleic acid molecule. Exemplary gRNAs comprise a DNA-targeting segment that hybridizes to a gRNA recognition sequence present within a CPVL genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5,from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the human CPVL reference gene are set forth in Table 1 as SEQ ID NOs: 57-76.

TABLE 1
Guide RNA Recognition Sequences
Near CPVL Variation(s)

| Strand | gRNA Recognition Sequence | SEQ ID NO: |
|---|---|---|
| − | GTAAAGCATGGAGAGCGTTGTGG | 57 |
| − | CTCATTGACTGCATATCCGTGGG | 58 |
| − | CTGTAGCCAGAGAACTACTGGGG | 59 |
| + | CCACGGATATGCAGTCAATGAGG | 60 |
| + | TCAATGAGGACGATGTAGCACGG | 61 |
| + | TCACCCCTTACATTGAAGCTGGG | 62 |
| − | GCATAGCCCCCTATAATCTGAGG | 63 |
| + | AGTGTTTCCATGCCACCTAAGGG | 64 |
| − | CTTCCCAGCTTCAATGTAAGGGG | 65 |
| + | TTGGACTCTTTGTGGAACATGGG | 66 |
| + | ATGCCACCTAAGGGAGACTCAGG | 67 |
| + | GTCCTTCTCAGTCTTATGCAGGG | 68 |
| − | CCAACAAGCCAATTTGGTACAGG | 69 |
| + | GAGGTGAAGATCAACCTGAACGG | 70 |
| − | TTCCACAAAGAGTCCAAACATGG | 71 |
| − | TGTAAGTCTTATTCACGGTGAGG | 72 |
| − | TGTCCTGAGTCTCCCTTAGGTGG | 73 |
| + | CAATGAGGACGATGTAGCACGGG | 74 |
| + | ATACTGGATAAACTACTAGATGG | 75 |
| − | TGCATTCATGGCACTGCTTCTGG | 76 |

The Cas protein and the gRNA form a complex, and the Cas protein cleaves the target CPVL genomic nucleic acid molecule. The Cas protein can cleave the nucleic acid molecule at a site within or outside of the nucleic acid sequence present in the target CPVL genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the CPVL genomic nucleic acid molecule to which a DNA-targeting segment of a gRNA will bind.

Such methods can result, for example, in a CPVL genomic nucleic acid molecule in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the CPVL genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the methods of treatment and/or prevention further comprise detecting the presence or absence of a CPVL missense variant nucleic acid molecule encoding a CPVL predicted loss-of-function polypeptide in a biological sample from the subject. As used throughout the present disclosure, a “CPVL missense variant nucleic acid molecule encoding a CPVL predicted loss-of-function polypeptide” is any CPVL nucleic acid molecule (such as, for example, genomic nucleic acid molecule, mRNA molecule, or cDNA molecule) encoding a CPVL polypeptide having a partial loss-of-function, a complete loss-of-function, a predicted partial loss-of-function, or a predicted complete loss-of-function.

The present disclosure also provides methods of treating a subject with a therapeutic agent that treats or inhibits skin cancer, wherein the subject has skin cancer. The present disclosure also provides methods of preventing a subject from developing skin cancer by administering a therapeutic agent that prevents skin cancer. In some embodiments, the methods comprise determining whether the subject has a CPVL missense variant nucleic acid molecule encoding a CPVL predicted loss-of-function polypeptide by obtaining or having obtained a biological sample from the subject, and performing or having performed a sequence analysis on the biological sample to determine if the subject has a genotype comprising the CPVL missense variant nucleic acid molecule encoding a CPVL predicted loss-of-function polypeptide. In some embodiments, the methods further comprise administering or continuing to administer the therapeutic agent that treats, prevents, or inhibits skin cancer in a standard dosage amount to a subject that is CPVL reference, and/or administering a CPVL inhibitor to the subject. In some embodiments, the methods further comprise administering or continuing to administer the therapeutic agent that treats, prevents, or inhibits skin cancer in an amount that is the same as or less than a standard dosage amount to a subject that is heterozygous for the CPVL missense variant nucleic acid molecule, and/or administering a CPVL inhibitor to the subject. In some embodiments, the methods further comprise administering or continuing to administer the therapeutic agent that treats, prevents, or inhibits skin cancer in an amount that is the same as or less than a standard dosage amount to a subject that is homozygous for the CPVL missense variant nucleic acid molecule. The presence of a genotype having the CPVL missense variant nucleic acid molecule encoding the CPVL predicted loss-of-function polypeptide indicates the subject has a decreased risk of developing skin cancer. In some embodiments, the subject is CPVL reference. In some embodiments, the subject is heterozygous for a CPVL missense variant nucleic acid molecule encoding a CPVL predicted loss-of-function polypeptide.

For subjects that are genotyped or determined to be either CPVL reference or heterozygous for a CPVL missense variant nucleic acid molecule encoding a CPVL predicted loss-of-function polypeptide, such subjects can be administered a CPVL inhibitor, as described herein.

Detecting the presence or absence of a CPVL missense variant nucleic acid molecule encoding a CPVL predicted loss-of-function polypeptide in a biological sample from a subject and/or determining whether a subject has a CPVL missense variant nucleic acid molecule encoding a CPVL predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is CPVL reference, the subject is administered a therapeutic agent that treats, prevents, or inhibits skin cancer in a standard dosage amount. In some embodiments, when the subject is heterozygous for a CPVL missense variant nucleic acid molecule encoding a CPVL predicted loss-of-function polypeptide, the subject is administered a therapeutic agent that treats, prevents, or inhibits skin cancer in a dosage amount that is the same as or less than a standard dosage amount.

In some embodiments, the treatment and/or prevention methods further comprise detecting the presence or absence of a CPVL predicted loss-of-function polypeptide in a biological sample from the subject. In some embodiments, when the subject does not have a CPVL predicted loss-of-function polypeptide, the subject is also administered a therapeutic agent that treats, prevents, or inhibits skin cancer in a standard dosage amount. In some embodiments, when the subject has a CPVL predicted loss-of-function polypeptide, the subject is also administered a therapeutic agent that treats, prevents, or inhibits skin cancer in a dosage amount that is the same as or less than a standard dosage amount.

The present disclosure also provides methods of treating a subject with a therapeutic agent that treats or inhibits skin cancer, wherein the subject has skin cancer. In some embodiments, the method comprises determining whether the subject has a CPVL predicted loss-of-function polypeptide by obtaining or having obtained a biological sample from the subject, and performing or having performed an assay on the biological sample to determine if the subject has a CPVL predicted loss-of-function polypeptide. When the subject does not have a CPVL predicted loss-of-function polypeptide, the therapeutic agent that treats or inhibits skin cancer is administered or continued to be administered to the subject in a standard dosage amount, and/or a CPVL inhibitor is administered to the subject. When the subject has a CPVL predicted loss-of-function polypeptide, the therapeutic agent that treats or inhibits skin cancer is administered or continued to be administered to the subject in an amount that is the same as or less than a standard dosage amount, and/or a CPVL inhibitor is administered to the subject. The presence of a CPVL predicted loss-of-function polypeptide indicates the subject has a decreased risk of developing skin cancer. In some embodiments, the subject has a CPVL predicted loss-of-function polypeptide. In some embodiments, the subject does not have a CPVL predicted loss-of-function polypeptide.

The present disclosure also provides methods of preventing a subject from developing skin cancer by administering a therapeutic agent that prevents skin cancer. In some embodiments, the method comprises determining whether the subject has a CPVL predicted loss-of-function polypeptide by obtaining or having obtained a biological sample from the subject, and performing or having performed an assay on the biological sample to determine if the subject has a CPVL predicted loss-of-function polypeptide. When the subject does not have a CPVL predicted loss-of-function polypeptide, the therapeutic agent that prevents skin cancer is administered or continued to be administered to the subject in a standard dosage amount, and/or a CPVL inhibitor is administered to the subject. When the subject has a CPVL predicted loss-of-function polypeptide, the therapeutic agent that prevents skin cancer is administered or continued to be administered to the subject in an amount that is the same as or less than a standard dosage amount, and/or a CPVL inhibitor is administered to the subject. The presence of a CPVL predicted loss-of-function polypeptide indicates the subject has a decreased risk of developing skin cancer. In some embodiments, the subject has a CPVL predicted loss-of-function polypeptide. In some embodiments, the subject does not have a CPVL predicted loss-of-function polypeptide.

Detecting the presence or absence of a CPVL predicted loss-of-function polypeptide in a biological sample from a subject and/or determining whether a subject has a CPVL predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the CPVL inhibitor is a small molecule. In some embodiments, the CPVL inhibitor is hydroxymethyl (N-methyliminodiacetic acid) boronate (hydroxymethyl (MIDA) boronate), azidomethyl (N-methyliminodiacetic acid) boronate (azidomethyl (MIDA) boronate), or an a-functionalized alkyl (MIDA) boronate compound (see, Table 1 of Adachi et al., Chem. Commun., 2015, 51, 3608-3611. Additional inhibitors include, but are not limited to, the following compounds:

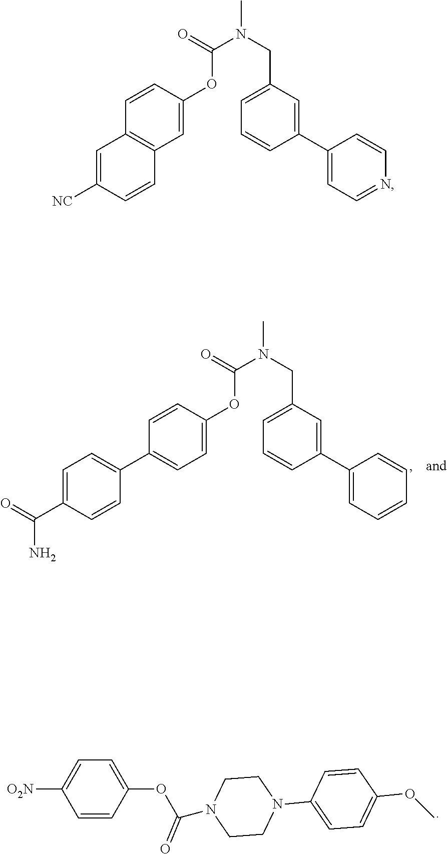 [SMILES: CN(Cc1cccc(-c2ccncc2)c1)C(=O)Oc1ccc2cc(C#N)ccc2c1 ; CN(Cc1cccc(-c2ccccc2)c1)C(=O)Oc1ccc(-c2ccc(C(N)=O)cc2)cc1 ; COc1ccc(N2CCN(C(=O)Oc3ccc([N+](=O)[O-])cc3)CC2)cc1]

In some embodiments, the CPVL inhibitor is an immune-oncology agent or an immune checkpoint inhibitor. In some embodiments, the immune checkpoint inhibitor is an anti-PD-1 agent, an anti-PD-L1 agent, or an anti-CTLA-4 agent. In some embodiments, the immune checkpoint inhibitor is an anti-PD-1 agent, such as for example, KEYTRUDA® (pembrolizumab), OPDIVO® (nivolumab), and LIBTAYO® (cemiplimab). In some embodiments, the immune checkpoint inhibitor is KEYTRUDA® (pembrolizumab). In some embodiments, the immune checkpoint inhibitor is pembrolizumab. In some embodiments, the immune checkpoint inhibitor is OPDIVO® (nivolumab). In some embodiments, the immune checkpoint inhibitor is nivolumab. In some embodiments, the immune checkpoint inhibitor is LIBTAYO® (cemiplimab). In some embodiments, the immune checkpoint inhibitor is cemiplimab. In some embodiments, the immune checkpoint inhibitor is an anti-PD-L1 agent, such as for example, TECENTRIQ® (atezolizumab), BAVENCIO® (avelumab), and IMFINZI® (durvalumab). In some embodiments, the immune checkpoint inhibitor is TECENTRIQ® (atezolizumab). In some embodiments, the immune checkpoint inhibitor is atezolizumab. In some embodiments, the immune checkpoint inhibitor is BAVENCIO® (avelumab). In some embodiments, the immune checkpoint inhibitor is avelumab. In some embodiments, the immune checkpoint inhibitor is IMFINZI® (durvalumab). In some embodiments, the immune checkpoint inhibitor is durvalumab. In some embodiments, the immune checkpoint inhibitor is an anti-CTLA-4agent, such as for example, YERVOY® (ipilimumab) and tremelimumab. In some embodiments, the immune checkpoint inhibitor is YERVOY® (ipilimumab) or tremelimumab. In some embodiments, the immune checkpoint inhibitor is YERVOY® (ipilimumab). In some embodiments, the immune checkpoint inhibitor is ipilimumab. In some embodiments, the immune checkpoint inhibitor is tremelimumab. In some embodiments, the CPVL inhibitor is a combination of any of the CPVL inhibitors described herein and any of the immune checkpoint inhibitors described herein. In some embodiments, the CPVL inhibitor is a combination of any of the CPVL inhibitors described herein and an anti-PD-1 agent and an anti-CTLA-4 agent.

Examples of therapeutic agents that treat or inhibit basal cell carcinoma include, but are not limited to, imiquimod, fluorouracil, cemiplimab-rwlc, sonidegib, and vismodegib, or any combination thereof. In some embodiments, the therapeutic agent is imiquimod. In some embodiments, the therapeutic agent is fluorouracil. In some embodiments, the therapeutic agent is cemiplimab-rwlc. In some embodiments, the therapeutic agent is sonidegib. In some embodiments, the therapeutic agent is vismodegib.

Examples of therapeutic agents that treat or inhibit squamous cell carcinoma include, but are not limited to, cemiplimab-rwlc and pembrolizumab, or a combination thereof. In some embodiments, the therapeutic agent is cemiplimab-rwlc. In some embodiments, the therapeutic agent is pembrolizumab.

Examples of therapeutic agents that treat or inhibit melanoma include, but are not limited to, aldesleukin, cobimetinib, dabrafenib, dacarbazine, recombinant interferon alfa-2b, ipilimumab, nivolumab, nivolumab, peginterferon alfa-2b, pembrolizumab, talimogene laherparepvec, trametinib dimethyl sulfoxide, and vemurafenib, or any combination thereof. In some embodiments, the therapeutic agent is aldesleukin. In some embodiments, the therapeutic agent is cobimetinib. In some embodiments, the therapeutic agent is dabrafenib. In some embodiments, the therapeutic agent is dacarbazine. In some embodiments, the therapeutic agent is recombinant interferon alfa-2b. In some embodiments, the therapeutic agent is ipilimumab. In some embodiments, the therapeutic agent is nivolumab. In some embodiments, the therapeutic agent is nivolumab. In some embodiments, the therapeutic agent is peginterferon alfa-2b. In some embodiments, the therapeutic agent is pembrolizumab. In some embodiments, the therapeutic agent is talimogene laherparepvec. In some embodiments, the therapeutic agent is trametinib dimethyl sulfoxide. In some embodiments, the therapeutic agent is vemurafenib.

Examples of therapeutic agents that treat or inhibit Merkel cell carcinoma include, but are not limited to, avelumab, pembrolizumab, etoposide (VP16), and carboplatin combination regimen, or any combination thereof. In some embodiments, the therapeutic agent is avelumab. In some embodiments, the therapeutic agent is pembrolizumab. In some embodiments, the therapeutic agent is etoposide (VP16). In some embodiments, the therapeutic agent is carboplatin combination regimen.

Examples of therapeutic agents that treat or inhibit dermatofibrosarcoma protuberans include, but are not limited to, imatinib.

In some embodiments, the dose of the therapeutic agents that treat, prevent, or inhibit skin cancer can be decreased by about 10%, by about 20%, by about 30%, by about 40%, by about 50%, by about 60%, by about 70%, by about 80%, or by about 90% for subjects that are heterozygous for a CPVL missense variant nucleic acid molecule encoding a CPVL predicted loss-of-function polypeptide (i.e., a less than the standard dosage amount) compared to subjects that are CPVL reference (who may receive a standard dosage amount). In some embodiments, the dose of the therapeutic agents that treat, prevent, or inhibit skin cancer can be decreased by about 10%, by about 20%, by about 30%, by about 40%, or by about 50%. In addition, the subjects that are heterozygous for a CPVL missense variant nucleic acid molecule encoding a CPVL predicted loss-of-function polypeptide can be administered less frequently compared to subjects that are CPVL reference.

In some embodiments, the dose of the therapeutic agents that treat, prevent, or inhibit skin cancer can be decreased by about 10%, by about 20%, by about 30%, by about 40%, by about 50%, for subjects that are homozygous for a CPVL missense variant nucleic acid molecule encoding a CPVL predicted loss-of-function polypeptide compared to subjects that are heterozygous for a CPVL missense variant nucleic acid molecule encoding a CPVL predicted loss-of-function polypeptide. In some embodiments, the dose of the therapeutic agents that treat, prevent, or inhibit skin cancer can be decreased by about 10%, by about 20%, by about 30%, by about 40%, or by about 50%. In addition, the dose of therapeutic agents that treat, prevent, or inhibit skin cancer in subjects that are homozygous for a CPVL missense variant nucleic acid molecule encoding a CPVL predicted loss-of-function polypeptide can be administered less frequently compared to subjects that are heterozygous for a CPVL missense variant nucleic acid molecule encoding a CPVL predicted loss-of-function polypeptide.

Administration of the therapeutic agents that treat, prevent, or inhibit skin cancer and/or CPVL inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the therapeutic agents that treat, prevent, or inhibit skin cancer and/or CPVL inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in skin cancer, a decrease/reduction in the severity of skin cancer (such as, for example, a reduction or inhibition of development of skin cancer), a decrease/reduction in symptoms and skin cancer-related effects, delaying the onset of symptoms and skin cancer-related effects, reducing the severity of symptoms of skin cancer-related effects, reducing the number of symptoms and skin cancer-related effects, reducing the latency of symptoms and skin cancer-related effects, an amelioration of symptoms and skin cancer-related effects, reducing secondary symptoms, reducing secondary infections, preventing relapse to skin cancer, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of skin cancer development/progression (such as, for example, a complete or partial avoidance/inhibition or a delay), and an increased survival time of the affected host animal, following administration of a therapeutic protocol. Treatment of skin cancer encompasses the treatment of a subject already diagnosed as having any form of skin cancer at any clinical stage or manifestation, the delay of the onset or evolution or aggravation or deterioration of the symptoms or signs of skin cancer, and/or preventing and/or reducing the severity of skin cancer.

The present disclosure also provides methods of identifying a subject having an increased risk of developing skin cancer. In some embodiments, the method comprises determining or having determined in a biological sample obtained from the subject the presence or absence of a CPVL missense variant nucleic acid molecule (such as a genomic nucleic acid molecule, mRNA molecule, and/or cDNA molecule) encoding a CPVL predicted loss-of-function polypeptide encoding a CPVL polypeptide. When the subject lacks a CPVL missense variant nucleic acid molecule encoding a CPVL predicted loss-of-function polypeptide (i.e., the subject is genotypically categorized as a CPVL reference), then the subject has an increased risk of developing skin cancer. When the subject has a CPVL missense variant nucleic acid molecule encoding a CPVL predicted loss-of-function polypeptide (i.e., the subject is heterozygous or homozygous for a CPVL missense variant nucleic acid molecule encoding a CPVL predicted loss-of-function polypeptide), then the subject has a decreased risk of developing skin cancer.

Having a single copy of a CPVL missense variant nucleic acid molecule encoding a CPVL predicted loss-of-function polypeptide is more protective of a subject from developing skin cancer than having no copies of a CPVL missense variant nucleic acid molecule encoding a CPVL predicted loss-of-function polypeptide. Without intending to be limited to any particular theory or mechanism of action, it is believed that a single copy of a CPVL missense variant nucleic acid molecule encoding a CPVL predicted loss-of-function polypeptide (i.e., heterozygous for a CPVL missense variant nucleic acid molecule encoding a CPVL predicted loss-of-function polypeptide) is protective of a subject from developing skin cancer, and it is also believed that having two copies of a CPVL missense variant nucleic acid molecule encoding a CPVL predicted loss-of-function polypeptide (i.e., homozygous for a CPVL missense variant nucleic acid molecule encoding a CPVL predicted loss-of-function polypeptide) may be more protective of a subject from developing skin cancer, relative to a subject with a single copy. Thus, in some embodiments, a single copy of a CPVL missense variant nucleic acid molecule encoding a CPVL predicted loss-of-function polypeptide may not be completely protective, but instead, may be partially or incompletely protective of a subject from developing skin cancer. While not desiring to be bound by any particular theory, there may be additional factors or molecules involved in the development of skin cancer that are still present in a subject having a single copy of a CPVL missense variant nucleic acid molecule encoding a CPVL predicted loss-of-function polypeptide, thus resulting in less than complete protection from the development of skin cancer.

Determining whether a subject has a CPVL missense variant nucleic acid molecule encoding a CPVL predicted loss-of-function polypeptide in a biological sample from a subject and/or determining whether a subject has a CPVL missense variant nucleic acid molecule encoding a CPVL predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing skin cancer, the subject is administered a therapeutic agent that treats, prevents, or inhibits skin cancer, and/or a CPVL inhibitor, as described herein. For example, when the subject is CPVL reference, and therefore has an increased risk of developing skin cancer, the subject is administered a CPVL inhibitor. In some embodiments, such a subject is also administered a therapeutic agent that treats, prevents, or inhibits skin cancer. In some embodiments, when the subject is heterozygous for a CPVL missense variant nucleic acid molecule encoding a CPVL predicted loss-of-function polypeptide, the subject is administered the therapeutic agent that treats, prevents, or inhibits skin cancer in a dosage amount that is the same as or less than a standard dosage amount, and/or is administered a CPVL inhibitor. In some embodiments, such a subject is also administered a therapeutic agent that treats, prevents, or inhibits skin cancer. In some embodiments, when the subject is homozygous for a CPVL missense variant nucleic acid molecule encoding a CPVL predicted loss-of-function polypeptide, the subject is administered the therapeutic agent that treats, prevents, or inhibits skin cancer in a dosage amount that is the same as or less than a standard dosage amount. In some embodiments, the subject is CPVL reference. In some embodiments, the subject is heterozygous for a CPVL missense variant nucleic acid molecule encoding a CPVL predicted loss-of-function polypeptide. In some embodiments, the subject is homozygous for a CPVL missense variant nucleic acid molecule encoding a CPVL predicted loss-of-function polypeptide.

In some embodiments, any of the methods described herein can further comprise determining the subject's aggregate burden of having a CPVL missense variant nucleic acid molecule encoding a CPVL predicted loss-of-function polypeptide, and/or a CPVL predicted loss-of-function variant polypeptide associated with a decreased risk of developing skin cancer. The aggregate burden is the sum of all variants in the CPVL gene, which can be carried out in an association analysis with skin cancer. In some embodiments, the subject is homozygous for one or more CPVL missense variant nucleic acid molecules encoding a CPVL predicted loss-of-function polypeptide associated with a decreased risk of developing skin cancer. In some embodiments, the subject is heterozygous for one or more CPVL missense variant nucleic acid molecules encoding a CPVL predicted loss-of-function polypeptide associated with a decreased risk of developing skin cancer. The result of the association analysis suggests that CPVL missense variant nucleic acid molecules encoding a CPVL predicted loss-of-function polypeptide are associated with decreased risk of developing skin cancer. When the subject has a lower aggregate burden, the subject is at a higher risk of developing skin cancer and the subject is administered or continued to be administered the therapeutic agent that treats, prevents, or inhibits skin cancer in a standard dosage amount. When the subject has a greater aggregate burden, the subject is at a lower risk of developing skin cancer and the subject is administered or continued to be administered the therapeutic agent that treats, prevents, or inhibits skin cancer in an amount that is the same as or less than the standard dosage amount. The greater the aggregate burden, the lower the risk of developing skin cancer. CPVL variants that can be used in the aggregate burden analysis include any one or more, or any combination, of the following in Table 2:

TABLE 2

| Variant | rsID |
|---|---|
| 7:29096102:C:T | |
| 7:28995873:G:A | |
| 7:29030645:C:CT | |
| 7:29096125:A:T | |
| 7:29066109:GT:G | |
| 7:29064221:T:TG | |
| 7:29120891:A:C | |
| 7:29071905:C:CT | |
| 7:29071905:C:G | |
| 7:29064060:C:T | |
| 7:29064167:AC:A | rs777604046 |
| 7:29086513:T:A | rs764980288 |
| 7:29064132:C:CA | rs748209844 |
| 7:29066026:G:T | |
| 7:28995813:G:A | |
| 7:29092703:C:T | rs201027082 |
| 7:29195076:C:T | |
| 7:29120892:C:A | |
| 7:29066070:G:A | |
| 7:29064236:T:C | |
| 7:29095143:C:T | |
| 7:29095143:CT:C | |
| 7:29071879:AG:A | |
| 7:29064077:AT:A | |
| 7:29064100:TG:T | |
| 7:29030600:GGA:G | |
| 7:29066069:TG:T | |
| 7:29064120:G:A | |
| 7:28995858:TA:T | |
| 7:29096218:C:T | |
| 7:29120903:CT:C | |
| 7:29112720:C:T | |
| 7:29195076:C:A | |
| 7:29030581:TGGAA:T | |
| 7:29096219:T:C | |
| 7:29096142:CAA:C | |
| 7:29071905:C:T | |
| 7:29121003:CAG:C | |
| 7:29096102:C:G | |
| 7:29064120:G:GC | rs774891146 |
| 7:29071772:C:A | rs201079331 |
| 7:29120995:GC:G | |
| 7:29066060:G:GT | |
| 7:29030645:CT:C | |
| 7:29064060:C:A | |
| 7:29066032:CA:C | |
| 7:29112740:G:T | |
| 7:29120989:G:GA | |
| 7:29112702:A:G | rs745744964 |
| 7:29030604:GT:G | |
| 7:29064150:TAA:T | |
| 7:29064087:A:AG | |
| 7:29064093:GC:G | |
| 7:29112740:G:C | |
| 7:29064060:CCTTAT:C | rs749642830 |
| 7:28995830:GCT:G | |
| 7:29064221:TG:T | |
| 7:29086483:C:T | |
| 7:29030760:C:CTGAAA | |
| 7:29096207:T:TCTGG | |
| 7:29066114:TC:T | |
| 7:29072425:T:A | |
| 7:29112817:C:A | rs770230527 |
| 7:29064115:TTC:T | |
| 7:29086488:C:T | |
| 7:29095087:A:T | |
| 7:28995863:CIT | |
| 7:29092645:C:A | |
| 7:29096130:G:A | |
| 7:29112766:C:T | rs368319631 |
| 7:29112757:T:C | rs776115114 |
| 7:29096177:C:T | |
| 7:28995863:C:A | |
| 7:29066115:C:A | |
| 7:29030726:C:T | rs776719397 |
| 7:29030584:A:T | |
| 7:29072407:T:C | rs200576601 |
| 7:29064135:C:G | |
| 7:29030749:T:C | rs375729583 |
| 7:29095089:T:G | |
| 7:29030710:G:C | |
| 7:28995876:T:A | |
| 7:29066063:A:T | rs770831396 |
| 7:29064068:T:A | |
| 7:29112774:C:T | |
| 7:29071894:C:T | |
| 7:29096198:G:A | |
| 7:29112810:C:T | rs138216401 |
| 7:29120923:G:C | |
| 7:29086539:T:C | |
| 7:29072410:T:C | |
| 7:29120922:G:C | rs774751381 |
| 7:28995851:G:C | |
| 7:29030642:A:T | |
| 7:29096199:G:C | |
| 7:29096169:C:T | |
| 7:29030738:G:C | |
| 7:29064134:A:G | |
| 7:29064173:A:G | |
| 7:28995809:A:C | rs757457600 |
| 7:29096199:G:A | rs188939784 |
| 7:29096150:C:A | |
| 7:29030599:CIT | rs760718602 |
| 7:29072321:C:T | rs778818106 |
| 7:29030737:A:C | |
| 7:29030578:T:C | |
| 7:29096172:G:A | rs752979580 |
| 7:29092663:A:G | rs117744081 |
| 7:29030682:C:A | |
| 7:29095097:T:C | |
| 7:29086497:T:G | |
| 7:29092632:T:A | |
| 7:29030611:G:A | |
| 7:28995872:C:T | rs771602383 |
| 7:28995816:T:G | |
| 7:29092666:C:T | rs751543732 |
| 7:29066097:C:T | |
| 7:28995859:A:T | |
| 7:29086486:C:G | rs376816938 |
| 7:29086549:C:T | |
| 7:29072416:G:A | |
| 7:29096136:G:C | |
| 7:29095103:A:G | |
| 7:29092638:G:A | |
| 7:29092660:C:T | |
| 7:29066058:C:G | |
| 7:29072329:A:G | |
| 7:29030727:G:C | |
| 7:29095088:T:A | |
| 7:29064125:T:C | |
| 7:29086515:T:C | |
| 7:29064122:A:G | |
| 7:29064090:A:G | |
| 7:29096171:G:A | |
| 7:29086548:G:T | |
| 7:28995873:G:C | |
| 7:29066117:A:G | |
| 7:29064101:G:A | |
| 7:29030740:T:C | rs763825813 |
| 7:29095089:T:C | |
| 7:29064164:C:T | |
| 7:29071803:G:C | |
| 7:29064168:C:T | rs761706669 |
| 7:28995864:C:T | |
| 7:29096144:A:G | |
| 7:29096172:G:T | |
| 7:29071780:G:C | |
| 7:29112725:G:T | |
| 7:29092680:G:A | |
| 7:29066071:G:C | |
| 7:29096184:G:C | |
| 7:29096112:T:A | |
| 7:29072423:A:G | |
| 7:29030722:G:A | |
| 7:29064098:A:C | |
| 7:28995852:G:A | |
| 7:28995846:C:T | |
| 7:29030609:C:A | |
| 7:29112754:C:T | rs200681795 |
| 7:29092683:A:C | |
| 7:29112732:T:C | rs769831040 |
| 7:29096139:C:T | |
| 7:29096136:G:A | |
| 7:29112719:C:A | |
| 7:29072347:T:G | |
| 7:29086548:G:A | rs756688128 |
| 7:29064092:G:T | |
| 7:29112750:T:C | rs771212555 |
| 7:29112735:C:G | |
| 7:29096121:C:A | |
| 7:28995814:A:T | |
| 7:29066108:A:G | |
| 7:29092693:C:T | |
| 7:29096169:C:G | |
| 7:29095101:G:A | |
| 7:29064212:T:C | rs778672021 |
| 7:29120929:G:A | rs771997351 |
| 7:29030705:G:A | rs773880380 |
| 7:29095086:G:T | rs540749696 |
| 7:28995812:C:T | rs147771477 |
| 7:29096143:A:C | rs147286046 |
| 7:29030577:C:G | rs768929266 |
| 7:28995822:T:C | |
| 7:29096174:C:G | |
| 7:29072380:T:C | |
| 7:29066105:A:G | |
| 7:29064213:A:T | |
| 7:29096138:T:C | |
| 7:29064174:T:C | |
| 7:29086498:A:G | rs147774594 |
| 7:29064152:A:G | rs775477781 |
| 7:29096159:G:A | |
| 7:29096160:A:G | |
| 7:29030750:A:G | |
| 7:29071781:C:T | |
| 7:29072339:C:T | rs775870649 |
| 7:29030600:G:A | |
| 7:29096120:A:G | |

In some embodiments, the subject's aggregate burden of having any one or more CPVL missense variant nucleic acid molecules encoding a CPVL predicted loss-of-function polypeptide represents a weighted sum of a plurality of any of the CPVL missense variant nucleic acid molecules encoding a CPVL predicted loss-of-function polypeptide. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the CPVL gene where the genetic burden is the number of alleles multiplied by the association estimate with skin cancer or related outcome for each allele (e.g., a weighted polygenic burden score). This can include any genetic variants, regardless of their genomic annotation, in proximity to the CPVL gene (up to 10 Mb around the gene) that show a non-zero association with skin cancer-related traits in a genetic association analysis. In some embodiments, when the subject has an aggregate burden above a desired threshold score, the subject has a decreased risk of developing skin cancer. In some embodiments, when the subject has an aggregate burden below a desired threshold score, the subject has an increased risk of developing skin cancer.

In some embodiments, the aggregate burden may be divided into quintiles, e.g., top quintile, intermediate quintile, and bottom quintile, wherein the top quintile of aggregate burden corresponds to the lowest risk group and the bottom quintile of aggregate burden corresponds to the highest risk group. In some embodiments, a subject having a greater aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with skin cancer in the top 10%, top 20%, top 30%, top 40%, or top 50% of p-value range for the association. In some embodiments, each of the identified genetic variants comprise the genetic variants having association with skin cancer with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with skin cancer with p-value of less than 5×10⁻⁸. In some embodiments, the identified genetic variants comprise genetic variants having association with skin cancer in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having aggregate burdens in the bottom decile, quintile, or tertile in a reference population. The threshold of the aggregate burden is determined on the basis of the nature of the intended practical application and the risk difference that would be considered meaningful for that practical application.

In some embodiments, when a subject is identified as having an increased risk of developing skin cancer, the subject is further administered a therapeutic agent that treats, prevents, or inhibits skin cancer, and/or a CPVL inhibitor, as described herein. For example, when the subject is CPVL reference, and therefore has an increased risk of developing skin cancer, the subject is administered a CPVL inhibitor. In some embodiments, such a subject is also administered a therapeutic agent that treats, prevents, or inhibits skin cancer. In some embodiments, when the subject is heterozygous for a CPVL missense variant nucleic acid molecule encoding a CPVL predicted loss-of-function polypeptide, the subject is administered the therapeutic agent that treats, prevents, or inhibits skin cancer in a dosage amount that is the same as or less than a standard dosage amount, and/or is administered a CPVL inhibitor. In some embodiments, the subject is CPVL reference. In some embodiments, the subject is heterozygous for a CPVL missense variant nucleic acid molecule encoding a CPVL predicted loss-of-function polypeptide. Furthermore, when the subject has a lower aggregate burden for having a CPVL missense variant nucleic acid molecule encoding a CPVL predicted loss-of-function polypeptide, and therefore has an increased risk of developing skin cancer, the subject is administered a therapeutic agent that treats, prevents, or inhibits skin cancer. In some embodiments, when the subject has a lower aggregate burden for having a CPVL missense variant nucleic acid molecule encoding a CPVL predicted loss-of-function polypeptide, the subject is administered the therapeutic agent that treats, prevents, or inhibits skin cancer in a dosage amount that is the same as or greater than the standard dosage amount administered to a subject who has a greater aggregate burden for having a CPVL missense variant nucleic acid molecule encoding a CPVL predicted loss-of-function polypeptide.

The present disclosure also provides methods of detecting the presence or absence of a CPVL missense variant nucleic acid molecule (i.e., a genomic nucleic acid molecule, an mRNA molecule, or a cDNA molecule produced from an mRNA molecule) encoding a CPVL predicted loss-of-function polypeptide in a biological sample from a subject. It is understood that gene sequences within a population and mRNA molecules encoded by such genes can vary due to polymorphisms such as single-nucleotide polymorphisms. The sequences provided herein for the CPVL variant genomic nucleic acid molecule, CPVL variant mRNA molecule, and CPVL variant cDNA molecule are only exemplary sequences. Other sequences for the CPVL variant genomic nucleic acid molecule, variant mRNA molecule, and variant cDNA molecule are also possible.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any CPVL missense variant nucleic acid molecule encoding a CPVL predicted loss-of-function polypeptide, preliminary processing designed to isolate or enrich the biological sample for the genomic DNA can be employed. A variety of techniques may be used for this purpose. When detecting the level of any CPVL variant mRNA molecule, different techniques can be used enrich the biological sample with mRNA molecules. Various methods to detect the presence or level of an mRNA molecule or the presence of a particular variant genomic DNA locus can be used.

In some embodiments, detecting a CPVL missense variant nucleic acid molecule encoding a CPVL predicted loss-of-function polypeptide in a subject comprises performing a sequence analysis on a biological sample obtained from the subject to determine whether a CPVL genomic nucleic acid molecule in the biological sample, and/or a CPVL mRNA molecule in the biological sample, and/or a CPVL cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of a CPVL missense variant nucleic acid molecule encoding a CPVL predicted loss-of-function polypeptide (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising a CPVL genomic nucleic acid molecule or mRNA molecule, and if mRNA, optionally reverse transcribing the mRNA into cDNA. Such assays can comprise, for example determining the identity of these positions of the particular CPVL nucleic acid molecule. In some embodiments, the method is an in vitro method.

In some embodiments, the determining step, detecting step, or sequence analysis comprises sequencing at least a portion of the nucleotide sequence of the CPVL genomic nucleic acid molecule, the CPVL mRNA molecule, or the CPVL cDNA molecule in the biological sample, wherein the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only a CPVL genomic nucleic acid molecule is analyzed. In some embodiments, only a CPVL mRNA is analyzed. In some embodiments, only a CPVL cDNA obtained from CPVL mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to a CPVL variant genomic sequence, variant mRNA sequence, or variant cDNA sequence and not the corresponding CPVL reference sequence under stringent conditions, and determining whether hybridization has occurred.

In some embodiments, the determining step, detecting step, or sequence analysis comprises: a) amplifying at least a portion of the nucleic acid molecule that encodes the CPVL polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a CPVL variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2× SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ion, typically about 0.01 to 1.0 M Na⁺ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to CPVL missense variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to CPVL missense variant genomic nucleic acid molecules, CPVL missense variant mRNA molecules, and/or CPVL missense variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the CPVL variant missense genomic nucleic acid molecules, CPVL missense variant mRNA molecules, and/or CPVL missense variant cDNA molecules disclosed herein. The primers described herein can be used to amplify CPVL missense variant genomic nucleic acid molecules, CPVL missense variant mRNA molecules, or CPVL missense variant cDNA molecules, or a fragment thereof.

In the context of the disclosure “specifically hybridizes” means that the probe or primer (such as, for example, the alteration-specific probe or alteration-specific primer) does not hybridize to a nucleic acid sequence encoding a CPVL reference genomic nucleic acid molecule, a CPVL reference mRNA molecule, and/or a CPVL reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The nucleotide sequence of a CPVL reference genomic nucleic acid molecule is set forth in SEQ ID NO:1 (ENSG00000106066.15 encompassing chr7: 28,995,637-29,195,276 in the GRCh38/hg38 human genome assembly).

The nucleotide sequence of a CPVL reference mRNA molecule is set forth in SEQ ID NO: 2. The nucleotide sequence of another CPVL reference mRNA molecule is set forth in SEQ ID NO:3. The nucleotide sequence of another CPVL reference mRNA molecule is set forth in SEQ ID NO:4. The nucleotide sequence of another CPVL reference mRNA molecule is set forth in SEQ ID NO:5. The nucleotide sequence of another CPVL reference mRNA molecule is set forth in SEQ ID NO:6. The nucleotide sequence of another CPVL reference mRNA molecule is set forth in SEQ ID NO:7. The nucleotide sequence of another CPVL reference mRNA molecule is set forth in SEQ ID NO:8. The nucleotide sequence of another CPVL reference mRNA molecule is set forth in SEQ ID NO:9. The nucleotide sequence of another CPVL reference mRNA molecule is set forth in SEQ ID NO:10. The nucleotide sequence of another CPVL reference mRNA molecule is set forth in SEQ ID NO:11. The nucleotide sequence of another CPVL reference mRNA molecule is set forth in SEQ ID NO: 12. The nucleotide sequence of another CPVL reference mRNA molecule is set forth in SEQ ID NO:13. The nucleotide sequence of another CPVL reference mRNA molecule is set forth in SEQ ID NO:14. The nucleotide sequence of another CPVL reference mRNA molecule is set forth in SEQ ID NO:15. The nucleotide sequence of another CPVL reference mRNA molecule is set forth in SEQ ID NO:16. The nucleotide sequence of another CPVL reference mRNA molecule is set forth in SEQ ID NO:17. The nucleotide sequence of another CPVL reference mRNA molecule is set forth in SEQ ID NO:18. The nucleotide sequence of another CPVL reference mRNA molecule is set forth in SEQ ID NO:19. The nucleotide sequence of another CPVL reference mRNA molecule is set forth in SEQ ID NO:20. The nucleotide sequence of another CPVL reference mRNA molecule is set forth in SEQ ID NO:21. The nucleotide sequence of another CPVL reference mRNA molecule is set forth in SEQ ID NO: 22. The nucleotide sequence of another CPVL reference mRNA molecule is set forth in SEQ ID NO:23. The nucleotide sequence of another CPVL reference mRNA molecule is set forth in SEQ ID NO:24. The nucleotide sequence of another CPVL reference mRNA molecule is set forth in SEQ ID NO:25. The nucleotide sequence of another CPVL reference mRNA molecule is set forth in SEQ ID NO:26.

The nucleotide sequence of a CPVL reference cDNA molecule is set forth in SEQ ID NO: 27. The nucleotide sequence of another CPVL reference cDNA molecule is set forth in SEQ ID NO:28. The nucleotide sequence of another CPVL reference cDNA molecule is set forth in SEQ ID NO:29. The nucleotide sequence of another CPVL reference cDNA molecule is set forth in SEQ ID NO:30. The nucleotide sequence of another CPVL reference cDNA molecule is set forth in SEQ ID NO:31. The nucleotide sequence of another CPVL reference cDNA molecule is set forth in SEQ ID NO:32. The nucleotide sequence of another CPVL reference cDNA molecule is set forth in SEQ ID NO:33. The nucleotide sequence of another CPVL reference cDNA molecule is set forth in SEQ ID NO:34. The nucleotide sequence of another CPVL reference cDNA molecule is set forth in SEQ ID NO:35. The nucleotide sequence of another CPVL reference cDNA molecule is set forth in SEQ ID NO:36. The nucleotide sequence of another CPVL reference cDNA molecule is set forth in SEQ ID NO:37. The nucleotide sequence of another CPVL reference cDNA molecule is set forth in SEQ ID NO: 38. The nucleotide sequence of another CPVL reference cDNA molecule is set forth in SEQ ID NO:39. The nucleotide sequence of another CPVL reference cDNA molecule is set forth in SEQ ID NO:40. The nucleotide sequence of another CPVL reference cDNA molecule is set forth in SEQ ID NO:41. The nucleotide sequence of another CPVL reference cDNA molecule is set forth in SEQ ID NO:42. The nucleotide sequence of another CPVL reference cDNA molecule is set forth in SEQ ID NO:43. The nucleotide sequence of another CPVL reference cDNA molecule is set forth in SEQ ID NO:44. The nucleotide sequence of another CPVL reference cDNA molecule is set forth in SEQ ID NO:45. The nucleotide sequence of another CPVL reference cDNA molecule is set forth in SEQ ID NO:46. The nucleotide sequence of another CPVL reference cDNA molecule is set forth in SEQ ID NO:47. The nucleotide sequence of another CPVL reference cDNA molecule is set forth in SEQ ID NO:48. The nucleotide sequence of another CPVL reference cDNA molecule is set forth in SEQ ID NO: 49. The nucleotide sequence of another CPVL reference cDNA molecule is set forth in SEQ ID NO:50. The nucleotide sequence of another CPVL reference cDNA molecule is set forth in SEQ ID NO:51.

The amino acid sequence of a CPVL reference polypeptide is set forth in SEQ ID NO: 52, and is 476 amino acids in length. The amino acid sequence of another CPVL reference polypeptide is set forth in SEQ ID NO:53, and is 406 amino acids in length. The amino acid sequence of another CPVL reference polypeptide is set forth in SEQ ID NO:54, and is 490 amino acids in length. The amino acid sequence of another CPVL reference polypeptide is set forth in SEQ ID NO:55, and is 298 amino acids in length. The amino acid sequence of another CPVL reference polypeptide is set forth in SEQ ID NO:56, and is 233 amino acids in length.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6Xhis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat, prevent, or inhibit skin cancer for use in the treatment and/or prevention of skin cancer in a subject that: a) is reference for a Carboxypeptidase Vitellogenic Like (CPVL) genomic nucleic acid molecule, a CPVL mRNA molecule, or a CPVL cDNA molecule; or b) is heterozygous for: i) a CPVL missense variant genomic nucleic acid molecule encoding a CPVL predicted loss-of-function polypeptide; ii) a CPVL missense variant mRNA molecule encoding a CPVL predicted loss-of-function polypeptide; or iii) a CPVL missense variant cDNA molecule encoding a CPVL predicted loss-of-function polypeptide. Any of the therapeutic agents that treat, prevent, or inhibit skin cancer described herein can be used in these methods.

The present disclosure also provides uses of therapeutic agents that treat, prevent, or inhibit skin cancer for use in the preparation of a medicament for treating and/or preventing skin cancer in a subject that: a) is reference for a Carboxypeptidase Vitellogenic Like (CPVL) genomic nucleic acid molecule, a CPVL mRNA molecule, or a CPVL cDNA molecule; or b) is heterozygous for: i) a CPVL missense variant genomic nucleic acid molecule encoding a CPVL predicted loss-of-function polypeptide; ii) a CPVL missense variant mRNA molecule encoding a CPVL predicted loss-of-function polypeptide; or iii) a CPVL missense variant cDNA molecule encoding a CPVL predicted loss-of-function polypeptide. Any of the therapeutic agents that treat, prevent, or inhibit skin cancer described herein can be used in these methods.

The present disclosure also provides CPVL inhibitors for use in the treatment and/or prevention of skin cancer in a subject that: a) is reference for a Carboxypeptidase Vitellogenic Like (CPVL) genomic nucleic acid molecule, a CPVL mRNA molecule, or a CPVL cDNA molecule; or b) is heterozygous for: i) a CPVL missense variant genomic nucleic acid molecule encoding a CPVL predicted loss-of-function polypeptide; ii) a CPVL missense variant mRNA molecule encoding a CPVL predicted loss-of-function polypeptide; or iii) a CPVL missense variant cDNA molecule encoding a CPVL predicted loss-of-function polypeptide. Any of the CPVL inhibitors described herein can be used in these methods.

The present disclosure also provides uses of CPVL inhibitors in the preparation of a medicament for treating and/or preventing skin cancer in a subject that: a) is reference for a Carboxypeptidase Vitellogenic Like (CPVL) genomic nucleic acid molecule, a CPVL mRNA molecule, or a CPVL cDNA molecule; or b) is heterozygous for: i) a CPVL missense variant genomic nucleic acid molecule encoding a CPVL predicted loss-of-function polypeptide; ii) a CPVL missense variant mRNA molecule encoding a CPVL predicted loss-of-function polypeptide; or iii) a CPVL missense variant cDNA molecule encoding a CPVL predicted loss-of-function polypeptide. Any of the CPVL inhibitors described herein can be used in these methods.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1

### Meta-Analysis of GWAS Show Protective Gene Burden Association of CPVL

Regeneron has performed the largest exome-wide cancer association analysis to date, which has led to the identification of CPVL. A meta-analysis of genome-wide association study (GWAS) carried out on UKB and GHS cohorts demonstrated that CPVL pLOF mutations were associated with a decreased risk of non-melanoma skin cancer (Table 3; phenotype=NMSC; chromosome 7, position 28995771 in CPVL). In addition, most of the loci associated with vitiligo in previous GWAS are protective for non-melanoma skin cancer (data not shown).

TABLE 3
Meta-analysis of GWAS in UKB and GHS

| Mask or | Cases | Controls | | Effect | |
|---|---|---|---|---|---|
| SNP | (RR\|RA\|AA) | (RR\|RA\|AA) | AAF | (LCI, UCI) | P-value |
| M3.5 | 30662\| | 470832\|40070\| | 0.0398 | 0.81 | 2.31e−21 |
| | 2138\|22 | 563 | | (0.77, 0.85) | |
| M3.1 | 32406\|416\|0 | 503306\|8139\|20 | 0.0079 | 0.79 | 3.19e−6 |
| | | | | (0.72, 0.87) | |
| M1.1 | 32765\|57\|0 | 510235\|1229\|1 | 0.0012 | 0.77 | 0.03 |
| | | | | (0.60, 0.98) | |

Mask definitions: M3.5 variant rs117744081 encodes missense p.Tyr168 in CPVL peptidase domain; M3.1 association driven by p.Arg464GIn missense in same functional domain; and p.Ser61Asn falls outside peptidase domain and contributes to M3.1 mask.

Moreover, GWAS carried out on UKB and GHS cohorts showed CPVL pLOF mutations are associated with a decreased risk of melanoma (Table 4; phenotype =melanoma; chromosome 7, position 28995771 in CPVL).

TABLE 4
GWAS in UKB and GHS

| Mask or | Cases | Controls | | Effect | |
|---|---|---|---|---|---|
| SNP | (RR\|RA\|AA) | (RR\|RA\|AA) | AAF | (LCI, UCI) | P-value |
| M3.5 | 4922\|340\|5 | 497544\|41972\|584 | 0.0399 | 0.82 | 3e−4 |
| | | | | (0.74, 0.91) | |

### Example 2

### CPVL Expression in Macrophages

Publicly available single-cell RNA expression studies were analyzed to quantify the cell type specificity of CPVL expression (see, Table 5, Jerby-Arnon et al., Cell, 2018, 175, 984-987; Table 6, Yost et al., Nat. Med., 2019, 25, 1251-1259; and Table 7, Hughes et al., Immunity, 2020, 53, 878-894).

TABLE 5

| Cell Type | CPVL Fraction |
|---|---|
| Macrophage | 0.83 |
| CAF | 0.16 |
| Endothelial Cell | .011 |
| B Cell | 0.02 |
| NK | 0.02 |
| T Cell | 0.02 |
| T Cell, CD4 | 0.01 |
| T Cell, CD8 | 0.01 |

TABLE 6

| Cluster | CPVL Fraction |
|---|---|
| Macrophages | 0.6 |
| pDCs | 0.08 |
| DCs | 0.08 |
| Melanocytes | 0.07 |
| Endothelial | 0.06 |
| Tumor | 0.02 |
| CAFs | 0.01 |
| NK Cells | 0.01 |
| Plasma Cells | 0.01 |
| Myofibroblasts | 0.01 |

TABLE 7

| Disease | Cell Type | CPVL Fraction |
|---|---|---|
| Normal | Langerhans Cell | 0.86 |
| Acne (disease) | Langerhans Cell | 0.85 |
| Granuloma annulare | Langerhans Cell | 0.68 |
| Psoriasis | Myeloid Cell | 0.66 |
| Leprosy | Langerhans Cell | 0.64 |
| Alopecia | Langerhans Cell | 0.64 |
| Alopecia | Myeloid Cell | 0.58 |
| Normal | Myeloid Cell | 0.53 |
| Psoriasis | Langerhans Cell | 0.46 |
| Acne (disease) | Myeloid Cell | 0.45 |
| Granuloma annulare | Myeloid Cell | 0.38 |
| Leprosy | Myeloid Cell | 0.34 |
| Alopecia | Endothelial Cell of Venule | 0.21 |
| Normal | Endothelial Cell of Venule | 0.17 |
| Leprosy | Endothelial Cell of Venule | 0.14 |
| Leprosy | Plasma Cell | 0.12 |
| Granuloma annulare | Endothelial Cell of Venule | 0.11 |
| Alopecia | Plasma Cell | 0.09 |
| Psoriasis | Endothelial Cell of Venule | 0.09 |
| Acne (disease) | Endothelial Cell of Venule | 0.08 |
| Granuloma annulare | Fibroblast | 0.06 |

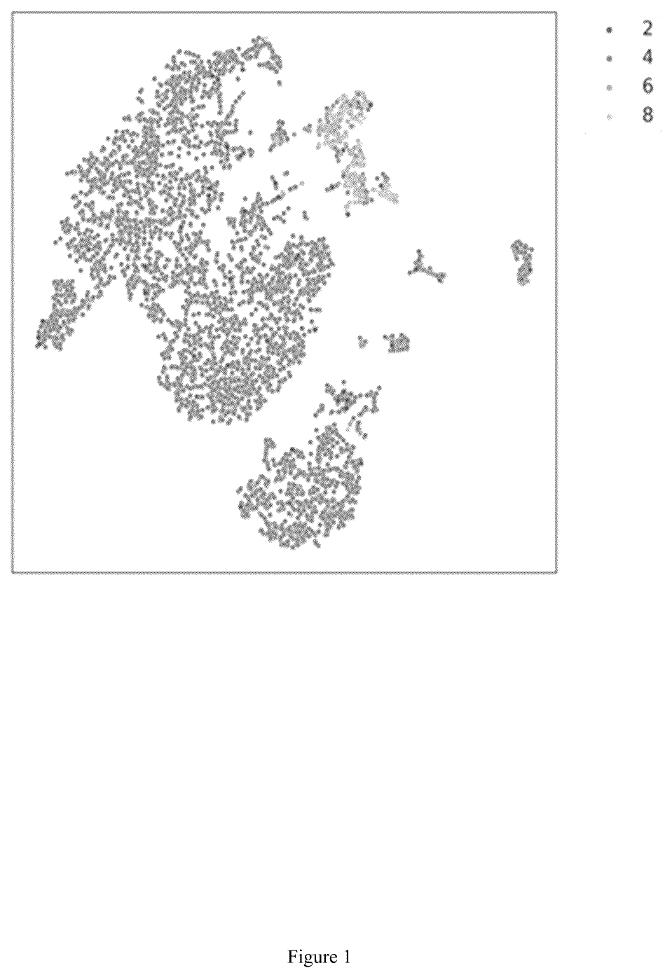

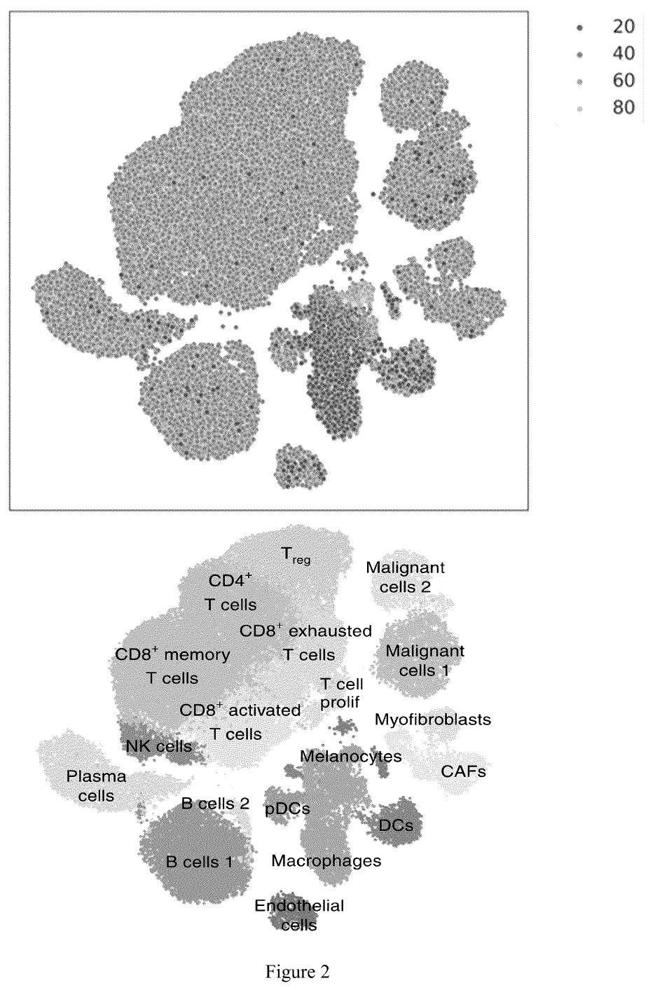

In the tumor microenvironment of melanoma tumors, CPVL was found to be expressed almost exclusively by macrophages. A subpopulation of cancer associated fibroblasts (CAFs) and endothelial cells also seemed to express the gene (see, FIG. 1). In addition, in a basal cell carcinoma (BCC) dataset, CPVL was found to be highly expressed in macrophages and modestly expressed in melanocytes (see, FIG. 2; bottom panel is from Yost et al., Nat. Med., 2019, 25, 1251-1259 for context).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1-21. (canceled)

22. A method of treating a subject with a therapeutic agent that treats or inhibits skin cancer wherein the subject has skin cancer, or preventing a subject from developing skin cancer by administering a therapeutic agent that prevents skin cancer, the method comprising the steps of:
determining whether the subject has a Carboxypeptidase Vitellogenic Like (CPVL) missense variant nucleic acid molecule encoding a CPVL predicted loss-of-function polypeptide by:
obtaining or having obtained a biological sample from the subject; and
performing or having performed a sequence analysis on the biological sample to determine if the subject has a genotype comprising the CPVL missense variant nucleic acid molecule; and
administering or continuing to administer the therapeutic agent that treats, prevents, or inhibits skin cancer in a standard dosage amount to a subject that is CPVL reference, and/or administering a CPVL inhibitor to the subject;
administering or continuing to administer the therapeutic agent that treats, prevents, or inhibits skin cancer in an amount that is the same as or less than a standard dosage amount to a subject that is heterozygous for the CPVL missense variant nucleic acid molecule, and/or administering a CPVL inhibitor to the subject; or
administering or continuing to administer the therapeutic agent that treats, prevents, or inhibits skin cancer in an amount that is the same as or less than a standard dosage amount to a subject that is homozygous for the CPVL missense variant nucleic acid molecule;
wherein the presence of a genotype having the CPVL missense variant nucleic acid molecule encoding the CPVL predicted loss-of-function polypeptide indicates the subject has a decreased risk of developing skin cancer.

23. The method according to claim 22, wherein the subject is CPVL reference, and the subject is administered or continued to be administered the therapeutic agent that treats, prevents, or inhibits skin cancer in a standard dosage amount, and/or is administered a CPVL inhibitor.

24. The method according to claim 22, wherein the subject is heterozygous for a CPVL missense variant nucleic acid molecule, and the subject is administered or continued to be administered the therapeutic agent that treats, prevents, or inhibits skin cancer in an amount that is the same as or less than a standard dosage amount, and/or is administered a CPVL inhibitor.

25. The method according to claim 22, wherein the CPVL missense variant nucleic acid molecule is a splice-site variant, a stop-gain variant, a start-loss variant, a stop-loss variant, a frameshift variant, or an in-frame indel variant, or a variant that encodes a truncated CPVL predicted loss-of-function polypeptide.

26. The method according to claim 22, wherein the CPVL missense variant nucleic acid molecule encodes a truncated CPVL predicted loss-of-function polypeptide.

27. The method according to claim 22, wherein the CPVL inhibitor comprises an inhibitory nucleic acid molecule that hybridizes to a CPVL nucleic acid molecule.

28. The method according to claim 27, wherein the inhibitory nucleic acid molecule comprises an antisense nucleic acid molecule, a small interfering RNA (siRNA), or a short hairpin RNA (shRNA).

29-34. (canceled)

35. The method according to claim 22, wherein the skin cancer is a non-melanoma skin cancer, basal cell carcinoma, squamous cell carcinoma, melanoma, Merkel cell carcinoma, dermatofibrosarcoma protuberans, or sebaceous carcinoma.

36-41. (canceled)

42. The method according to claim 22, wherein the skin cancer is basal cell carcinoma, and the therapeutic agent is chosen from imiquimod, fluorouracil, cemiplimab-rwlc, sonidegib, and vismodegib, or any combination thereof.

43. The method according to claim 22, wherein the skin cancer is squamous cell carcinoma, and therapeutic agent is chosen from cemiplimab-rwlc and pembrolizumab, or any combination thereof.

44. The method according to claim 22, wherein the skin cancer is melanoma, and the therapeutic agent is chosen from aldesleukin, cobimetinib, dabrafenib, dacarbazine, recombinant interferon alfa-2b, ipilimumab, nivolumab, nivolumab, peginterferon alfa-2b, pembrolizumab, talimogene laherparepvec, trametinib dimethyl sulfoxide, and vemurafenib, or any combination thereof.

45. The method according to claim 22, wherein the skin cancer is Merkel cell carcinoma, and the therapeutic agent is chosen from avelumab, pembrolizumab, and etoposide (VP16), and carboplatin, or any combination thereof.

46. The method according to claim 22, wherein the skin cancer is dermatofibrosarcoma protuberans, and the therapeutic agent is imatinib.

47-105. (canceled)

106. The method according to claim 22, wherein the CPVL variant genomic nucleic acid molecule comprises 7:29096102:C:T, 7:28995873:G:A, 7:29030645:C:CT, 7:29096125:A:T, 7:29066109:GT:G, 7:29064221:T:TG, 7:29120891:A:C, 7:29071905:C:CT, 7:29071905:C:G, 7:29064060:C:T, 7:29064167:AC:A, 7:29086513:T:A, 7:29064132:C:CA, 7:29066026:G:T, 7:28995813:G:A, 7:29092703:C:T, 7:29195076:C:T, 7:29120892:C:A, 7:29066070:G:A, 7:29064236:T:C, 7:29095143:C:T, 7:29095143:CT:C, 7:29071879:AG:A, 7:29064077:AT:A, 7:29064100:TG:T, 7:29030600:GGA:G, 7:29066069:TG:T, 7:29064120:G:A, 7:28995858:TA:T, 7:29096218:C:T, 7:29120903:CT:C, 7:29112720:C:T, 7:29195076:C:A, 7:29030581:TGGAA:T, 7:29096219:T:C, 7:29096142:CAA:C, 7:29071905:C:T, 7:29121003:CAG:C, 7:29096102:C:G, 7:29064120:G:GC, 7:29071772:C:A, 7:29120995:GC:G, 7:29066060:G:GT, 7:29030645:CT:C, 7:29064060:C:A, 7:29066032:CA:C, 7:29112740:G:T, 7:29120989:G:GA, 7:29112702:A:G, 7:29030604:GT:G, 7:29064150:TAA:T, 7:29064087:A:AG, 7:29064093:GC:G, 7:29112740:G:C, 7:29064060:CCTTAT:C, 7:28995830:GCT:G, 7:29064221:TG:T, 7:29086483:C:T, 7:29030760:C:CTGAAA, 7:29096207:T:TCTGG, 7:29066114:TC:T, 7:29072425:T:A, 7:29112817:C:A, 7:29064115:TTC:T, 7:29086488:C:T, 7:29095087:A:T, 7:28995863:C:T, 7:29092645:C:A, 7:29096130:G:A, 7:29112766:C:T, 7:29112757:T:C, 7:29096177:C:T, 7:28995863:C:A, 7:29066115:C:A, 7:29030726:C:T, 7:29030584:A:T, 7:29072407:T:C, 7:29064135:C:G, 7:29030749:T:C, 7:29095089:T:G, 7:29030710:G:C, 7:28995876:T:A, 7:29066063:A:T, 7:29064068:T:A, 7:29112774:C:T, 7:29071894:C:T, 7:29096198:G:A, 7:29112810:C:T, 7:29120923:G:C, 7:29086539:T:C, 7:29072410:T:C, 7:29120922:G:C, 7:28995851:G:C, 7:29030642:A:T, 7:29096199:G:C, 7:29096169:C:T, 7:29030738:G:C, 7:29064134:A:G, 7:29064173:A:G, 7:28995809:A:C, 7:29096199:G:A, 7:29096150:C:A, 7:29030599:C:T, 7:29072321:C:T, 7:29030737:A:C, 7:29030578:T:C, 7:29096172:G:A, 7:29092663:A:G, 7:29030682:C:A, 7:29095097:T:C, 7:29086497:T:G, 7:29092632:T:A, 7:29030611:G:A, 7:28995872:C:T, 7:28995816:T:G, 7:29092666:C:T, 7:29066097:C:T, 7:28995859:A:T, 7:29086486:C:G, 7:29086549:C:T, 7:29072416:G:A, 7:29096136:G:C, 7:29095103:A:G, 7:29092638:G:A, 7:29092660:C:T, 7:29066058:C:G, 7:29072329:A:G, 7:29030727:G:C, 7:29095088:T:A, 7:29064125:T:C, 7:29086515:T:C, 7:29064122:A:G, 7:29064090:A:G, 7:29096171:G:A, 7:29086548:G:T, 7:28995873:G:C, 7:29066117:A:G, 7:29064101:G:A, 7:29030740:T:C, 7:29095089:T:C, 7:29064164:C:T, 7:29071803:G:C, 7:29064168:C:T, 7:28995864:C:T, 7:29096144:A:G, 7:29096172:G:T, 7:29071780:G:C, 7:29112725:G:T, 7:29092680:G:A, 7:29066071:G:C, 7:29096184:G:C, 7:29096112:T:A, 7:29072423:A:G, 7:29030722:G:A, 7:29064098:A:C, 7:28995852:G:A, 7:28995846:C:T, 7:29030609:C:A, 7:29112754:C:T, 7:29092683:A:C, 7:29112732:T:C, 7:29096139:C:T, 7:29096136:G:A, 7:29112719:C:A, 7:29072347:T:G, 7:29086548:G:A, 7:29064092:G:T, 7:29112750:T:C, 7:29112735:C:G, 7:29096121:C:A, 7:28995814:A:T, 7:29066108:A:G, 7:29092693:C:T, 7:29096169:C:G, 7:29095101:G:A, 7:29064212:T:C, 7:29120929:G:A, 7:29030705:G:A, 7:29095086:G:T, 7:28995812:C:T, 7:29096143:A:C, 7:29030577:C:G, 7:28995822:T:C, 7:29096174:C:G, 7:29072380:T:C, 7:29066105:A:G, 7:29064213:A:T, 7:29096138:T:C, 7:29064174:T:C, 7:29086498:A:G, 7:29064152:A:G, 7:29096159:G:A, 7:29096160:A:G, 7:29030750:A:G, 7:29071781:C:T, 7:29072339:C:T, 7:29030600:G:A, and/or 7:29096120:A:G.


SEQUENCE LISTING (76 sequences)

SEQ ID NO: 1 - DNA, 199640 bp, Homo sapiens
  source 1..199640: mol_type = genomic DNA
gctcctttcgcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggtaggctctgtccatcccaggctgcggggcgagggcagtggggcgagacccggcgcggcagcagcgcccgacgggctgaaactcaccggaggacaccgacgagccggacaggctggagttgctggacgctgccatctccgcgcgcccctcgcgtcgctgctcgctcagccccggtgcggacgccgccgccgctgccctcgcccggccgctcgcagcctccgcggcccccagcccatggcaaagtcctggggacgcgcagaaagcaccagcctccggactctgcctgcacttcctgctccaccagatgacgcgctgccgccgccgctatttatttgtggtttccgtcccgctccgcgcctttgcacgcttttctatgggtgtcagggggtaggtgggagcagagggaggccgaggggcagccgggacgggcggatctcggctcccgccgagcccctctgccgtccacgctcccctgtgcgcggctgggaccagggcgtcctggcagctccgagccgccccgccagctcccgcaccctctcctgggctcccgcctctcccgccgcacttcgagtagaccgggcagaggcggcgggagacggacagacagagcgacctcccccttctccctccgccccaactccgctcgcgcacacacaaaggaagaagaggagacgcggtcgctcggggggtggttcggctgctgcgggcttgacagctcccgccactggggctggagggggaggctgcggactgctggacagggggaggcgggacgggggaccgggtccccggggtgctggggaggccggggccgggcgcggggcgagcgcggctgctgcgcgcagcgctgctcctaggatttgaaggggatggggagggagggtgtgggggaaggtggtggggctcaccgcgccccgccgggctctcgagcagcgagacagtgggggtgggggatccagagggcgaaggcgggatcactagggccacggtgctaggcagaagggaacctgggatgacagagccactatggagcgctccttccactcctctccaaattgtcctttgttgctctggagctgacggtccctgccctgcaagaaggtgcgtttcctaaaacacccgggaagccaacccagctacacttaagtaacccagccacggacagtgcccgacgcgtccgtgggcctctgaggttggccttcaaagaaatagagccgtccaaacgtgtcatcaggcccacacaaagctggtgtcaaagagaaagaggttctccgtgccttgcaaaaaaagattctcacaaaagacttaagacttggcagcaggtccgactgtgccctttcaggataggaactgctcaggtgtgtgagagaaagagactcgagcacaaggtgtgtcccttccccctgtatttaaacctaaattaatttcactgggaggaaataccatgcagattttttttaagtctgtttcacgctcccagcgggctaatcggttctttcaatcctaaagcaacagaaaactgtgaaatttgaagagaaattgttagatcaaaggaaaaagcaaacattaaaggaatgaaagtactctcattatatagcaaaaatggaaagtgttcacattggggtagttcaactttttaaaacatggttaaaagaacatagtcaatttaaaaattatcaggagtgacacagaacagcagctaggccctggggatccacatcaaccaaggacctaacatacagtgaataatgaatgttaaataatcacagtacgcacccacgtcacattttctagttgattagaacttaaaaaaaaaaatccctccaggatactatcccagtgtgtcttctcattaaggtccattttttaaaaatacaccttgctaaactgttaaaggagtgtgagcactcttcagtcacgtgggactgcgtcttcctacccctcaactgtatggaccttgaaacagccctggctggtctaaggagacccaagcgcccgcattttcccagcccctaccactatccttcattttgtggactgcacgaaatgatgcagttcctaatctggagccttcagggacacgtgacccatcacacacttggcttgttgatcagatcagaaggaacttactgtttccccagagttcttctgatagaaaacagcacttacttgtttgactagtaatctatgtagccccccagatctttagatggtggactatctgacttggctatgtcgcccggtagaattttaccatctgggcttcgaagacaactgggttcaaactctggttctgtcacttagctgcgaacttgagcaaattacttagacctactgagcctcagtttctacacctgccacaaaaatctgataacacaggatgctgtgaagattaaataagatgctatgtggaaagctacagtgtacctggcatgaagtttgtgttccaaagtgattgtaagttgctcactttaaggaaggaatgtatagtttgaaaaatttttgctttcttcattgcatctggagataactaatcaactgtgacctgttctactagggggaaatgtcttatcttcaatctatggcttatatttcttgtcccaaattgtgcaatagggtaacaggtttcattattcatctctgaaatttcacaaaaggacaacagattatgtttctgcagttcggatactcatatttcccttcatagtgttttaactggagaaagtaaagcatggctctgtgaggcagattatttttattgtcctaaattgacagatgaagggacgggggtatttgctgtgaaggcaccacctgaaggccctgtgctgtacagaagtagaaaaaagctgagactcatgttttcctggatcctggggccagaagaggaatacaaggccaacaaacacgcttgctgaacagagaagtgctgatgggggaggcccagtctgtcggtgtgatatgagctataactttgacacccattattttgaataccaaacagaaagctccaggttcagaactaaaatctgaaagttgattgatcatctggattgataaagccataaccaatgcagaaaactcaacatgaataatcattcactatcatccttcttagattcaggagcctgaaggctggctgctccttgcaaaaatgtttagtatcagtaagttatcaggacatgctggaaataaaatgctaacatccctctttggtaggctcagggacctgtttgtggaccttctaaagtatttctccaaatgaaacaatgaaaaagttatcatctattttattcatttatgtgttgccttaattctaggtaatacctatttgccagtggaaagaatattggtgctacagaaatcttaattgtttggaaagtagaattttaaaagacatttctgccttttttcttcaaatattaaaagacttatgggataaacggagtcttgtggataatgtgggcttcttaagaataaaaattatcattttgttttaacttagctttcacctggcattcaaagccttccaaacttacctttccaatctaatctctgttcccccctttcacaaaggcaaccctctttcatggtggcctatgcccttggggctagcagcactggattctgccattccttcctgcttcccatcctacataccaaaagggttcaaccagaatgcaccttctctggaaagttttccctgattcccaaggcagaaaataactcttcttctgttggatttctctagtccatctactgtacctctcaatgggcagatctcatgctctgtcttattttaatggtacttttggtatatatttcattaccttagtccattaatagcgggcttggcctggtggctcatgcctataatcccagtgactcagtaagttgaggtgggaggatcccttgaggccaggagtttgagaccatcctgagcaacatgaggcctcatctctaaaaacataaaaaataaattagccaggcgttgtagttaacctgtagtcctagctacttgggaggctgaggggggagaattgcttgagtccagaagttcaaggatgcagtgagctctgttgatgccactgcaccccagcctgggagacagagtgaggccctgtctcagggaaaaaaaaaaaagcatggtgggggcaggatccttgtttgcccagcccctgtattgtctaccatactttgcatacaataggtgctcagtggatgttgttcaattgtttactcattgagtaaatgagcaaattaaggacatctgtaaatggttctcttgtatgccagttcctttccacgttagtagctgtgaccactgagctacaagacaatgagtaacaacacatctgcagcaaaagaaagaacttgacggtcatgtaaggatactacctttgctgtgatcaaaaaagactgaaacggtgaaacataagaataaaatcttgatggagtaaatatttgcaatccccaccccaccctccttcaccggctgttacaattcacatgatttattgtttgcagggtagctcttatggtggtgtaatttgtttattttgtttcaccattcttttgaagtttaaaagcagacaagaaagcactgcagggtgaacgtggctgtcatagcaggagctgtgctccctcactgcaggcattctggagtgttttttagtgaaggtcattccctttccctgtgctaaacacagaatgaaatactgtattccttactttcaagattaatccagtggtgggcattcacaggagaatagcagggctattcgtcaactagctttaaatattttatggcaagtactctaccgtaccctgaaagtggtccttttgccatcaatttctcaacatcaaaatgctcttttgctgtagtgagaaatatttcgctctgtagtttaagccttatttcaaatacaaagacacctagtcctgagaaagagaacgcattggctcttctgaggcaggcgtcactaggggcaggagctgagaaacccgctgtttcctcaggaggtgcacagacggcccaggatgggttaatgagcaggcagctcagacgccgcaggtagcggccctgcctctgaacaaagaggagtccacacctggggtgcagacaaagggaggccgcctctgctacccaattcccagttcacatcgcctgctgctggggacccctagtggcggcacacgacgcaggcacaggtattcctgcatcgtctttctcactgagctgcaggggggcttgggggtattgcctggagtatcctatttggccacttagcttgggcggatcagatcggcacatgcaggcacttaaggcatgccatccgctgcagtctctttgactggaaagacagagagggagaaggggagtgtagggggtggggggccgagagagagagagtttttctttctgtttgaagagaaaaacaggatagcattcagaaactcattgaatatgttttcttctgcatgttgcaccttctggctttgttccctgacacttcaggttacaaacatcttttctgtcttcaatgtcctaagagaaatctaggataaactaaagtgaagccagctgtaagcctgctctgggcaaaagcagggagtgagtttgtttttcgtgtcttctgcgggggttcctatggctttatgctctttttctatgaaaatttgctttcataacgatatccaatatattcattctctactacctgttcatttattcaagcattcattaattcactcagtcatgttgactaaacactcattatgtgccagggagtatacgaggcctggggaataaagatatgagggaaactggctgggcgcggtggctcacacctgtaatcccagccctttgggaggctgaggttgggcagatcatttgaggtcaggagttcgagaccagcctggccaacatggtgaaaccccgtctctactaaaaatataaaaattagccaggcgtgatggtgtgcgcctgtaatgtcagctactcgggaggctgagacaggagaattgcttgaacccaggaggcagaggttgcagtgagctgagattgtgccactgtactccagcttgggcgacagagcaagagattctgtctcaaaaaaaaaaaaaaaaaaggtatgaggaaaactatatccttgctgtgggggatcctacaaaacaaccttgactccttcctgtgacatttacatttcaactgggacaaatttttaaaatagctatagaaatgatttctgtttgggaattatagctattatccatgccggtgacgactttcttttcagcagttttgttgttgttgttgtttttaacaaaacgtccaaaatcaagcctcaaagactatttctagacagctggcattttcttatccacagttttagcttgtcagcccagggaaccgagcagatcctgtactctgaatgtcctctgccttcactccccaagaaaatctgaatcaatattttcatttctttccctgtagcagctatacgttcaataacaaaacagaaatcctcttttttttaaaaaaaaaataaaaaaaggagagtaaatgggcattgaaggaaagctaacagacacaatatctgagccaatcttctgatacagaaaacatattttggccttttacaaatattccagattgtctgtaaaacaaagcatgagaaaatgattctaggaaagtttaatatgtatttatggagtcaaaaagacctgggcttcactcctagcttgtcatttattagctgtgtgaccctgagcaaattatttaatcttatattcttggtctctaaaatgggttaataatccctatttacagactttgtgatgatggaatgaagtgatagatgtaatatagtgtctaaagcataaaagtccagttaggaactctaggtggcctctgttccttagagcaaagtatagcgtgctcacatctgcttttctggagtaatctgtgagaattaaagtctgatgcaaaagtaattttcatctctccctaccacccccctgttacatcacctctatgcgagtatattaagacataggagccaggcctgggctttgaagataaatctgagttcaaatcctaatttagctacagatactttggtgatcttggacaaataacctttcacattcacagtctcctcatcaggtaaaatggagataataatatacctcatatttatgtaaatgaatgtatgcccaacaataatgttattagcactcaattatattctgcacggcaggtagtttttaactgtgtattaattcctggggagagagtggatgttgacaatacatcaggatctttttttattttttatttttattttttgagagggagtcttgctcttgttgcccaggctggagtgcaatggtgcaatctcggctcattgcaacctctgcctccctggttcaagcgattctcctgcctcagcctcccgagtagctgggattacaggtgggattacaccacaacacccagctaatttttgtatttttagtagagacggggtttctccatgttggctaggctggtctcaaactcctgacctcaggtgatccgcccacctcggcctcccaaagtgctgggattacaggcgtgagccaccgtgcctggccatacatcagaatctttaggaagtggcatgtataatataacagagtacactcaagtgctctctgtctctctctttgtcacacacacacacacacacagacacacaaacacacacaccactagatgataatgtcttacatctggacatttccccacccagtcccattgggtgagaaacctggtggaatttcaaagtagatttcaataaaagtatagacagattacttaagctctctgccaaatttggcccctataagataaaggagtggaaaccaacatttgatgaccactactactgctccaggtattatgaccactctcaccatgctttctgccattagtgatatagaaaagtcactctccttaagtaatcatttcctatgtgccaggatcatgctaaacactttgtatacattgtcatatacccataacaatcctgaagtgagaattgttatcctcattttccatttggggaaactgaggctagaaaacttatataatatcttaattaaaatgctcaacaactccagctgtagatattatcatccctgctctttttggttaagaaaacaaaaaattagggtaagtaatttatcaaggttacaggcattaatttgaacccacctttggctgattcattccaatacacactaaaaaaaaaattaccctttagtttttacttgaaatttcagcctgctgttatggtaagtattttcctgtttgagactttagtcagttgagaagtctttaaaatacatgttctttgcttgaatactcatgtttttatttttatttttccttccttctctccctctccctccctcccttcctgcctctcttccttccttcctccctttctttctttcctttcctttcctttcatctctgtcacgcaggctgcaatacagtgacacgatcacagctcactgctacagcctctacctcctgggcttaatccatcctccctcctcagcctcccaagcagctgggactacaggcacgtggctgctttttaaaatttttagtggagacagggttttgcaatgttgtccaggcttgtctcagactcctgggcccaggtaatctatccaccctggcctcccaaagtgctggtacaggcgtgagccaacacgcatggcctacttatgtttttcaatttgaacctatgatgatactgtcattttttttttgcctaaaacataaactttggggcaggccaggctctggggaactcataggacttgtactcctgaccccaagggccctggcaaggacaggcacagtataggaagactaagaagtcatttgcctgagtgcaggaatttcaagggatcaaaattattccctaatcaaaaactccatcctgttcaacttccagttgctcagagttccatagttagaacttttacttcttaaacttactcaaacaatacagtgagcatctgccataatttgatcacctctggggaactgaaggaattaaataacgattttcacatgggcgagtttccctatccctaaaagctaaatttaattgcaaggagggaccaacacataggacagggctgtgcaacctttaatatatctacatattggtcttgctaaaatgcagattcacatccaggggcaggcccctgaatttccaacaagcttggaggtgatgtgaataattttgaataataaaggtcctagggcgatggtttgcaactcggggtatatatgagaatcactcagaaggcttttagaaatcccaatgccgagatcacgctccagaataaacaaatcagaatctgagggtggcagaaggtagaaaccaaatatcagcctttttttttttaattcccaggtgattttgatatgctgctaaggtttgataagagaaatgagattcagagaacagattatattgttgaagagcttgactggaaatggtaagagctaatgttgctccatgtttttgccgggattgtctctggtcatgaaggaaagagagggaatcaaattcttatcaagagcgtgtaaccacagacatcatgatatgagaaaatagaataaaatctaacattagcatgacattttctcagtcaatgtggggcaattcttcttcaaattttggtccatgttatgatctttaaaagacacttctcaatgaataagcataaacaataggcacttagggtcactcccacaagaacagccagctgtcagtgaggcagatgttttccagcaaatctactctatcatttctcaatcataaagaagattagaaatgggattttcagttcagatggggttgaaaggtgatgtttatgcctgagaaatacttcacaccacagctagaaaaattggtttggagaaaaaatatctcacaacttcttaggtgttctgaatatatgaaaataaaaccctcagcttggacagaaagagggggtatcacagacaaatgcacttagagacatctactctcaatggtggcagcaacagagaggcgaaagtattttagctgactctttatgcccaaagatttttgtccttatccacataaactcccattcctcagggctggcattgaaagggtgaataattaagtcaaccacaatgtttacagtggttaactcaggtgtgatgcttaattttatgcgtcaacttgattgggctgtggggtgcccaggtatgtggtcaaacattatttctgggtgtttctgtgtgtctttgaatgaagttaaatttaaatcagaacactgagtaacacagattttcccccagtatgtagatgggccccatcacatcagttgaaggcctggagaaaacagaaaggctgaccctcccttgagagtaagagaaaactcttgtgatggcctttggactgatcatcagctgttttcctgccttcagatccaaacggaaacattggctcttcctggggttccagcctgctgtcttgacaggaactataccatcagctctcctgggtctcttacatgccaagtcatcctgcagatctgggactcactccgtaatcagtgagccaattgcatatactaaatctctcttatctgtatatctcatacagtcatatattttctcatataaatatatatatgagatacatagatgtatgagatatgagatataccatatataatattcctaaatatatatatcatatatatccactatatatcttatatatagattgtatatgtatatctcctatatatatttcttctataaatatctcttttatctatctatctatctatctatctatctatctatctatctatctatcatctatctgtatcttctgtacagttagtagtcctttggtatccatgggagattggatccagaacccccactcagatatcaaaatctgcatatgcccaagtgtcttatataaaatagtgtagtatttgcagataacctatgcatagcctcctgtatactttgaatcatgtctagattacttacaatacctatactatgtaaatgctatgtaactcgttgttatgccccattgttttttatttgtatcatttttaattgttgtttgtaatttttattgttttttctaacatttttggttggttgaatgcatgggtgtggaatctacaaatatagagagctgactgcatatgtatctcctattaatctatatctaatctacatatatcatatatcctatacatatatcttctatatatctctcttattggttctctttctctagagaatgccaccaattcagcacttaaaaaattctgggccaggtgtggtggctcacacctgtaatcctagtactttgggaggccaagaccggcagaccacttgaggccagaagtttgagaccagcctggccaacatggtgaaaccctgtctctactaaaaatacaaaagttagccaggtgtggtggcacacacttgtaaacccagctacttgggaggctgaggcaagagaattgtttgaacccaagagacaaaagttgcagtgagccgagatcatgccacctcactccagcctgggcaacagagcaagaccctgtctcaaaaaaaaaaaggctaggcacggtggctcacacctataatcccagcactttgggaggccaaggcaggcagatcacctgagatcgggagttcgagaccagcctgaccaacatggagaaaccctgtctctactgaaaatacaaaattagccagtcatgttggcatatgcctgtaaccccagctactcaggaggctgaggcaggagaatcgcttgaacctgggaggcggaggttacaatgagccaagattgcgccgttgcactccagcctaggcaacaaaagcgaaactccgtttcaaaaaaaaaaaaaaaaaaaagtctgccatgttttccagttgccttcgtacctgtctcatataatgggaagattctggagggcagcaatggtgctctgctgttttcatatatcccttctttaacccaagaatctttcctgtaatagaatttcaaaaatgtagaatccagctgagtgtttatctttgttctggattctcaagccatttaaaggcagcaggctgtcatgattttccacacaaaagggcaaaaacgggattaacacaataggatgctgtctctactctcatctaccctgtccaaggaatttaatttcctataagcctacctccaattctccttgggacagttatatttaagaaccttaatgcttcagaccctcttttgacattaaatctttgtctgctgaatgctaacaatcttgagggacattgggtttactcaatttgtatttttagacatgagtgtacgtaattgtgtagacaaagtatttgaacgttgaattgatattttaaagttttgatccaaatgtgtgatattggataaaaatactctaggaggcttaacgtgggtaatacagactaaacttgtcaagtagttaaataggatgatctggcaggacttatgaaaatgtctgaagaatattacaactttctacgttttctttttgatctgggtccccatagccctattctgctacactctatgtgtcatatcacaatgaggagcttttgctttttactgctgcttatgatcttatttcattattggtaggtggaacactatcagattaatacaactcttttaaggcattgaatagaaagattccaccactgctaaccttcctctgcgctcctatttttcaaactctatcattaccttctcttctgtagagcatcctgtcaccatcttataatagaagttcagttcatttgcatatgcatttgcaggttttcattaagaggaagacagcagagtgattaatgagtgttagttctcattttgcaaaaggaatattgtctttttaaaaaatattccatctttcagagcactgaaagaaatagttataatttaagtaagaaaaagtcatttacagcatttatgttatttaagatgttttgcttataaagtaatccatgcttactgtagatatgttagaggcaaaaaagagataagtaaaacaaaaaaagaaatcatccatattccctccaccatgaataaatacttaggtacttttcccccccttaggaattagcagtttagcagcctgatttttgcagtttacattacatagcaatatgaacattcatgtggaaaaaaagtttgttaggacctatataaaaaagtgtctattttggaatgttaagatcattagtgtctactttatttagtttttctagattgtgtaatttttaaaatgaatacattttacttttctagttggaggccataataaagctactttccttttggaatgaacttgtacaattatttcctatgtcttatagattcctaatgtgacttttaatggcaatataaaattcctttctattaatctacaataattcatgttgttattcctccattgattgaaatttaggttgtttcacagtttttggctttttctgaattactcttggacaaacttccttacacataaatctatatgtgcctttctgtttattttcttaggcttgattgctgtaagttgaacacgtcagtggcatgggcaccagctgcacaaccctaggagccaccaaacacagtatattctatataaatgttgctgccaggagccccagttcaaactacaatgtgagtaacgcctgctggagtagagcaatagctgaagccttggtcaaaggctatgaaaatgtttaaagcactagataaaaattgccaagctgccttctggaaaaattgtctgaatttatattcattccagaactcaccacatatatcatgactgagaattagaatatattttaaaacacaaattccctaggaaaaaatggcctcccattgctgtattcatttgtatctaactgattactagggagattaactttttgcttatgtctactagtcatttgtatttcttcttttgcaaactgcctattcatctcttttggcaaaataatagtaagtactgggtaccagtgattggtaccaagagttgtttatgtatgatcttacgtactcttttcaattacactgcaagcagtcatgcttattatctccatattacaaattagaactccaagaggtatacggatgatacgagtcttgtccaacatcacacagaatgtgacagggcctggatttgaacccagctctaactccagagcctgagctcttaatcactggactctagtgccccttaattagtgtgttagtgttcttcttattagtttgtaataactctttacatagtaattataataacttttaattggtcaaatttgtgccaaaggtatttttattggctcatcatttgccttaatttgatttatacagatttaagtcatttttatatcaatttagatgacttttttttttttgagatggagtcttgctctgttgctctgtcgctctgtcgctctgtcgcccagtgcagtggcgcgatctcggctcactgcaacctccgcctcctaggttcaagtgattctcctgcctcagcctcctgagtagctgggactacaggcatgtgccaccacgcctggctaattttttgtatttttagtagagatagggtttcaccgtgttagtcaggacggtctcaatctgtcctgaccttgtgatctgcccgcctcagcttcccaaagtgctgggattacaggcgtgagccaccacacccggcctagatgtcactattggtacgttatcacattttaaaaatattctaaatttgattcacaatattattaaaatgtttgatgtgaaatattttcctttctaataaaaaaactattggggaacagattgacttcggaatattaagaatgaaacacatgtaagcataatatgtgaatatttggaaatgaaataacctacatcattgatgaaaggacatcttatttctcattgatatttccactgtctgcagggaaatataatcatttgaaaatacaagacaaatagcaaaagatcaatagaatgcttagcaatgtaagttatcttaatgctttcaaaataacagtgaccagattttttgtttgtaatacttaagttgatttgcttgcaataataaataaatcctcacatgcactaactcagttttgcaaagcataagaaaacaaatgaacctcataaatgctatatagcccctccacaaatgtccagttcttacccgtgttttgcattgatataaaatagaatagctattaagtattttgtaatttgagaaataaaaaacctggtaattattgatggtttctctacttagaaaaaggagaatatttatttacatatatcagagtaattctatttggcaaaggatatatagcaaaataatcatttgtgttactaacatctgccttgttcttgcttatgaagaagacttaggacaagtatcagaatctttccttctcactcctgaagaatttgtgttgagtctgggttcctggattttctagcatctgagagtggtcaaaaaattctcagactgcaaaacttcagcctgagtttctgtgcagagcattctggagtcttttcttcaacaaggctgactgaagaaggctgacactgtgtgggaaggaaggcagaactgatctagcaaagagtgaagggagcactcttcctcatgacttggcgtgggatggggtggttcttaaggatggcatcccatgcccactctcatgcagtgctgtgtgaggggatggagccttctgaagggttctcccaagatggcagcatggcctaacacaaataagccttatgcccaagcttggcaaaaagaaaatggaggggctgttttcaaagggtccatgaggactgtaagtgtgtcagcatgatattggaggatcacctcctgttttctgacaccaagagagatgctgggtcccctataatcctaaggaggcaggagacctcaaaaccataactgaggaggtgcaaatgctggttagctctggtaggtggggtcctgcctggactataatgtcccttaaatggtgtgttagtgttctttttattagtttgtaataactctttacatagtagttaacctttgattggtcaaatttgtggcaaacccatttttattggctgattatttgccttaatttgatatatcatagggatttaagtcatttttcatatgaatgcagatggctaattccactccaagttgtgaaattaattaattaatgccaaacagtcattcaacaaacaataactgagcccctactatgatccaggcataggtatagaccctgagaatataacaataaacaaagtccctgcccttaagaggcacagacaatgaactgctttttttaaaaagataacttcaaatgctgaaaaatgtgttcagatgaacataaaacagcatgtcaggcaacgtctctcggaggatgtgacatttcagcagagacttgatcttgcaggtctggaggagggaacagcagatttaaagggctgagtcagacacgagctgggcatgttagaggaacaggaaaggcggccagtggagctggcgtcaaaggaatgaaaccgcacgcagaggaagaccaggtcagaggctgagtagatgtcagatcctagcagccacgtgggtcatagtggagactttctgtttatttctagacacaatgggaagccacagaagcttttaggaaggggataacatgttctgattcatactgaaaagatcactttggcagctatggaaaaggatagtaagaagagcagggtgcacggtggctatgtgactattccagtggtttcaaagaaaggctgatggtgtggacagaggatagtagcagtggagatggcaagacaccatcagacttagggtagaatctgacaacacttgctgatgatggaatgtgaggcatgagataaacaatcatcaaggtgactcctaggtttttagctgagcagttaaatggctggtggtgccacttgctgtgatggtgagggcttggggagggggaactttgtgtggggatggaaccgagggttgtgtttcggacatgtgaagtttgaggcacctatgaaatgtctaaggagatgtcaagtagcaaggtttggagagaggtctgggttggaaaaaaagattttggttataaagtgaatgaatgaaagaacagatagataggtgggagcataatgagatgggtaggtgtttagaagtaaagagggttggggtatagctggtaaagtggatggatggatggatgactgcacagatggatggatggacagatagataaataggtagatattaatagatggacagatagataaacagatggaagaaggaaaagagagagagaaaaaatgaggggacagaggaaaagccagtaaattaaaaaaaaaaaagtgagggggccaggcacggtgggtcatgcctgtaatcccagcactgtgggaggccaaggcaggcagatgacttgaggtcaggagtttgagaccaggctggccaacatggtgaaaccccatctctactaaaaatacaaaaattagcccagcattgtggtgtgcacctgtaatcccagctactcaggagattgcggcaggagaattgcttgaacccgggaggcagaggttgcagtgagcagagatagcaccactgcactccagcctggatgacagagtgagactctgtctcaaaacaaaacaaaacaaaacaaaacaaaacaaaataaaacaaaaaaagaaagaaaaaagaaaaagatctcctatataggtataaattactttaagcaatctgaagattagaaaggaataagtttcccagtgagtaaattttttagtgtctctgtgctagaagacaatgttcactaaactaaaaatattttatcttaaggagataactattttatatttgcctcttccatctatattttttcccctctctttcttgctttcttttgtattaaatgattttttttcattcccattttctctgtatttatttggaagttataaacactgtttctattcatttagaagttaacctaggcattttaacatataagctaacgaagcctaatgtttatccatattcttaccattctgaccaatgatgaaagagcaagaggttgctttccctttccttcctggcttcctatttatatgctcttccttctagtatttcagctcaatctcgttttttaaacaccagaagttagccattatttgttgttttatactgtcaacgttaatttaggtttaaacagaagtttcccattttcttggctcaaaattctttcttgtatattagatcttccttctgtattcttttttcttcctaaaaaatacccttttaaagcttctttgtaagggtgtcttagtagtaatccctttcaatttttgtctataaatgtctttactttgtctttcctttggaatcaaagttagatttgtataggattcaagctgacagttaatttccttcagcaatttgaagagacatactgctaatgaagacatacccattgtgttctgacatgttttcctgccaataagaagtccactattaatctagttattatttctttttggcatttttttttctttttcctctctgtttgggggaatctgaagctttcctacagtgtatctaaatgtgactttatttttatttatcctgctcaggacctattggccttcttgacttctcttctggatctttgtagtcagaataaataaatagtcatggtcttttttttcttttatctgttttttttttcttgagacggagtcttgctctgtcgcctaggctggagtgcagtggcgcgatcttggctcactgtaagctccgcctcccaggttcacaccattctcctgcctcagcctcccgagtagctgggactacaggcgcccaccaccacgcctggctaattttttgtatttttagtagagacggggtttcaccgtgttagtcaggatggtcttgatctcctgaccttgtgatcttcccgcctcggcctcccaaagtgctgggattacaggcatgagccaccgcgcccgtctggtctttttttcaattatggaaaatagtcatgatctctttgaatattgcctaatctgtcctaaaatgtcagtgaattagacatgttagactttctcactcaatacttcctaacccttctttcatatttttcactgtttttttgttttgtttttttccctgtgctccgcttgggtaacttatcttctactactctccttcccgttggctatcaatcaatttatctgtgttagtctgttctcatgctgctaataaaggcatactcaagattggtaatctacaaagataagaggaaaaattgactcagttccatacggctggggaggcctcacaatcatggtggaagagcaaggaatgtcttacatggcggcaggcaagagagtatgagagccaagtgaaaggggaaatgccttataaaatcatcagctctcagccaggcatggtggctcatacctgtaatcccagcactttgggaggccgaggcgggcagaaaacccgaagtctggagtttgagaccagccttgccaatatggtgaaaccccctctctactgaaaatacaaaaaattagtcaggcgtggtggcatgcgcctgtaatcccagctactgagggggctgaggcaggagaatcacttgaacccaggagtcagagactgcagtgagcagagatcgtgccactacactccagcctgggtgacagagagaggccccgtctcaaaaaaaaaaaaaggaaaaagaaaaagaaatcatcacatctcatgagacttattcactgccatgagaacagtatgggggaagcactccaaagattgaatcatctcccactgggtccctgccacaacatatgggaattatgggagctacaattcaagatgagatttgggtgtggacacagccaaaccatatcattatctaatctgctgcttatcaactgagatgaatacaatttttatatttttcatttatagaattttgtttattttgtgtctctctctcttctagcttcttgttcctgtatctctctaaaaattcttttttttttttttttagatggagttttgctcttttttgcccaggctggagtgcgatggcacaatcttggctcactgcaacctccacctcttgggttcaagcaatgcttctgcctcagcctcccaagtagctgggattacaggtgtgtgccaccgtgcccagctaattttgtatctttagcatagatggggtttcaccatgttggccaggctgatctcgaactcctgacctcaggtgatccgcccaagtcggcctcccaaagtgctggattacaggcgtgagtcactgcacctggactctcaaaattgtttaatcattgtgttgggtattttctttctgccccttccaaatccactctaccatgttccatcctgctttgtgcccccatggctaatcagctgagcaacctgtcccttgtctggacaggttcctgtccctcttgggggagaagcagaccttggctatgtactggtgtagcctccaggcctcgaaaagctttactgtctctcctcagggactgatgacttttgcttctgtcctttccccagaagcagatatgtgcctgcataccaaaatatggaaagacgggattgctggcccccctccagaggcagagggctttgcttctcaagggcttgctgcttcttcctgacagcttaattaaggcttttgctcccatgagaggagcatcaaggtgtgggggcagccacctcctcctgcatcctgtgccaccaaacgggggggtctctccagccttttgccctgccctagtcttcctcaagagcaccccctcttccccatggagactatgaagagaagcttgggagtgtgtgtgaactccgcttgtgtctgggcctctcaattattctaaattaatatgagatatgagtgcatactcagtctttagaaattcttttttttttttttgagtcagtctccctctgtcacccaggctggagtgcagtggtgtgatctcagctcactgcagcctccacctcccaggtccaagtaattctagtgcctcagccccccaagtaagatggaattacatgggattacaggcatgaaccaccacactcgactaatttttttttttttttttggtagagacagggtttcaccatgttggacaggccgggcacctgacctcaagtgatctgcccgcctcagcctgccaaagtaaggaattcattatttttaaaaaaatatttcttcttactcactccatggcaacaaccccctccttcctcccatactctgccaaaggtgcaacaatttcatatcctgcctctcattagagatgctttttatcctttgaagtttactttcctggttgccttgtgacctcaagtctctgatgagtttaaggaaagaaagttataatattgtacactgttcctcattttcttgttgttaggatgggggtgacattctctcgtagctttctatattccaagcaggagcagaaatcctcatgtattattaatagcaccacctttcacttcataatcactgtgggctggaggataaattatatggctgcagaaagttaggcccccactcgaggctaccagcacctggcagatgcattcagggcaatgccagctacagggctttttttcctctgggttcctgcttagctttctggccttagaagatttccttgactttcctataagcttaggtgtgcatttttaaaaatgtttttttaaaaactatctcatccagctttttttttttttttttgatacagagtcttgctctgtcacccaggctggagtacattggcgcaatctcagctcactgcaacctctgcctcctgggttcaagcgattctccttccccaggctcctgagtggctgggattacaggcacccaccaccatgcccagctaatttttgtatttttagtagaaacaggtttttgccatactggccacgctggtctcaaactcctaacctcaggtgatccacccacctcggcctcccaaaacgctggtgagctaccacactcggccttatccagcatttttaagtggcagaaaagcttctccagatatctagtgagtcatacagctggaaacagaagttgaaaaaaaaagttttataatattaaaatgttaaggttttacttctttcccaattaacattaatgattcttgaaatcgttggtcagatgactgtatgtcaaaaagcaatggttaatatgaaattaatatttaaaaaaaggaatctgttaatgtcaaagatggactttactaagcttttcttaaatattctaaaaattcaatttatctggagggtgttaacatgaataatttaaaattcatttcccaactgcaagggaaatcaaattgattccaatggttttcctgaatctgaaggcttggttatttttaataattttttttggatgataaatgggtggcaatatgatgatagatgagacagagattattgttacagcatattaagaaaactttagacactgatgctatcataaccaagggtgagcctaggtgtagaagtcacaatgttcctgaagcagaatgttggtcaactggatgagaccagtcaactaacagaaatcagctgtgatgcccacagcaaccatgctttcaagaactcagtgggccggatgctggagagtaggtggtgctgttttaatgatttacagtaagatgccccattaaaggactgaatcattgcaggaatccccccatatatggaaacctaggtactccctgggttccttctaggtggcaatgtaatccaagcgtataaacgtgatactgctgaccctacatctgatttggcccatctggctgactagggggctgtctccaccattccatgagttttttctcctgtgtaccacccactgcaccctgcattgtgcactctgttgaggaggacaattacttcagatacagctggattgttctttaaagtcaaacatatgggaattgggggcttccctccaaatcagttcgtaagtccatttgtaagaactatatgtgacggcatgcttgaaagcttcagctacaacaacttcatttcttaaaaagtgatggattcaataatgttagatggatttgataggcatgtattatattatggaaaaatgaaaatgcattctctggagacacactggtagatttctaaattatgtcatatgcttggatttagcctgcaacaactgctatactctgaattgttcatctccggacaagtatttagtttgtggaatttgatttaatagtgccccttaaggtattagagtttgtctttccggtccacgttaaaagtctctgaccttaaactctgtacaaagaaagcccttcaaagaaagctcggggctgaattgaggaggattctgggtagaatcctctccgtctgggatccccaccctattagtttacttgaggtgtggcgctccttctaacaagttcagcttgaagactctgaactggctaatgccgttgtttattccattacatatgtattatgttggtttgtatccccacagaatcaccaagcccagtttacaggttgttcttctttatagacctgtgaaatcccctgggtgttccaaagagctttggggtttctgggaatgtggcagaaagaatacagtaggacgcgtattaggtgatgtctgcgctgttccttcatgtgaaagagcagtgcgtatgtaagcaatgttagggctctgcaggcaagagtcagaactggggggaaaccggcagttcattatgtccagcaccttccatctcaattgacacatctcataaagtcgttttttaataaatcagctgagtcatagagaagtgtaagtgatatggtttggctgtgtccccacactaatctcatcttgaattccgacatgttgtgggagggaccccgtgggaggtaattgaagcatgggggcaggtctttcccatgtttttcttgtgatagtgagtaagtctcacgagatctgatagttgctataaggggaagttttcctgcacaagctctctctttacctgcagccatccacataagatgtgacttgctcctcctcgccttccgccatgattgtgaggcccccccaccagccatatggaactataagtccaattaaacctctttcttttgtaaattgcccagtcgcgagtacgtctttatcagcagtgtgaaaacagactaatacagtaagtcatttttgcctgagattagatattacagaaagtcacacagaagtgttccatgaagtttagaaataaaaattctagaagacatacatttgtcatgtaatggttgatttttcttcaaaatattcagtgaatgtacaaaacccattttaaactaaggacatgtacaaattacttaaattcgtccccagatgaacaattggagaaaatatgaataatgggggaaaatctaacatgtgcagtattcaaaattaattaaagaaagggcaaatctttggcatcaaatggaacaatgttttactagaggtatgcataaaaggaacactgacatgaaggaaagcatattaaaatcttgtgtttgaagattcactgttccctagagttgctctagtaattaagattagcaagaaatttgtcagatattaaaacaaaagaagagtttcatcagtatcaccagcaatctattgttaaacataaagtagccaaggaagcaccttaggaccatgtttaagagctcagggaatccccctccaccccataggattcctgtcagtttacctccagagaatgggcttctgctagaagtctgggagcactggattagtcaagaaggccaaatacaaggatcaaaaggacaccacagctgggcatggtactcacacctgtaactccagtgctttaggaggcagaggcaggaggatcacttgagcccaggggttcaaggctgcaatgagctatgatggcgccactgcaccccagcctgagcaacagagcagcaacctgtatatacatatatatatacacacacacacacacacacgtatatatacttatatatatgtgtgtatatatatatatacatcttatccctgccaggtagccatggaataaggatgtgaaggtctataggcacaccagctctgactgagcaaacagtggtcagaagggaggaagaagcttttctggatcaaatgtgtcaagcaggccctgagaagaaaagacagagcagcacatgatggcagccatacataggacatgaaaaggcagaagccaaagaagaataggtagaaaaagccaatggtcctgttttggtcttttcagttactcttcagtacaccgtcagctacttaatctccctttcctcttgtttaagtgaaagataatataacaataacaaaacagttgtatttatttttaacataacattattgtatataaacatttatataaaaacaaaataggacaaatcactaatggtaagtttttaaaaataattaaaatggtgtgtttagaggggtatgaaaatggaattgttaaactctaaaagagcacactgtacatgaatgccttagaagaagcaactcaataggatgatatattttaagggattgaataatagaatgttcttcccagaatcttctacaatggtaatatcagcatagtgcatttattcgtagtacaaccatgtattttgagtaacatgcctttcaaaaacagtactgaatatacaatgcatagaccacctagaataggtatgaaagaaagaaaactgaaatgaataaaatggggaaaattccatgttttattctatgggagataaatttcttatatatccattagaattgcctatgaaaatcaaaccacaatttgagtccattttcctcatgatattctaataaccaggacttccattttcatcaaatagaatttattatacatttacttttgaatcacattgggctgggtattacttaaaacatggcgaatagaaatagttcatacctcttagaagcttatagattaaatggatagtggtaaacacgcatatgtaaaaatcatgcacacataatacatacttagtagtacatgtctaaacaacatgaatacatgctaaactaaattacttctagtgcttttagcataatataatggaaaaatagtttttaagtgtgtttagccatctgattagctgagacacaaactctccctggaagcctacagctgaggtttgagggggaagagggaaagctttcagcctatacaggttgggggattggaacagagaccctcacataacactggacctccaaattgcaaagagttcttacctttagagatacacagatgaaactataggatgtctggcattttcttcaaaataatccaggggtgggaggaatgggtagagattacagatgcaaaaaagattgactatgagtccataattatagaagctgggtatggctacatggaggcttattagaatattctattttgtatgcttgaaattttctaagataaaaagttttatttaagaaccattaaaagaaaaaccaaaaattgtgtaatggaattgaaacaaagctgacacaagatgagaatccaaaatgtctaaagaacttctagaaatcaataagagaaagacaaagaaacccattgaaagatggtttaaacaaacaaataaaccgacattttatgaaataaaaaaatgcccagaccaaacgtcagaagatgtgcagtctcattagtaagcaggaaaaagcaaattaaaaccacaatgagatattctgtatgttatgatatgctaggtaatattgttgccaccaacacccaagaatcataccaacaaaattttacttctcactcacacaaatgtccagtttaggtcatctgggaactgtggtcagtcatctgttcattatgatcactcatgttcccagggtcacacaacagttacctccgatgttgtaggcctccttgtcagagggaaggaaagcatgggggtttttgcatcagtaagtaattgctccagccaggagctggcagacattccttcacaattcattacaagtcaaactcactcataacaaggatttaggaagtgcaattccttctactatgtgcttggaagcagagaaaactggaaatgtttagggaaaacaaatgactttcacagatgccagtttttttctatcagattggcaaaactatatagtctgacaaaaccaaatattggtgaagatgtagagcaataaaaacacttacacacttttgactgaagagtaaattggtaccactactttaaaaaacaatttagtgttatttagcaaagttgaaaattcatatagtcttcgactcagcaataccactctaagatacatactttagaaatagttttgcatatgtgcaccaaagacatattcaaatatggtcatagaagcactgtttgtaccagcgaatcaaacaagcaaaaagaaaaactggaaatagctctcatgtccgcagatagtggataattttttgaatattcatttaatgaaataccatttaggatagaatatgaatgaacagatacatgttcatctggttgaatttcagaaacatattgaatgaaaaaagtaagcctagaattattgtatttgacaaaggtctaaaaaagttaaaattaatatattattttaggattacatacttaagcaaaaataaataattaaaagttagtttaacacaaaattcaagatattgtacctttgagtacactttgagtgtatcaaggtagggatgttaccagggacagatttaaggtattgataatattcttttcgtcaagttgtttggagttgtaaacattgttcactttgttattattctttaacttgtacatatacagtatatgtactctatgcatgcaaaaaagcatagaaatatatcttatattgtggagaaaaatcagtcaatggaaacagaacaaaaatgacacagatagtagaactagtagtcaaggacattaaaagaactattataaatatgtttcaaatgttcaagaaggtacaggaaatagaaaaacaaaaaatagagaatatcattggaataaaaaactggttttttgagatcagtgagtgataaacctttagctggattcatcaagaaaaaaatagaaatgatacaaattattaattatagaaatgagagagaggataccaatacagttgtagatactaaaagtataagaaaatattacaaccatcttttttgccataatatttgacaacttagataaaatggacaaattctttgaaagaaaccacctgccagagatcactcaagaagacatagattacctggatagctcaatagctattaaagttattaaaggcttctaactataaagctattatagttagaaacctccccataaaacaaacaaacgaacaaacaaacaaaaacccagaaaacacatcaagccaagatggcttctctggttaattctaccagacacttaaatcagaaagaatatcaattctacacaaaacttcaagaaaatcaaagaggataaaacactttgctatccatgttgggcgcagtggctcatgcctctaatcccagcattctgagaggttgagatgggaggattgaggccaggagtttgagactagcctggtcaacatagtgagaccccacctctacaaaaacaaaaacaaaaaaaaactggatgtggtggcacacacccgtggtcctagctacttgggagggtgaggcaggagggtcacttgagcccaggaaatcaaggctacagtgagctgatattgcaccactgcactccagcctaggcatagagtgagaccctgtctaaaaacaaacagacaaacaaaaacccactttgcaactcaatctatgaagccagcattaccttgataccaaaaccaaagatattacaagaaaagaaaatctacaaatcatgtcccctgtgaacatagtgacaaacactgtttataaattttaccaaataaaaggtttatctcaagaatgcaaggttggtttaacatttgaaaatcaacccatgtaattcaccatattaatacacttaaaaagaaacaccatatggtcacctcaatatatgcagaaaagcatatgacaaaaattcaacatctattcatgatgaaaaaatttaaaaaagaaaagaagaaaattcaaccacataaagggcagctatgaaaagcctacagttaacacctcacctaatggtgaaagactgaatgcttttcatttaagaacagtgcaagtatatccattctatcacttctacttgtcattgtaacaaagattatggcaaatgtattaatgaaagaaaaagaaataacaaaggcatacaggttaaaaagaaaggcataaagcagtctctattaacagacaacatgactgtggaaaattctttgaaccagtataggtgatttaaaaaaaatctaagaaacctacacacacaaaaatattggaactattcagtgagtttagcaaggttaaaggataaagagtaatatataaaaatcaattttgtttctatacagtagtaaagatcaatccaaaattgcaataaaaataccatttaaaatagcagaaaatatgaacttaagtattggtttgaagaaaacttatgcaagacctttatatcaaaaaccataaaacattgttgagataattgaaaagatgagatatacatgcccataaatcaaaaactatattatttagatatctattctccccacattgatctttagattaaatgcaatgccaataaaaaaatcagcaggcttgttttttagaaattgacaaactgattctaaaatttatatggaagttcaaaagatgtagaatagtcaaaaacatctgaaaaagaataaagttgtagaactcacactatacacttgagtttatatttagggtttatcatggagctatggtaatcaagacagtgtggtattatcataatgatagatatacaaagatcaatggaacactctagaaagtttagaaacagacccacacatatatatcagttgacctctttcaaagggaaactgggtgaagaaataataatctttgttttttttttttttttttttttgagacaggtcttgctctgtcacccaggctggagtgcagtggtgagatctcggctcactgcaacctccgcctcccaggttcaagtgattctcctgcctcagccaccagggatgctgggattacaggcatatgccaccacgcgtggctaatttttgtatttttagtagagatggggttttgccatgttgctcaggctggtctcttctgagttcaaatgatctgcccgcctcagccccccgaagtgctggaattataggtgttagccactgtgcctggccaaaattgaacacttttgctcttcaaaaaaatgaaatgaaatgaaaaagaggaaatgaaaaggcaagccaaaatctgggcaaaattatctgcaaaatatatattttatgaagggtttgcattttatataaaataaacccttacaacttaataagaagagaaacaacctaattaaaatactgtgcaaaaatcaaatagacattttacaaaaaagaaactgtacaaacaggaaataagcacataaaaagatgctggacatcattagtcattaggaaaattcaagtttaaatcacggtgagttgacacctagcatgatggcaataatcaaaaagacacaataagtgttgggaaggatatggagcaactaaaactctcatgcactgctggtgggaatggaaaatggcacaacacttttgagaacaattttatagcttgaaaagttaaacattctctactatatgtgtcaacctaggtatttacccaagagaaacgaaagcatatgtccacataaggacttgtccacgaatgttaacagcagctttatttgtaatagtcaaaagctgggaacaacccagatgtccttgaacaagtaaatggacaaatatatttcagtccaattcagcaatgaattcagcaatgaaaaggagtgaactattgatatgcataacatagatgaatcttaaagtaactacacaaagtgaaagataccaattaatataattgtatttatttaaaaattttaaaaatgcaagctaatctatagtaatggaaagctgatcagcaattgcctggtgacagagatggcaggagggaggaaacttttgggggcaatgagaatgtttgttatcttgtgatgatgggtttcatgggttcatacatagctgaaactgatcaaattgtatactttatatttagcttattgtatgccagttataccttaataagattgtaaaaaaaaatggatattgtctcttaggtaataattgctcatatgcatatgcccattaacatatgtgtcaattttcagggtaccatattctttgcttagagcaggaaatagaaatataatatgaatcacatatgtaattttaaattatctaaaagacacatttttaaacatttttaaggttgaaaataattttaataatatattttacttaattcaatatagagtattttaatcaatataaaaactataagcgaagtattttacaatgtttttcatactaagtcttcaaaatccagtatatactttacacttgtagcacgtcttagatttaataacatttacaatggagaaacagattcatatatcaaacatgcttaaaaattttcccataatagaattgagtatctttttttaatttttaattttgaaataattacagactcataaggagtaaaagtagtataaacagatcccatgaatccatcaacccatggtaacgttttacataactatagttcattatcaaaacaaggatattgattggtacaattaactacacctcatgcatatttcactgttatttatatgtattcaaatgttttatctgtctctgtgtatatttctataacgttttatcacatgtacagaattgtttaagcacaatcacaaccaacatacaggactgttccatcacacaaatgatctcctagtgctgctctgttatagttaggctctccctcctcttccctaacctttggcagccatttcatttatctgttttctctgcaactttgtcattttaagaatgttgtgtaaatgagatcatacaatgtgtaatccttcgagactggcttttgtcacttgtcataatgttctttttttttttttttttttgagatggagtttcactcttgttgcccaggctggagtgcaatggcacgatctcggctcactgcaacttccgccttccgggttcaagtgattctcctgcctcagcctcccaagtagttgggattacaggcatgcgccaccatgcccagctaattttgtatttttagtagagatggggcttctccatgttggtcagactggtcttgaactcccgacctcaggtgatctgcccgctttggcctcccaaagtgctgggattacaggtgtgagccaccacgcctggccatcataatgttcttaagatccaccctagctgtttcatgtatcagtagtttgtttcttttttattgctgaatagtattctactatatggttgtgtgttcaactattcaccttttgaagagcatttgggtcatttccggtttttggctattacacatacatttgtgaacagtttttgtgtgaatataagattttacctgcctaggataaatgcccagaggtactgttgttcagttgtatagtaaatgtgtgtttaactttttaagaaactgccagatgcccagagtggctgtactgtttaacattgccaccagcgatgtctgagagatccagtttccctgcatccatgccagtgtttggtattattagtattttttattttagctattctaataggtttataagtgatctgtcttggtgattttaacttacattcccctgatagctagtgatgctgagtatctttcatgtgcttatttgccaactgtctatcttctttagtgaaatgtctgttcaactctttggcccatttaaaaatcagattgttttctcactgttgactttagagaactctttatatattctatagataaatcttttacatatgatttgcaaatattttcttccagttggtggctcatcgttttcatcttcttaacagggtctttcacaaagcaaaagtttttaatttttaagtatctatttttaaattcaaatggatttaaattaaaaattcagtgaaaaggccagtctacccatcgtggttacctactgaatagcagaggcttagagaatacatggaatttggttagggatcattccaggtccgggaccacaataaccaggaccaggagcatgaatgggtatgaaaagttaaagtgaatagacagacccaactggcaaaaatgtggggttcgagataagtagagggggtccagctggaaagggaagttgggtgtagattgtgaaagttgctgcatgctaagaaaactgttgaaatcttatctggcaggctcagtgaaatccttaggttttcctgaccaggagaaagatgtgctatagggagtgttttaggaagattcacttggtaacggggttcaaaggggactggaaaaagagtaaccatgctgtccttcctttgcttcagtttccctgcacaaaccctcatgtgctaagacatgagcctggtcaaggctaatgttgaaaacatttcagtttttcccctaaccttgagcaccttcctctatcttgctcatcatagataaggtagaacgaaggggtttaagataatatctggtaagaagtcaggtagcttctaggtattatacagaaaataagtttatagagttgaaaagaagggtgagaaattaaagtttaaaaaagcaggaaaaactcaatcagaggaaagactcaagaaaatgacacttgttcttaatattatctcaggggtgacagatatttataaaattaatctgtagcttttcacgttaatcaaaaaatagaaaagactatcaacctagtgtcaatgattaatagatattatcatttgaatgtgataaataagtcaagaggtagactggtctcagggtgggtttgttagatcaacgtcattaagagcccaggctcttttagcccttcctctctgcctcagtgcatatgtggtttttcctcatggtcacaagatggctaccacagcaccaggcatcatcacgttttcacaacacaccacccaaattaggagtcaaggtgctggggttagggctgaagattgtggtacttcccctaccttctgacagggagaaaaatctttcccaagccctccattagaattctcattatatttcactgaccaggactggattacacacccacttgtagaccactcagtggcaaagggaaaaggggtagccatgactgactagggtcaaacatgactcaactcctgggtacattattgcccaaactaaaaggagataccaaacaggaaggggaatgacatggataagccagtaacgttgtcagccacatacaggaacaggaaaattgatgggagaacatcagggaatcagataagttttatgaaacattagtctaataggttccagaatgtactgctcatgtctgtcaccgtgaacatctggcaccaacttcacacactgtgggtggccagtacatattttagatcaactgactctagaaagtagcaaactgagtaagatttaaacatgtagaaatattatgagggccaggaaaccatgttgatcttaggaaagtgagctttctttatcagagagccagaaggacacaatgtttaacttttagtgaggcaagataataatgaagattggctctccacaacactgtgtgctgatggacagggcacatctagtcagaagatgaccaccaaccagaaaatgacgtgcacccatgggcacacaaaggatgccatttggggacaattaattacgtccttccacaaatacttaagcattattttatgtcaggcactgttctaggcacttgcattacactagttgagacaaatcacaataatttcctgttttcatggcttattttctagcataagaatgagtgaattacatagtaagtcagcaggtgataggtgctttgaagaagagacaggacagagtaaaagggatcagtaatcccaggatgtgtgctgtgaggtaggttaaataggggggtcagggtagatctcactgagaaggtgacatttgagcaaaatgtcctctgcacgaagcggggaaggaggaggcataagacttgtgtgggaagaggttttctcgaattagccaccccatttcatcactctacataggagaactacaatgataacacatacatgttacatgataaaacacatttgaagtgctttactttttaaaaataataggttttcattcatctgaagcaactttcaaggcagatagttttccaatactgccatccaaaacattattctggtatagaataattcatcccacctttaagaccatgaaggctctgtgatgccaggatgctgcaggcatttgggtttcaacccttattctgcttctaaccagctgtggatagtggaaaaccacgtgacctcttctgtgctcagtttcctcttctgcaagtggaagaagctgaataattttcaaggtccatctcaggtctaaacttctgtgactcccagataaagaaatctgcttgcaggagtctgcacaatgctttctcactggctctgggagggctccttgtgctgtgtagcccattaggaaaacactgaagagccacctaatgacttaatgggctttaaaatgggtggctaggttttaattttctctgatagcagattccctgagggaaagtgtactttacagtggataataactacataggatatttcgattgccgcagggtatcttatttggaaatcagttttcagttacattcctctagcaatttttactatcagaacaaaatgaaagcccccaaaggaatgtttatacagagctgaaatgtgttggaatgtcagaagtatctctctaagagttaagcatctgtaaatatttaaaatagggtgaaatcacaaatactgctactactaatctctttcacatttgattacagtccaaaaagaacaagcaaaatctccaggaacacttttatattaaactgttaactctacggccatgtctaatgactcaaattataaaagaaagttgggtgaaatgcataattcacctattcaggcatacagtcagctctacacatgtgaaaacagatctcgtagaggatgttgaaggaaatattttcctctctggtaaacttaagtatcaatattatcacataaagactaggctacacaataaaataaagtgagccattctgcctgaattttcaggattcagctatccccactcttacccacacacatcatagctttacacattcctctagttttctttggttatggatcagacatcatgttacatgttttagtcacatcatctgatttaatctgctctacagcaataaaaagtagcaattatttgctctattataggatggtggaaactgaggtcctaagaaaatatgtgtgtgctataaactgcttttccttcaaatggcaaagaacactgaaggaagatagtccttgattgtagacattcattcattccggaactcatgctcttcttaatatactcaactctaaggaaaccttcctaccaatacagcccactcccctcttcttaaccagtgttcagtggagatgggaatttggaggagtgatgagctcattgtgtcttttttctcctttatttgattacctccctgccatcttgccttttctgggctaaatgataacacttccttccgtttctctttggaaccattttcaaaaaacatctctttaggagtggaatcacaaatgagaaacagtattttcataaggaagagtaaaatgaaaggactgccggagcatttttacattagcacatgacatttctaataatctacatagaaggattttttttttagcatcctcctaagtgcctctttgaaacctctgtgacatatgagatagctactgactaatttgttctggcagaaaaatgagtttagtgttattgaaaatatgttttctatcaccagatcttgttcaggtcattgagtggcttttcactggtgaaggggagtaggatttatctccaatgaaacattatggagcggagagaaataacatttgacctatcccaacattagcaaaatgcaaatcatagtcaattttccatagggtgatattttcatactttggatttttgtagttgtgcatcagtctgaacattttaaagcaaggtgaatgaagggtccatgaaagagactgtgaataagccactgtgcgaagctaactttaaagtttccagcttagtgactgagataacgccatttctctagagaagttctaaatatagaggaataatggatagcagtttactgcaagctatgtagtgccctaaaccagcaggggctgggcattgagtggagaaggattgcaattttgaggttgattaaagatgcagtactgacaggttccaatgggtgtagcagggtgggggaggggtcgcatctcattctttttttcctccccactcagaggtgtgggtagctgcagtgtaaactgctgatgtgggtgattaggatgttaagtgggagaaaagacagctcttcattattcttttggaaataaagagggataaaattcagcaatcatcaagccttttcatttccctacaatttgtcggtgcagctaggaggctggcttcctttccacagacgagattacttcctgctgacatcaagtgggggtagggtggaccaatgtgtgaaaaagaaaattgagctcgaagggtagcttttgttttgatgtttgttagatgtcttttcttgtgttaaaattagatgtatcacctactggcagccacaggattttcatgttgagaatgcaaggcagattttgctagatagatctccaggtgcataatccactgtcagttttgataaagaattatgacttcttggactttttctttagctatgcagaagctaaaaatctgataatttagcataatgcttgtgtaagctgtgctggagagtaatacatagtcgctttcatactaacataaagctccttggtcaaaattacaccaataatgcatatttaattagcgtttatagatatctaaagaggaaaaaaaacccacaaagtttccctcacagatgacctttatcggggatgccttagctgagttccttcagatgcctcagggcaaaatttcagccccaataaaaggagatgacatatggctggttatttcacaagatcgccatctttagattgaatttaacttttttttcctcagagctagagctttatggagttttcgggttttggaggtagcttgatggtacagtgcctcagatttataatttctaattaaaatgtgtccttcatatcaattttagagaagtaccaagcttaattttggcatttaatcatcgaaatttgttttgacatttttgaaacacagatcccaatactaagatgtaattactttgcaacagatttgcaaggaaggcgacaggccacagaagaacttctaaacacaggcaaaaccagacgagtccagtgggggaatggaagatagacctgaatgtcaaaaaactcaaaagtaaatatatggttttccagttttctctgtctgagggctcaccttgcacacattaactcttccctctctcaccatgcttcgccggtttatccctcagcactcttttgatttaagggaagaaaaccaatattaaatgtaaaagtaagttttctcactaacagcccaaggggcacagctggctaggctgtgcaagagcacgtggccgtctctagatctatcggctcagggcctatttcccagctcacagaccctgacaggacgctccgatgtggtctgaaggacagagggcatttagaaatgtcctcgcctttccaggtgtcctgaggcctagaaaaaaaaaatccatcacaaggatgagatttgtcaacaggacccctccagccagcctaccctcctggctcccgatgagggaggactgtccacggttgaccttgctaaaggacaggatttaaaaacacggaagcaggacgatgattggaagaatccaaagcttgtattccaagcttcagattggagtggaggcagctagacaaagaggtgagaaaaatggcagttttggtgtcagagagacctatgcttgaattccagttctgacactcagtagctctttgatcttggacacatgtttgaacatctctattcttctgttacatccattagaaaatggagaatataatatctaccttttcgggttacagtaagggctgaatgacctcatgtgtgatacacttagcatgactttggctggtagtaaggatttaataccttttaatttctttcttttctctcagccttgtctttcctcttagcattggaaagagttctatttatttatttttgataatggctttttaacttatgttgttaaaatgtagttgttttccctgaaatggtgcaacccaagtttagataataataatacttctttattttcatttatttatttttttgagatggagtgatatggtttggctgtgtccccacccaaatctcatcttgaattcccacgtgttgtgggagggacctggtgggaggtaattgaatcatgggggcaggtcctgtgctgttctcctgacagtgaataagtctcacgagatctgatggttttaaagaggggagtttccctgcacaagcacccttctctttgcctgccaccatccatgtaagatgtgacttgctcctccttgtcttccaccatgattgtgaggcttccccagccacatggaactgtaagtacaattaaacatctttcatttgtaaattgcccagtctcgagtatgtctttatcagcagtgtgaaaatggactaatacacggagtctcactctgtcgcccaggctggagtgcagtggcgtaatcttggttcactgcaacctctgccttccgggttctagagattctcctgcctcagcctcccaagtagctgggaccacaggtacgtgccaccacacctggctaattttttttatttttagtagagatgaagtttcaccatgttggccaggctggtctcaaactcctgacctcaagaaatctgcctgcctcggcctcccaaagtgctgggattacaggcatgagtcaccgtgcccagcctaataataatacttctttaaatatcgatcaggtgtgaactgtttcatgacatctagagtataagaagtgtcatctctttttatgtttttgaaaaagaatccagtttgagctcacacctgctgagtttggcaggaacatccaaaatctacaggtgaagggaggcaaagagaagggatggggaagccccacccatttagctcatacagacatgctagaggctgattcactgcatacgaatggagaggggcctgatctccagcatttacactgctgttgcccttattgttccttcacttactcagagctgctggctggggcccctcctctgtctgctggcatcctgttccacagattctgcaatgagaagcccaccaccctcctccccaggggcaactgtcagaataaatgaggctgtgatgtggtgacaaacaggtcctagatattagttcagttgactctcgagaaatgcagaggttagggctgtcaaccccctgtgcagtcaaaaatgcacatataacttttgacttctccaaaacttaactactaatagcctactgttgaccagaagccttattataacataacaattaatatattttgtattttatacatattatatactatattattacaagaaagtaagctagagaaaagaaaatgttactaagaaaatcataagggaggccaggtgcggtggctcatgcctgtaatcccagcacttagagaggccgaggcaggtagatcacttgaggccaagagttcgagaccagcctggccaacatggtgaaaccctgtctctactaaaaatacaaaaattatctgggtgtggtggcacacacctgtaatcccagccactcaggaggctgaggcatgagaattccttgaacctgggaggcggaggttgcagtgagccaagatcataccactgcacttcagcctgggcaaaagtgagtgagactctgtctaaaagaaaagaaaattataagggagagaaaatgtatttactgttcattaagtggaattggatcaccataaaggtcttcatcctcattgtcttcacattgaataggctgaagaggagaagaaggaagaagaggggttggtcctgatgtctcaggggtggcaaaaccagaaaaggtagagaggtgggagagaaggtggacacactcagtgtcacttttctagaaaaaaatccgtgtataagtggacttgtgtagttgaaacacatgcgttcaagggtcaactgtagtttaaatcaacagatgtttatttatttccaagcctgcctctttctttggaggcttggggcaagagtacacacagaggcccacataccatatgtctagatatgtaacacttataaaccagggaacaaactgttaaatgaaatataccctcttaccttgaaagatataccttgatagcaacctagataatcaatttgagtttataattctgagacttcctgaagttctgtgctacaacatggtggtgtagagtgctacctttacacctgtccttctcctggacatccttccctgtgcatcccagctctgtaagcatggcctttggccacccctcagatagaggatgactccaaggaagagtggcatgcaggctctggaagcaagcttgtggacatttgggcaaagaattctgaggtccttgttattcagaccatggaggagaatttgcaaatatggaatccacgaataacaaggatcgagtgtattttgttttgcttgcctgtttcttgtcaaaatttactgagagaaacttaacaatggggtgcaccgaaagagacaaaatggcgttaactcttccaaccgtctcagagcaaaagagcccagaatttaacagctctgtgtcaggtcacttttctactgagaagaagggataattccttttttctttttgctgatagatctctgggtggggtggatgggaacaaactaagcccaggcaatttatatcacattagtgatcatggtgtcacatgtggcataagacaggcactgtatcatttagctagtgcattcagactccttggctcagtaaaggtggatagagtgggtaagttgttatgacttccattttcaaatgaggaaagcaagaaagatttagccaggaactgagaatagagaacaaagagacctggggactggctttctggcaccttttctctttctcactgcttggtgctcctaggtgtagctgtctcagaatcaagggggaatcctttaaatccatagcagttaacccagcaagaagttctgagcagtctagcctgagctgccacgcataaaaagcaacataaggttttatgcaattgcttgagcattcctaccaagaaataccttttgcccattttacttgtattcctttatttggtgatgtgacctagttcttgaagttctggaaaacccttcagaggctatagagtcacttttctagataacccactttggatcatgcaactcttcataggcacaatatgagtaagcagagcacctgcactaaccactatttccattcgaagaaaataaaataaaacctaaacaagagaaaaagaggagactccaaatactacttcacctaagggagtttgtttactatctgtttgtcctttgcaaatgtctttccttcaaccagtgaatgacctgcttgctatgggccagacattgtgttgatggctatacatacaatttcgtcaaatcctcagatcagccttgtaggattggtggtgttctcttcccatacatgagaaggctaaggcttagagaggttaattaacacagcaaaggtcacacagctaatgagtggctgagctgggattcagatgccatcggtctactcctaaggccatgcccttaatcagatactatcctgagcacactggcaggttcatcataatatcaatatatacctcgcagggctgatgtgaaaattaaaagagatgatatatataaaaagtgttgggcaaaccataaaatccttacaaagtttcactatgattatttctcttggttttacacagggcttgctcagctgtccttagtggccatgtctatattaatcccataatttatccttaaatcaccgaagtcttatttgcctggctttttccatagccgaagggaaactgaaaatatgccttttgtaagaaagccttaacacctgctgctaaagggcgctaaaccatctggatcgtcaaaggagtataggctcacttacaaaagacgtagattaatttggacattgtttccctacataaaccaggcataaggaggcaagcaacccagggaaggataatcatgctaagagtggaagcacttattagaaataattaccatgtaattatccacaacttccctggagtctgtagcctgtggctagtcttaggaggtgccccttccttaccaagtacaaggagactgaagcacttcaataagttgatcagggcatgtgtctgtgtcttagagcttggttaccgccacctgggcctacttctcacttaatgaagccttcaatgcttatagctgaggtgcagaacagctgtagactttcttcagctggatctaccacccacccctcccttttttaatagcacagtgttgttttgcaagtgtgtgggtgtgacacatctggcttcgttccttaagaaaagtgtttattgaacaattaattgtggtgcatgggggctgtgacatttctcttttcgctgtgcctctgagctgtgtttccaggctacccggtggccccattgaagatcagtaacaacactgaaaaggaacaatggctatagaagccttgacaagtgcagggagagacctagacacttccaagaggggcatgattgcctacaaggttaatcaattccccttgtttccattgcagctccacatttatcttgctggcagcagctgcaaatgtaaaatcagacctacaaacttagacaattggaattgtatttacctgatcaagatgtgtaaatgatctattctctgtgtcttcttaactggaatttaaggagttcagcaaagctccggctataggtcagtgagaagcatgtttttgtgtggtgtccagatacaatttagttgtcttggatagacagttggaaagaactgcagtcacagcctagaatcttgattgtcatgatcaccattattattgttacagctgccattttttgagcctttatatatgacaagcactttatatgtactactatcttatttcttcctcttgataataccgcaagatagctaatagtatttttatcatgtacagatgagaaaactgagattaagtaacttgcttatggtcattaggtgcaaggttctagattcaaactgagtctgacccatgctctgaaccactagggcttaacaaacccaaacctcactagaatatgcaggtgcagagaacacaagggcttgtgatcgacctgacagttttaaatcttgttttcaggaaagccaatcaaagaaggcttaacgaatctctttgcaaaccgatttgttggccaacctttgaatattctcaatacaagagaagggctaaaaatatctattgcagcagtgaagtggccttattttgaaaatgagtcttcatagaagtaatcaagctaaaatgaggtcattagagtggaccctaatccaatgtcattggtgtcctcataaataggtgaaattgggacacagagacagacacatggaggggagacactgtgaaaagacacagagagaaagccctggaaggctgaagcagagattgggttgatgctgccacaagccaaggaacacctggggctaccagaacctagaagcagcgaggaaggatccttcccctccaggtttcagagggaacctggccctgctggcactttaatttcagacttgtagcctccagaactatgagacaataaatttctactgttaatagctgctcagttacaacctccctggaaagcacatactcctgtcctatggttctattctgttctgtactattttgttctattttattctctttcattaagaaaatggatttccaggctgggcacggtggcttacacctgtaatcccagcactttgggaggccaaggcaggtggataacttgcggtcaggagttcgagaccagcctggccaatatggtgaaaccccgtgtctactacaaatacaaaaaattagctggctgtggtggcgggtgcttgtagtcccagcttactcggggggctgaagcagggaatcgcttgaacccgggaggtggaggttgcagtgagctgagattgcgacattgcagtccagcctgggcggcacagtaagactctgtctcaaaaaaaaaaaaaaaaaaaaagggaaacagacttcccaatctactaattttgaacaacatcagtctctagagctcagcattcttgccctttccaagctggctccagtctaattttctaacctcagtattagttaacccccgacccaccctgaacacgcctggtgtcgcacagtccatctgtccaagagcacagcatcaacctgagtgcaggtggcctgctcattgcctgtgctgccacctttcccctcagtggctattcattttttaatgtctagcttcaagtcacctcctctgacaggattcccctggctcttcgggcagagtcatttggtccctgtcctctgttcccccacacacacactttatcagccccaattacagctcttatattgtatgatgggatcaatctgtgtgtccccttcatcccttccccacaactaaacttcaagttccctaaagaagactatcttttttcttgttttttctttttaatcaagtgtctagcacagaacctgacacacgacaaatccgaatacagggtaccccaaaaaggtcagtgcagttttaagctttaataacttcttaagtcgaaatgctacaaacttacaaaactcatttgaacgtttacttcttttgactctgttttaattattttaataaatgtaattttctcttctggttaatgtatgccatatttcactcaggggaactgaaacaaaaattaaagttaaaacgtgttattcaacattcacagagataaatagtgttaaattaactttcaaatggtgttcttataagttgcagcatttatagccttgaggttactaaaactgcaggaagacttttgggacaccctgtttttgaataaatatgcttacagcatggctcctcctgtgtttactgtaaaattcagtttagtttttaagtggttccacgtgggcacaggtattctttggccagtagttgtccttatacttagacccaaggagcccttgtccagggcccacactttagaaggcaaattccaaattccctccccaaatggcactgagcctgcttcaagggtctgcacgccagcagcagtgttatctgctaatgtctgtactcccatgggcgaacattgctgactctggtgttcatactcttgtgcccaagaggtggctgggggacagctgcttctgggaagcttgaccgagcttccaccaatggactagagccctgtgggaagagaggaggtggctgcaaaccaggaactggaaatcgcctctccctgcaccatcatatttaagtgcagatcatccaaagtttcaatctggcctttcatgtcaattcccaaggtatatttgtctaggtaggaaggtagaacttattgtacttaatcatttgttaactttgtttataacttttaaatatttaggtatctggcctacagatctctgtttgtacttttgccctgggtcctgcatatattaaaggtgggctgattttggccttaaaagaattattttcccacaaacagatttttctccttaaagagctaggtgggtaggaagtacttccatatggaaggtatgatagcaaagaatgactgcagtcactgagcacatactttgtgctgggtgatttacacatagtttttcagtgagtcctcacaacaacccaataaagtaggtacttaggagaaaacagatattcagtgacattagtttgttccagctcgtaaaactagtgctttagttagccttagttgcataacaaactacgcgaaactgaaagtggcttaaaacaatttttccccctgactgtcgggttggctgggctgctctactgcgggtttccctgggctcactcctgcagctagctgtgaggagatggaggttggcccgggacagtgcagaagattccccgtggcctctcccacctgtctggcagttgacgctgctgttgctgataatgatgaccctcctttattttgtgttatctacttgccattgcaacctccttgccttttttagttcttgctcccgtactgtctggctcgatgcccagcacctggtggtggctaataatatacactcaggtcacttacaagcaatgtgaccctggatgggttacttaacctcacttagtttgtgtcccccaattgcaaaatgggtataatggtacagaacacatcaggtggctctgagactccagcgcagtgattcaggagagtgcttggcaggacagtggcacttggtgcgagcttactgctagtgcagctggaatgtagaccatcagcgaacgtgtgttgcctttttaaacagtgggaatcaaacaccactcgctttccaaatgtaaatcctgggcttgcaagtaagtcctagagtgcagaaataatttcaaaatacagggttgaaattttaagatagcagcttttaaaaactgggagacaaaatcaaaataaaacaaagggacaaaccaacctgaatggcaccccgccgatgctcttcattaatttttaagattccaccctggggctgcttgcttcttccagcatgggaagcgctgacttgatgcggccaggtttctgggaccaggaggttgggacacgacgacgcactttttgccctgtgggtctgggtcatgtgagcctagaggagcgcttaaaggctcgagttctgcagggtcctcgtgactgggtggggctgcctcacttctgcctgatttgggaagcgctgcaaggacaaccggctggggtccttgcgcgccgcggctcagggaggagcaccgactgcgccgcgtaagtgccgcctgccctgcgtgggtcgtgccagctcagcgggacaggtcctcgcctcggtccctcggacttagggagcgcggggcaggtagtctcaaaagccctcagcaccaacaggggagaaagcaccgtagagtgagactggagtggggtggtcgtggccgagtcaaagttcaaattggagcattggggatgtactgcgtgcgcccagccgagggaatactcttgaagtttcctggcacaactgtcgcacccggggttcttccaggctagaagccgtctcctactccaggagctcctccggggaagtacgaagctgtggggtcctctgccgctcttagcgcgggagggtggcgggtgcaggacactttcccttactccctcaacgcgtgttttggggatgtgttgatttcgtgggtgctcagaagcaagagaagcggaaaataccttctattttgaacgtgcccaaagcagggaaacttggcttctgcctaggaacgcataggaagacttttattcttggaatgtaggattagcagaaaccaagggcacaggagacaggtggaagcagctccctgtgaagtgatacagaaacaaaaggactttggaatatttgtggatttaaatcccagttctggccttggctagtgagtgacctcgggcaaattatttaagcctactaaacctcagtctttccatctgaaagatggagataataagtttttctttttaaagaattgttttattggaaatagaaataacatacagaaaatgcctccctagcacgggacttggcttttcagctaagtgtaattttccttatcagttatgattgatgatgaagtctatggtgaagacagaacaaaataatgtttaaaactctggccatataatgactgtggctatgcatgacccgtgtgcagtcacctctgagccccccaccttcactgcaaccccagtgggtcccaagcaaggtgcttgggttttggagctggacaattctgtgcaattcctggttccggtatccactacttggatgacatcagggacgtaacttatcttccacagcctcagttttggcatcagtaaatgaaaaaggagcccctcagtacttgcctttcccttccccggctaaatgtacaactcattttttttccattctttgctttgggatgagtaagaacaaagatgtaagcagagtaaaaagtccattgagaatacattttattagcttggtaaataacgtttaagaaagttgtccaaaaggagaattggagcacttttggaaaagtacactgcaccggctttgttggcatatgaaagtctgcatggtcgtcaagaccgtttggctaactgggcattctagatgtgtacctcatgctagtctaagtacaggggcaaccctttataatgtagtgtcagtgttcttaaccagatgttgttttattaatcttttataggagatgattgtttgggaggggcaattgatcccccaatttatataaatccagtttataaatcactttatagtgggattctactagcctgagaaataggtgggttttgactgacagtttgaatgacttagaagagtgttaagaaagtccactgggtttaccagttcactccccacaaaaaataaaaacaagaagcaaataatataacagaattctaaaaatgagtttgtttttgttttgggaagcatctagtttgtcagaggaaacactgagtgttgaatactaccctgggatgcacagaactaaggcaatgagaggggaggaactcagttcttgccccaggtatcatcagatatctgtttattagcacccaaatactcaaggtaaatagtaatgacccattctttctttttttgagacaagagtctcactttgtcacccaagctggagtggcaccatctctgctcactgcaacctccatctcccgggttcaagggattcttgtgcctcagcctcctgagtagctggaattacaggtgtacaccaccatgcccggctaatttttgtatctttggtagagatggggttttgccatgttgcccaggctggtctcgaactcctgacctcaagtgatcctcccacctcggccttccaaagtgctgggattacaggcatgagccactgtgcctggccagtgaccccttcttaataggaagtatggttcactaaagagcaaacatgattgttttagtttaccatttccgatggctttataatgtgagattttcattgactgaactaagaacatttaaaagaagtaggcaagcttctcagaattagaaagatacctctagaacaatgactcattaacgtctatattgacttgtgagagaggtaagctgggcaaaaagctaagaactgtactttcctggtaaaaacagtagtggagtttgaacttttatactatatttgcaagctcaaagatgtaagccttaatagacagttcacaagacagctgttacagcgtatctgaactgaaaccagggtattgagattgctctcccttaagagatattagaggtataaccttgaacatgaaatcacatgctttcattgctttaactcagaccctggggtgcaggacagggtggtggtagtgaaagagggggaaggactgtgtccttagatcttggagcctgaaaaagcaaattcttagcagcttatctttccaatatagttggaacttacctcctacctttttctctctagtctgattctagttattggttgtctttaaattgcttagaattgtttacaggccaaaatatttactttgaaccctaaaggtaagctcttcctcctttcctttttgggactctgtatcccattacccctctttcaaaaagatagtgccacatatctttttgtactgtatgattcttgggagagaatgtaacccaggtcccaagcgattcattttctcatttatttattcatttaactaattttttcccagtgtttgattatgaaaaggtttaaactttcagaaaagtcaaaagaattttgcaatctagaatctacctagattctattattgattagaagccagggtattgatttcaattgattaatcaactggcattgattagttcctactgtttgcctggaggtttgctcagcaaagctgtaaaaacataaaaggcagggtagggttagggaacaaggagagttttgatttggcggagcataggctaagagtttgtgaaggaatcatgtaactttggtttgaaaagttatgttgaagttgtatcatggaggacattaaatttcatcgtagaaagtttatactttttcctggttgcaggtgggagccattgaaggcatgggaggaatggagtgccttgtgttttagaaagctcactgaagcatcagtgttaggctaaattggagggaaaaggaccggaagtctgagagatgtgttaggaagctttgtaaatgtctcggggcaagtaagggttgaactgtggatagaatggagaaaaaaggaagtagaatagcacttggtgacagatttgacttgaggacttctggttttagaactcaggcatctgcaattataagggagcatcattaagaatctggaagtcgctgggcatggtggctcatgcttgtaatcccagcactttgggaggctgaggcgggcagatcacctgaggtcaggagtttgagaccagcctggccaacatggtgaaaccctgtgtctactaaaaatacaaaaattaaccaggcgtggtggtgcatgcctgtaatcccaactactcaggaggctgaggcgggagaatcgctgcaacccggcagcagaggctccagtgaaccgagattgcaccactacactccagcctgggcaacagagcaagactgtgtctcaaaaaaaaaaaaaaaagtatctggaagtcatagaaggaggaagtttgaggagaatgatgacgtcctgttatagagtcagtgggtttgaggggctgattggttatccttttggaggtgacagctgacagtggtgatactgtcctgaaatgcccaatagcaatgtcaggactgaacaaacatttggactagtgattaatactcaaggggtggttgaatgggaatgtacggaaagagaaaaaaggataagatcttgaagaaaatctgaatatatggagtccttggagcaaaaaataaatacaggtgttcaatgatctgttagtattttaatgcctgtttatctacagtgttactatagctgcagttctcattaataattgaccacagttcctaactaaaagaccatatctgcactagtaaagtgaagtaagtctttccttttcccattctgggaagttcagggggtaaggtgaggcagaggaaagaagaagagaaagttaaataaatttttttatctaggacttttagagcatacaacgctttttccaggtaacccacttgcagatcagaagccaggtaatttaatgaacttgtctatcttttcacaaggcaacagctactttttttttttttgagatggtgtctcactctgttgcctaggctggagtgcggtggcacaatctcagctcactgcaacctctgcctcctgggttcaagtgattctcctgcctcagcctcctgagtagctgggactacaggcgtgtacccccacgcctggctaatttttatatgtttagtagagacagggttttaccacgttggccaggatggtctccatctcctgacctcatgatccgcctgccttagcttcccaaagtgctgggattacaggcgtgagccacaacgcccggccaaggtacagagtttttaagtggcagaagccacatttgaagctgtgaaggcatactttgtttttttattttttatttttattttttgaaatggagtttcaccctgtcgcccaggctggagtgcagtggggcgatcttggctcactgcaacctccgcctcctgggtaaaagcgattctcttgcctcaccttcccaagtagctgggactacaggcatgtgctaccatgcccagctaagttttgtatttttagtagagacggggtttcactatgttggccaggctggtctccaactcctgacctcatgatccgcccacctcagcctcccaaattgttgggattacaggcatcagccaccgtgcccggtctgagggcatactttaaatctttcatgctattcccaccggactgcattgccttcctatatgctactgactgtatgctaggtactgtgttaggcgtttgcacatgtaggctccttactgtggcccctctgttttgagaaggtgatcagcagggaagctcaaagcacattgcacaattggttgttgcacaattggcaggatgtctgccctctgattgtgccttgttttcacccaacttcttgaactctacaggaagggccagcctctctcagaaatgccagcccctaaagctggtacaaaggtcatgcaaccttcctacttccctgagttcaacaatcacttcattcattctccttaagatcatacctaaaatcctggcttatttaagggtgaagtggagaaaagctctattttcacttatgcatttgttttaccccatcaatcttctgccttccctcattgtcccacagttgtgcctgtctaaggtcactttttgcttttttcttattttccttccctggggagtctttggagagagggtgcgatagaatgtgtagcctggagcaagattacccccagttgactatggaagccctgagaattatgctgcttcctcaatgcttttttcccaaagttagaagtagcaagttgtatagtgagtaataggatttgcattgtgaatggggtcttcctccagtgaggactttactaagtgcagtgtttatttgtagtggggcacagaactgcattcctcatgctttatgacagtatttagggtttactggttttcatgcatgtctctctttgctagaatgagggctccttgaaggtggatactgtccccttttcatctttaactactccacaagttttagcaaagtgtctcaccctttactgtatggttagaaaatctctgaagagttgagttctgttaaaggagagaaaactatttgtatagaagagtgtgggtagaagcctgtttgtttgtttgtttgatgtgagacagggtcttgccacattgcgtaggctggtcttgaactcctaggctcaaataatcctcctgcctcggcctcctaaagtgccgggattacaagtgtgagccactgtgtccagccgaggcctgcatgtttgaatggatcaagaaatgtgattgtttagtgctgagggagggtaggtaggttgcagggaaaggcctggctagggaaaagctcatatgctcctaagtcaagcaacaagaaatgtagaacgtgaaataaagatgaagccattctgagtcatgtgcagaccgtgggaaatatctccctgtatttgacaaaaggggaaatggcagccatagctaattctgaagatgaaaagtgttcctgcctgggttacttgatgaagaatattattagcaatcagcaagtcaagtggaaataatttgttttgttttgttttctccccttctaactattggcatgttggtagaattaacttggctggcaggagaatgaaccgcctgtgccgttgggacacaggagatttctgagctggcagactgcacactccatgtaaaaccatgactctttccaccattcttgtgtacacgtgtgcctgaacattggtgtctgcaaaatccgagacgtctgtttcagagaaaaattgctggaagccactgatttagaattttaataggatttttaaaaaatctgtgcttaaaaaacatgtaatttaatcagtgttgtagcccccctgcccccccaagaatagcctttcttaggtttcttaggttgtcttacgaacactgacagaaatgtgtaggttcagggcacctagagcagatgtggaagttctttcattaaggtagcctgcgtgaatcatccctgtgactcaaagcctgaagcagttttagcctgatctccttggctgcatttttagaatcttagacattccaagctgaaagggattttgggaattatatagtccatctaatctccagaatttacaaatgaaggaactgaagtccaaggggagaagtgaattggcttgcccaaggatatacctgttggcaaagtgaaggctggaacttttgaccttgtacattcaggaattgtggcagagcctttcctgctcgtcttagaatgtaatgtactatagggagcccgcagccatcctctacggtctggctttattgtctcttttctccctattttttagtctccttctaccttttctcagcacacctttcaagtgggatggaagttgaaacccactcagcagagaatatgagaaatacatgtgtaatgacacaaagttaggtgtgttagcattatgtccagcaaaggaggacacatacccaaaggttgggagaagtgggaagaggcctctgaaatatttggccttgtgctgaatgagtcgaaggagggatcagggaagaggtacccgctggattagctgctgccaagtgcccgtggcagctcaggaacttagactctcagcagtctctgttcaggagatgggagggacaaagcgctatcattggtaagaatacagcagttgctcagaaaaaaggactgtccatcatactgcagctgcagagtggccttgggtatatagaaacattgttaatgttctgttgggagcatcttagactgattagcagcagagctgattttcactcccagggatctgggtctctctctttctcggttaatggcaccactgtccacccacccagccatctaagtcacatctgggatcatcctagattctttgttctatttcaactctaatatataatcacaagtttcttccagttttactttcttcttcttcttcttattattattatttggagacgtagtctcgctcttttgcccaggctggagtgcagcagcgtggtcttggatcactgcaacctctgcctccggtgttcaagtgatcctcccacctcagcctcctgaatagctgggattacaggcacataccaccaagcctggctaatttttgtatttttttgtagagacagggttttgtcatgttggccaggctggtctggaactcctgacctctggtgatcttacccaccttggcctcccaaagtgctgggattacaggcgtgagccactgctcccagtcaaccttctaattatttttaagtctgccctctccacgctattattactagtttccagccctagttcaaactcctgtcatctttattctagattattgtgaaacatattaactggccttcctgcctgctgtctttctggcttaagtccatccttacattgatgttcatagtactcttttcaaattatcaagcccttcagtagttcctagtcactagaggataaagattcttagcatatctagaaaactctccctgatcagctctctactgacttctctagattccatcctatttctccacttcctacttatgcttttctcctgcctctgtattggttatctattgctgcataacaaatttttaaatgctataaattctatgacctcccaaaatagtgatataggcgttaagaagaaattatttgggcagatagtgagagtacaggagtcctcggtaaggttttccttttaatgaaaagcagaccccaaatcattttcttttctaacaaagagcagcctgcctataaaattgagctacacacatagacaagtaagctggaagcttgcacaggtgaatgccggcagttgtgccaataggaaaaggctacctgggctaggcatgttcaaaatggcagctccgtcttcccttttcctttccaaccatgtgtacagtagggaacagacagtaatgctggccaagtggaaagcccatttgcttaataaggttagggtgaggtggccagcttccctgcatgctatgtaaatgtcacacctggtccagccaatctgtgggccaatctttgtctatataaatcagacaccacctcctcaagcctgtctataaaatctggtgcactccacggagggccagatttcccattcagatgcccctctcttgcaagagagagaagtgttctcctttctctttcttttgcctactaaacctctgctcctaaactcactccttgtgcgtgtccatgtccttaatcttggcatgagatggcgaaccccaggtatttaccccagacagtgatgccacctcaataggagagacagcagcacatgagggaagtatagagggattcatttaattaggagttaaagtcaaggttggaaactagtttggaggtttgcagtagtccaggcaagtcacaataaagaactggattaggccatgagagtagatttagagaggtgaaggtgaatttgaagaacatctaggagattcagttggtgattgattggctgtgggaatgcagggccagagaaaaatggataaataaagaatacatcctggatttctggctttagtacttgagttgatgggagtatttccaaccaagatgaaaaatagggaagtaaggacaagtttagggaaggagataataagtttagtttttgagttcttcttaactacttttgatttttgtcaatataataaaagtaatgttataattctcatgataaaaaattttagtcttcttctccccacctccccatccttctcttatctctgagacctcctctcatacttttacctgtctgttctagcctttaattccaaatttccaagtaatctgcctaatctgtttcttgatttattggttttagggttacaccttttatttgaggtattttctattgacttattcaacaaggtgaaaattcaccacttttacataccttacctcctgactttcctaccctccaacctgtccaaatggtgatataattttctcttttgccaaatcaatatctaatatttaccttattatgtaaatatgctcactgctgagccaagtagtatactataattgcaatccatgtcttgtacagttttattttcttcatcaacttaaaaatttcctctttggattgttctattttctctatcactcttataacttctgtccaaacttgctgacagaactgtaatatccttattgatatgattttttacaaaaactcatgcggtagtcagttatagatatatttcttccttgaggacatctcatgtggatccccctctatcttactgctccaatatgcagtggttgctctttaaatataactgttattctgtgatttcacttcccctcttcagtcctagttcccctgttagtgggctcccatgtcttcctctttcttggtttacatccttgctttggtggaccacatttgccagtagctttctgagaagtggtatatgagaaacaaacaaacaaaaaaaactggctggttcccaagctgtatatgaggtgaaaaaaattaaattaaaaataaaaatcaaaatctagctgggtgtggtagctcacatctgtaatagcagcactctgggaggctaaggtgggatgattgcttgagcctgcaagctagaagctgaagtgagctgtaattgcagcattgtactgccactgcagcctgggtgacagagtgagaccctatctgtaaaaataataaataaataaaaataaataaataactcttatgtatttctgaatatttatttattctcatgattgatataggctgggcgtggtggcttacacctctaatccaagcactttgggaggctgaggtgggtggatcacgaggtcaggagttcgagaccagcctgatcaacatggtgaaaccccgtctctactaaaaatacaaaaaaattagttaggcatggtggcgtgcacctgtaatcccagctactcaggaggctgaggcaggagaatagctttaacctgggaggtggaggttgcagtgagccaagatcgcgccactgcactccagcctgggcgacagagcaagactccgtctcaaaaaaaaaaaaaatctaatacaattttggcttttgattctttttatgtgataccattctctttctgtcttcttctctggaaagtttctaatagtcacttctgaaatgtgttcttcatcttttcatttcttgatctgggaacttgatggttcctttaaatctgaaaaatcatatctatacctctgcagatattttcgcagtatttctttgataatttcttaccctgatttttttttttttttttttgaggcaaggtctctctctgtcatctaggctggagtgcagtggtgcgatcatggctcactggagccttgacctcctggggcccaagtgatcctcccacttcagctccctaagtagctgggatcacaggtgtgtgccaccatgtctggctaattttttaaaccttttttgtagaaatgaaatttcattatgttgcccaggctggtctcgaactcctgggctggagctatccttctgccttggtctcccaaggttctgggattacaggtatgagccacctgatctcccctgaattttatctgccatctttttctggaactcccattagtcagatattggtcttctggaaccaatcttctaatttccttatcttttttcttcctattttttaaagttctattatctttcaattatattttctgtgagatttctataacttaattgcactgtccttttatcaaattcttaagatttcttaagattatgtttttaattttcagaagcatgttcttgtattttattctgttgtttaatggatgcaatatcttctcttatctccttaaagacattttggtttttggagagttattctgcttcatacattgctatttcttgtgagtttttatttttgtgcagtttcttttggattttctaccaagttggaggctttgctcaaatacccaatggttccttgaagtctatttacatattaagtgtgagaaactaaattgctggttggacactgtgttgaagggatttgtcagcagttcactggctccctgcagttgattggtggattcaacatgcttcattagaggatcctacatgtcattatgtgtaggttctttctctaagagaatgcagttgttgcaaagaagaattcttcagtctcctgcagaggctgaagaatgaaggtatagagtccttactgcctacattctttctgctgagtgttcaaggggctggggtctgtcagtgtgtagaatttctgagttttcagtagggtatctcttccccaactttcactgtttcttatattcccaagtctagagtgtctctactgaaaatgtccaaataatgaatttgctgcagaataaggctttcacctctcttttagactggggagggacttgaggatctaaatgctgttatagactcttgccaattcctccattttcagccccatactttacttttgccttctgcagtaaccagtacttaccatttttgaccctttctgtagttgtatggtttgaattagcttgtttcttatttatattctgccttcattctctgcaaactatctgttcactttccatctaacaacaaaaatttgatctgccccatttactgttatctcttctattttcttgggtattgggatttataactttaaaaaatgtctttactctcattttagagaggtttcatgaggggacagaaataaacaaatgtttctgattcaccacgtttcagcaggattaaatgccattttgacatgttgagtttcatatacccatggggcatcaggtaaaaatgttaggcaggcagttggatggatgaggcagaagcttttgagaggcactctcattttgagtggtgtccacagaaagtgctggttaaagtgatgagaccactctttagtgtgtggaagtgtgataagctgtgggccaaggatagacttctagaagaattttagaagatctatgaaaacacaaattacagaaggcaaggcatcgataaatttgccaaaggggcactgtcagcatgtcagaaaagtcaataaagataaagacagatttttcttttgaatttagaaatatggagattattaatgattttggcaagatgatatgcacagaagtcagaatacattaagaaagaaaacagcgttttttgttttttttttttttgccttcagaaactgctgacagaagcaataatagagggtattcagtggcttatgaagcagatgaaccagcctgtgggctttttctgttgtctgcaccttggatactatacgtagtggattttcaaatacctaatgagggaatgcatgcagtgactttgttgcccttgcattagctgtgtgaagacattttggctggtttgatctgagatgtcatttctcatcctccactttctcttttcagggttggtgtgagcaagccagactgtatggggctttaaattctgggtatgactgatgaagctgatactattaaccatgtctttcttcaattgtatatatgatgaaattttgcaggataatagagacagtggagtataggggttacactagaataagacctaaattccatttttgaggcctctgagggagctcaaaacacagcactttgtctttctttcatgagttctgaaaaaattgagaaataagaaagaagagcaaggcaggaggacagtgaaacatcagcttgattaacgagggaggctgggctggaatggcacttttccttttgggcaaatgggtttcctatgactgatgcctgactgagcagacttgcccagcaatcctcagattcacacagggtttagagagaaaagcaccgtttcatgtggggatggggagtgaggtgagcccaacatgaccagtttatccaaatgtacccataaggtgaacctttctgcctgacaccctacccctacggagggaagaaggcagcagcaaggggccagcagtgttacactgttggacttcctggacactgatagagatatccagagctgggaagaagcagtgccctaaaatacaggttagctttcaatacccagcacgagcctgtacctgatcccccttcgaaccactccgaccatcgctcacctggcccccaccatgggcttccatctgcattgtttcctgttaatggcctttgtagtcactgttctcctgccaggaactctcttcttcctagacactgtctcatgccctcagatctgctcagatgtcacctactcaggggggccttccctgaccacctatctcaaatagtgccactgcttgacccctttcctctctattccgtttatcctgctttgattttcttaaaagctctcgcctatctctcttcctcagttcatgaaggcagatgctgtctgttttattcattgtatgagctaaggatatatttattcagtgaatgagtggatggtccagtcaacttggagtctgttccaggtcagctgctgcctggagtattatcacttgcaaagtggcccagtcagcatgggcctctattgaagtgctacactaactcctttcaagagtggaggtgggccaaggcgcagtggctcatgcctgtaattccagcactttgggaggccaaggcaggtgaatcgcttgagcccaggagctcgagaccagcctggccaacatggcaaaattccatttctacaaaaagtacaaaaattagctgggtgtggtggcatgcaccggtggtcccagctactctggaggatgaggtgggaggatcatttatgcctcaggaggcggaagttgctgtgagccaagattgtgggtgacagagtgagaccgtgtctcaaaaaaacaaaacaaaacaaaaaaacacaaagagtggaggtgaagcttgaatgagacaacatatggaaaagagctgagaaatgtactagaatcatggaagcgagtcctatagagtggttactgctacaaatagtagtcactcttttagaatcagggagaagtttcttgtcacttcagggagtggtggttaaactgatgacattgccacatttactaggattatcctcatataaaatgttctcattcattgttcccaatcttagcctgtatttgccacccggtgtatcctaatgttcttatttgaaaatgctaattatagtaggcaatagaaaggaggcaattctttttttttttatttttgtttatttttattttttgagacggagtctcactctgttgcccaggctggagtgcagtgatgcgatctcggctcactgcaagctccgcctcctgggttcacaccattcaaaaggaggcaattctaaagaagaacagagctacactgacttgagcaaaactaacaaaaatttcttagtggaagacactgactttgccatgagattttaatgtcctaaagagtagagagcttcacttaccccaaaaagtaatttctgaaactgaaagaagtacactagaaatccattatgaagcaatctgtggacctgtgattttcacttctttacagataaatatgcctttcagtcatttgcaatttcagtacctataaattctgcaaatggacaaaacaatccagtttattcagaattccgtttttatcaacagttgcattgaggcagatttcgttatattctgtcgtttatatagtggtatatggaaaatacgtggactttgacatcacctagatatggcttcagattccagctttgcaactcaatagctgagggggcatgagaaaattatttaacctgggactgagtttactcatatggatatgtctcatataaagctctggccttagggcttgtctaagaatagtgtcacagtcaagaaaaggtgtgctgtctgttagtgaacggctatgagatgttctgtaacaagggagcccatttaattgtatttaacccagactttcccaaatgtttttccttataaaatctgttaatatagacagataaaatctgtctatattataaaatctgttaatataatacagaattggtattccataaaggaccctgtgggaaatcctggcttgcataatgccagacatatttgatgtaagaaatcagttttgcttcccttttccagcttagtgttggccagtcagaattttcactggacgcctaccaggcaacttaaagtagaactcttggggttttgtgccttcacaccctccatcccaactccttccatagtcttcctgatctccaatcatggccccactattcatgtattagtttcctagggatgctgtaacaaaacaccaccatctgggtaacttaacagaaatttattgtctcacagttctgaatggtagagtccaaaattaatgtgtcagcaggaccatgctcctcctgaaacctgtaggctacgatcttccctgcctcttctagctgttgatgtttaccagcaatcctgggcttcttggcttgtagatacatcacttagtgtctgcctcttcgtcacatggcattctcctctaaagttccctcctcttattataaggacacgagttatattggatgaaagccctgccctaatccaactccagcataatctcattgtagctaattattaggttggtgtaaagttacttttaattaaaagtagtgtggctcacacctgtaatcccagcactttgggaagccgaggtgggcagttcacaaggtcaggagatcgagaccatcctggccaacatggtgaaaccccacctctactaaaatgcaaaaaattagccaggagtggtagcacacacctgtagccccagctacttgggaggctgaggcaggggaattgcttgaatctgggaggcagaggttgcatgagctgcgatcgtgccactgcactccaccctggcgaccgagcgagactcgtctcaaaaaaaaaaaaagtaatggcaaaaaccacaattacttttgcatcaaccttactacgtctgcaaagaccctattttcaaataaggtcatattctgaggtactaggagatagaacttcaaaatatcttcagctcagcacataacaattcatgcagttactctgggcagaaaacaggtgccatccttgtacttcttttctttcccttagccattatgtctggtctatgagcaaatattcatggatctgccttcacaatgtagctgaccatttctcattacctcccctgctactccttgaccccagcctttgtcatatctcttgggttataacagtggcctcttatgaggtatcatggttccatcctgattctccttaagcctacttgttagatagcaaccagagcaatcatgtcacagtgtaaatcagatgatattcctcccttgctctaaactctccagtggtctcccattatcattagaataaaatgctaacacagatgaaggaagataagactctaggagctctgctttatctgtctctccagcctcattgtgtaccactccctcccctattgactgtgcccgccaacccacagtaagcaccttgtttgcactgcagggcttttgtactgtttcctcaaggctggctccctctcagtctcagatcatctacccagagatgtgtttcccactcattgttagctgaaacacttgcaccccatctggcagagagcagagaaagtttttttcttttttttaattttttttttttttttgagacagagtcttactctgttgcccaggctggagtgcagtggtgcaatctcagctcatgcaacctccacctcctgggttcaagcgattcccctgcctcaacctcatgagtagctgggattacaggcgtgtgccaccatgcctggctaatttttgtatttttaatagagacagggtttcaccgtgttggccaggctgatcttaaactcttgacctcaagtgatctgcctgccttggcctcccaaagtgctgggattacaggcataagccatcatgccgggcccagagagagttttgtcaccactcatgaagcgaagcactcaggaaacaaaaggagagaaacaggaatgagatagctgtccctgggagggaaaggatcaataactaatgggtatccccaaatcagatttacatgagtcacaatccaaacaaatgattttcttctgctaatctgaatttggaaaggaaatccctacctaggagcagagatccaagagagctgatcttggtttaaaaaaaaaaaaaaaaaaaaaaaaaaaaactcttaactttttgttgtttttttattggttgtcccaggatctcatctgcaggttctggaactagtggggtgtcacagccaccccatgttggctgccagaaactgtaaggggagaaaaagaatatcttttcctcatccatcacaaggttcatggctgagcccctataacaaataaaaagaaaaaaaaaaaagattaaatgggaaaagtataaaagttatatttaatgtaagttttcagtgacacagaaaccttcagaaaacgaagccccaaggacccagggaaaaccatgtgtgtttagggacagttgtgcagaagtgtaattggaggacaaaagagtgtgatctaatagtaataatctggggaactcagcgaggcctatttgttcagattcctttcagcctcggtgtgacattccttccctctgggtatagggcaggacccctctggaatgagggtcttatgacctactttcaggggaagtaggtcagagaattctttcatggccagctctcaggagaaaaaggtgggaaaaggtcagagagcagccttcctgcttctgctgttttttcaatttcctgctgtttaaaatactcactgtgccaaggtgccattattttgggttatcatgttgtgagccccgacacctcagttccttgctttaccacttactaactgtgaacctgagcaaatcacttagattctctgaggatcggtatctcgtctataaaatgagatgacaaggtaaggttgtttgtaacccttaccttcctggttagaaataaaaaatataaagcaatcagtacagttttttgtacatagtaagcattcagtaattggtagctattaactggtatcattatcatgatggtgcctttagtcacaggcatggcccttctatttcaaaactgttgaagaacatgttgggataatgtttacattttactttccaccacatgcccacttaaatttcccttctggttggagtgggatttcaaggaaaagctctagactgtggtgtcagattatattattttctcctcttcctgatttttctcattctctccctgccacacacctctagtttccttttcctgcacatccaaggatatggtggcagagacagggatgactaattcgttagagagattagacatcactgtctgatgccagagaggaacctgtgaaaaggagataggcgtgtctcagtgccacttcagcaaaaggttgccctttaccttgcatgccatgactgtgtcctccctgggaaaagagtacttcaagagtgttgtccagggtgtcaacatagtgagactctgtcccttaaataaataaataaataaactcctgggctcaagggatcctcccgccttggcctcccaaagtgccaggattacaggcatgagccaatgtgccgggcaataactattttaattgcagctacaatggttattatatgaaagatatgtatcctttgattggagtaatcgatagtgtaaagtggggaagtggaaacgagtgaaggggagaatataaggctggtttaaatacctgctctttgtttgtttgtcctcatcctgcaggttttgtttttgattcttttttctttggcaaaacacacacctccagtggcattttcaggtttcttttcttgaggtcttgcaaatctgattatgctactggttgatcaggtaggggagaactggccaggattttccctttttgtctcttctgatggtcaggtcgattggaattggagtgtgagctaatggaaaatagcatctagctcattttaccaataatgaagatgagcccagtatgtgcaggtaaatgtttaacaattggcagtctgaaaaaaagaaggcctgatttctggtgtttgctggttactgtggtataaatactcctaccatagttgatttcaagctaccaacaagattactgaacacatagttggaaaaagatgcccagaagcaaatcatcatatgtgtaatctgtctaccacacagatacaaaaaacataactctagatcacagataatagtaaaatgtagtaaaatcattaggaaatgataagttttgagtactttgaacatatgttttaaaatgtaacttcttcaataacagtttaattgttaacggtggctctgttgaacaactgactctcaatatttctgaaaatttaaccatcagctctcttgcgctggcttgagcacatcaggtgggacccagagaagttactgtcttaccaaggtcctcggatgagtcatggcaggacctgggttattacagagttgtttctaaaaaccagactactctagacagatttacttatcaacgtttctttttattcatttgtttatttatttactccatcctttaacaatccataaaagggatttaagactattacaggctgttattatctattgagacagtaagagtcagaatatttgaatatatctggaataaaggtgggagagtaggccgggggcggtggctcacgcctgtaatcccagcactttgggaggccgaggcgggtggatcacctgagatcaggaacctgaggtcagcctggccaacgtggtgaaaccccgcctctactaaaaatacaaaaattagctaggtgtagtggtgggtgcctgtaatcccagctacatggggggctgaggcaagagaatcgcttgaaccgaggaggcggaggttgtagtgagctgagatcgcaccactgcactccagtctgggtgacagagcgagactctgtctcaaaaaaaaaaaaaaaaaaaaaagacgggagagtggaaagcagatgagatttagagccataagtaaattctggctttgagtcttggccttgtcatcttgatcaagtcagttactccttggtaaaagttttctaatctgaagatgaaggaactggaccacatgaacatttaatatcttgtaggtctaaaatatggttccttatttagattcatcaattcaataatatttgcttagtaccaactatatgcactctcattatgaactgtactccaggcagaatgaaattcagatatgataataaatattattataatagcatctcacatttatgcagagccttctgatttgccgaagagaaaatttactttgagtctctgggattctgtggctttaggagatcaggacatatcttaacagaatgttagagaaagaagaagctattctagaagtacaaatagaaccttattcctaggaaacagaagaggtcatacccaggtagtaataaggaataagcactttttctgccatgtctagcagttgcttcactttgtttttttaaaaaagcaacaaaattaccttttttctgagtataaaatttttgtagaaaatttagaaattattaagaagtattaaataaggaaaaaaaccatatatccaatctaatatcctgcttttctttacctaataatattttatcatatttttcaaaacattcttagtagaatcattttaattgctgtatgtataataattttatctttttggtatatagtaacttatctgatgtcttactgttggaaattttatttattgacaatttttattattctaaagaacactgtggataaatagctttgttccatatcaaaaatcatacattggacacatgattttcaactttaatatttaaggtttagtaagacaattttagcaaattatatacttttgacctttagagtctttgattctgttgatattgttttgtatttacccagattttattctgtttctaaacatttattaagaagaacattttgtttgaattcttcaatatgatatgtttgaactggaaatagcaccactgttttcatattatttcaagtaggtatgataaagtgtagggttaacaattttctttttcaactttctgtgggagatatcctctgtctagtataaggtttgttggtttagaaattgctcaataaatgttcaataaataaaaggactcatgctaatttaggataagcaaaaacatatgagagggtgtttaaaaaacccacttctccatttatgataatagctttcattctcataaggagagataacagctaagcacagttcaggaattctgactgttgaatgagaactgtaaaatgaagtatgtcggtgctttaattcacctgaatgggaaaccatgactcagatagttatttgattaaatgttaatgagatctactaaattgacccaattcttacttttatgtattagatttatatcaattaaaacaggaccagttttacctgttttccttttttaacatctttgagtcaacatctgcttacaagggaagtgctgtaactaccctggttatttaaatattaatggcttccattaaagggagctagggattggcagcaggaagattgagaaattagatggcatgattggtttcttgtttctgtccccaggggaaaaaaaaaaaacaaaaacctcttactggaagagcttcttccattatttttttttaatattgcatatatatatatatatatatatatatattttttttttttttttttttttttttttttttctgaaccagttagagagtaagattgttgatttcaggcatcccttgaaagtagctgtgctataaaagcatcagtgtaaacaagttcaagtaatttaaaaaaatgttttcacagtgccatagaggtcattaccctggggcttcttgggaacctaatttcagttcctaaattcattttgttctttattatagtcagtaataggcaaaagcattttttagctccatcctggatttggttggccttatttacattaagtgtggggaattctagtaagttaaccctgagagacatgacaagggttatatattcaatcacattataattataaacctttatcttactggggaaatgtgcctgaaaagcttttaaaatttgctataaccaatattttcatgttatgccaaattgggttgtaataagcatgtcgttaaaagatttttacacgttttgtatttttaagatgttctaatggcttggcattttaaagcactgaacaaaagcaggcattatatttgcataattgtgtatctctttttgcatccagaaatgtgaatgtgtggagtaccctttcatttcatgtatttgatttagaatttacagctaagaacatagatcatgcttaaatagcaggagtgcagagtcatgtaacggccctggtgtgcttgtcataattccctacttagcaaggtcaggattagggtgagacaagagaggcactacctcaggtgtaaaacttaagaaagtgtcaaaaaaaattcaagactagtaatatgttaatgtaatagttaaaaaactgtataatgcagagaaaaattaatgataataaaaatggtcaacattttagatcaagatgagatctgaccctgcatctgtacctccctcgcttctccctagtcctggccttctctactttcatgtccttgcatgagacactcttttgattcaagttttccttacctgaatttggtgacaatagtcctcactttcctggtgacaaggatttttttccttggccaaattttagtcaggcttctgaatctcctgctaggcctgtttgtgcacgtccttgtaaaatccagttttagcaaaggacactagctaagtcgttttaccaagaaccagcaacatcaacatccgatcgcctcaatgtctgaccaggtttctcatcctgcaccatcccccaggtgatgtctgatcaccctggcctgtcttcagtgagaatcctgttaggtttagccagatccaccttaccccaacgcttcctttgagtaattttccatccactgacccccacactgctccttggctataaattcccactggcccgtgctgcatttggagttgagcccaatctctcttccttgctgcaagactgttgactgcaaggtccctgtgcctgtcattatcctgacaaagtctttgtctccatgctttaacaaataccattcagtcgtttttctgtaacactggtggaattaggagattcaaatcatctacaaatacatgggaaagcccactataaatataatttacatattgatgttcaagtaaataattatacagttttatgccattattattataatctcagatgaatattctaagcaaacatcagatctgaagatctttggaaagctttgtgctaatggtttggggcctaatgtctttaatttacttatcagctagcaataaggggaggaacctaagggtagttgtaggaccctcccccctttctttcttccttcctgtttttgtttttgttgttgttgttctttgatacaaactggggctttataagagatagagaacataaccagaaatccattgcttctttgaaatttccctgaaaggtgtgcatggatgcctgaggtctcacctttgcttaagctgtttccacccacccatgatgcctttcccatttctcccacctgtctgagaaccattcaccttttatccatatccaacttggataaggcctttctgtgatctttcggcctgaaatgtgctttcaatcctctgcccttctgtggtggcataatggtttctaccacccatttatggtcagttacagagttcttgttgttctgagtctttatctaatcctccattgaatttactttccaaactgcattttaaaaaatgtcagtggttcactcctataatcccagcactttgggaggctgaggtgggaggattgcttgagctcaggaggtcaaggcaacagtgagacgtgactgtgctactgtactctagcttgagcaacagagcaagactctgtttaaaaaaaaaaaatcagtaattgaaaaattactgacactgtaaatcccttgaagagtctcccattagcctttgaatttaagagtaagcagggattgggtgtgcttatttatggtgctatccaaggtccttgcagagggcctttacacacaatattttttttaagtgaataaatgaatttcttgcatgtaaaagggccaggtaaatatttactcttatgattcatttttggaatgctgtttttatcctagaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggtaagtaagtttaattaaatcagaaaaccactggcatgagttcaacagtttctcttttttttttttttttttttgagacgaagtctcgcctgtcactaggctggaggtcagtggcgcgatctcagctcactgcaacctccgcctcccgggttcaagcaattctcctgcctcagcctcccaagtagctgggattataggcacaagccaccatgcccagataatttttgtatttttagtagagaggggtttcaccatgttggccaggatggtctcgatctcttgacctcgagattcacccacctcggcctcccaaagtgctgggattacagctgtgagccactgcacctggccaatagtttttcttaaaacaagtaacccataggatgcaaaatatatttcagtatttttactcagttgttttatttcaaatatatatatatatattttgtttgtttgtttgtttgtttgtttgagacagagtcttgctctgttgcccaggctggagtgcggtggcgcaatctctgcttactgcaacctctgcctcctgggctcaagtgattctcctgcctcagcctcctgagtagctgggattacaggtgcccaccaccatgcctagctaatttttgtatttttagtagagatggggtttcaccatgttggccaggctagttttgaactcctaacctttggtgatccaccctcctcagccttcaaaagtgctgggattataggcatgagccaccgcacccagcctatgtcaaatattaatgacttgatgtcatatgttgtctgagtttttagctaaaatgagaattagccggggttgaattacctgcatggttgagttgcagcattttatttataagactagtagagtgcctgtcccccagtggtcacttaatgctaatactatccttaaaacttactatgagtcaatcactggttccaagctttctgcatgaacttatttaaacatctcaacatcccggggaggtggacccattttacacatgtgaaaccaaggcagaaagagattgagctactagccagagttcatgcagatatcagctggtgtgtaggcttcatactcagcctcactgcagcctccctgctgtgccccgttatccagcaagtctgggccttagttttctcattaagataagatggagctttaacaacttacccaacttacagagatgttatgaaaatccaatgagataaggcacgtgaaaatcttttgtactctataagttgttctccaaatatgagtgtttgcaattggctttatttagagattgtgggtttttctattgttattaccaataggaaaaaaggtatcacttagtgttatcagaggctaaaatttatgacctgcatctgctcttttttttttttttttttttttttgagacagaatcttgctctgttgccaggctggagtgcagtggtgcagcctcggctcactgcgctctgcacctcctgcgttcaagcgattctcctgcctcagcctcctgagtagctggaactacaggcatgtgctaccacgcccggctaatttttatatttttagtagagatgggattttgccttgttggccaagcttgtctcaaactcctgacctcaagtcatctgcctgccttggcctcccaaagtgctgggattataggcgtgagccactgcccccggccctgtatctgctcttaatgcttctttgcgcttaatcactgtgcgagtaaatgatttcaactttctggacttcagtaggttcctaagctataaaacagtagacaggtagctattctgaggagctgtttgcaaagagcagcagaaaaatggggtagtggctggagggagcactggggtccaggaagggtctttaacagaataggagagaatgtttgtttgctgatggaaatgatgagtggggaattggtttggtttggggaaccattgttggagactgtgtttaagaaggtgagagacagggatctggcacacaggggccccaaggttgtcccttgtcacaggtgggaaggcagagatggtgcctgtgaagctggcaggttggcagatgtgatgggaatgaagaagaacaccggaaattttagacaaatggtcattttaaagatcaaaagcaaatctaaatactctataaagaagtttaaaataaagcaagttttattcgttcaaagtgttctcacagccaataggatgtcattgttttttggttttcgttttacacgaagacattcttgtattttaagggtatgtgtaagtgctgttcatatgtctttttctgaaatacacattggcttcaagtttcaccatctcttttctaaggaaagatgaaaaactttgataatgtatttagtgtattttaaaaaggactgatttttacctcacaagagaagtcttgggaagatggctgccccaccatatgggctgataccattaagatgcttgtgaagaggcaaagtgtcgttaaaccctccaaattggattacatgcacgtatttcagcttccttaacaagtggattctgatggcttagatttctgttcattaacagtaaatcaaagcaaaccatttaatgtagggatgaaaaggagttaagtaattgttataaccaaattctaccaatccacatatgaggaaattgagactgaaatactttatccgagttgcccaacgggatgtgtgcagtagagttgggtagcagagatgggtttaggacccaaatcttctgacttctgctctgtgatttgcctactataatttagttctgtttgtttcagaagcttctaaagtgttcttctaaagtgaccagatgcatacacacaaaacaattaaggaattaggcagtgcatactaaagtactaaagtattacatttaaagtaagtgcactggagattcggaggaggaacctatccctgtcggttggcataatcgagggagaaaatactagagctgtcgtagttgtaaaaataataagtagctaacatttagtcattatatccctagtacctagcatagtacctatacataataggtatgtataaatatgtatggaatttaaatttgggtaccaaccatgtgccaggcattgtgctttacatccattagctcatgtagttctcagagcaaccctaatgaagaatccataagcattgttcccattttacaaatgagaacactgaggcccggagaaatcaaatatcttgcctagggccataaaactagtaagtggcagagcttgaattaaaacctatactggccaaccccagggcccatggtatgaaccactatgctatatgagcagctctgaattaaaccctatactggccaaccccagagcccatgatatgaaccactatgctgtatgaccagctctggacttcagagtgtcgggtaagatttacgtaaatagcgaaaggtctgtgcgtaccaaatgagagaaacagcttaaacaaaggcattaagtgaaagtgaccatggtgtttctgtatcatacatctggtggaaccaagtaaaaagaatacagagaaatccatctgagagactactgacatctctcctgttcttgtctttcttgcctatgagcatttctcagttatgggtattagcagcagccaagaaagattccaagggaggtagttttatcttccaccctcccatgtcaagattggtagatcttcatctgatgattgacaacatttaaaatattaaatctgtgtataatatcacttcctgaagttatgttacacagtcaaattctttgaacagaattttaagtggctgattcaaaatctggtacatctgggtattcaaatgtagccctgacaacataattaaggataattttattcatccgctttgctgaaagttcaggaagctctggacagatggtcagctcactcccctaaatcccttttgtttatcgagtctgtcaaaaccctaggaacgtgagtcttattattgcaatggaacattttcgagataaacagtagctcgcaaaaaaacaagggaaatttttaatttttaaatcctcatttagctggcttagttttatgtaattgtaattgcatgtttactttctctgaagctagaataaatctttactccctgaaacaaaatgaagtaggaggcacattttagcttgtttataaacactgcattttcacactttaaagactattttactggtaggtcctaatttattgttacttatgatctgataatgtctgtccacactggtgataacttgagtgtttgtgaatctggcatgcatgctgattagctggttcaagccctttgttggtcacattccaatgtttttgaaagtgttaatatcacaaacattggttgcagcagtaaaaaacaaggaggaagtaaaatcacttcaaagaaaaccttttcctttttaaaactgttctaaacatttgtattgtgagtcatggtgctggaaagatagaaactcctccatcttgggagtttgttttccacattccattaatagaaggatgcacaggacatttgaaaatataaaaagtcattttagttgatgacattttaaattattttactgggatttagttagtggaaatttaaaatttgaaacagggcaaaactttttttatgggctagagaaaagttctaaatttcttaagatcttttagcaaggtctgccttaggaggtttttgtagaaataagccaatcttggtgccttgtcagtagggcttgttttagttcattcatttattgacttgttcatccattcatttgttaacattcattcattaaaaagtatttattgaatgtcttaggtgtgtaagtcagcatgccgagtcccaaggtatgcaaagttaaataacacaaattccccactttccaacctcctgacctaggagggaaggtctgaaacatactcaagtaaaaggtagtagagaagctactttggcatcctcaggaagggagggatgagtgaacatctgaatcatttgagagcagtctgtggaccctgtgtggtgctggtcttgggagagatagattgattgaatcatcctttcctctgttccattccctccagctcctttctattatatattttctctttttctcattatactttgacctttctcatttttgtgttgtctttctcaaatcctcttcttcccatttacctctctgtggtccacatctcactcatttccaatccacagtttttacttctctactctcctttttttttttgggaggcagggggtggtatctgtttgattttgagaagattaatagttttgctaaaaatatttcaaaagcattttaaaggctgcccagccctggggggtgatctttctctgggctaattaagagtggaagtgaagaaagggagaagaaaaacgacagatggcgggaagagtgggaggggaggagtggcaaaaaccactttccccaaacacctcccttttctttctcataagatcctcacctctctctctttttctctggctttttttctgatggaggagggatagacctcattgggaacagaatgtgaagaccaacagaggttgcattaagcaatcttccctgggagaaccaggctgagccagaaaaaggaaattcttctagacttcacttggagtggccaaaggctaatgataactttcccttccaattgactgaacccatattttatgtgcagtgctaggctaagggttcatatgcatttttgttgtttgatcctcacagaaagcccaggatagcggaatcagtatttatattttgcataaaaggaaagaggctcagagagatggagcaacttgctggggagtcctaatctagttaggattccaaacccaggtctgtcaggctaggaaacccatgctcttctattgtgctagcctaacaggaccctcaaggaccacctgttgagacctttcttttgtccccatgacctcctctgctcagaggtgctccctgttcctagtcagcaggtgcatttcacagacagcaacagtagttctgctccctacctgccagcttctttatatagattttacatgtgggggctcttttaatccaggtggcccacaggcagtacatcttccatagaggagaaactgaagaacagggaggttaaatgcctgtatggtgggtcaccagctggtctataggagcactaggatcagcaaaggaactgtgtggtctagatgagaggttttccttttgagaggcatgcctatatatatgttttttttttgagacagggtctcgctgtgttatccaggctggagtgcagtggcatgatcatggctcactgcagccttgacctcctgggctcaagtgatcctcccacctcagcctcccgagttgctgggactacaagcactgccaccatgctcagctaagaggcatgccactacttcccttcccactccttgccacctgcaccttctgactcactctagttgaaagccctgggggaaggaacaaactctgcagcctgactgctgtgtttaatcccagctctcctctactagctggcaagtcacttaacctctctgtgcttcagctttcttatctgtaaagtgggaatgagaactatacttcccacatcatgtaaagccctggtattctggccttgtaaaataggaactagtggtcccaaaacagcagtgtgctggtaaaagtttagcaaccagcccttgcagtcaggaccctgacttgtagcatctattcccttgagtgttaatattgtcaccttggcttattttatcctgtcaatgtctctgaatgctgagctgggaaaagatgctaacaattagccttccagagcctaagggctccacctaccgtcaaaggcatctttagtcgatggcatgtgaggagtcaagcttcctgacctactagccagtctccaacttaatatggctcagacttacatcgcttttcttgagtgactggttcctactggggacacattctttgtcaccactaaaggattcttcttcacccaaactttcccttttacttcgccaccacattctgctgttttctgttcctttgccactcccttcctgaccactacttcccttcccactccttgccacctgcaccttctgactcactctagttgaaagccctgggggaaggaacaaactctgcagcctgactgctgtgtttaatcccagctctcctctactagctggcaagtcacttaacctctctgtgcttcagctttcttatctgtaaagtgggaatgagaattatacttcccacatcatgtaaagccctggtattctggccttgtaaatgtcagctcctagtagcattctttactcctgggcatccttggctgtctaagcatccccaattactccttccctgtgcctgtcccaatggtgccataagaacgccaggacccctggcccccacaactgccccattgtcctatttggctccccctgagtagccatgaggtcactcccagtctccccactgtccaccactgtccacctttccccaatgtccacagccacccacactcccatatgcctcagcccactcctcttagtagatgtgacaggctttttgccttcatgaaaacaagagcgtgaaggacacaggccttctcggggcgaggccgagagctgcttcaccacattgttactcttgcaccccactgtttcaggtcaggttactttatcctgctcctttccatgtggcccaaggtgatcaccttggtttatgcaaggtgtaatgataggagattttcttttcttctttttaaaagaacttaaaaaaaaccttattacagcttataaaatatatttgggcacccctccccaaaggcttaaaactttcacatatccttggctaaattgtgaacgtggactatgtgggattggccaggggcaggctggaatttgctgaaggaactacctgtcataccagttcccttttagcttatctaatcactccatttcatccagactgagatagatggctcacatttttgttgttattgttttctgtaatccttgggtaaattttttttcccccactaggaagagaattgagtttggtcggccctttcccaggactgaacatgaagagttatgccggcttcctcaccgtgaataagacttacaacagcaacctcttcttctggttcttcccagctcaggtaggtgcccaacagaaagaacaccagtgttcaagacctggcttgccgtttgagggtagaaatgttagcctaccgagctacaggtctttaaaaaaaaatctcatagattttttctaaaaataagatttttgtttttctaaaaataagcaaacctgcatggtgttcctatttatgggcaggaaacaaatgttttctgcgggagtgggaagtgtccaacttctaaatttcttgcagccttatttggagtgttaatactaccaccttcttcctgccacagtcttcttcctatcattttgttgtatttttgttctaaaaatacttagggcagggatgggtcaggggagcccaaggaggataggaagtaaggagattcctgaagtgaacaggtaatgcgtggctaagtatgtttatatctagtcagccatggccttgtatagctgcgctgtctgtggactccttcatgcactcagtacccacttattgagcatattctgttctgtgggggccacagaggagacagaactgaactcagactctatggatctttagaagattgggaggattgccgatctaggaaggcttctggcaggaaaggccatcactttcccaaatctctatagacagaggtgggtatgtttttctacttcaccaatcctatgtagttcacctctagattatgccattcttctgtatgaacaagacatttttagcttagaggtgctgttgcgggtattgctggaagcgctgagaagcaaatcttattccactgcagagtaattttctgttttcatcctccaaattggatttgttttacaggctagagaaagcaaagaaaaagctatccctatggagggtgttatttatattttcatgtgatttgcatatagctgggttgcatctgtagtaactgagtaaattgcttggtccagggtggttttcaagttacgttgtttctcctggtactattgagttatattttcttgacttttctttcctgctctctcaactacatggcctctgacatagttcaccccaaagcagcctgtggtgggatgtggtcaggttggggcatgtaagaaatggtgaaaaattccagatcctttgtcgttgggctcacagtagccgtcagcctgtcgtcttttctggaaggctttcttggcccctagagagggcttcttatttcttcgtcagtgttgtgttgagtgaggcaagtgagatgcctagagcttgcaatgcaaggagacactcaccctgagggtcatacaagtgcaaggtcagcactggagcctgagcaccctccagtgtcttgcttggtgcctcccataggtagagcccaactgggagccaggtggaatcgggtggtctagcccataggggtctgcctaacagggcacagagcgggatggaggagagtgataataggccttcccagtctcttaaactttatgcctttcagaaatattctgtatgtgtgcattttttatatcatatctattgatgtcattgaatgcagtcatttatctgcagtaacttctttatattagttttctcttgctgctataacaaattgccacaaacttaatgtcttaaaacaacgcacattttatttcacaattctgcagattagaagtctgacacaggtctccctgggctgaaatcaaggaccagtagggttgtggtacttctggaggctctaagggagaatctcttttcttgcctcttgcagtgtctagaggctgctcgcattccttggcttatggtcccttcctccagcttcaaagcctgcaaaggcagcctgagtccttctcacagcacataatctccatggtcatgtttccttctctgactcttgtcttctacctctgtcttctaaaaggactcttatgatgacatttgggccacctggataatccaggataatctccctattttaaggccagttgattagcaactttaattccatctgcaacctgaattctcttttgccctctaacctaacatattctaggaattaagacactaacatctttagggagttattattctgtacaaaatggatacttgaacatttgcctagattattgagtctcgatatgttttctctcagggattctcacatgttattgcatacaagaattctctgacttcccccagagattctgacttgtaaattggcagaagccagcaggttgcagaattaccaagtgaaccattctctgtgaaaccactccagcctatgggccactaccctctgcctgaagctcctggctgaaaagtgaccaacacgccttttaccagaaagaaatgcctacagcagccaaacccagacagaggtgatgtgtttcccatgttgagggttgaaatgaggggagtgaagagacctgtgcagcctgtggaggttggcttccctaatccagcttgcggattctctaagaactgttaggactgcttccccatcttctttccactctagtcctcctcccccactctagctggagaactccaattccttactttcccttctgctttgttacttgtttttgacaccagtcagttccttgttaccctctgaatgttgagtcttctttggtaatgatccagatttgtaaaaacaggaactaagggtgagagaagagatcagatggaaacaaagcaaaaacccagtttttttctttctcaggttgcctctgggcaggtgaacagctatgtctagaatgtaggtggtgctttggcaccgtaaacctgttcatggaaaggaatttagtttgcatgctctactggttgccagactgaagtctgaaaacccccttcattaaacaatgactggactgttggtttttgctttttccccttaattaaatgaaaacctagctgtgcatcccctgaccaggttatgttcttttgagtccagtactgtagccataaacacaacctaagatgatcccaggagacacaacgggaaagaatgtctttaagtgtacatggtgttctggtgagagtgatagatgaagcttcgggagaagaggggcagagatgaaaaaacggtacccacagatgggtaccaccatgtggcagccaagatagattgttccaagaacctcttaccaggaagcccacatgcagagccagatatctgaggcttctggctataaattatggagtcacaccagtttgggtgaaggagataaagcaggtgtgacaagaagaagcagtggtcactgagacacatttggcagagatattagttcattgtctgtcatagggaagcagctgatattgtcaccaggggagaaatgacccctttaaaaaactgaaggtagagaaaataagagatccaggaaggatggcacacttgtcatatcctctgagtattttttggggggatgctgtttcttaaattagttttgttttcctgttattgctgtgagactgccctgttattgtatacaaatcagtgcagaataaattccagaacttgaaattggtgtgcagtgggaatacaacctgcaagagaaaggagtaggcattttgcatgggataaaaagtataaggaaactagcagaaatgtagcagttatagctgctggtaattgaacactgactatgtgctaggcactgtgcattttcttatctaatccttacctgaaccccatgaggaaggaattgttatcctcattttatagatgaagagattaaataatcagccccaagttactcaactagaaattggcagaactggaatataaaattaggcctccgatgtggaaatccttgagcttggccgtcatgtgttagatccagagaaaacgagtcatggaaggcacttgagtatagtggccaagactacagtctcttcggtaagtcaggcttggatagaaatcttggcttcgctgcttaagctaaaggtgaccttggggaatttcttagtgtctctgagcactggtttcctcattttaaaatggggaccaaaatacctacttggcaatggttttgagacagaagagataataagggaaagcattagagtggcacctaaaacatagtaactatagttgctattgtcattatttcattatgattacgattattatggttattattacttttaaaattattaacacaaataatgtcaagaactaaaccagtcacaatttgagaggttacagatgacagcaaatagagggaaattgtaaattagaaggacctgaaccccagccatcaaggtggattaattgctaccaggtcaggaaattactaagtgaaattgggatttttttgcactgaagaagataaaaaagacatagagattattactctttaaagtcagcatccttctcattggaaaaaaaatagctagtattggactttgccttatagatgtaggaacatagataattcattggacacatttagccaaaacctgcagatcagttacctcaaatgccatggagccaaaacctggggcatttagagtcaggcctaggccccttcacggaggaccccaccttgatcctcaggccaagaggaaaggcccagattcctggatttgagaatctagtgtctatgaaagatcaggattacactgctgaggcccgtgaggtgctggggcaacctacaggaagggctccctctcaggatcctgcaggctgggccctgctgaatcagatactaggatgatcagatagcacaatcagcagtcaattggagagaaaatgaaccgctctaatgctcctggtccaaaacaaaaggcgttctctctggcagtagaagagcatgtcccagaggtgcctggaagccaacagaacagcccttcctgggggtgaaggcgtggaatcaggccccaccatacttcagcatggtgccatgtacactccctcatctgcacatctgaacttctccagtaacacagttaatgtggaagaggacaggaattcccaaatgtgctactgctcaccagaaatacctggagagctttggaaatatgacagatctcccagctcattgtctagctgcacccagcagagattctgattctgcagatctggagagggcccagggatcagtgtttgtaatgtgctccacagtggtcccagtgcagccagtcaatgggctggaatttgggaaaagagatctagaggtttggatacagggattctaattccaagtctgcgattcccggctgtataacttgggagatggcattcgtattctctgggcctcagttctctcatttagaaataaagatgttaaacctagtggattgctaaggctccatcccagcattacattctgtggttctatagttaagtataaaaatatccagtaactccaaatcaggatgtggattgaatgaaggagcccttgaacgtgtggcctgtgtggtagccagccttcaggctagttcccagtgctcttcattcatcccttggtgtatgtgcccttgggcagtcccctcccatactgaatcaaggctggctttgtgaccaacatgctagcagaaatgatgatgtttgacttccacggctaccttataaagggcaatattacttgtgccttggtctcttgaatctttcagtctgaggaaaggatgccatgttgggagggcactcaaacagccccatggctgagcctctgtggagaggaagtgaggcctccaccctccagtgggtaccaaccttggaagaggttcatctagtaccagtcaatccttctgatgactgcagctctggctgattccttgaccacagtttatgggagaccctgtgccagaactgcccagccagacaccctcaaatttctgacccacaggaatcccgagagatcaaaaattattattgttgtcttaagccattaagttttggagagatgttgtgcggcattagataactaacatagcttcttgtcttgtacttctaggctgtggctgtgagatcttgtgacaactagcatatactccctgcctgggggaaggatgaggacataaggagtggggattactatgcgagctacttttaatttttgtttttttttgcctctcagctccaattctaccctcttccctgttctgccatagtagagctctcagcatttctcctttgcagcaagcacaatattaagctttgtcagtagagggtgctggagagacattgcaggaggaatttgtcttctctttctgtgcctatgtcctgcctttggctttttcttggccccactgcctagtctgtcagcagtgtgttgaggtggggggtggggacatccagtgacactctgccttagctatgcacccagaccaccaagactcttagcaagatgatatccctggctctgtccaggtgatcacctggctgtggccctcccagtatgaacacaatataccctaggccttgccggtgagcaggctccctacagccctgctctctctgcagccctctcactagccttggccatttgcaccccagagggtcgttcccttgtccctcccagtgtggacaccgtgtgctccaggctgtgtgcaacctttcaggcaagcaggctcccagcctcctgagtctctctgtgtccacctgccagtcacagcccacctgttcctatttacccaaggatggttgaccagctttggaccagggtactcagtgaacttctcagctactcagaaggctgcaaccataccttctccaccgaggtctggacagcagccttgggaagggtcccctccctaacttatgggcttgtccatctgtgagcactctccctagcccagggcattctttggaattctctttgtatatgtatgtttagttctctgtaataatgaatacttctaggtattaacatttccttgttccttagtggacataggaagcctacccagcattctggtctttggccaacactgaggatgtggctgtgggagaacaggcccctctggtgtttcatcttcacttttgcccaagggtgtctgtgatacccatggctggctggggccagagtaaacactggaacttgtaacagaattaaaggtgaatgtttccaagtaaaaaaataaattaaataaaaaattaaaatgactctgtggtttttgacttctgattgggccttaactgatgcaggtaccattctaaatctttctccagccctgagacaccaagggctggacatgtggtctggcctaaggaacgagtaatagaaaagcctcgggaggaaaggcagtcagtctttctgtctcagccctattctgtctgagaccacaggattccctcatctgtgcttcattcttctcccttgacagatcaacatgctttgcttctccaggcactcggccaagtatggccaacctttcatttccaagtttacctgtccttgcatcatgtagccagcagagattaactagcacccatggctgccacagtctggatcacgtcttcttccttggtccagtcagctgagactgaaggtgctgggtattagccatgatataaacatggctagcaggctagccatggatgaatggagactcatttcttagagaaggggacatggactggacagatttattaaaatatatctcaaaatgtatctaacacaggtggatacaaatccaccagcagtgcaacaaagatttggagatcacagataattgtcttcttcaaactgactctatttatcttctctttgctacgtacttaatggcatcagagatatagaagaatagagggtgtcagagacaaaataaaaagaaagtagcttaaggtgatgttatagatcaccacccataggtataggtatatttgaacactctgcagaaacttttgcatgcctgatttcctttgtggaaatgaaataacatagagagaaagtaactaacttttgcatcaagaagggaaagtaaggtgtcttctccacacctccctgcatccgcccacttccctattccttataaagtataactttctggcaggaggcacaaagagaatagccataactatctcatgaaggaatgagccaggtagaaaggttaggaaggagaaagttaggattctaatggggtgtaagctggtacaaatcaatgagacaagccaaaacttgatgagagtcaggagcttcggaatgatgcaagaaggcctcaaaacaaaaaaagattttgaggctgggtgcagtggctcatgcctataatcccagtacgttgggaggccaaagcaggtggatcacttgaggtcagaagttcgagaccagcctggccaacatggtgcaaccccgtctctaataaaaatacaaaaattagccaggggctgtggcaggtacctgtaatcccagccactccagaggctgaggcaggagaatcgcttgcactgggaaggtggaggttgcagtgagccaagtcgtgccattgcactccagcctgggtaacaagagtgagactccatctcaaaaacaaaaaacaaaaaagattttgaaaaaattaattttctgtatgatggtgccatggcagcagtcatagtaaaggaagatttggtgagtagtcaacaccaaccacttgaaataaaatttctttgaaaaacatgaatgtctttagcaacttgtgtgtatgtgttgagtgtgtgtgtgtagaattaaaatgttatgtacctatttaacaattctagtcacaaaattaattgctttaaataaaaagctctgccttgattaataccattagtgtagcagctaaagtattagctgaactgggatctgtatggcaaaatgggagtccatttaaacatacttgtgggaatttaaagacatttacttggggtactaatgacttcaattaaggtagaacacattacttaaaccaagcaaatgatcagcctctgcatatagaagtgtgaagagggccttaattaatttactaattgcacttctcatcttaagcagtgccattgatttggatctgttagtggtttagttgctgttttctctaaccaagcgtggcattccttattggagataggaagaaaggggtgctagggatatgtaaccagtcctgaagattggcagatgcctgtctactttaagtgactttccggtccaaaatgttgccctaacacttgttgagattcctaaaatttccaagggtcactccaataaagtcctcttataaaacacaatcatgaggctgggtgtggtggctcatgcctgtaatcccagcactttgggaggccgaggcaggcagatcatgaggttaagagttcgagaccagcctcaccaacatggtgaaaccccatctctactaaaaatacaaaaaaaaaaaaaaatagctgagcatggtggtgcacacctctaatcccagctactcaggaggctgaggcaggataatcacttgaacctgggaggtggaggttgcagtgaactgagatcacgctattgcactccagcatgggcgacagagcaagcctccatctcaaaaaaaaaaaaaaacaaaacaacaattatgtttcttaaaagtactgaaattggcaagtttttagcaccccccccccccccccagtatattaacttactgtttcagtgataggcatatggtctgagcagaaggttatctctattctctctttctctctgatgcaaaggagggcctgggggataagggctgatggaagatgagggtggaaaggaagactcagggcaaaggtgatgctgtccagggagggtatatcaatctggggaacaatagggcagacaggatttagtactagttttgtttcaatgagtttcactttctttgaatgacattccattaagacatggagtagatttttcatcatcattgtgaataagcaatggaatttgctaatgtgagaaattgtgaaattcctttttgtagagattctggcagaatagataacttctctctttctgggccgttccttctggaagaaaccctgaggaaagagtgtccaagttcatgggttggtcaccaggggtcgccagaagaatcgaaagggcttctgaggcaaagccatagttgcttcttgtgctccaataaagggcccaggtttgtgggacccgcgggccgaccctcttctgttgctagggagacttctcacttaagtatttaggattcctttgttctttctttaatatttagaacttttttgtccgttcccttttaatcatcagttcttgctgcaggactttgacctttaagtcattagtagaggttttattttcttcttaaatatgtagtatgagggaattactcatattttagatctcagctcaaatgttaccctaccaccagaggccttctttaagtacttaatctagtgtctctccattcctctttcatatcactcatttatttagttgcttgtttactgtcttcccacaccagcagaaaaatcatcagagctgacacttaaatagatgtgccaggcactgttctcagtgctgtgcatatatcgactctaaaactgcatgaaaaccttaggagacagacattaatagtatccatattttcaatcagaggacgctgaaacacagaggagttaagtaacttgcccaaggtcacagagctacaaaatagcatagctggaattcaaaccttggcattctggctccagaatccatgctcgtaactataaagtattctgcctgctcatgggtaggagttggtctgcatccacagccttagacaatccctgtgagtagttggcgttgtatgttaaatgaattaatacattgtggtaaattcgtagagtgcatactgttgagcaaaataatgatatgtattgatataaagagatcttcacaacatattgttaagtgaataagcatatatagagtatagcctggttctatttttggaaaaaaaacgtgtgtatgtatattgcatatataatagctataggatatgtctatatgtgtatgtgtagaggaggaccatagagaaaaatctggaagggtaaacataagcatctccatgatcatctctaggaagtagaatttgtcggggaagaggggctgttaaaaataccttcactgaagaaaaaaaaaaacctttagttaagaataagtactgtttttataatagaaaaagttttaagaaaaattttaaagctttttttgttttgagacggagtctcgctctgtcgcccaggctggagtgcagtggcgctattttggctcactgcaagctccgcctcccgggttcacgccattcttctgcctcagcctcccaagtaactgggactacaggcgcccgccaccacgcccagctaattttttgtatttagagacggggtttcaccgtgttagcaaggatggtttcgatctcctgacctcgtgatccacccgcctcagcctcccaaagtgctgggattataggcatgagccaccgcgcccagccacattttaaagcttttgaaaaaaataaatgcattaattgtcttgcttcatacactcttgaattacaacttcaatatggtaaatgtttttaaattcctgaattttgtaatgctgtaaatgaactaaagtggtcatttcttcattcggtcttaagtattgcaatgtggttttgttcaggaagtcttttattctttccaaagttgagagttggctaaaggctgcattctcacaattgacagctgaatatatcaccctaaggattgtcacataataacctcatgccattactgtggacataattaatattatactgcatatcatgacatttgatgccatattatattacaaaagtctgcatgctgtgacacaactgactcttggggttttggcattgaacacatatacttttattttccagatctgacttttatgttttattacagataagggtgacattagttcacaggttggttggcccatcaaagtagaaagatgtatgtgtgggcttttctttaaaaacaggcttcttccagggttattgagcctcattcacctcctccaggaaagtaaatcaggagcaaggtcagctgaggctggaggtgtgtacacagatcttgttatttaagcattccaagaatgcagtgtcctgtgcttcaaagttgcataataaagatatttggcactgaggtgtttgacttgactttgtagtgtgggctctcagggtcaggctatttttctgagtccagaggtccggttcattgtgccaggtcacaggaagggacatggaaggccaagggaggatagggtggaggataggggaggacaggctggagaccagaggcctgagatatttagagggcaaactctccatccaccttcagacccttggcgccgcctgcacattagaaccacctgggagcttttgagaaagactgatgaggcctcctccccacagaccaatggaattggaatctctgggatttgggcctaggcagtagtataaaaatactactctccaaaagagaaaagctctccaaaatgttttaatgagcacattgcagcttctgggagttcacatgtagtttaggaaggaagtggggctggatatccgctggaatcagttggactcgcagcatctcacactccctgggaggctgggaagagcaggggctaccttccacccagatcccattgtttttctacctctcatatgtgtaatttgctcctactgctgagtgaatacaagtggaaggcagtactggtgagtggatctgagaggagataaaaatcacttatttaaacaaaacacagctccacaggcaggttggggcagagctggttatttttattcagcacattggagtttacagcatcatttcatgtacatttgctcctcagagccttcaggcaatcctgtgaggcaggcattatggtcatttgacagacgaggaaactgagggccaggatggcacagctgttatgtggccaactggaaccacagcctgtctggctcccaattccacggctcactctgcttccaagcagcagcttaaaaaggctgtcattgattttcattttttctgctttgacttattattattattatttgagacagaatcttgttctgttgcccaggctgaagtgtagtggcatgatctcggctcactgcaacctctgcctcccaggttcaggtgattctcctgcctcagcctctaaagtagctaggattacaggcatctgccaccatgcccagctaatttttgtatttttagtagagatgcggtttcaccatattggccaggctggtctcgaactcctgacctcaggtgatcttgctgcctcggcctcccaaagtgctgggatcataggcatgcgccaccatacccagcctctgctttgacttataaaacaaaaccctcattctgttgacttgttcatagacatttgaacacttgaggcagggggaataactagtaaaactgatcagggaagttgtgggctacaggagcgagaataaaaccaccacacagcccactttcctattctgtcatttacagatagattgtgttcaagccatctacaaattcctcgtgattatctattatagcaatagctaatatttggagaacacatactatacagctgaggtttatgaaaaggaattcatttaatcctcccagcaagcatacagtgtagctactattattgatcttttttcaatttttattttatttattattattattatttttttgagacggagtttcactcttgttgcccaggctggagtgcaatggcgcaatcttggctcaccgcaatgaccgcctcctgggttcaagcgattctcctgccttagcctcccaagtagctggtattacagacacgtgccactaggcccagctaatttttttgtatttttagtagagatggggtttctccatgttggtcaggctggtctcgaactcttgacctctggtgatccgcccgcctcgacctcccaaagtgctgggattacaggcacgagccaccgtgcctggcctattgatcccatttttataggtggggaaactgagatccagagagcttaggcaacttgccaaaagttacacactggtgggtagtaaagcctagatttgaattcaaacagtttggggcaagagcttccatgcttatttgttatataataccgcgctgacccctggagcttcaggctgctcatcagaggcctcagtcggactcttggcttctaccatttctacctgtttccttagtcataataatcatttatatttattgtgcttttatcatacattaggcattgttcgaagggctttaattatgttgcttaatttgttaacactcaagaatgagcccgagaggtggcgcctcataggaggggtgtgccccagctcatggccattcttcccagcacctatcttgtagcaggtccccctgggcccctttccaaagccgtgaaggactctatcaggctcttggtgactgggaaagttcctctgtgccctctgtggccatggttagaactctgctcagctcttcctgcaggtcttggcggtggcatcccagtcaccttctcttgagtcagtcttgccttctgcctcagccttctcccgccatccaggtcaatggaacgcatccactcagaccttccaacaacaccttgatttgtctgtatagccaggccaagtcagaggtggtgggattttattctgggctgaagccaggggatccttgctgactaaccatccaacgagccatgccatcatgcaagggcccactgttaggtcctgctagaagccaagcaggggagccaagtagagctttttcaggtggttctgggggattcgcctggcgactgtgccttcctccctttctaagtctgttctggtgagagggtttcccttgtcatttcccttgtgcacagctctgctgtctggctctgctcctggctggatccttctcagctttcagggctcagggcagcgctcagacctcattgaagccttcgctaatcaccttctccttttcagcctccaatgactcttaaatcacccatatttccttttcacactctccaatcattgtctttaattatttatttatttgagacagagtttcactcttgttgcccaggcttgagtacagtggtgctatctcagctcactgcaacctctgcctccctggttcaagggattcttctgcctcagcctcccaagtagctgggattacaggtacccgccatcacgcccagctaattttttgtatttttagtggagatggggttttgccatgttggtcaggctagtcttgaactcccgaactaaggtgatccacccgccttggcctcccaaagtgctgggattacaggcatgagtcaccgctcccagccttgtctttatttcttacttactgcctatattgcccatgcaattctaagttccaagaggacatgcgttttcttcttcactcagtgttctgtgtacttagctcccggcgctatgcctggcccataatgggtgctcaataaatacacattgaatgaaatagttgaaaaggtctctgtcattgaaggtgatggtattgacactacggccagtagctcattttcaacatcaccgccctgccaatacccttttctagtttatgtctcccacatctcctaaataaatctgtaccttgttcagggttttgtcgttgttatttgcgttggctaaaggcccctggggctgtctgctcctcctcatttttccaccatgttgaatgaagttgtcctggtccctatggacacattctttttttttttttttttttttttttgagacagagtctcgctctgtcacccaggctggagtgcagtggtgcaatcttggctcactgcaacctccgtctccagggtttgagcgagtctcctgtctctgcctcccaagtagctcggattacaggcatgtgccaccacgcctggctaactttttgtatttttagtagagacagggtttcactatgttggccaggctggtcttgaactcctgaccccaggtgatctgcctgcctcggcctcccaaagtgctgggattacaggcgtgagccaccgcgaccagctgtgaacacgttcttagtgagtaaaatcagaaaggcgtttgggctctcagtgcagaagggaaggtgtcactgagaatggattctttgttgtgcctctgttgggaatatgtgtgttaaatggaagagtcccaaattttagtttaaacagctaaaaatatataaccatgccccaggcagtatgctagatgtctggcagcatgcacaagccaagcatctacatttaaggagctttctgtctcttgttgggaaaagacttgactgcatgataagaagacaccagcatatgaggcacagcctcattcaaagaaccaaagatgatcagtactttgctcatagtggagagtggttactgaggtctggggaggttttgtaggatttagtcacagccctgtaacacagagaggatttgggtaagtttgcttgtgtgtgttctgtagccttaagtgagtgttctgtgtggtcactggttttactgtttcctctagatacagccagaagatgccccagtagttctctggctacagggtgggccgggaggttcatccatgtttggactctttgtggaacatgggccttatgttgtcacaagtaacatgacctgtaagtatcccttgcactgtatttcctatagaatctttcatctgagcctcatacctgctccaaaactaatttagagtaaacactgaaccactatgaatagcttaaccgttccagattatcaaaacacaactttctggcaatatccatccgagaaggaggagcctcggggtgagtgtcctgactgggctgctttctcccttgaaatttgggtgaaaagttctaatccgctttcttgcagtctgctgctgactcaagcattctgggtttgcaaaacagttggtatttattactatttgggacagctctttgcagtggactaatgggtggaattatattcttaattactcacatgaactaaacactttatggttcagtcacactcattatctgataacttggaagcaagacgctattcccggtgggggctgaggcggaggaaggataaagaaagagcaacatctggctgtcactaagtttaccttcatctcattttgtttttttttttttttaattttgtttacttcccctgggtcagaatttggagtgtgagccagtttgtgactcagtataaaggcttatgtgccagttttgttgaagtagttctgttcaagagatggagcattaaggatgattgatttaattggacatatggatattattgcagaggtggttggtgggtgttttttcatgaaccagctttgttccaagcttttgctgggttttgtttccaagctttatctcaggtgtagaaacactctgggctgggggaaagtaaaggagaggagtggggagggtaaaggtgcaggtgcctgttccccaccatgagagtgtgcagcactgttagagggtacgccaacagctcatgaggaccgtggggtgtgttgggcttctgaccaccatgaggcctcggtggaatgcttgtccacgactctatctcacccctcttctttgttttcacagtgcgtgacagagacttcccctggaccacaacgctctccatgctttacattgacaatccagtaagtccgggatgaccctgtgagctgtcagtgctggcgtctcctgtcttttattttttcctttttattaagtacatttaaaaaatagatttcgtttttccttttgaaaatagaatttaatctgtaaaattaaaaccattttgagatttagtttttctgttaacatgtataatcatatcggagtttggcaattttccttgctgccttctctcatttcctcattttttttttttctattgagatggagtctcgctctgtcgcccaggctggcagctcactgcaacctccgcctccccggttcaacctggttctcctgcctcagcctcccaagtagctgggattacaggcgtgtgccaccacacctggctgatttttgtatttttagtagagatgggagcgtggggtggggggtgttcaccatgttagccagtctggtctcaaactcctgaccttaggtgatccacctgccttggcctcccaaaatgataggattacaggtgtgagccaccacgcctggcccatttccttatttttaatcctatgagatgtcctgttcactgaattaggtaacccaggaggaggaattcatttcttttcactaacagtttgggttccttatatcacaggttattatatagtgtcagaacttgtaatctttcacaatcgtctaatctttcacatagagttatagatataaaaagtcaaaatgggtctccactttttttttttaatttttatttttttgagatgaatctcactctgtcacccagactggagtgcagttgcaccatctcagctcactgcaacctccacttcccaggttgaagcaattctcctgcctcagcctcctgaatagctgggattacagacttgcgccaccatgcctggctaacttttgtatttttagtagagatgggatttcaccatgttggccaggctggtcttgaactcctgacctcaagtgatccgcccacctcagcctcccaaagtgctgggattataggcatgagctactgctcttggcctccttcacttgttaagacaagcaacatgcttcatctctggacaaaaagtgatactctccttggggaggtcttctgatattggttctattaaggtgcaaaataggagagaaacttagggtcagcttctacagatttggagtatctttgccctcttaactaaaatctttaacttctctcacttttcagatggtccaaaagaacacaaataaaagcggaaaaaaatgtcaacatgctctttcccccaccttgttttgtcagccttccattttcctcatccataactgtgtatttaagtaaaagatgaattaaacatccttgggtgtttggaaaacaaaaacacaaaaatctttataccagatagggagtgcattccttatgcaaattgttgacataaaaatagtagcttcttcttttgaatttgcagttcagtttcttttgaatttgcagttcagttgtctgtgaacttctaggttctaataagaaaatattagtttaaaattagtcttaattatgttaaaactccttacctactgaagatgaaatggcttaatggccatatttcttgcaaataaagacaagaagggggaggaggaggaggaagaagaagaagaggaagacaatgtacaactacctttttcttcagcctaaagagtttctttctttttttttttttttttttttggagacggagtctcgcttggtcacccaggctggaatgcagtggcatgatctcagctcactgcaatctccgcctcctgggctcaggtgattcttatgcctcagcctcctgagtagctgggattacaggtgcccgccatcatgcccggctaattttcgtatttttagtagaaacggggtttcgccaggctggtctcaaactcctgacctcaggtgttccgcccgcctcagcctcccaaagtgctgggattacagaagtgagccactgcgccaaccaagagtttcttcttggcaggacagagccatgcactctccaggtttcagagaggagacaactttggatcttttgatactgtagcaggggatggaaaagatgaatccttcttccttaaatcaacatctggcagggtctccactgaggcacaggacagctcagccactgaccccagggcaaagctctgtgttgctgttttatttgggctttagggggtggagagaataggtggcacctagcatcctcctgggaggagggctaaaggcataggggggcaacggcaaaaatctgaagtctgagcttggcctttggaacaaggtcaccttccagtgtgggacctctctgcaggtccctaaggcatgtgtttctctgtgtttatacctgcgtgtttatttctcctgcaggtgggcacaggcttcagttttactgatgatacccacggatatgcagtcaatgaggacgatgtagcacgggatttatacaggtaaaatgctcctgctttctcttggctttcctaagaccaaacatttttctatctcaataggaaaattattctagtgttggaatttttattgactgagccttatagtccaattcctgaagataatttaaggatattttcacatccacacacacagagcagaatttttcaattaaagattattttttaaacctttcaaccagacatctgaaaggtttctacttgttttccaaatttgtatttaactttgcatttgcagggagacaacatcttttcagatatttgtttgtttctcaggattcttcttttcgtggcttttggatgcaattttttactttttgcttctatttcatgacttttccaggctgaaaacttagcttgttagtctctagtattctctcttcttcttatgaaagttttgttttgcttaaaaaaaaaaaaaaaaggaaaaatgtaaagttgtaagtctggccaaacagtgtccatataatttctcccttttttcactcatattaatcattgctatttttagcatttttaaaaaaaagcaagtgacctttcttatttggtcagatttttccagtctacaaattattactagagaccaagtcctagattctttctggtcactagggcaccgctttatgtattataataatttactaaaagaaccctagtggccagaaggagttaactagctccctaccccacacctccaccaagcactctgttaattactaccaatcctcaaagcgaaagtgcatctaataggagtcagagtttgctgatctctgacaaaagctttccgttttacttttctttctctctgaggcagataggtggccggggggagagaagaaagaactacttccaagcccctttcccaaagatctctccctctgcccacccagaaaagaatatggctgttgtcttctccacttttgtttgtttttcacatgttccatgattcgatcacttctattcagtttctagggggatgcccagggccccacacttctgcttccacatacagtggggtcctagggacatatttgccaagatgctgacggggtgttccccccagcacctcctccccttccctccacagacgtaacggacggcagtcctcctcacaggcttcttcagagccagggccagggctctcttggcactgtgtgctctctttcttcagattagaaacctactccccgcccccacctcccatccctctttggtttaaatccaaacaagctcagtggttaactagcaaacaccctgagggcagaggttctgtattgaagagatgcatcgggccccctctaagacagagcatcactgccaagccgtttctgtctttccttcccctcccccaaagacagttacagcttccagttgcccctccatatggggatgtcgactgctgacatgtggtccatctcctctttcctgacatgtaaagggaacctatttcaaagcatctattgtttatagctgttgtagttctttttgcttcgggctcttagaactggaaaatagctaatcaagggtggtgcagaaaactgatttcagctgtttcctttatagtttacactattcatttccctttgtgaccacattcttctctccagctctccagatgggtgtttggaataagagggtcgcttcaaaacctgcaaatgggctactacaggtttaaagagtggagctttcattttctccctttgaaaaccaactttcttttttcagaaaatctcattctttttgactattaatagtgcatggctgtaaggagacatttaccgttcacagttttgctgcttttgtgtttggaaataaaactttttcacgaacaagatgttaaaatactctcccacttttcttatttggaatatgctatttagtcacaatgcctctctggtttaaaacaaattatgaaatgtattaactttgatttggagtacactagctggtgccaatctccttctgccattgtttaccaattgcctattatgtgccagctgctgttcttggtgctggtgagacagtagtaagcagtcctcaaaccccctgccctcctggtgctcagtctagtggtggacctatagttattttcttagcagccaaatggaagtgctgctatagttttctcctacatgttggtttgcaatgggaaatgatatgtagtgttgctaaacaaagcttcatagcaatgcagctaggaagcaggcttcctatattaaatgagaaaactctgtgtcctgtgccaggtggagaggatcatggctcagccttgctagatgtagagtgaatgcagaagaggctcccttcacgggttccaacagagcagcactgccaggattcctgttgctgagcttgtatcggtccagtgcaaggcagatgagccccaaagtggggcttagcctgtgaggggttttggatttgcccaggaaagaattcaagggtggccaggtgtggtggctcacacctgtaatcccagcactttgggaggccgaggcaggcagatcacctgaggtcaggagttccagaccagcctgaccaacatggagaaaccccatctctactaaaaatagaaaaattagctgggcttggtggtgcatgcctgtaatcccagctacttgggaggctgaggcaggagaatcacttgaacctgggaggcagaggttgtggtgagccgagatcacaccactgcactgcagcctgggcaacaagagcgaaactccatctcaattaaaaaaaaaaaaaaagaattcaatggtgagatggaggtagaagaaaatagctttattgaaggggcagtgttacaactccgtgtctgctcctgcagggctactgcctagtccgagtagcagcttagggcaattttgcagttgtatttaaacccacttttaattacatgcagattaagggttggttcatgcagaattttctagggaatggatggtagcttttgagtctttgggtcattgccatggaaaggggtggtaacgcctgggtgttgccatcacaatggtaaactgacatggcccactgataggcatgtcttatggaaagctgcttccgcccaggccctgtcttagctagtcctcaatttgatccagtgtccaagccctacctttgtagttgagtcccacctcttacctcaagtccactgccagtgactgtaggtacactttgcttggcttttttggcatggaacttttgcttagattatctgaaactcgattatcctgaatgttgcttgtgtttatgattatgcttctgactctattagaactgaccagattccagtgctgtctttgggtctacaatgcatgatcatgtccactcaccttttctgccctgcatccacccaatctctgtttaaaaacttatctgaggaataattttctcctagtcattcttccttgcaatctgtttctctttttggtcctttaaggtacctttctgcgagcagagagcccttaactaaaattctgtttatgagacaagtccttagcacaatgccagcaaggtggccaaatggaatcaccaagaagaagcatgcttcttgtgccatgtagctcttgtacctgaataaatgcatggagtgtgcacttgtgaacacacatgcttatgtattattattattattgagacggagtctcactatgtcgccaggctggagtgcagtggcgtgatctcggctcactgccacctctgcctcccaggttcaagcaattctcctgcctcagcctcctgagtagctgggactacaggcgcccaccaccacacccagataatttttgtatttttagtagaaacggggtttcaccacgttggccagaatggtctcaatctcttgacctcgtgatctgcccgcctcggcctcccacaggtgtgagccaccgcgcccggccacttatgtattatatatatgtttagcagatctgcttctaaaatttgtaatttcatgtgggcttgattctagattctattttctagaaaatgctcaccatgtaataagaagagttcttgactaaaatacagttaaatgagcaaatggcatatgtaaatctaatttactaaatttacttttttcattcttcactctcttttaagaaacaggagtgctaccacgctactaagtgcttgcttaaaaaaaaatagccagttttttggctgtgaaataaaaactgaaattgaaactcgatgataacttcctcaagtggaacttgagctggagaatcctgccattttcgagatagaagtaacctgggagattatctctgaccaagattatcagataaaacacaaaatacccagttaaatttgaatttcaggtaaacaacaaataaaattttaatgtaagtatattccatgcaatatttgggacatacttatactaagccattatttgttatctgaaattcaaaattaagccatgtcttatgttttcatttgctaaatccagcaaccctaatgactctccagcaaatactacaaggaaataagctttaagctaaagccaaaggcttaattgagatttactatgctaaattatctcctgccattccaacaagcaggaagagagaaaaggaagtagattttagcctgaaggttggagagggaagggtggagaatgaatgcctatttttatctgcactctaaagccagcttattgccagcacaaacatagaagctataaaatgcagcttctcagatttagttgctacaaaggccactataaataatgaatacagaaatacaccgaaactctacagtagtgtttcatggtgaatttttatgcagcctttgaaaattaaatatagctaggatcatggtgattgtaatgacccaattttgcctctttgtaacccttgaacttcgccatcctcctgggttttcagagcctccttgcctttcatcttcctggatggttcatggcttttagtctctcagcaggaaactgggccaacaacctcccctagcttcgcaggcactgcagtctgggtcaaaacactcatagagctactgagtttcacaagtttttgttttcctttttttgaaatattttgttcaggtaaagaatcatttaaaaatccactgtcttcttgcttattatgaaatcactccttgagtctgttaaaatctatttcatcactgaagtggctattggcattctttttttttttttttttttttgagacagagtcttactctgttgcccaggctggagtgcaatggcgcaatctcagctcactgcaacctcagcctccctggttgaagtgattcttctgtctcagcctcctgagtagctgggactacaggcgcgcaccaccatgcctggctaatttttgtatttttagtagagatggggtttcactatgttggctaggctggtctcgaactcctgacctcgttatccgcccaccttagcctcccaaagtcctggtattataggcatgagccaccgccctggccggctgttagcattctttagtaaatgtgtttttcttttttttagcccagagcctactgtacatttatttcaatatctgtgaccatttgctgtttttcttttaaattatagtcatcttacagtttttctgttctttggttattaaaatgtggcattcttggagctccatcagaaatgttcagagtcccccacagtgggggttggtaagcaactaagacttcccttacttcaccacaaacccgggctaatgggatcataaagtttgcagaactacctccaagaattgctacgtaacatgtgaaacctgtattatttgctcaaggtgccctcaacagttagatgatccaaactggtatatcagtaatttggaaacatgcccagagtattgatgtccacttgtccaaacttcttattatcacaaagtaagcccacatgtattgtgtaaataaaatgtcctacaggagctcatagcagagtggtatcttatttcaagtgtgaaacctattgccaaagagttagatttgatatcctagcatgaagagatcctgaatcatttttctgattaatttgtgttgttcttgttcttctttttgcagtgcactaattcagtttttccagatatttcctgaatataaaaataatgacttttatgtcactggggaggtaagtagaagtcacgtttccttgtgtgcttcaaacatcaccagaactgacatatattatgagtgtagtttgttcatttaaaatctatatatgtatatacatgtacacatacatatcaatattgattcagcctttattcaaaattctcccaattatcaaaatgcccttcatatatatatatatttttttttagttggagttttgctcttgttgccaagggctggagtgcagtggcgtgatctcggctcactgcaacctgcgcctcctgggttcaagcaattctcctgcctcagcctcctgagtagctgagattacaggcgcctgccaccatgcacggctaatttttgtatttttagtagagatggggtttcaccatgttggccaggctggtcttgaactcctgacctcatgatctgcccgcctcggcctcccaaagtgctgggattacaggcatgagccaccatgcccagccctttcataacattttttaaatcccagggcccaatgcatgctcacatattaccatgggtgcactaggtaggctcagtcatctgccttgtttgttgtcctcatcattcatgatgatgatttgaagagtccaggccaggtgctttgtagaatgtcccattttctgaatttgtctgattttttcctcagtgcttaggttatgtcaaaacatttgagcaagatcattacatagatggtgtgtactttctggtgtgtcacatcaggaggcacatgaagtcagtttgtcccactgttggtgatgctaaactggatcactgcgtcaaggtggtgtctgccagatcttgccattgtgaaggcacctgtttatgtttgtaattaactcataattaaagaccattcaaacactgtttcctcaaaaaatgccatccagtagttttagcatccactgatctctgtctgaatcagttattacactgggggtgataaaatggtgatttttctaatgtatctttctgtggatatcacctggcattcttccatagggaagatttttcccttttcctcctattacttacatttaaaaatagtactgtggattcacagattgttatttctatttgatctgttaaaatttgttttttatttttgatgtttacattgctttaaatttggtcactgggaaccccttcagttggctcctgtgcccttttgaccggtccccattagcctccaagcatgtccttgctttttgacacaaaggtgtgttccagaagcatcttgtacttcttgtactttccctgccctagccttggaatcagtcagttcttcacagaatgctgtttctttttcctggggaatggcatttaaaaagcaagtgctaagcactaagtggcccattgctgctgggatgtcattgcttttagacagtctgtgggcagagctaggaaacaaaatgacttcatactgatacccataattctaattccataccacaaggttctcttctcttttctctgttctgtatttttttaccccttctcccacattgagggctgtggttcctaatggcatcagtgtgtttcctcttttgattgcttttacaatatgcccaatatggtttcagaattactacatgtgtaccattaccaacaagaaacatcaaaattacttttaatattttttgctgcaatgatttttatccttaaattgtatctcactacacacctataatcaaagtactgtgtttaaaagttaaaagaattctattttttaagggagattatgttaaaatttaatacacagttacattcatttgtctatgttggtttcagttttagggtttgcttttttcatcctttttaagattattttaaaaatgtgtaccagccaatcatttgagaacacaggtgcagttctaggtgctaggaacataatggtgaataacactgaaaaacaacaaaaaaatccatgccctacattctagagaggtaggcagtgataactaaaatatgtgctaggtaccaccatcaggcagggacaagtgtcatgaaaggatgcagtagggcaagggctggagagtgaaggaggtgctattttggataagattgtctgacgagataatgagcagaaccctgaatgatgtggtggggtaatttctgcagtatctgtaagaagagctttcctggcagaaggaacaatgaatgcaaagactctccagtggggacaggcttggcaggttcaaggaaaagcacagagctgctgtggtaggagctgagcagaaagaggccagtggatcctgcagggcccggctgcctgtggcagagtttggactttattctaacggtaatgggaaaccattgagggtccagtgttcactgaaagaacatttagaattgaattcaactgagcacagatatattgagtatctgctatgtttgaggcactggacaaatattaggaattcagtgatttatgagatattgactctgccctaaatctgaaggtcgaataattacaatacagatgaaaggcacaattagagaaatattatctctgatccacgagtcttgaaaaaagaaaaaaaaaggaaaaaattagacaagtaactgtaaagactaatagccccaggaaaggtaccagtagctacctcagtgtgggatcctcaggaaaggccaagcagaggaggtgacaattaaaccgggttttgaaggataaataagggtgtgttagttaaggctcttggttgcaagtgacagaaaccctatttaaagcagctttagtgaaacagggcattgattgatttaacgtcaggtgtggctggacctgggtgcccttactttgtcatcaggattctgcctgtcattctttcctgatctatgtacttggctttgtacttttctggtttcattctcaggcaagttcttttcatgtggtactcctgcttctccatccatcccagggcaggtatagcctatccattcaacttccctttcccagtaattccagccctccagattcctgaatctcagatgatccacttggatcccatgcctatcctagaaccaccagctggacccaggtggttggaatatacaaattggccagaatggtgcctgaggcccactccacaccctgggagggtagggaccaggatggggggccctcacactctgaatcataaggcctgagagttggaaatgagtggtctccagaggaagatccaagtgatgatgccagaggaagggctgtaaagatgctggcaagcacatctgcaatgtccaccttcagcgagggaaagagaaaaatggtccgagtttttccatgggaatggcaagtaactggaactagcaacttctctctttcttctctttataagctatttggaagggatgttctgactgattagcatactacactttaggtttttttaagccagttatgcagtcaggacaacttagaacctctcctggtaatttctgcctgttccttgtcttttaaccaatttcagagccaggttagggaagtgaataggaagtgacactcccattatttggcaacaaccctggaaaatgcccattctccacctcccacagaagagtggtctacttcgtgtgtaatgccttgtcttggaaataatgtggtgatgttgttaaccatgatgttgatgttgatacaaaccatgatgtttggcaaaactgccaaatcccagatgaaaaatttgacttggttatttcacatctgccttgtggttttatgcatataatttcctaatgtgtgtagaaaaggacagttttctttttaccttccttgctgccaggatgaatacttttaaggaaacctgtgccttctgagtggtgaaggaagatgtgaggtagagatgccattgtcaggagatatcatcctccccgaactacatgggatacgttcagtctgggggcactgctcttttcagaagaaaaaacactaaataatgatgccttgatggtttattatgaatattagaatattatgaatatttattcagtctagtttgtcccatctgggttgtcttctgttttaaggccatgtggcagaaaagagaagtggaaggcgttctggattaatcatttggagacctgggattcagggctggctttgctcctccctggatgatttcagaaaagtctttcagcctccctgggcctctgcttttcatctaacaatcagatgagttgttaaactagatcacttccagggatattttgctttactagaaagttcaaattcagttatctagactcactctttaagtatctgtatgtaattgcagttttaacgtaatacctacccattttccctctcgctgctgccaccacacaaaaaaaaattgttctagatttttcaagaaagaaaattcctttctcttctaaactcttaattttctcacactcttaggaaagctgaatagtgctgctgagtttgtagagccatcttcaaaaaataaatgcacaaattcagaaatggttttctgcttttcaatgtaaatgcaccagacctaaatatcttcacatttgtgtgctatgaatgagaggagcctgatgctcaaggggtctggagcagctgttcagaaccttgatttggtgtgaagcagtctggaaataataatagtacctactgcatagggttgttttgagcatttaattagataaccctcctcagatattagcatagtgactggctgtggtgagtacccaacacatgatgactattattaccattcatgctgaaaagctgggctaatatattatgactcccaagcatacagcctaatgcctggagacaagagctgaagtgttaatagaagctccgctaactcattgctgggatatggcatttccccctctactctgtgtctattattttgaaatcatttttaaaagcaatagttactttgtctaaaaaaaaatggagcttagcagttgacattagaagttgtgcttgttttttatatggacagcagcaaaatgtgtcaggacccaccccaggctccctgcctcaccgtgtggacgatggtgtggccacaccagagaaagtggcgtgggcctgccctcagtgcaaaggaaaggggagaagctgctttgtcttcttcctcctcctcttcatttctgcccagaacaccaactggaattaattttctctaaaaagagacaaatgtgtctgtaaatcaccctcctcaaggcatgaaaaaaacatacttcaaaaagagaaataagctgtgtttaattcccataattaactttctttataagtgttatcctttctgagccagtagctattagtgagattgcccttgttcttgaggcacggacacatttagcatcctctcaattgtacatgagcccaggtagaagttaaagcaaaacagccctcacgggaaggcaggtaggtgacaggatcagggggaccgcccattgttctctgagcctgtggctttatgtgccgctcaagggaaagagtcagtgcacgtgggaggttgtggcagtggatgggaacgcttccaaactagcagggcatgtggttaaaacagaacatctcttctccatcagcaaaaacgggcagactctctttataaacagttttgcaaccataatttatttgagaacccaatagagacaaaactttgaagctatttaaaaatgaattagggagcttaatgggacactttatcttcatctgcatgggtgaagtccctgttggaagtacagtgtctgacaacctttattgaaatcatcatggcccattgggttaggtggcaagctcccagtatgcgagagccatgtttgtttcttattcaatactaagcacggtgcttaatgaatattatagaatggagttcatgcatggctgcctttagataaaataaaaatattgatatttatttcctgcatcttttcacctacttttttttttttttttttttgagacaaagtgtcactctgatgcctaagctggagtgcagcattgccatctcagctcactgcaccctccaattcccgggttcaggtgattctcctgcctcagcctcccgagtagctgggattacaggcacgtgccaccatgcccgcctaatttttgtatttttagtagagatggggtttcactatgttggacaggctggccttgaactcctgactgggtgatctgcctacctcagtctcccaaagtgctgagattacagatgtaagccactgcgcccagcccctttctttttttaaaatttttattttaggtttgggtgtacatgtgaaggtttgttacatagacaaacacgtgtcataggggtttgttgtacatattattacatcacccaggtattaagctcagtacccaatagtgatcttttctgctcctctcgcctccctttcattgcccttctctccctgtactatgtcccaggccctggggactctcctttggcctccgttgttttccagggccatcttcccacctcagccatcctctcctcagtctattgtgcacagcttctatttctccatgcttcttccatctgtatcatgattgttctgtttctgtcccccattctgacttgttgtcaactcattgttccatagaaaggatctgataaattaaacccaatagggtggactctcaacatgtttgcatttgagtagagagcagagcctacctgatgggtagatatttgtaatagggtaatttcaagcactaacatgcaaacagaagggtggcaaccaaggtggggtgggagagcgattctagaagtaggcttataattattagcattctatgtaagcctgtaccatcatcctgttagcctgggcttgggatcatgtaatttgatggctagaagtgatacttcaagaacatctagttcatgtattttatctgtagattttactagagaatatttattgagaatggactgtatggtagacatcatgctaagcactgttttatgcattacctcgtttaatcctaacagctgccttatgaaataggtgctatgattattcttcccattttacaggtgaggtgaccgaggcttagtgaggttaggtatgccttaccaaggtcacccagccagaaagtggaggagccagcattggaacctaagcagtcagactccagggcctgaagatggtggtgaagaaatgagctagtggtgcagccttgttcaccaaaatgcttgttcaccaaactgggtgctctcaccttgaacttgcactgtctctaggacctgcttcagattcactttgtgtctgaaggggagtcaaacatccccttccatattttatgctgaatctctaaggttattaaccttacacaaacagaaaagaagttctctagcttcttgaatgtctctgtctttctggccaagactgttttaatcataaagacttggctttgatcattaagttttatagagtaagtggaagtttcaagtgatataaggacttgaagtctttggaatgcaattatttctcaaggcttggaacttgctgaattttattagatagcattcaagctgttttaactcttgaacctatgacaaatctctattttcagttatgttcatctgcacttgccaatattttctttttgaattgaaaatggatttgtgactatttaatgattttaatattacaaaaatcagggtttaagctgcaaccgaagaactgaacaagggcatatggaaatgtttgcaagttggatctctggcttttggacagcatatccaaatcaaatcgatacatatgtactgaggttgcacagaaaagaatatgagtaaatcagaagtcattataagctttgttggaaatgaattaccatgctggagcaggcagcatagagacataaatagtacctgattagatgagatgtttaggttgagaatgagaaatgctaagttacatccattatgcagcttcatccttctgaattgtatctacagtgagcaaacaaagattcagtttgttgactgcggcccagtgtgttcagactctttatttaaaagtcaaattaactgtagtctccattttaaacaaaagggtttattcatctcattgagtcacctggcttcacgctggatttctccaaactggcactgaactcagagtaggtgggaaaaggatgcatcttgttcacctatgcctgtcaatgatggctttttcttttttaaggagtatcttctcaatagaaaataagaatatatcatttgggaagcagcatgtatttttgcataagtttatgtgactaaaaattggctatagatcaaaatgtttttgctatatgtctatgcctgtagattttgcaagtttctttttaagcccatattaaaaaagaaatagatactttgataaagtcatattcttgttggaaggctcttttttttaattgcagcagtatggtcttgaaattagatgacgcatggtctggaaatgtctaggaagttatagcgattaccacaacactgaaattccagctttcctcattcattaattttctaaactggccattttaaggccacacttctctcttcagagaaaataatggtactgaggaagtaagattctaccctaggctagatgaagccaaggtaggtcgccctagcctagtcttgccctcatggtgcccattacaccgaggttttcaccaggttttgtggctttcctggggtgttcagtgtgtgtataaagttctctgtgaggcattgaagctgtcttcttccctctggtcttctcccataaatgggtggcagctctattgtagtctgtgcctgggatgagctggagaacaagggaatgatatgactgattccagaagggcagtgcgccttgatgccttctggggatacaaaggagctctcaccagcccagtagtgaattcttcaaagaaggattttttttagttctgtgtagctggggagagtggggttcaagcagggcagggagcttagaatgaccctcagggtcctcagtgtaccattgctgaagttgacaactccctctttccacctgccaggcaaagggtaggaactaaggcctgtggatggtgaaactttgtgtctaagtttagctgtgtctcatttcaaaagccattgctcattatcactgacaaagttctgaattttttcaccatgaaatgtgtacctagaagcaaggcagagaatgagtgtataagtgtatctattttttttttctactaaacttcagagaaatgtagtcaactgagagtagctgaaacaatagtaactgctaccatatgtgaaatttctgtaagaggacattgtttccattttaatattattctctgacactatttgttttcctctggaggagctgattgaagctggactttagctatgaaatctttttcccttccttgaaagtaaccaaatggcaggttacagtaagtaggctgtgaatgttgttgaaacataaaagaaaagagagattcacgcaaaagacacgattacaggttttctctacatccaatttcattgtgtttttacacatctggataattaaccaaacattgcattctctctatttttacaagtacaatgtgctcctcacgggatttccattaggctggaaaaaaaaatcgcaaaccaagattcaaaactactattcagctatgatgttgaggcctttttctctcctgggagaaataaaagattgagagctgaattctgagatccttctgatgtaggaggagaattggaagtttatagcctctttaagctccagcaaatacaagcatgtacaaacacaccctaatacctctggtccttcttgttggaggtgagctcgaaatatggtcaccatgggaaaaattaaccaagtaagagcagtgctggaaatcgcagcttgaagctcctttaattattagaaaggaataataagggcaccctaaaaattctgatgtgttgagtgctaacctagtttgaattctgttagatgtgttatgtttatccggctgagcttctcattcgaaggtcactgccaaatcctaaaataatatgtgttcagttagtttctggccacacttttcctctttctttttaaaattgctttggtattgctctgagcttttatgagaccagggtctgatcccatgttaggttagtgttaattcacagacctcaggggcttcattgaccagctgctgcccacactggcgaacaccatggggaggtcagagaagccacgggaccaagagttctggttgttgttgctgttttcattagaactcagaacaaaagaagcaatagttcataaaattacagaatcactgttataagcctaagggaccatttagttgatttctaatccataaggcagtcacacctaaattcttctcctgaaatatggggttctataaaatatatacaggaaaaggcaaatgaggtaaaataaaacacacttgatttattacctggttgtcttttgaagtacttaatggcggtcagagaatcattaaattgagaaataaatgattcaagaagaataaatgagtctgatcttgccaaaaaaaaaaaaactatctcaacaaggagagtgaaaataactgaaattttattacaatatttcagtgcctgttttccataaaaataattctataattgtcttctctgtgacagcctgagtcactcaaccaatggaatgattaatgaaaaaggcaatgacggatttgtcacactgacacaaagcaaggaagtgtgatagtcattagaaagttaaaaaaaaaaaaaagctaagatcatcactgtgaggttggcagatgtggactgaatattgaagaatttcattttggatatggttatagtactactcatctgttttgtttttcataaatgatcagcttagcaagcttggagtagataaacccaagatgtagttccaccttgggggacctcagacccgtggtatattccaggaccattgactgtcagtgatggcttttttttttttttttttaagaagtatcttctcaatagaaaagatttcgttgactgaaatgcaattagatatgacaaaggggagctgcagagacagccaagccagggtagaattccagccaggcatgtgaatgaaatgcacgggcccttgttttcatcactgtaaaatattggaaaacaatgtttatcttaaattggcttaaagtaggtgaggtagcctggctcatagtaatgctcaaaaactgagtgggtgttccttcttaattccctgcctggatatatcccagatacccagatacacatgatggtcactgggggaaagtgccacaaaaatgcagatgtttcagcctacgtttgtcatgactacctacctagcaacttaaagtacatgcccttccatgtcagaaagggtcaagagagatgttttattaccaggtcttataacaagaaaattggagtgataatatcactggaagataaactttttggcatttgtaccaatttctgataggggaaaagtatttagaagtaatgccagataagtcattcatttatcaagtgtgtttttatttacctattttgtgctaggcaatgtatacattatagaaattctaatctaagaaatcgtaaactttgctctatcatgtgtcagtcatggtttccataatatagttttgaaagaaattgtgacctttatgagggcaagttttgtgctttattcatttttgtgtctcctcaagtatctagtataatgcctgtattagttcattttcacgctgctgtaaaggtactacgagagagagagagagagagcatgggcaggggaaactggctcttttaaaaccatcagctctcgtgagaactccctcactatcatgagaatagcatgggggaaactgcccccatgatccaatcacctcccaccagatcccttcctcaacacataaggattacagttggagatgagatttggttggggacacagaggcaaaccatgtcaatgcctttgacagaagaattgcccaataaactatcattgaataaagactatataacaatacatttttaccagtaaaatggttactcagggaaatgtgcttttttgtcattttacagaaattgtatttgataaatgttcacttattgtcactaacaagagaatgtagcacagttagacaacaaaccaaatttcagacatatgaacacacctttatattacacaaggaagctaaaatgtacttacaagtatgaatggataaattgctttgattcaatactttacctaaataattattattgcacaagagagctgtatcaatgaggagttcttggataaattacataaatgatattgttttatcattgcttggaaggagtttaggttttggagtcggtcagctcaggtgtaattctggctctacaacttaacctctctgagcctcagtgtcctcatcttgaaatgggagtaatgattatcttcagcatatgattatggtgtggataaaataagagctttcaggggttcccagcctttgaactacagacccctggagggccttggagatgctgtgaagggtctgtgaactcacaggtatccacataatgtctctagtctgttttgaacatagatgcattgctctttttcagatgtctgtagattcattttaagattcattcattcaacaactatttattgagcactaacaaatatacgcatcagtcatagtggactcaagtaaacaaaacagacaaatgtccctgtattcacggatcttatattctagtatgggtaggagaataacagtatataagataagtaagtaaaagtttgttggagaatgataagtattaaggaaatacttaggaaagaaaaaatataggacattgagggaatttgccatatgggggcctgtgagaagagcattccaggtggagggaatggctggtccaaaggcctggcagacatgtgcctggtatgtgcaaggaatagcatggaacccaatgggctggaaaggaatgagcgagagggtggcaggggttgagggtagagaagggagcagatcatgtcagcccttatgcctttgcttttgggaagatgggaagtcattaaaggataagggcaaaagaaggaggccaatgaggagtctactgcagtcatccaggccagagatgatgtgatggagaaggggttgtgagaagacattggactctggacagattttgttagaataggtagagctgacaagcttggctgattgacttctgtggaatacgagacaaagagagatataacgaatggcaccaagattgttggcctgagcaggggagaaacagaattaacatgagatgaaagggaggctgcctcagggttggggattgggagctttattttgaacatgtgcagtttaatattagacatacaagtggagatatcaagaaggcaacttttgatggaaagaatgcaaaatgcaaaattatttgaaaaatactgctgagtcaatagtagctcttccagcacagtcttaaagtagaaccgttaccatgtttgttaggggaaatatatctacaaaatattctgaccttgaaaaaggaccttgtgctggaaaagtttggaaatagagatgagttcaagtgaaaattttagtggccaataaatattattttccttttctgcttctcctattcctttagtacatgttgatttcaaaatttgcagcattatcagattttgaaatcttaattttaaacttgagatttttttccctctaatctttgaatagagctttctggtaagagatgcctaacacatggcagaattgtgttctcccaagttattttattttggaaggaaattagaatgttcacttattacatgttcacttgtgattttttgtgcatcgaacctgttcatgtcatgtggtgctatttgagacgacaaaagaattggaagacagaggtttttgctattatggaatctataatgtggggttaagaaacagatggtatacctcatgtggaaagattgctccttcattgttattttaattagcttaagtacatttaattttactagcatccatttgtgattggccatttcaacatctgtccttctcagtcttatgcagggaaatatgtgccagccattgcacacctcatccattccctcaaccctgtgagagaggtgaagatcaacctgaacggaattgctattggagatggatattctgatcccgaatcagtaggtttctccttctgactttctattttgtctcttagggggattaaatgcctttttatcagatggaaggctcctcttagaggtctaacttttgataagggaaacaccagttgactttatgacttaaatctcctaatttttaccaaatgtttcaacgtatcaatttactgtgtcagcttacattggctgtcagtgcaaatgtgtgtgtgtttgtattgtgttgtgttctgttgtgtgtgttgtggggtgtatatgtgtgtgtgcatgtaattatcctacttggctaaccagcacaatattaacaattacaaaggattatttcaccaagcaaatgggcttccttaaggtctctttctctccctttgtcacattgatgtgtgtcttttttgtcctcagattatagggggctatgcagaattcctgtaccaaattggcttgttggatgagaagcaaaaaaagtacttccagaagcagtgccatgaatgcatagaacacatcaggaagcagaactggtttgaggcctttgaagtgagttctgggggccctgtgctgcccttgagcaattaaaaccatctggggagggagggcgggtgagcaggaacaccttggtgctcaggcatttttttgttaagaaggtatatctgttattccattggagatcgggaggcgagttgagtggcctcataatgaagaaaaggactgtctgtgttcctttgaatttactttggctatattttaagcctgagatcatcttggagtgtttatctaactgtgtctttcattaaccgttttgatttctttaaatgctgtggaggccctcaaggcagactctatcatatggaaatcagaagaaaggcaacccaggaagtagaggcatatgcttcaccatctgctctaaaccatgtagaagagttttatatccacaaacagggtttcagctgggagaaatgaccacagtgtgtaacagatgacatgtgacacacgtgtcaggaacaaagattataaagtgtagtaatttgaaactcttacaaagggccattggtatttgtgcatatcctttcattgctcccctaaaattaaacttgagaatcaataattacgttcttcactcttttctttctcagttatttaaaatgtaagaatatactaaatattaggatacaggaagttgggttttttttgtttttttttgctttttttgccaatgcagacagagccatgtgtaaacaatctccctgaggggctttttgccgtatttactatacagccatcatgtttgacatggtagtaaatgaggcaaggtctgaatttcgagggctttaatttgtttgttattagacacatattctgtaacagtatatctaatgtcgttattgttattaaagcagaaatctgatattaagtcttaagggcattgcagatcagccagttcagatcaaaataggagacatgcaaccttggagaaaagggtagaggataatacaaagttatttttcattttacaagtagggtgttgggacactatcagaaatcccccaaggcagtgaccgcccttgccgtgaattctgatagcagctagctgtgcaaatggatggagcaaagggagtcctgagccatcctacagagctccaccaggctctcctttctctgtgtcaggacactaaatgtggctgcaaagtgtggattcagaatttcttatgcaattgtaaacacaaaggaagaggggtctcctggggcacatgtaggatcatgcttccacttgcaatgacagtattttcaaggctttctgctgtggtagtcacagaggggacgaaggagtgaaacttcagtatttagaattaataccaaattttatgtggcatgagaccaatttgcaattgaaacatttgatatgtttttaaagaagcacacaggataaagcaattgtcaagagcaagagcatttgagaggcccatatttagaaaaaaagcagaaagatgactggtacataatgcagcaggctaaacacctacaggattagtgaagctgtagaaaagttcaggtgtgttgtgtattcagagataatgagaacatgacccacgaggtgagaatctctcccaaaccggatacgtcagaaaccggaaccccaagcaggtaaaacgtttgaaggcttggaatgtctttttgaccattggtcccgtatcttgagtgatgaattaaaacaagacctttgctttttcaaggaaaatagataaaatgatacttaggacagtaaacaacttgtggttgaaatgtgtttcagtgactgcaaaggaagactccaacaaatcctgaaacccaggggaatataaatgtcagaatctcaagcctaaaatgataaagcctgtttgacgctctgatcctttaaaatgatttaattaatcaaagagtagtaactcagagctggagagtactctggagataggtcatctactgtccctatgcatttacatgcacacacacacacacacacacacacacacacacacacacacacacacacacacacacatttactggtgagtggagaggatgccgagccccaggggatagcaatgaattgccgaaggatacacaaatagattgtggcaggctgaggactggaattgcttcccccatttccaccacagtatttatcctatggtaattttcaggcattctgtaaaaatcctggatcgtcttagatattctgcaagatgtgaaatgactgatcctctgtccagtattacctccctttccatgatgattttctgaatcacaaagttcttagtttcacaggtcttgaccacccatgttattttttgacaacaactgactttttcttttttttttttttttttttgagacggagtctcgctctcttgcccaggctggagtgcagtggcaagatctcagctcactgcaacctccacctcccaggttcaagccattctcctgcctcagcctcccgagtagctgggattacaggcactcgccaccacgaatggctaatttttcttatttttagtagagacgggttttcaccgtgttggctgggctggtctcaaactccggacctcaagtgatccgcccgcctcggccttccaaagtgctgggattacagctgtgagccaccatgcctggccaacaaatgacttttaactctttctaaaagcaaagttattcttcaaggttgactttatattcccactggtgttgaagataatttctaacaatgagttccaaaaatgatgtgtgcattcagaatgaccctggcctatggtgccccatctgggtgaacaaattagaaaaagacacctcggccgggcgcagtggctcacgcctgtaatcccagcactttgggaggccgaggcgggcggatcacgaggtcaggagatcgagaccacggtgaaaccccgtctctactaaaaatacaaaaaagttagccgggcgtagtggcgggcgcctgtagtcccagctactcgggaggctgaggcaggagaatggcgtgaacccaggaggcggagcttgcagtgagccaagatcgcgccactgcactccagcctgggtgacagagcgagactccatctcaaaaaaaaaaaaaacaaagaaaaaaaaaaagaaaaagacacctcctctgtggtgatgcagtcctgtgggttcaggacggggcaggcagatgcctccattgtgtgaggataagaggtggccacttctccagatgcagccctgagccaggctctccagctcagcgagcagaactttggctggttccctgtccctttgcaccccttagccacaggcatcgcatggagctgcacaaccagatgtggggccctgagcaatggtagcattggtagagaatacttactaccacctccacccacaccgccttctatggtgataacccttttcattcttggcctctagccccatggcaataggaacttgaataagttctttcatgtttgaaaaagaatatgaatacaggccgggggcggtggcttacacctgtaatcccagcactttaggaggccgaggtgggcagatcacaaggtcaggagttcgagaccagcctgatcaacatggtgaaaccccgtctctattaaaaatacacacacacaaaaaaaaattagctgggcgtggtggcaggcgcctttaatctcaggtactcgggaggctgaggcaggagaattgcttgaatccgggaggcagaagttgcagtgagctgagatctcaccactgcactccagcctgagtgacagtgcgagactccatctcaaaaaaaaaaaaaaagaatatgaatataataattagtctattttctcaagcattgaagcatggccagaaggaattggcaagtaggaagcattgcaggctgactgctgggagccagtgtgtgcgtggttgagtgtgcaggctcaggagtcgtggcctaatcatctcatctctgacattattagtgaggtgtcctcggcaagttgcttaaactcatgtgtctcagttttctcacccataaaattagaataataataccttatctgataagagacctaaatttgtacttgatagattgtttttaacatgtgctgacttcatttctttgaattcttttgtaagaactccttacaacttaatatcatcatcaacaacccccctccatgtcttcaacctcttctgtgactattttcttctcttcatcttcttgctttgccagaaacctggctctcccctgatgctgcagtccctgtggtcctcttgagtgggggctgttctctctcttagcttgtctgtatcacttggttcataggtggagtaggtgtcttccttgctcatcatgtgactttcagatcatgttccctgcctcctcctaaagcattgagctttgactctcctgtcctcagacagggcctctgaccacttccaactgtgtctgcctaccaacccctggttactccctctcatttgaatagctgagctccttcctccctgtcactctctgcagcattattcttgtcataattcctggtaactgcaggatccacattgacgatccttttgggacctggtccttggtgcctcagcctcctctttttcaatgatcttgttctcctcctcatcttagctgctgattctcataccctaaaatgctggcagctgatccacaatcacacattcaaacatctttctccaactaccacctcctgtccgaatctcttctgctaatgtactgatcttacagccctgcaacctcactacctttcaatccattgatcctcctgcctctccactatctctaaccccctcaagtctccatttctctaattacctgtcttcaacccagggtcagtcattccatacatttccaagcccaaagctcttttgcatttcttccaactcctttggctgaaccacagccctggctcaatctgcctctctgtcaggctgtgcttgtctccacacagctgacaatggctggaggaaagcatgcaaccatgctgactgctctcccgctcagctggtgactgggttttttaatgtcagcccaaagataacttccaccagctccaggaacctgcatctgcaccatatcctgcgtcttctctccatgtcatctggtgagcagttcatgatccatgttaaggcacccttcccacaaggcttactcccataggcaaggcctttgagcttactgtgtcctctgcctggaaccttccttcccctagatgtctgcatggctctattcctctcatccaatatgtctttactcaaatgccaccttctaagtgaggtcctctgtgacctcctctctacattgtcccatacacacatgcacacacatgacacttcctactcctctttagttttctccttgtcattccctaacatactcaagagtttatcactgtctaccatatgatataatttacttagaaatgcttgttgtcccccaccagaataaaagctccatgggggaggaccattggtctgttttgttgatggccatttctccagtgccatgacagtgtctagcacagagaatatgcacaataagtgggcgctggatgaaacaataaaggaatgaaaaagttgttgaataaatgaaatgacaaatgtctgtacatttttatccacactgatgttttctttctttctttctttctccctctctcccaacctagatactggataaactactagatggcgacttaacaagtgatccttcttacttccagaatgttacaggatgtagtaattactataactttttgcggtgcacggtaatgacattttaaaaaccataataatgtttgcttaaaacttttggcaaaaccgaacttcctttgatttgggaaatattatagatgactttatactagaccaacatgtccaaactgtaccgcgtgatgtggtctactaacccatagaatcttttcctcatcagtgttgtatacgttataatctgaccactgagaatgtgtgatatttcagataactcaaaacaacttctgatgaacatgatttcccccaagtagagcttgttatagaggcacttttgtgaaggacacaggctgggattggagttctgggtctcaggtcccagagcttcaagtctgtttacgagtctcttgaaaatgcccacattctttgttctgtacttaagacgcttggataatcaaaactgccaaagccttaatgaaaatatttgctttgtcttacttttaagatgtttaatgcatttagtatggccctcacttcaagccttctggaatatttctcaaagtattcaagtagtttgtttcataaaagtaggccaaagttgatatcttttgtgtagaattagtcaaacagaaggagccaaagtaggtgtttgaatttgtataatctttgtctaatgcttatgacaatgcattctgtttttaaaattgcctgatttatgaactagtttgtaaataaagtataacgttagtttcagtggaattcgccaatgctgcagttttggatttagattctactaagagaaaaacatgtggtagacaagacagaaacaaaatttgtgatgatacctttttttctttacaacaatatattcacagttagtaaaaattgttgatgaagcacaaagttgtcaaactcaaactttaacagtttttttaatccttgtgtaactgttttttttttaaagcatttactcctttctttcagtataatgattattacagagactttattgcagttttttttttaataagcttcatggacaacttttcttctgtcttgtgtccttcataaagtatcatctcttttcacatttgacatattttcccctttattatagtcaatccaattttgtgtgtgtgtgcggttttttttattttttaattttattattattatactttaagttttagggtacatgtgcacaacgtgcaggtttgttacatatgtatacacatggacacaggatggggaacatcacacaccggggactgttgtggggtgggggtaggggggagggatagcattaggagatatacctgatgctaaatgacgagttaataagtgcagcacaccaacatggcacatgtatacatgtgtgtggttttaataaacagcagaacacagaaaagaaattcttagctttaaatccagcacaccaccaacctggcccttgtgatccatttgaccttctttccaaagagccaccaacaaggagtgaattgattttgataatctcatctcttttgttttctctctctttgcctctagtggggttgctaatgggagtcacagattgcaaaagattagtttctgcaaaggaaaatcctgtatttgcaaattgggtattttaagagtattttaagggtatttcagatttctaattagtaaaaagattccacctaaattatctcaaaacctccgagttaaaatttcaagaatggcgacaagttaatagtcccgtcacgtttctccatgttgtatgttatgagaaattcacagcttttcccaacatactgttcctgccaccaatcatgttccctatgtctttccaatgccccttctgccaggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggtaagagagctacttcagttactattttaggaactttcagattacccagagcagaagtgatttgtaatttgagtcctgccttttttttttttttaacttttatgatgtgtcattagcataaataacagcccctttctttgtttgattccaattcaaagttttatattgttccttttaatttaaccacttttgccgttgtggtgacttatacctgtaatcccagtgctttgggagcctgaggcgggtggatcacctgaggtcaagattttgagaccagcctggccaacatggtgaaaccttctctctactaaaaaatacaaaaactagctggacatggtggcgggtgcctgtaatcccagctacttgggaggttgaggcgtgagaatcacttgaacccgggaggcagaggttgcagtgagccaagatcgcaacattgcactccagcctgggcaacagagcaaaaactctgtctcaaaaaaataaaaataaaataaaataatttaaccactttcccctgattattccttaaaatgaaattccctttggcacacaccacttctcatcctttcttgagaaaggaagagttgcagagccaaaaagatgactggccgtgtgtaagagccagatttctcttatgtgttgctttgttacatgaatcccaatgacgtcagggataaatgaaatcaactggttattctgaaaatgcattttgcctaatggttgtattcattcactttacccaatcacagtgttttgagtctctccttagggtgtgaggttgaaatgatgaatgagaaagggtccctgtttggaagtaggtcaagtcaagccattgtgctgtctggttgaaatgctctgcctttgctgccagtggaggtgccagtggagagtgaggtcacagaaaaggagccctggaggttcaggcaaagaagacatgggccctggaactcccactgggaaggaggacaccaagagcaaatgacatggggaaagtcagcctgtgtattatttctttcctggtactacgctctcagctcctcctctggtcctccagtgctgttagcctttgcccaagggatcgtctctagggcaactccgaaggatgcctgtactgatgactggggagaccaccactgagtgatccacttcctccctatatcctgttttgaatgaacaaaaaacagggcagtcggcagcccataggatttttgtatgaattagaataaaggcatcctctcctcaagatactttatttcgatctttcagaaattgtcctgatatactgccttagttaaaaccagactctttactgccatttgctggcatgctgaatattcatttctacaatgtgtatattaggaatggtgtgtcttaggattgttcagtgcacaatctgcaatactatccaggcagtccctggccaatgcaccctaactagactgtggtttagaatatgccagacctaggaggcccatttctgggtcctgcttctagtacagtgttggacacgtaaccattgttgaaaatagtttaagaccaattatatgccaagtctacaaacccattataaacctgcaaaacttgatgaaatacttactttcttggaaagaaagagtctaactaggtcaggccagcatttcagccatccttgtgtttggttttcttggggacatgtagtggcccttagtgcttgctgcttcaagacttctcagaggacctccagaaggcactgctgtttgtcctctcctctgagctctcagaacgcgttgtctttacccccataggtgtacatggcacactctaacgtgcagactctcagtctgacacccataccaccaccagcctggagcagatgtcaggaatgaatgaatgattagtgctgtatcaaaaatatatctataaacctgtccataatccattaggtgcttattatccacaaatttattaaaccaaaaagaggttagcgttcatgtcttaaagatgatgttctgtaatgtgatgttaaggaatggaacgagtgaatttttttgagaggagagcagataagtttgatatgtgttatttgtgcccttgggagatatattttctcactgtacacactttttttttttttttttgagatagagttttgctcttgttgcccagctgtagtgcaatggtgcgactggctcactgcaacctccacctcctgggttcaagcaattctcctgcctcagcctctcaagtagctgggattataggcatgtgccaccatgcccagctaattttgtatttttaatagagatgagttttctccgtgttggtcagattggtctcaaacttctgacctcaggtgatccgcccacctcggcctcccaaagtgctaggattgcaggcatgaaccaccgcacctggcctcactgtacaaatttaactagaatatgattcctttctcagccatgtgagggtaaggtgggacaaacagcttacctgatgacataaactctccttatgttttgactgataaaggagaatatattgattagaggtgatcctgcattttgatatttaaacttctctgtccctcttgcccatgtatcctaaacttaccactatgaattccaataaaatcaaaatccagtattttctaaacttttcttttcttttttgagacagagtttcactctgttgctcaggctggaatgcagtggcacgatctcggctcactgcaacctccacatcccgggttcaagtgattctcctgcctcagcctcctgagtagctgagattacaggcacatgccaccatgcctggctaattttttttccgtatttttagtagagacagggtttcaccatgttgcccaggctgatttcaaactcctgacctcaggtgatccacccgcctcggcctcccaaagtgctgggattacaggtgtgagccaccgtgtctggcataaacttttcaactctgaatcggttgccagaaaaagaaaagagtttagtggtatagataaaccatatttccataaagaaacagaacaaaatatgccaccaaaaagtggctgctgctaaaacagacagcatgttcactcacgcagctggcaaagcgcttgggatagtgattatctttggcttgtctgttaatgatgttgaacctcagtgttcagaacgggaatcagaacaacctaggttggcttaaaacgttagtaccattaaagtcatatttatgtaagactcggtactaatttgtcatagaaaatagcatgcctgtttctattcataggaactagaggtctttttccagtactgggttcattttggccaatgtggagcatctctgatggttgaaggaagaccttcctgggtttctggaattacgttttcatgtagtttctgtataaaaattagcttctctgtaaactacgttagtttctatgtaagctacgtgaaaacataatccttttcttacagtgatatacaggagatgcatactcaaaagtcattttcctttcagtaatttcagatgtttgggtggtaaattgaaatatcatattggcagtttcaaaaataactagaaagagaaacaatctgttgggctatttgaattctttatgactgaatgaagtcaaaacatgatcaggacttttaggagttagctggttaatagagtatggtggttatgaacatgttcttaagttgcatatcttgagtttgagctctggttacaccagtcactggctatgctcctttgggagaggaaacttaaactctctgcgctttggcttccttgtctataaaacaggaataaaaacagtatttatgtgtgagtttctgtgaggtttaaatgagattatatatgtaaacacacttagaagagtgcctgatggttagaaggtgcttatttatatatatataagctattaccaaaaatgttcaacaaattttgtgaaagtctttaagatcttgctacattcaaaagttatacaagaaaccctggaaaacacaaaaataaaaaagaatggacaggcttccagtttccattccagtgtgtaagaagcttagaagtctccactccattgtaacaacagtaaaaagccaaacaaactgaaatataatcaagaactctttttagatttgttgaaactgaggtcacagggcaaattgctgccctccaaaattagagagagagatgaatatagggagtcaaaacttaacaggagcaaacctgtaagtagaaccctccatggaaacaaatggtgaggaagggaaacctgaacagtaattggtaaattactagaggctccatagggacaagtctgagagtttaaaacaccatgggtggggagaggactcagccaaaggtcacttgtaaatgacctttattatgttgagggacttttcttgtatacccagactgttgagtgtttttatcaagaaaggatgttggattttgttgaatactttttgtgtcaattgagatgatcatgtggttttgtctttcagtctgtgatgtgtcactttgattaacgtacctatgttaaaccagtcttgcatgctaagggtaaatcccacttgatcatgctgtagaatctttttggtgtattgttgaatttggattgctaatattttatcgataatttttgcatgaatgtcagagttacagccttgcagttttcttttttcttatgatatctttgcctggcttaggaatcaagataatacttgccaaaaaatctatgattaatatgctaaggattttaatggaaaaagtagacaacctgcaagaacagatggatattgaaaatagagatatggagattataagaatcaaaaggaactgttagagatcaaaaacactgtaacagagataaagaatgtctttggtgggctcactagtagactggacacagctgagctaagagtagcatgaggatgtaataaataaaagcttccaaaactgaaaagtaaagagaacaaagacttaaaaaaattttttttgaatagccttaactgtgggacaactacaaaagatgtaacatacacataaagggaataccagaacaagaagaaagagaaggaaatggaagcaatatttaaagcaataatgattgcaaactttcccagattaatgtaaaacatcaaagcacagattccagaagctcagaaaacatggagtcagataaatgccaaaacaaaataatcaagaaaaggaggcaagtcatatggaagcttcaaaaaaatcaaagataaagaaaaaaagtctcaaaagaagccaaaggggagaaaaaccttacctatagagaagcaaaggtaagaattacatctaggttctcctcagaaatcatgccaacaagaagagcatggagtgaaatatttaaagtgttgagagaataaaacccaccaacttagaattctgcaccttgcaaaagtattcctcaaaagtgaaagagaaataaagactttctcagacaaacaaaaattgagggaatttgttgctagtagacctgctttgtaagaaatgttaaagtaagttttcaaagagaagataagtgatataggtcagaaactcacatctatataaagagaagaagagcattggataatgattaaatgaagatgacataattatttatgtagaaaatccagaagaatcaacaaaaaagcctcttggaattaataagtgattaatcataacaaggttgcaggatataaggttaatatagaaaagttaatcactttcctatgtactcccaataaacaagtgaaatttgaaattaaaacacaggactatttacattagcacccttcaaaaatgaaatacttagatataaatacaacaaaatatgtataagatctatattaggaaaactgcaaaactctgataaaagacatcaagaacaactaatagttggagaaatattttgtgtacatggatatgaagactcaatattgtcaagatgtcagttcttcccaacttgatccatagattcaatacaatcccaatcaaaatctcagtaacattttgtagataaggataaattgttctaaagtttctaaggagaggcaaaagacacagaataaccaacacaacattgaacaagaggaataaagtcagaagattgacattacccaacttcaagacttaccataaagctacaataattaagatattatttattgtaataattaagtatggcattggcaaaagaataggcaaataggttgatggactagaatagagagcccagaaatatacccacataaatatagtcaaccgatctttgataaaggagcaaaggttatacaatgtagcaaagatagtctttacaacaaatggtgcaggaacaaatggatatatccacatacaaaacaaaataatgaaatagacacagatcttacacccctcacaaaaatgttctctgaatggattgtagacctaaaggtaaaacataaaactataacacttgtaggcattaacttaggagaaaatctaaatgaccttgggtatggtgttgatgtttcagatacaatgctaaaggaacaatccatgaaagaaataattgagaagctggacatcattaaaattaataacttctactttaccaaaaacaatgtcaagtaaatgagaagataagccacagagtgggagaaaatatttgcaaaagacacatccgataatggactgttaaccaaaatatatttttaaaaacccttatagctcaacaataagaaaatgaacaacccaatttagaaatgggaaaaagacctgggcacctcaccagtgaaaatatacagatggtaagtaaacatatgaaaagatgttggctggttgccagtggctcatgtctataatcccagcactccaggaggccaaggcgagaggatcacccaggagtttgaaaccagcttggacaacatagagagaccctgtctctacaaaataaaaataattaattaatttattagctggatgtggtggtgtgcacctgtagtcatggctacctgagaggctgaggtgggaggatcactcgaccccaagagttcaaggttgcagcaagtcatgactgtgccaccgtactctagcctaggcaacagagtgagaccctgtctcaaaaaaaaaaaaaaaaaaagaaaaggaaaagatgttcaacatcatatgtcattagggtattacaaattaaagccatgagataccacttccagacctattggaatagccaaaatccaaaatactgacaacactaaatgcttacaaggatgtggagcaataggaactctcattctggtgggaatgcaaaatgatacagccaccttggaagacagtttgaaaatttcttacaaaatttaatatactcttaccataccattcagcagtcattctctttggtatttacccaaatgaactgtaagcttgtgtccacacaaaaatctgtacagggctatttataacagctttattcataattgccaaaacttggaaacaaccaagatattcttcagtaggtgaatgaataaactagtccagacaatgaaattttattcagtgctaaaaaaaacgagctatcaagccatgaaaagacatgtagaaaacttaaatgcatatttctgagtgaaagaagccaatctgaaaaggctacgtactgtatgatcccaactatgtgacattctctaaaaggcaaaactatggagactgaaaagatcagtggttgccaggagttgggggagggagggatcaataggcagagcacagaggatttttagggcaatgaaactattctgtgtgatacttcaatggtggatacaagatgttatgcatttgtcaaaacccatggaccgcataacaccaacagtgagttctaatgtgaactgtggactttaggtgatttgatgatgtttcagtgtagctttgttagtgtaacaaatacatcgctgtggtgagggatattgatagtggaggaggttgtatgggagagggcataaggggtgcatgggaactctgtactttacactcaattttgctatgaacctaaaactgctctgaaacacaaaatttactaatttattaatttacctttttttaagttaattgtcttcctagtaaaggggatagatgctagtatatttgataatgggtagattcaattcctacatccagcagactggtaaagtagactctgtgaaggaaaatacaactaaaatacaattgaattcaggataaatgacaacaaacataattgtaatgcctttaataaaatgaaagtggatctgaaaacaaaaacaacaccagaaaaacaaaaggcagagaagggaattctgtggagcaagagcaataaggcagggtggccctgagaaccagtgtcctcagtgctgggctggcagctggcagctgggctgtgggcccaagtaaagtgacactgctgacctgagagcccagcaccagggctgggacatgtggagggcccaaactggtcccacagtgctaggaactctggcaactagcagaagcaaatacacttaacttagtggtagggccgggcacggtggctcatgcctgtaatctcagtgctttgggaggctgaggtgggcacatgacctgaggtcaggagttcaagaccagcctggccaacatggtgaaaccccatctctactaaaaaagtaaaaaattagccaggtatggtggcgggcgcctgtaatcccagctacttgggaggctgaagcagaagaattgcttgaacctaggaagcagaggttgcagtgagccgagattgcaccactgcactccagtttgggtgacagagtgagactccctctcaaaataaataaataaataaatgaaaagaaaatagaggtgtttacagtctctcagatttttcaaaaatataaagccattttcaacaaaaatgtaagcttgtaatcaaaagtcacctaacacaaaagtaaacaggctacagcctgtgaataaacagaaacaaatataagagagaaagatagccaaggcactttcagatataggacatatgagagataaaacaactatgtatgaaatatttgaaggcataaaaatggagttacaaaacaagcaataaatgagaaactaataaatatggctaggcaaatttgaaagagaacaaattgtaatcattaaaaatgaatgatacagttgttgaatttaaaactcagtgcatgggttaaagagtagattagacagtgctgaagataaatttaatgaatcagaagatagatctgaagaaattaccaaaatggaacacaggaagaaaagataaaaagtttgaagggagattaagaggcattaagaagagaatactatatgtctaagcagagtcagagagggtaagaacagaaagaatgaaaaaaagccatttttaagaaacagtggctgaaaatttgccatatctgataaaggttatgaatgcacagacacatgtactgaagcacatatacaataagcagtgggagagagaaaataaattcatattttaggtggttttaatgaaattgaataatagcaaaaagcaaagagaatgttttaaaagtcagaaggaaaaggcaggccatctacaaaaggaggacaatcagactgacagacttgtcagtgacagcaatggaaggcataatatcttcaaagtgctaaaatgagataacaatctagaactacatttcaagtaaaatggtagaataaaagtattttcagatttaacaaaaaaattatttcaagtgcatttagaatcaacagattttcactaaaggagcttctaaagaatatatttaggaaaaataaattactccagaattattccaggaagaaaatctgagctacaagaagaaatactaactatagaaattgatagaaataatattttatcaatgttaatgttctggttttgatcattgtacaatggttgtgtaaaatgttaacatttgttaaggctatgtaaagggtatactatttatttaacagattttgtttgttttgttttgttttgttttgtgacaggatcttgctctattacctaggctggagtacagtggtgtggtcatatcttactgtctcaaactcctaggctcaagctctcctccagcctcagtcccccaagtagctgggactataggcatgtgctggctaatgttggcttttctttttcttttttctttcttttttttttttttttgtacaaacagagttttgttctattgcccaggctggtgtcaaactcctggcctcaagcaatcctcccacctcagccttccaaagtgctgggattacaggtgtgagccattgcagccagcctctgtagcagattgactgagatataatttacataccacagaattcactcatttcaaatgtacaagttagtggtttttgcaactgcaattttctctgcagccatcaccactatctaatttctgaacatcttcatcacatctgaaagaaaaaagaaaaaatcccctattcattagcaatgatttccacttcctcttccagcccctggcaaccactaatctactttctgtctgtatggatttgcctattttggatatttcttataaatgaaattgtgcagtatatggccttttgtgactgtttctttcactttgtatgttttcagggttcatacatgttatagcatgttcattctttttactgcaaaataatattcctatgtatggttataccacattctattcatccattcatctattgatggacatttggcatgtttccactttgtgattattgtgaaaaatgttgctatgaatattcatgtacagtgttggtgtgaacatatgttttccattctcttgggtagatagatacctaagagtggaattactgggtcatatgttaactctatatttaacattttgaagttccagtttctccacatccttgtcagcacttgttattgtctgacattttgattctagccatcctagtgatttaaagtgctatctcattgggctttgattggcatttccctaatgatgaacgatgaacatctttctatgtgatttttatttttcatttttatgttttttggagaaatgtgtatccaaatactttgcctattaattgggttgctaattaattgggttatttgttttttattattgaggtgttaagagtttttttgtttgtttgtttgtttgagatggagacttgctctgtcgcccaggctgcagtgcagtggcatgatcctggctcactgcaacctccacctcccgggttcaagcaattctcctgcctcagcctcctgaatagctgggattacaggtgcatgtcaccacacctggctaattttttatatttttagtagagacagggtttcaccgtgtgagccaggatggtctggatgtcctgacctcatgatccgcccgcctcgacctcccaaagtgctgggattacaggcgtgagccaccgtgcccagcctgagagttctttatacattctgaatacaagtcccttatcagatatgattttttttcctattctctgggttgtctttttattttcttgatgttgcccatcaaagtccaaaagttttaattttgataaaatccaatttgtgttttctttcatcatttatgcttttggtttggtttttttttttttttttggaactgaatcttcatttcattttagtcaagttaaaaataggtttaaaaactaatactcgagttggttattggaaaacatccaagtattcacaatttgggtgtgtgaatctacttttttttttctgtctctattttataaaaatttttttccccataggttattggggtacagttagtatttggttacatgagtaagttctttagtggtgatttgtgagattttggtgcacccatcacccaagcagtacacaacacaccctgtttgtagtattttatcccttaccaccctcccatcttcccccaaagtccactgtatcattcttatgcctttgcgtcctcatagcttagttcccacacatcagtgagaacatatgatgtctgattttccgttcctgagttacttcgcttgatagtctccaatctcatccaggtcactgcaaatgccgttaattcattcctttttatggttgagtagtattcatatatatatattcatatatatatttggaatatatatatgtggaatatatatatatatttatatttatatataccacagtttctttatccactcgttggttgatgggcatttgggttggttttggttccacggattttgcagttgcgagttgtgctgctataaacctgtgtgcaagtatctttttcatataatgacttcttttcctctggacagatacccagtagtgggattgctgatcaaatggtagttctacttttagttctttaggaaaccacactgttttccatagtggctatactagtttacattcccaccaacagtgtagaagtgttccctgatcactgcatccacgctaacatctactgttttttgattttttgattatggccatccttgcaggagtgaggtggtatcgcatggtggttttaatttgcatttccataatcattagtgatgttgagcgttttttcatatatttgttggccatttgtatatcttcttttgagaattgtctattcatgtccttagcccactttttgatgggattgtttgttttttacttactgatttgtttgttgtagattctggatattagtcctttgtcagatgtatagattgtgaagattttctcccactctgtgggttgtctgtttactctgcagactgttccttttgccgtgcaaaagctcttttgtttaattaagtcccagctattaatctttgtttttattgcatttgcttttgggttcttggtcactaaatcttgcctaagccaatgtctagaagggtttttccaatgttaccttctagaatttttatagtttcaggttttagatttaagtccttaatcaatcttgagttgatttttatataaggtgagagatgaggatccagtttcattctcctgcatgtggctagccaattatcccagcaccatttgttgaaaagggtgtcctttccccactttatgtttttatttgctttgtcaaagatcaattggctgttagtatttggatttgtttctgggttctctattctgttccatcagtctatgtgcctatttttataccagtaccacgctgttttggtgactgtggccttatagtatagtttgaaatcaggtagtgtgatgcctccagatttgttttttttttttgcttagtctcactttggctttgtgggctttttctgttgttgttccatatgaattttagaattggcttttcgaattctgtgaagaatgatggtggtattttgatgaggattgcattgaatttgtagattgtttttggcaatatggtcattttcacaatgttgattctacccatccataagtatgggatgtgtttctatttgtcttgtttatttgtatttatgatttctttcagcagtgttttgtagttttccttaacattttgaagaactgccaaagtgtttattgcaaagtatctgggccattttacaattccaccaaaatggatgagttccaatttctccaggtcttttgcctccttggttaggtgtattcctaagtattttattttttttttttgcagctatcgtaaaaggggttgagttcttgatttgattctcttgttggtcactgcgtatagaagagctactgatttgtgtacattaatcttgtatccagaagctttgtcgaattattttatcagttctaggagctttctggaggggtcttcagtgttttcaaggtaaactatcataacatcagcaaacagtgacagtttgacttcctctttactgatttggatgccctttatttctttctcttgtctgattgatctggctaggacttccagtactatgttgaagaggagtggtgagaatgggcatccttgtcttgttccagttctcagagggaatgttttcaacttttccctattcagtattatgttggctgttggcttttattacattgaggtatgtcccttgtatgccaattttgctgagagttttaatcataaagggatgctggatttgttgaatgctttttctgcatctgttgagatgatcatgtgatttttgtttttaattctgtttatgtggtgtatcacatttattgatttgtgtatgttaaaccatccctgcatccctggtatgaaacccacttgatcacatgatggattatctttttgatatgttgttggattcagttacctagtattttgttaaggattttagtgtctatgttcatcaggtatatcagtctgtagttttctcttctggttatgtcctttcctggttttggtattagggtgatgctggtttcctagaatggattagggaggtttccctctttctctatgtcgtggaatagtattaaaaggattggtatcaattcttctttgaatgtctggcagaattctgtgtaaatctatctggttctggacttttttttcgttggtaattttttaattaccatttcaatctcgctgcttgttattggtctgttcagggtatctaattcttcctgatttaagctaggaggattgtatttttccaggattttatccatctcttctaggttttctagtttatgtgtgtaaaggtgttcatagtagccttgaatgatcttttatatttcagtggtgttggttgtaatgtctcccgttttgtttcttaatgaggttatttggattttctctcttcttgttaatcttgctaatggtctatcaattttatttatcttttcaaagaaccagctctttgtttcatttatcttttgcattttttcttgtttcaatttcatttatttctgttctgatcttggttattgcctttcttctgctgggtttgggtttgggtttggtttgttcttgtttctctagttccttgaggtttgactttaggatatcagtttgtgctctttcagtctttttgatgtaggcgtttacggctatgaactttcctcttagcaccgcctttgctgtatcccagaggttttgataggttgtgtcattattgtcgttcagcttgaagaacttttaaatttccgtcttgatttcatttttgacccaatgatcaggttatttaatttgcatgtattcacatggtttcgaaggttccttttggagttgatttccagttttatcccactgtggtctgagagagtgcttgatataatttcaattttcttaaatttattgaggctcattttgtggcctgtcgtttggtctatcttggagaaagttccatgtgctgttgaatagaatgtgtattctttggttgttggacgaaatgttctgtatatatctgttttttccaaggtatagtttaaatccattgcttctttgttgcctttctctcttgatgacctgtctagtgctgtcagtggagtattgaaatcccccactattaatgtgttgctctctgtctcatttcttaggtctattagtaattgttttataaatttgggagctccactgttaggtgcatatgtgtttaggcctttgactgttatataatgtccttctttgtctcttttaactgcagttgctttaagtttgttttgtctgatataagaatagctattcctgctcacttttggtgtctctttgcatgaaatgcctttttccacccctttactttatgtgagtccttatgtgttaggtgagtctcctgaaggcagcagatagttggttggtgagttcttatccattttgcagttctgtatcttttaagtggagcatttaggccatttgcattcaatgttactattgagatatgaggtaccattgcattcatcgtgctatttgttgcctgtgtaccttgttttttttgccttttatttttgccttttaaattgtatttttgttttataggtcccgtgtgatttatgctttaaaaaggttctgttttgatgtgtttccaggatttgtttcaagatttagagctccttttagcagttcttgtagtggtggcttggtagttgtgaattctctcagcatctgtttgtctgaaaaagactgaatctttccttcatatatgatgcttagtttcactggatacaaaattcttggctgataattgttttgtttgaggaggctgaagatagggccccaatcccttgtagcttgtgtggtttctgctgagaaatctgctgttaacctgataggttttcctttataggttacctggtacttttgtctcacagctcttaagattctttctttcgtcttaactttggataacctgattggacaatgtgactaggtgatgatctttttgtgataaatttcccaggtgttctttgtgcttcttgtatttggatgtctaggtctctagcaaggccagggaagttttcctcgattattcccccaaatctcttttccaaacttttcgatttgtctactttctcaggaacaccgattattcttaggtttggtcattcaacataatcccagacttcttggaggctttgttcatattttcttattttttaaaaatttatctttgctggactgggttaatttgaagtctttgtcatcaagctctgaatttcttttttctatttgttcagttctattgctgagactttcgagagtattttgcatttctataagtgtgtccaatgtttcccaaagttttgatgttttttctttatgcaatctatttcccttgacttcttgtatcgtgcttaatacatttgactttatttaatcttcccttttccaaccattctgaaactgcatggtttctaatgcaaatggctccctttgcattagaaacacctggggtgctctaacaacacctaatcaggcccactccaccaaagattctgatttagctacctactatagcaaccagcacatagtagtatgcaataaagttggaaagaaggaagggaaggaaaagaaaggatggaatgacggaagacaaatggcttgttcatttgtactataaaccacctttacaataaacatatattcaattataagtgcaaataatccaccacctcaagtaccattgattatatcatgtatattccttagtccttagtcttaatcagcagcattaaaaaaatgcgaatgcactctcatacactgattgtaagatgtatcctcattttaaaaataattaaacatggtgatgaaaggatagggtatctctagaatctaggaaatgcaacccacagtctccaggagctttggctttgaaatctctgctctttggagaggtcccctggtcccgtattccttgtgatatagaatttacattgttattgtttttcttgaagattctgaaaatgttttcaacccccttttctctgtctggttatagttcctgccttgcttaaaaaaaaaatcatttgtgctgtattttcaaagaccgtgtatgagtatgtgtgtgtgtgtgtgtgtgtgtgtgcacgcgcgcacgtgcatgtgtgtgtccttgaatttgaggttgtttgtgtttctaagtggtctggaaggacactgcagttttaagaggaaccatctgcaaatgccaattcttacttgcacttgtgcttttgaaggttaagttgcctttgggtagggcttaatatgctgtatgtaaaagtttacctggtcgggcgcggtggctgacgcctgtaatcccagtactttgggagtccgaggctggcagatcacaaggtcaggagtttgagaccagcctggccaatgtggtgaaacctgtgtctactaaaaatacaaaaaaattagccgggtgtggtggtgcatgcctttaatctcagctacttgggaggttgaggcaggagaagtgcttgaacccgggaggtggaagttgcagtgagccgcgatcacgccactgcactccaacttgggtgacagaacaagactccatctcaaaaaaaaaaaaaaaaaggttcatctagtcaggaaatggtaaatataaagttacatttccctgtcacttatcattctacagctcattaaatttcacctaattgcatcttcagaattcaggatgttagtgtttcttagcttcactaccagactgaaatcttaacacccaggtcattagagttctatttacttatcccaggctatcaaaagatcttcctttaggaatggcgagttcctgagtaataaatgtgacttagaagtttgaaaggtaaatattccagcaaatctaactcattttgttagtcattctaattgcttgtggcattttccatcataacattttttgatggcaaaatgttgatggtaatcatggccaggaaactagatttgataaatgtctagcatatcaagaaaaatttaacacttaaatgatactaattcttcaactgttagttggactttaagcttctgtcaaaccatgatgggaaaagtctgactgtgttgactgtgagctgtatgtgatttccccctagccctttgaagacaatgaagagaatgccagtatcatttcacgaaaacttctgggaggaggaagtagctcccagtgaagcatgacttctgcagagaaggaagctaactctaggacatttattttatgtaactatagtctatagataagcaactgccacattataggcagataaaaatagtgtacattcgaaaaccaatttatactgcagagttccttgtgagttcattttatctgtatccgttataatccaaatgcaagagtagcattactttttttaatgaggcatacttctgggcttttttgatttttaagcatttcagagaataaagggaaaagagaatacctataaattatgaaattgatttcgattcacctaaacttgccatgctttcgacatcttctcttgttcatactttatgttagatgaaatgttcacatgataacccgtgtgtgtttgtattttctcttcattctttcattaagccatttaataatatattcttccccatgagatggtttaagttgtttttcttggtgcacaccaccatcatgtcataaaggctttattttgttgttgtttgaaagtgtctcatgcttaaaacattagaaagtacacttgtgattgtggctacaggaaatgtagaatttcatctagagttacttaaacatggcatggtgagagtggttcagagagaaagataataaatgacaataccatttgctatgcagattccagagatctaaaacccagtaagctaagaaatgtatatttaaattcagttgctatgaaacccaactggtatgaaacacaggcgattacatccgttgactttactttgaaaagacaaatcgagaaagggtggagagagtattcttttctggatacaattagtagtatctgttgtgcaataaatgtcacatgctgtagtggctactccatctttattccttatgtcatagtataataggagatgtaaaaagtcgacgtagatgttttaaaagactgggccactgggaatcactgggaatccaaagcagtgtgtgcccaccaccatctaataaagtgtgagaggatatacgaaaggtttaattttttaatttaaaacaaaaggaaataaaataagctttgctgactctttttattattatttttttgagacagagtctcactctgtcacccaggttggagtgcagtggcataatctcagctcactgcaacctccacctcccgggttcaagtgattctcatgcctcagcctcccaagtacctaggattaccggtgcctgccaccatacctggttaatttttgtatttttagtaaagatgggattttgccatgttgaccaggctggtcttgaactcctcacttcaggtcttgaactcctgcctcggcctcccaaactgctgggactagagggatgagccaccatgcccagcctcatgtataatgtacagattggtcctagggggctgatttggtctgtatgtcacggtcacacatcagatggtctagagccagaatggaaagccaatgcttttgtctttgtttttccatcatcatcatcatcatcatcatgatttgagtctgtttgccatacagtctgactccaggccccatgctctttccactccagtttcctcttctttcgtgtgagagtggatgacttaattctgtggcagttgaccctggtctgttatcaaaaagagaaagccacgttgtcacacttcccactgccgacagaagccccagtcatatgttctgggctcttcctagggctttttcgttggatttgcctggtctatgttgtagaagttgtctaaccacccacactcctgccatgagtcaatctgtgccatctacttgcttatgactgttattattatcattgttcccaagtcttgtcattgggtaatgatcaacgcagcagcacaaaagcagaaactttttgctctgggcagaagctcctgatgccctccttcttgatttctggctttcccctctactcttttcactctgaagattcttgtctgtttcttcagcctcctgacagtccccatatttgttacagtgatttgcacattccttctaagcctgtttgacattctctggaaagaaaagggaaaaggatgctttagtctcccaacaggggagagttgtgtttttggaagaaaaacaacaccaaaaaacctaattgcaaggtaatggggtggctttgatgaatagaaccattgagagcatggggcagaaagtcttgcacctgactgataccaggttttcctgtctagataaaaacataaagaacacagagcacagactttaaaaaaaaaaaaggtaaagaaaagtaagctgtgtaaccaggcacttgccttcttttatgaggagatcaacctggttggaaaccttccactaactttgctatctaccagggagggggtgtggggattgataacaacaggtgggaattagactcctggcagtagcttttctggttctttggatgtgtgcctactactgtcgctctcagtcctcttctcagagaaaggcaaagtaacctttattcaaactggaattttctcaagctggaaggcaagtttgttatgggaggagagaatgccaatgggtcagccagaatccaaggagggctgcactttgcggaatgctctgctccctgaccacactagggcctccccgtccactgtccatatccgttttgagagcttctgagcatctctaggttaatgctttttgcaggctgtttagcatttctcctgtgatgaggctgtcctgctctctgctggcattcgggaccactttagctgggaaggatcatttccttcccattagtgaaagggagcacagctttcctgttttattttttccctttcttcataatccttaatgtattcactcaattactagtggtactattattttttgttttttcttttgttgttttgttttgttttagagacagggtctcactgttgcccaggctagagtgcagtgtcataatcatagctcactgcaacctcaacctcctgggttcaaaagaccctctcacctcagcctcccaagtagctgggactacaggagcatgctgctgtgcttggctgatttttttaacttttttgtagagagaggatattgctgtgttgcccagactggtctcaaactcctggcctcaagtgatcctcctacctcagcttcccacagtgctaggattaaaggtgtgagccatggcacctggcccattactattatattttgaacttaagtgacagtagtaatgttggggagcagagagctggcagggcaagccaatagcaagcaggttttcccatagttcctgtctgctcccctgtaatgatagctattatgggagtatctgctgctcctcctccacatggacatagtttccaatttccttaatccagcttcatgaaccgcagaccgctcctccgagagtggctgtccttggaagttacttacttctgttacttcgcagctattgacagagaggaccaattttctttccagtatataccaaacccaaggtatagcagtgctgtccaatagaaatataatgcaagggagatatgtgcatttaaattttctagaaggcacatgaaaactataaaagaaacaggtgaaatcaattttaacaatatattgtccttaacactatatatatataatgtcatcatattaattttaattgatactaagaaattattgaaatattttacattcttttctttgtactgagtcttcagaatcctgtgtgtatcttagatgcacataacaattttgactagccacattttaagtgcttataatacactttattgaagaatgtaggtcgataccatagatttaactgtctataaattaaagcatttttttctttgctcttgcagggccgtttcttattggttgggttgtatgcaaatgtgtgtttgttaattaaatatatttaatgatgtggagaaagaaagaaattggctccttgacccacctcctagtagggtaataactttgtcaagcacattgcagtctttggagatgacccttaggtgtgaccttgaagctgaccttctgacaggcaggggtacattcctcctctggagagatttgtctgtgggagtgggtgagcttgctcctgggacagtcagtttattaaaattcagttattgggccaggcgcggtggctcatgcctgtaatcccagcactttgggaggccaaggcgggtggatcacttgaggtccggtgttcaagaccagcctgggaaacatggtgaaaccttgtctctaccaaaaaatacaaaaattagccaggtgtaaacccgggaggcagaagtcgcagtgagccgagatcgcaccactgcactccagcctgggtgacagagcaagaccctgtctcaaaaaaaaaaaaaaaaaaaaaaatccagttattgatcatggtcagaggagattcttgagaggcaaggggatcagaaagcagccatgagagcagaccctgaaacaagaagcagagtaaaggactgactagaaggggccccaggacttttatattcaagcaagaggacaagaattattctggatcattcttaaaagtcgtttaagtgtgctgttctaggtagggagcttttttccaaagatcattgcatcagaatgatggctttctggatccttttcaagttggctttgttgttccccactaaaagcagcaaaataatttagagttaaggtgcaagaaagcaggaataattttttttggccaaaagacattatttatataatcgctggacactgactcaaacttgctgttccacttccattcttctttattcatcttgacaaaaccagctggtagggggatgggaggcacatggggctgtttattctaagttcacttactctcttgtcttcccttttccttaaaacgtgaaagcaaaatatgtctctttttaagaacaacttggttgcagaaatgactactgtttcattcaatttttctaccagtcttcttcaagcaggccttggggactcaggcctcctgagtattacatccctaagactctgcacaattaccaggaagaagtatggccaacagtgagagtgatggacagctgaggtcaacgcattatccatggacagataacaagtctgcccccagtcagggacatttcttaaatgttatactcctcagctgaattaaagttttcttctgcagctgagcttttgggaaacccatgggaattattgtttgttgagctggataatagtgaaattaattcttttctgcttagggggaaggcactggtgggacatgctaattgcttcagtccaaaagaactcctggccacattagagtttgtaatagaagaatgataagtaaagcagccagaagaggaagtttggagggctgatttgcaggagtttcattgatttagcatctgtagggaaaattcccttaaaaaagaattttttttttggattagtgaacaggagaacattcaaaactaagacagatagaggccaactgggaatgctgtgcagcctggaatagcttaggctgctgttcaatcatttccccaaacaggaaaccacagaatgttagagattaaggtcactagatcattgggaaaaaagcataactttgtctgcaaagtattatgatatattgtaaatggaccagaatataagcataaatctttcgtctaaaaatgcaacaacaaaaatccatccttctcacaaaagcacaatttgctttcctgttgtgctgagataccaaagcctccttctctggtatttctgtaattttcctaataagtaagagctgtaagcagggaataaagagaaaagttttgtgttctctgtcagtaacatgtgtgctttttttttttaaccacaacaaaaaatggttaaagtttagaacagtaagtggacctcacatgatttattttcagtcttagcctttcatgctagcaacaatgaaaataaactaaaatgcacacttatatacatttctgtctctaatctctcaggctgtattaaaagcacagagttcttttttcacctctgattatcatcatggcactgcctgacttaaattttaagtgaaaatctgtgcttatagagtgtggaatctttctaggtgtctgtaaacgtactcttctgcaggtatctttggctaaaatggttgctttatatctgccttaaattatttacccctcagaggcatactctatttaaaataagggttaattctcatctgattctgttcattaggtctttactaggagaaggttttcttgctgagatagttaaactcatgaataaactgctgatttatgtgcatctagaacctgaaattcaatttatgatttgaattgttaaaaggactagcattttagaaaggtttaagtgaaactgacttttcttggcccctcttctaaggaaaacttaaagaatgaaagcataaatttccctttccttttctgagtattcactgctttgaaaagttaaaaaaaaatctgaagcatttgaaagacaggggtcacagctttccatacctgttcccatggtacttagtccttagtctctccataaacgtgtctgttgaataggcagagatctcatatgagtaaactaaaaaacaaacaaaacacactacatacacaagtgagcatgccctcttctccaactcagacaaaaggccacttcccttccatttcagatggagggaacacttaaccacctgcaaatccaggaaggaagtgaaataaatggatagtgttggattgagacctactgatcttttttacccatgacagaggtatggatacagagaatatttttctcttctaagaaaaacagtagcataagcattgaataaatgacaaccaccctgtgcctgtgtgtgtgaggataccagggcaggggtgggggtgtgcctcagggttacagctccgcaaagtgttagctcattgtggctgagaggaaatacaggcccagcctggtcagatctccgaatatttaaatgctgactgtcttagttcaggctgctgtaacatagtaacatagactaagggcttaaaaacaagaaaaaattgtttcttacagttctggaagctggaaatctgagatgagattaccagcatgatcagttctggtgaggatctcctgggttgcagactactgacctctttttgtatcctcacatggtggaaagagtaatagagagctctctgaggtctcttttgtaggggcactaattccattcatgagggctctgctcttatcccctaatcaccctccaaaggccccatctcctaacaccatcactttggctgttaggttttccacatttgagttttgggaggatacaaacatttggtccataacactggcatctaaatggaaaaacagttacaacaacaccatttggactaaccaaatacatctgcatgctgaatgcaagcctcacacagccaacttaagacctctgcactccagtataggaatttattttttagtttatctttgataaagagcaataaaaacattggtaaaacctgctcttcaagttataggaaatctttaggtttggtatcctatttgccctcctgacagctgtgggatgtagctatttccactatttagaggagaaaattaaagctcagagtgagtagatgacatatccaagatcactcagtgagcacaggtcttgcggggctgaggcagggccagaggtcaagacttctgtctgaaagtgtcggctacttttataagcttctgctgttagttctgtccagtcagtggcgtgcctggtgcagttatttatggagttctaactcctccatcctaagtcacaattctaatccataggtaacatttcatttggaaattgttgtaccgttgtgtactttatcatgcttcacatgcatcatgtttcataaatactcccgattcatatgtgcttttcttttcttttcttttttttttttttttttttgagatggagtctcactctgtcacccaggctggagtgcagtggtgcgatcgcagctcactgcaagctccgcctcccaggttcactccattctcctgcctcagcctcccaagtagctgggactacaggcgcccaccaccatgcccggctaattttttttttttgtatttattttagtagagacggggtttcaccgtgttagccaggatggtctccatctcctgaccttgtgatccgcccacctcggcctcccaaagtgctgggattataggtgtgagccaccacgcctggccacttttcttatacccattgtgcagatgaagaaactgagctctagatgggctaactcaattaagataatactgccagttagtggcaggggtcagattcaaacccagccatagagtttatgcacataactataaacatatatccttaatgtcaaagagtcacactttaggtttgccacttgcttgtggaattacaaatatagaataaagcacctgttcaatgtgttcttatggaggttagtgaccttagacagtagtgaggcaatgtggaagagcacttagaagcacacagtttgggctcaggtcgatctgaacttgaatttcagtcacctgttgggggatcatggtacactgacttacctccttacacctctgcctccttgtgcctaaaatgggataaataacacctgccttctgagaatgctgtaaaatgcttagcacagttcctggaacataataagaaaaacaacaagagagtcttattattactcaaaaacttccttttaaaagccatagtctttaccacacttttctagcatttaagtgtgggggaattccatgtgattttgaattccaggcttcagaattagaagttcctttaaaagagtaatacatgaatttatggctctaactttcaagaaaagactgagatgcagtggccaatgcatgcaaaattagtagtcaagggtgtgttattatttcgagagcttgaatatatatagagttcaggtttatcctggctggatttcttcctacagtcggcctttttctgaaagagtgagattgacaaccaagtatttgacacagaaagaccatccctatattctgaaggtttatttagaagaagtgctgtgtcgagtctctccaatcattatcagatgatcaacttgcttgagaaggaggaggacctaatactcactgacctcttattctgtgtcaggcactgtgctgggaactttgtgtacatggtctcattagttctcgttacaattctgtatagttcgtcgcatcctcatgtccagaaaaggggctgaagctcttggaagttcacccacatgtctacgagttccagttatggtgtgttggcatgcactgcactggtccgaaggtgtatgagtttggatttcatggtttcagtgacagaaataactctgcttacttgaagcatgaatggaattgattggaagacaggataggtcttagaattcatggaaaaactgaagaagtggggtgaagaaagaacagcagattggggaaaattctagggatctaggaagcaaaactaatgagtagacttatcaatgaactctgtgtcctctgcccatgattccaattccagggaaagagcatttgagtgcctttacttaggtaatgtgcttatcctttgccagaggaggcaggacactgtgattgacaactcccaccagggctgtatccactgaaggtgtgggtgacccacaaagtacagtttccaggagaagggagagtggacctgggcatgcaaaagcagcagatggtcactatacagcaccacatgaaaccattaaagagcagaatccataccctgtaattgtccatcggttttatggggtaccaggcactgtgcaaggcctggggatgtagcacgcactcgggagctcctgtttttaaggcactgctggtatttcacagcggtttagcaaggccactccccaccaactgtgagtaatgccctcacacacatcctgtccatagagccaaagtgagaggtgactaagagatgtcaccagggccagacaaacagagcaagattgtttctgaaccggagatgtgaactgtgtctcatatgcaactaactctcagtttccagcagcttttggtagcactaaatggttagctgcagggagtcaataaatgcttgttggtttgaaggaactttagctcttttcttaaggaaaaattttaggtagaagggtggaagtcgggatctaggactcattctgttgcctcacagtatcacagtttccataacagtaaagtggaggcggacagtggaacacttctgttcctgaccttgtgaagcagtacaagaataaagctctatgcacatgaaagcaggttgaaaagcaagaaagaattaataataataaagatgcttttctcatctctgccttttgacaaagaggaagaaaaagatgcttgtggtccaagtcaccaaatgccgtctcttggaaaggcttgaattctggaatgagtttttaaagactgtctatgaattaaaattaagctttttacctagttgtcatgaaaaaaaaaatgagagctggtatggtagtatttgaattcttacaaattatttctccaaaagactcatcttttgccaaactgaggtatccaacctcatagcattattgttcttttttttttttttttccattataaaagcattctaggctgggtgcagtggctcatgcctgtaatcccagcactttaaggaggcaggtagatcacttgagcccagaagtttgagagcagccttggcaacatggtgataccctgtctctaccaaaaatacgaaaaatagccagtctcataacctggtctctaaattaattaattaattaaactttttaaagaattattttgtaaaaaacgatgagaacacatggacacatggggggaacaacacatactggggcctgtcaggagggtgcagggggagggaggtaatggatgctgggcttaatacctaggtgatgagatgatctgtacagcaaaccaccatggcacacattgacctatgtaacaaacctgcacatatacccctgaacttaaaagttgaagaaaaataaatcaataaataaaaagcattctatggccaggtgcggtggatcatacctgtaatcccagcattttgggaaggaagctgaggtgggcagattgcttgagcccaggagttcaagaccagcctgggcaacatggcgaaaccccgtctctacaaaatatacaaaaaaattagctgggtgtagtggcatgcacctgtagtcccagctactcgggaggctgagatgggaggatggcttgagtccagcagttggaggttgcagtgagccaagattgtgccattgcactccagcttaggggaacatagtgagactgtctcttaaaaaaaatttaaaaaaaggattgtaaccacatttgccaaaaacaaaacaaaacagaaacctagaagatagccaatctaaaataaatcaaccatagctcaaataactgtattttgatttgttctttccccaggtttgaagcatttttaaatatgtgataattagagtacaaacaagtgaagtctcagtttttcattgatttttttcataagataagcatttttctgtgacattgcataaccatcagtttcactaagctaagcagccagccctctaaggagtaaaagaagtctctaaaagcagtggctttgctcccttaccagaggcctacagctgaccagaaaggaaagtgaggaaatgcatgttcagggcactgcctagtacgtcgctggagaccaactccaaccagctctttccttgttccttgttttgttccaatcactattgacaagggaagtgtagctaaaagctgcattcatctctgtctcctcccttctgtctttagaccttgttgcttaaaataccagcaagccttaggggtaagtttgctccagagtgtcccagttttagcactgcatgtcttgctgggcaactcctgtccagggcaaactgggacagttggtcaccctaccgggggagaagtgggatgagtgtgtagacatatttggccaacctgtccttgtcagagctaagttcctccctggaacttttctatcaggatgtgggtagccagcgtgtgcccttgggcttcaggaccacccctatgccgtgcccacctgcttctcatgctcagccactgggtcctaggctatctcatccactcacccagcctagaggcacctaactgggatattcctataggggctcatgagagtaaatgtgacatttaggtgcaaataagagctctggaattcttatatttaatgctggcctgcctttgaaatcacttctcatgatcgttattctgatttcttttttgttgcatttgacttatgtgcccttcagaaatacaaaatggtttccttgctttgcaaaggaaggatttccatttgtacccaaaagtagaccttctacacattgcctgtgcgagattgtgcaattcagctctttgctgtttcttggtccagagagttggtgctcttgttatgataagtcttgactaacaagataagtcttgactaacaactcagggatggtcctcattccatgtcagttcaacatgtaatataatgctgtacatataaatgtgtttttgctgtaacatggggactttgctattgtcccttaaggatgaaaaaaacatcacattgatttcaaaatatagtaatgattgtcagtaaagaaagctgtgggagacacatgagagatgcaagctgcctgtagtggccatggagagtaaaattaaatggatgcactatgaatttgctgattgatcagcatcttttacactgaattctagaagaagaatcactttatgcttgatgatctttgctaatcagctataatcaggtcttcgtagaatggtgaagtaattaagtcttgttcactggtactaatcatatataattgtttccatactgttggccaaaacaactataggaggttttgcagaggccatgagtactcaaggtctttgaggtaatttctctatataaacaatgcaaatattttattactggagttcagttgaatttttttaacaggctttaaatctatcaggattttcctgtgggtttcttcttaggagagtgggggtggtgatggtgactgagtaacatgaaaggccccgtgttcctgcagtgggggcccagggctctttcatcttagtctgactgctgtcccactgctgcttgaggatttggagtgtgtaaaagtcctgggctttttaattatgtttgagccttttaagaacccgcaccgtaaagggaaagacggaaacttatgcagtgaaatattttatatccaatttgaggattcaaccaaaggtctccactcgggccaaatcagagccttcaaatagatcaaaagtagcatggagttgaggccgacctctttgttggaggtggagtggcaatactttatgataattaatatgttagttgcataaaagtagcgtaagagtagaaaaaaaggagagtatattactcagatttccaccatctaaatattaacatttttcaaactttgtattccatatatgtgaatatgtacattgatttttatagttatagttgtgatatacattatacttgactttctgctttcatacttaacattatgtgataagtatttcccacattgcatcatggttcaatgaaacattttaaatggtcttataatatcctatagaattcttatactgttgtgcatttacctactcaattataggacttctggattgtgtttgagtttttcattataagactcaatgaatatatataattttttaaaatttctttggacctgttttcctagaatatattctacaagttgaaattactgggacaaggaatttcaacatttttaagtttgggggcagaaaattggctactgattttctgaaaagaagagccattttatactgtccccattagttctacaactttatcagaattgggaattatctgaattattttttcaagcaaattgaaaattgctttaggattaatgaaaagagttacctcagtgttgtttttcctatgataaacatcagtttgacacatacaaaaaattgtattctaggaagaaagaacattcctcactgttgacaagcagctatcatatttttaaaaaggaagttttgcaagtggcatggcaggcatagttgcttccaataagaaacaaatttgaggcacctgaatctctctgtctgctgctgcaaaaaaattatacttgctactgaaatcaatgtaggagaggagagcaatatggattagcaatacccaagagaaagctcagcttgccagcgactgcactgcctgaagttgaagttaatgaaatgcaggcttcatgaaaatcctagggggtagtattccttagttccactaaaatgtaaccacagtcttccttcttaacatagccataagtccaagtaaaacctgtatatatgtatcactgagtaatacaacacaaatgtttttgcaaccaagagaggcctttatgcagattactatgaagaattcagtggtagagacaaggcttgagtttatttcggattgttattctttatggctaactagaagaaaatctcagagagattcatgtctctttttctctctcactggctcactatttatacctagctcgattcatgagcttcacctcctgaatcttgcatttgctgatggcttgatttcatttcaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaggaagatgcttgggagcaggcaggttgtgggaatgggagagcgggattgaaaataaagcctgaatagcaatgagacatggccgaccttaagcaaagccatacaacagccagtgtgtgtcttctgtggccctactttccacagagaagagtggtctgagttcagacatacataggactaaatataaaaccagacaaaagtttaggcattgttcaagaggtaaggagacaattcattaaaaagacagtgctcagtccaaagaggcaaatcattaatgagagaattgcagtctatttggggagagaacttggcaaattataaaggaagtttcaacacaatctcctctggcaaatgtatgatcacatttttatattacatgtggagcctcaggacaaaatctctctagctgacaccctagtgcaacttccactgcggggtcagatgttactctcatcatcttcctttcttgcaatgtcctcttcaagtgttggaatcatggttatactggcctcataaaataagttgggaagtgtttcctctttctgttgttggaaacggtatctttaagtgttaggagaattcactggtgaagccatctgggcttggagatttgtttgagggaagggttttgttgttgttgtcgatatgtaagtgtacatctttatagaatacatgtaatattttgatacatgtatttaatgttaatgaccaaatcagggtatttaggatatccatcacctcaaacatgtatcatttcattatattgggaacatttcaaatctcttctagccattttgaaatatacaatatatcgttcttaactatagtcaccctactgtgctattgaacactagaatttattccttctatctaactgtctgcaccccttaaccaatctctcttcgtcttcccacccatccccacttcccagcctctggtaactatcattctattctctacctccatgaaattaactttttacgctcccacatgtgagtgagaacatacgatatttgtctttctttgcctggcttatttcctttaacataacgacctccagttccatccatgtcactgcaagtgataggatttcattcttttttatggcttagtaatattccattgtgtgtatatgccacatttttaaaatccattcatctgttgatggacacttagggtgattccatgtcatggctattgtgaatagtactatgataaacatggcgtgcagatattcctttaatatactgatttcttttcctttagaaaaatatcaagtagtggaattgctgcagcatatggtaattctatttgtagttttttttagaaacctccatattgttttctataatggctgtactaatttacattcctaccaatagtgtatgagttcattttctctgcatcctcatcagcatctgttattttttgtctttttggtaatagccattataactccaatgagatgatacctcattgtggttttaatttgcgtttccctgatgattagtgatgctgagatttttccatatacctgttgggcacctgtatgtcttcttttgagaaatgtctattaagatccttttctcacttttttttttttttttttgagatggagtcttgctctgttgcccaggctggagtgccgtggcaggatctctcggctcactgcaagctccaccacaccggttcgcgccattctcctgcctcagccttccgagtagctgggactacaggcgcctgccaccacacccggctaagtttttctatttttagtagagatggggtttcaccgtgttagccaggatggtctcgatctcctgacctcgtgatctgccccaccttggcctccccaggtgctgggattacaggcttgagccaccacgcctggcccttttctcactttttaatggtattatttatggtttgtgctcttcagttgcttgagttgcttgtatattctggatattagtctttggtcagatggatagttggcagatattttctcctattctacaggttgtctcttcactctattgattgttccctttgctgtgaagaaactttttagtttaatatagacccatttgtctgtttatggttttatcacctatgcttttggggtcttagccataaaagctttacctacaccagtgtcctaaagtgtttcccctgttttcttctagtggttttatcattttgagtcttatgtttaagtctttaatccattttgagcttattttacatggatatggtgagagataggggtttcgtttttgagcttattctatgtgtatatggtgagagataggggtttagtttcattcttctgcatatggatcccagtttacccagcaccattcattgaagagggtgtcctttccccagtgtatgttcttgtcagctttgtcagagattatttggctataaatatgtggatttatttctgggtttactattctgtgccattggtctatgtgcctgtttttgtactaataccttgctgttttggttgttattgctttgtagtatattttgaaatcaggtagtgtgatgcctccagctttgttctttttgctcagtattgctttcactatttggggtctttttatttctgtgacaaatgccattggtattttaatagttatgcattgaacctgtagattgctttattcagtatgatcattttaacaatattaatttttccaatccatgagcatgagatgtcttccatttgtttgtgtcctcttcaacttcttacatcagcattttatagttttccttgtagagatctttcacctccttgattaaatctattttaaggttatttatttcttttgtagctattgtaaatgggattgctttcttgatttcttttttagctagttcattattagtatatagaaatgctagtgagttttgtatgttgattttgtatcctacaactttactgaattcatttatcagttctaaaattgttttggtggagtctttagttttttctgtataaaagatcgtgtcagctgcaaagagaaacagtttgacttgctctttttcagtatgaatgccttttgtttctttttcttacccagttgctctggctaggacttctggtactatgttgaataggagtggtgaaaggggacattcttgtcttgttccaattcttagaggaaatgctttcagcttttccctattcagtatgatgatgtttgttgtatatggcctttattacgttatggtatgttccttctatgcctgatttgttgacagtttttatcatgaagggatgttggattttatcagatgtttttcttaatctattgagatgaacttacagtttttgtctttcattctgttgatgtgatgtatcatgtttattgatttgcatatgttgaaccatccttgcatttctgggataaattccacttgattatggtgtattatctttttgatgtgttgttggattctgtttgctagtattttgttgaggatgtttacatctatattcattagggagattggcctgtagttttcttcttgtgtgtgtccttgtctggttttcgtatcaggatgatagccttgtagaatgaattagaaagaattgcctcctctttaattttaaagaattagtttgagaagagtttgtgttagctctttcatataagtttggtagaatttggcagaaaagtcatccagtctagactttcctttgttgggagaatttttattactgattcaatctcattattctttattggtctgttcaagttttctatatcttcctggttcaatcttggtaggctgtatatgtccaggaatttatccattcccctaggttttccagtttgttaggctaaagttgtccataatagtctctgatgatccttcgtatttgtgtggtatcgattgtaatgtctccttttccatttctgattttatttatttgggttttctctctttttttcttgattagtatagcctagtctagtttattgattttatttatcttttttaaaaacaggcttttttatttcattgatcctttgtatttctttttagtttctattccatttagttctgctctgattgttattattcctttgttctaattttgggtttagaacaaaagataatgggtttggtttgttcttgcttttctagttttttaaagatgcatctttaggttgtatatttaaacactttctacttttttgatgtaggtatttattactataaacttccatcttagcactacttttgctgtatcctataggttttggtattttgcgtttccattttcatttgtttcaagaaatgtttaaattttcttcttgatttcttaattgattcaatggtcattcaggagcatgtttttaattttcatgtgtttatacagtttccaaagttttccttggtattgatttctagtttaattccattgtagtcttagaatatacttgatatgattctgattattttacatttgttgagatttgtttaatggcataacaagtgatctgtcctagagaatgtttcctgtgctgatgagaaaaaaatgcattatgtagcagttggataacatgttctgtaaatgtctgttagttccattttgtctaaagtgcagtttaaatctgacgtttctttcttatttttctgtctaaatgatggccaattctgagactgtttgttgaagtcctcgactatgattgtattggagcctatctctccctttagatccaatactatttgctttatatatttgggtgctccagtgttggctgcatatatttaatataattgggatggttatatcctcttgctaaattgaaaacttttttttacaatttttatcttgaaatctgttttatctaagtacagctactccttctcacttttggtttctgtttacatggaatataattgtttattcctttactttcagtctgtatgtgtctttgcagctgatgtgagtttcttgcaggtagctgatatggtttggatgtttgtcccctccaagtctcaagctgaaatgtgattcccaatattggagatggggtctgatgggaggtgattgtgtgatggggaagattcctcatgaatggctttgcaccaatcccttggtgataagtgagttttcactcagttcatgtgagacctggtatttaacagagtctgggacctcctccatctctgtctcttgttcctgctcttgtcatgtgaaacacatgcacccactttgccttctgccataattgtaagcttcctgaagccctcatcagaagctgagcaaatgttggtgccatacttgtatagcctacagaattgtaagccaattaaacctctttcctttataaattacccagcctcaggtatgtttttattatgatgcagaatggcctaacgcagtagcatatagttagatcatattttatttttatccgttcagccggtttatgtcttttatgtggggaatttaatccatttacattcaaagttattactgataggtgagaacttatttctgccattttgttcattgttttctggttgttttatatatcctttgttccgttcttcctcttttattgcttatcattgtggtttgatggttttctgtagtggtaacatttgaatcttttctctttctcatttatgagaagccagtgagtttatactttcatgttttcttgatggtaggtatcgtcttttcacttccagttataggactgccttaagcatttcttgtagggccagtccaatggtaatgaatgtcctcagtttttgcttgtctgggaaagactttctttttccttcttttttgaaggatggctttgtgggtatagtaatcttggttgtcagttttgttctttcagcactttgaatataccatctcattttctcgtggcttataagtttctgctgagaaaactgttagtctgatggggattcccttatatgtgccttgacaattttctcttgctaattttagaattctttctttgtctttgacttttgacagtataactataatatgccctggagaagacatttttggattgaatccatttggagatttttgagcttcctgttctagatgcctatatctcttgcaagactttggaagtttggggctataatgtcattaagtaagctttctatgcctttgcttacctcttcttcttctggaacacccaaaatgtgaatatttggttgcctgctttatggtgttccatatgtcatgtaggctttctttgttctttattaaactttatatatatgtacttttttgtctgagttatttcaaaagacatttcttctacttcaaaaagtattctgcttgatctctattattgaagctcttgattatatttttttgtttcattcattgaattcttcagttacaggatttctgattggttcttctttatgtatcagtctctttgatgaatttctcattctgattatgaactgtttttctgatttccttctattatttatctgtgttatcttgtatctcgctaagcttctttaataacactattatgaaatctttaccagatatgtcatagatttatttttcattgaaatctgtcactagggaattattgtttttttacacatatcacatttccttgctttttcatgtttgtttgtttttacattgatatctgtgaatctggtgtaacagtcccttcttctgattttttgaattgacttttgtaggaaagatgttttccaatagatgtgtctgtagggttggttgtgtagggcactttggttttgattttgggtgggcacagtaatgtagtcttcagatgacttcttcagctataatcagtgtcagtggtatcttcgagttcctcagtgggttaggctgtggtttttagtagacactgtagtgaggttttgctatggattgggacaccaggtgggccagttctttggcaccagtatgctgtttcttgggttcccaggtggcatacatggaaactggtagaagcaggtctgagtgggctgatccttgggccttcatattatttggctgagtccctacccaaatctcatcctgagttgtagctcccataatccccacatgtcatggaagggacccggtgggaggtaattgaatcatgggagcaggtctttcctgtgctattctcgtgatagtgagtaagtcttgtgagatctgatggttttataaaaggagttcctccacacaagctctcttgcctgctgccctataagatgggcctttgcttctcctttgccttccatcatgtttgtgagcccccctgccacagccatgtggaactgtgagcccattaaacctctttcctttataaattacccagtctcaggtatgtctttattagcagcatgagaacagactaatacaggcttccaggtgacttgctaaagtgctggcagtggcagcagtaggccaggctggcaagtggatcactgaactcctgggcagtgtgcatggcatcaccaatggcgatagcagtagtgggcactagcactgatggcagcaaacttggtgggccagtcccccagccccaaggtggtacatatacaggtgactgctcgtggaggtgatggcaacaggctgggagtggacatcctcaggtccctggaaggaatgcatgggctccagcattggcgggaaaggtgtattgatctctaggcctctggataatgtgcatatgcactggcaggctggggaggcctgtccttagcacccccaggtgtggatgtgcagggcaggcatattcccaggcctctggacaatgcccacaggcactggtgagggtggggccaggtgagctggagctgtcctcaggcccctcaatggtgtgtgcatacaaaggctgtggcaggcagggctgatcaattcccaggcccccagacaacatgcacaggcactggcaacctgtgcgggcccattgtcagctccatagaaggcatgtatgcatgccagtggcagtgggtggggtaggtcaatccctagactcctggataatgttcatgggcacaggtgaaggcagtgcagaggcaggcctatgcttaggcctctagatggcatatgtgggtgatgcgtgtggcagtgtgtgtggttgatcaatccccaggccctcaaatggcacactaggatgccagttgtagtagcaatgggtatctttaggcactttgatgatgtatgtgggcaccagtggcagtgggggtggtaggtcggcctcctggttcccagatgatgcgtgtgggcactggtggtggtggcagacaggcagacctgtttttaggtctcctggtggcaagcagaccgggttgatctccaggaccttggatggtgtccttgggccaagaggcaggctagatgggcctgtcctcaggtccttggacagtgcctgggtgagccagttcccaggccctgttaagatatgtgcaggtgtgcagccaccctgctgctggggggtaaggatttgctgtcagtggcaacagccccaggcaggcagctctcaggctctggagagtacatgctttggctccctttgtccctagggcagcctccccagtgtgtttcactacccattccttggggcgtaagacattgcatgagctaaagttttggggacgcatctgcactgctggctccagccagtatcgtgatgctgtaaacctttgagtagatgtaggggggtgtctgcaggggttcagggatgtagatatgtagtgggtattgggccccaaaggtgtgatgtagtctggtgggggccggcctctcaaaatggcaccatgccgtagctgcttgaatctcaggggatgtgtgggacccagtgtgaacatcttctgtagaacaatgtcattacatggactccaggcatctctttatactagtctcagggcttgtgagggctgaggggctctcctgtggctaaaattataggagtctgtggtgggaatatggactgctgactgctgggaatctctcatttaccttttacccacagtgggagtttcccctggctctgagttgatcttggctgggttagctgcttcacttccctttccttctatgcctcagaagttcccagtcactttcctgctgaattccattgttttctttagatgttctatttgacatctaattatctatttgccattctggtccttcttcgtgaaggaggtgacagccaggtgcgtcaggcagccatcttgaagcccctccaacggagggttttcaattgcagattcaatttctaaagtagatatgggattacttaccttttctgtttcttcatgtgtcagttttcataggctgtaattttttttcaggaatttgtctatttcatccaaaatttcagattcattgacataaagttgctttataacatcatctttttttttttcttgagacagaatcccactctgtctgccagtcatgatcttggctcactacaacctccgccttctaggacatgaggtcatatctcagcctcccaagtagctgggactacaggcatgtaccaccacacctggctaatttatttgtatttttagtagatacagggtttcaccttgttggccaggctgttctcgaactcctgacctcaagtgatccacccaccttggcctcccaaagtgctgggattacaggtatgagccaccacgctcggcctgtaacatcatcttaatatgatttaatgtttcttaagatctctggtgtttttagtaattagcacctctcttgcctgaggtttaacaattttattcttttcaaagaaccaatttttatctttatttttcttgattatatttttgctttctcttttattaatttgtgctttatccttattatcttcctgctattcttttcattgtgtgtgactattgtttttttaaaaacaacatgaatttttagcctttatttctaaaatatgcatccaatgggagaaataaattcctgttgtttaagccatccactatggtatccttataggactgttgagagggtttggataagttaataagcttagagcaattcctggctcatagtaaattttgctatttttatttttattattattaccctggattgcttacaagagtacatataacgaagaaaaaattataaatagtaatagtcaggattcaggaaaatatgtttattgaaagtcaacatataagggaaagttggaatattaatattcaggccattaagggcatatttgaatgttgagctgccagggagtggtggacatgttacctccccccatctgctacactactttgtgataaacatctcaccaggcacacacacgatctcctcaagattatttgatggctgcctccaccacctgttattttatatcccctgaaccagccttgtgaaatgctcccaggcctggcatcccacatgtgtcctgcttggctctttgcataaaacgtccagcttgggaggccagattgctgcttcagggcgacaagtcccacagaggtggtaaaaggaattggccgccagtcagaaactggtggcaaatatcacactcaacccgctgactcttcttgtgaatgcaaaacacatttagagatgaaatggtactcctctggctgccaaaaaaacaagtaacaaaagaagcaaggcccacatatgtttgtgatagtgagtgttttaccacgcagcaagggatctgcattttgtataggtgattttaaaatgttggcaaagatccttcccccttagtgcctcagcgttggtttggggtggtttgtggagatgagggttatgaggagggaaaagcactcccgtgtgaacaaaggcattctctgtgaggtttatctaagaatactgtccaagtgtcaagcgtctgataatgaagaaaacggaccttagccatggacttgcttttcagcattagacacacaaaacagaagcatgggtatgtgtctgccaaaaaagcaacctcattctctgccaaacactacagctcttaatttaaaaaattgaaaccaaagtagacttgtctctggatggttacctaaaatgtccacttaaagaaatgacagctctcctaggggagcagtacggtaccgagagagggagcaattttccctgtgtattcacatcaagccatgcccagagaaatcaaacacagctcccaccagccttcagagatccgatgaagagcaagtgcttcttgttcctccttgttggctctgacctagaaggtaataagtttttggtattggtgccattttaaggattggacactgccctgttatataatccgagcagcagactttctgagcgtctgtcaaatttagaagttgtctgtcaaatgcaaggtctggactaacttatttctaagatatcacctaacaaagatccaaaatatctatatttcattaatgcccagaggggctcagccagcaatgcaatgtggaatagtgtagtgtctttcagcaaatgtcataatgctctctatgcttacatagatatgtaaatatatatatatatatatttatacctatctctatataccatatacctatctctagccactgagatagaaatatacacaaaatgtaaatctcaggctacgtgataaccaaagtcatgggtagcctctgcactggcattgaccataaacccttacaagtgttatctctgggtcccagaagttgagtcctctttctccttgtctgttgacaacccatggccattgctctcaaggccacccttttgggatctttctgtgcctttctccaaatatcaaagttgctgttcattatctgagttgatattgtcactgcatttgaaatgtaggacattttattcttaacatgccagccctaaggaagttttgactccacattaagaaatttaaaactttggccgggcagggttgctgacgcctgtaatccaagcactttgggaggctaaggtgggtggatcacttgaggccaggagttcgagaccagcctggccaacatggtgaaacttcatctctactaaaaatacaaaaattagccaggcatggtggcacacgcctgtaatcccagctactcgggagtcagaggcatgagaatcgcttgaacttgggaggcagaggttgcggtgagctgagatcgcgccactgcactccagattgggcaacagagtgagaccctgtctcaaaaaaaaaaaaaaaaaagattaaaaattttaatcggaataataaccatactgagaaattgctttaagatttttttttttagaataaatgtcattaagctgaatgtcctggagaaagaaaacacttctgtttttttcatcgttattttattgaaaatatctttattgacacaaattatttttcatataaatattgaaaaaatgttgagtcattgagcagttttcatttttaccgaatcttactgggtgtagcttctataatcatgagttttgcatgatctccaacaggctgattttcctgattgcaatttgagcccttactatttttattttatgttttcactgtcaatatcaattccattttcttccaaaactacaagatggattatctcggctaggttgtatttaatggctggaatgaattttaggtctcattttccagggaagggaaactgaaacccagaggttaagtgacttccctgaagtatcctacccatttcatgtctgagctgaggttagagtcttggcttacagcccagagattctctacttcacaagcatatccactactggaataatggaatcacagcagtatgctggtacagttaaaattggagcctatatgcatgcctaccataattaaagcagggccgagaaataatgtatttcacatctagtgttagctctgaatccacacccaaggtgaccacctcctatagacagagagaagattggggttttttgtttcattttgttttgttttttgagagggagtcctgctgtgttgcccaggctggaatgcagtggcatgatcttggcttactgcaacctccacctcccaggtgcaagtgattctcctgtctcagcctcccaagtagctgggattacagacacacaccatcatgcccagctaattttatatttttagtggagatggggtttcaccgtgttggccaggctggtctcaaactcctgacctcaagtgatccgcctgcctcggcctcccaaagtgctgggattacaggcgtgagccaccacgcccagccgattgggttttttacaaaggcactaagatttggtggtgaggccaggttcatataatgtcaagaaaagttatggctttatcggggaacttgtgatattaaaagaatgcatgctgaaattggccaaaatgagcagagagaatatggtgggcagtgtcatggataagagaatatggggcagggcacagggcagagtgcatagtctggctcatagcctgcagcttggagagtgagaggtgggagagaaaccagaatgggaacatcaggcctggatagtagacaaggagtgtgtgttccattgtttaagcagtggggagccatcagaggtttgaaagcacattttaaaagctgttctttgggaagtttataatggtaactttaggatgaattcagggtgaggaaactctagaaataggaagtcatataaggagctttattggaagaagattctccatctatttaatacgtccaccttcttgatattggtttggaagtcctgaaatagaggtcgtggggaataaggtttggaatgcccaagctgtgaggactgattccctgtttgagagatggtgttcactgtgtgtccccaccaacttaagatcactgcatgctggcgtccctggcacttcctcaatggccctagtaggcatcctcagccccctaaagcacaagccctgcaccaagacttgcaccttgaccttgacctgggtttagtatatggagccctcaggcccgtggtctgccacactgtgctcaggggcttctcccaaaatgtcctggtttccaacaggaatcagaccttcagctagaggttacatctagggaaaccaaagggaaggagcacgccaccctgaagcttgactgttctggaaagccctcaccaattggagggggtctatgacatgagaagaaggtggagttagacaaacctggtttaaactttcattttgccgcttgctagctgtgggctggttactggtcctggaaactttttttttttttttgatatggagtctcactctgtcatccaggctggagtgcagtggcatgatcttggctcactgtaacctccacctcctgggttcaagcgattctcctgcctcagcctcccaagtagctgggattacaggtccccaccaccacatctggctgatttttgtgtttttagtagagacagggtttcaccatattggccaagctggtctcgaactcgtgacctcaggtgatccaccagccttcgcctcccaaagttctggaattacaggcatgagccaccatgctggaaactcttgagtgagaacagttacttcataggggtgatgggaaggtcaaaagagatgataaggatggcgaccgatgattccgcatctgtgagctctcaggccacggtctaccacacacttttttaatccacaattttgtggaatcacagtagctatcctataaactatgtcatgttatttactaactcataactgaagaaacagaagcttcacctgaaaaacttgcttaaggctttgaacagtaatagtatctgagatttgaaaattcaaattctgacctttccaccaccccacagtgtctttaagtacctgtgaggggagtgcagtggggaaggatgaactgtagccggatctaggctacatgccccgctgtattagtctgttcttgtactgctataaggaaatacctgagacagggtaatttctaagaaaagaggtttaattggctcacagttctgcaggctgtacgggaagcatgacggcatctccttctgaagaggcctcagggagtttcacttatggcagagggcaaagctggagcagatgagagctagccggggagatgccacacacctttaaacctttagatgacaagatctcgtgagaactcactcagtattgccaggacagcaccaaggtagacatggtggggaaagcaccattcatgagaaatctgccccatgatccaatcacctcccaccaggccccacctccaaattggggattaccattagacaagcaatttgggtggggacacagatccaaaccataccactgcccttggggtatgtggtgatggtggcaaggcggtggtgtggtggtggtggacagaatatttggcagcacatgctaaccaccatagaatacttgttccctaaatgaaagaagagttcttaaaaggaggaggatggaggaggcatggttcatctgcagaaagcagtaacgaccacagcttcctaattgtgcttttctaaaactcacattaacttttgtttgtttcttgactacacacaactagaatgtggaggaaagaatgactctagtccttcgagtggacgatcaggctaggatttgaacattttcacagatagtccacagaaggaatgacagacagctagccacctgcctttctcggaattactgctgacaattcattttttgtgtgttttattaagaattttaaacagcttaggaagcattatgaattgtttctgaaggaacagttccagaagggtcttcagcaggtccttgataacattgacatggtcagaggaagcaacaagcaaaaaaatgtcacttttgcatttgactagatgagagggaaagagaagttttcaaaaacatattgaatggaggaaaataactgttaaataggcttaaggcaatgataatcaaatatagttaattccctggaaaaatgttttagctttggctgggcatggtggctcacaactgtaatctcaacactttgggaggttgaggtgggaggatcgcttgagcccaggagttcaagactagcctgggtgggcaagatagtaagactctgtccctacaaaaaaaaaaattaaaagaattaaccaggtgtggtggtgcaggcctgtagtcccagctacttgggaagctgagatgggaggatcacttgagcccaggaggtcgaggcgcagtgagctatgttcacgccactgcaccccagcctgggcgtcagagtaagaccctgtctcaaacaaaacaaaacaaaacaaaacaaaaagctttggctttgttaaggaaatcggcaaacagagtcttcatctcccccaccagcagtcgccttctacaatgatggatgtcctctttcacgggttctttgtctctatcttccagacaggggcaaagttcctctgtccacttatggaaatctgactgtgggcagaaaaccagaacacagtaatcaaaccacaccccacctcccagccagagagcttctctgaagtcacaggttaggtcctccaaaagcaggcactgaggcaaagtttggggtgcaaatagtttgttagagaccaacacctgtggaaggaagggtataggagcaagactggaccaagggagaaataaaactgtgatacaggccagacaaaccaggcagggaactcttgggagcatattttctatcagagtgtcccacactgggccaaaatagtgaggccttttatgcccagctcattgactcaccaaatttgggctgccccaggaaggtcatcatcttgggaaaggcagctctttgcagctgaggcagaccccgaaggagctgacagctgggggctgtctgctgaatgcactccctgcagtggagcagcaagtacatccttgaagagaggtccagtggggtgcatctctatgtctaccacaaaggccacccagataatgtacttgctagctctcatggatttgggggcagggagttgggagcagcgggagctggtggtttgcctaatatgggaacttggtctttctttgtttgcatgacataaaattttgactaagctctcagtgtaattatgtcttatatctgccttagaaagatggatcaggccttggtttttaactcatatcaagattccaagataggcaagatggatctttcaggaaaaggattaaagaatcatcagaaaatatttccctttttcagttatttatcatggaataataaatcactccaaaacttagtggcttcaaacaatttattatttctcacaaatatgtggattgagtgagctcagctgggtgggtctgattcagatggggtaggctgaggtcacatgtgcagctgcattcagctgggagtttggctaaagctgggacatccaagatgatctcctctcctccagtgctctctccataccacctctaatcatgtagtgtcctggcctggacttttccacaatacagcaggctggtgttccaaatggaaaagcagaagatgccagtccttttaaggcctgtactcaaaagtcccagaacattatttccatttcattctctcctggtcaatgcaaaacacagagcaaagcagattccttttggtgggaagaatggcaaagaatctggagccatttttaatctcctcctttgccctaaaccaatgtgtctcaacctttgcagatacagatttccacacctgctctgtagataccctgattttattggtctggggtggaacctgggcattggtattgaagcttcaaagaggtttctaatatgtagccagtttgggaaccctaaactatgatgtcttttgtccctctattgataatgacatggcttccatctggcaggaacccccatgtggcatatgtttggagaaactttcattctgattcctgaagtatgttctaaatccatttttacttacaagctaaccagaaatttgagaacacagttttactctttacaaatcagccattgacctgaagcattgtctctgtcttccagtcagtagtatcaactggtatggcactggttctctgaaatcatcagccaaaaccttaatctaaattgtagacttcagtccctgccatgcactgcaggtttacaagtggtctttgttctttttaattattgatagatcactggcaatttagacttgtaagaaaactcttttatttgtttaaaagcattagtttccttttcccagcagttttgtgctgtactgaaacaacgtgttcctggtgtgcagtgctcttagatagtagtgtgtctgtgttgcctttcaacaaagaaaaaactaccaagcactgtctgctttaaacatcatccagagtgatggtttgcagcaaagatgtccactagccacagtgaattttttttctggataatagattcatacaattccatgtcaaagcaaagaaaaaaaaagtgccacattttagactgtgaagtcttattataagtaagctcagtatttccattagttactcattaggctatttaataaggattcttaacctggagtaagggtctgtggttaggattcaaggagttgtcagtgaacttggaagggaaaaaactgcatctttattttctctaacctctaactgaaatagcatttcctttcattatgaattgtgaaatttcacatttccttccattataaatgtaggcaacaaatcacagtggcacagcagtacctttgacttttgtcaccaatagaaagcaaagacattctcataccaaataccacatacactgttatagatatgttgaaaaccttttatccttatcactaaaattaaaattatagtagttttcagaccctctggtttctcctactgaatgtgctaatatacaagtatgtatgtgctatctcacaatttttttttactgttttgatgatttcagtggagtgtttcatttgcaatcctagcaaagcctaggatttgattttaattcactgtatattatctatgtatatgtgtggatgcatatgcactctgtattaattatgtatgtatttattttaaggcagggacttgccctgtcacctaggctgaagtgctgtagcaccatcatggctcactttagccttgaactcccaggctcaacaaatcctcccacctcagcatcccaggtatctgggaccataggcacatgataccacacctgactaatttttgtattttcttgtagagatggggtcttgccatgctgttcagcctggtttcaaactcctgagctcaagtgatcctcttgccttggcctcccaaagtcctaggattatcggcatgagccgccatgcccagccagcatgcatttagaaacattattgtgagaaggtagtccacaggctgcaccagacagtcataggagactgtagcacagaaacagattaagaacccatgcattaggctatttgaattgttgaaaataatttatagattctccccagcccaaatcacctatcacctttggtgataccagacagattttatgagctaaactatcaagtaggcttgaattttgtttctagttgattacactggcaattctaactacttttgttaccatattcaagtgacattatagtatagggattaagagaatggatactgtgttagacttggaatagatccctgctctaccatgtaccagctgtatgaccttgggcaagttacttaacctctccaagaaccccctcacctgtgaaatggggataatattatcacccaccactctcacaggagggttcattattaggatgaattgagataatgcttataaattacttgaccctctctctacctggcacctaataagcataaaataaatgttggtttctattgccattagggagctctcttagcattaatctattttcacagggcaagaacctcttgctctgttcatagagtccttaagagacattttgtattaaagtttcagatgatgaaatgagttcaatattgtctgtcagataggcctgaaactaccacagctgtatccagttttaacttgactttaacaaagagactgtcttgtcaaaaggtagtagtagcagagagggatggggcggtgttggacattttgctatatgacagcctaataggaaataaaagtggctgaattgagtaatgagttgtttctaaagcattgagtcccttctagtttaacaataatttgaaacatccctgttgacaacaaatgaggtgcatccagcctttttttttttctttttctcctgtttgtactgagcttctaacatatggaagacaggaaatataggagcttacatttagagagtaaaaaactatcaaaacaaaacaaaaaaaaaatttggcaacttattgctcttctgctgtccagcatggagatgtgggtatttatttttgttcctaatatttatttttttatacagcaaccagagtcccaaggtttagattctggcaccggctctgtcatcttagagctgcttgccagagggggacttcccacttttgactttgtttcagttcaattgcttctccaggccttccccacatactgtttttcagacatggggccaatctcatcatctctgtgtaggggagaagaaaaaagcacagcagagaacatgttaaatttcctaaagattcatttttgcaaggatggcagcatcagcaggtggtttttcttttcttcttttaaatttaaaattgaatacataccaaagaatatttaccatgctgtctaagataaaacagtgataaatgaacacttttgtactcaccagctggtttaaaaaatacgacattgcccttcctaattacccctggaggtagactactgtcctgaattttgcttttaccatcccctttcttttttctttgttaatagttttactgcagacatacgtatccatatataatattttacttttcatgtttaattttatagaaaagaaattattctgtatgtattttgcaaaaacatacttttgctattcaacgctgtatgtgagattcacccatgttgatatgctcaattgtgtcttattctttttcattgctgcatagtatttcatcatatacgtgttattcacccattccactgccgatggacatttgggttgttttcagtattttgcttctacagatagtgctgctatggacatttttgcacatatctccttgtgtctgtgtcagcctcactagataatgttaaactgttttccaaattgattattcttgccggtaaagtataagagttctgattattccatgatctcagcaaacacttggacttgcctctagttctttcacctgagcttattaaattaaaggaaggcttactgtcctttctcacgtctaaaagtcatggtttgtattctctttccatagctgtctcctgtctttttttttttttttttttttaatgccatcaaattgaaggactctggcatacacacacacagcgaaggcagagtggacatttcacatccaagtccgggtggttatgtaggttcatttttcttctgggtgtcctgtgctgaggttggctagcaaaatatccagctcaaaaaaaaaatcagaaagttattcccttagcaccttttcaattcaactctgccaacagtggctcagtaagaagcagcaagtaaataaaatcaacagatttcagcttatgccttgggctcgccagcaatccccagaccacagggagaaaatgaatctttttctcacaaaattcctctcactcccatcacttttccattgaaacaacaaggcagagaagttactttattgacttgctttcctgtgagtcttaccagaaaatgaagaataatagtaattggcaaatactacccacccagttccttagctagcacttctcacatctgctttatgatatccttgctggcctgattttattcatttcctagtgctcggcatagaaaccacaagaactagcaaaaaagaaaaacactgcatttatataaaccaataaaactagtgagcattttgtggacattaaaagaaacatgagggctatcccacattataaatttcaaaatgcaagtttttgccttgtggtattttaaaagttttaagatgctattcaggaacactctcaataactgttttaaattgttgtcgtttaaacgttttttccttttccaatatcccacttgcctagtcttaactagtctccttcctcaccaatctgcccttctgtgctttttccaaaatgcaagtttaactgtggttgttttccttgtttaaaaccactcactaactctgatacctgcaggataaagtcctaactctacctggattaatgaaaagatttgatctggcctctgtttcctttcccaaccttaactcttaccctctccctcctttgcaattaaaatgttgaaaaacaaaataccatatcatcccccaaacatgcacccagaaattcacatgggattccctcctcttgccttgtgaggccctttgtactcctctgacacacctggaaaactcccatttgcctttcattcttggcaatcttggtatgcttgatctttccccacattacctccttcttggcaactctctccatctacgtacatctcctattgtatgtactacactggggtttcacacttcaactctttaattgccttcctcctctactcgatgctaagttccttgaggacaggaataatttattttaaaaatattcatttatttttgtagcataccccttagcacagttcttggcactgagtaaatgttcagtgaattaaaatcaaatcggcctctacctcattcccatgcccctctcctttgcctctgactgtgtgtgtgtgtgtgtgtgtgttttaatactacatctctcaaggagtgtcatgattgttcaccagttgtacacaccaaagtgtcttggaggaaacagagtagacatgctgagataatgaacagttcaaaacaattcccttcaaactaaagactacactgtttatgtcagctaaagactatttcctttctgtgttgcatgggagaaaaaatgagaaatattctgggacacaaatttataaaaggagttgtagaaatttcattacctcagtgctgactagagcatcctctcctttaaaaataattgaattcctggtttttgtttttttcccttctcttaaaaaccacacagcttgcttcctgaagacaaaggaaataataaacaatagcaactcccaatttgactctgattatttgccaaaagtcttcttagttcatgtggttctatttcattagagaggaccgattagcataagaagaatgcttctaaattaagctacccaaatatgcacttagttagtactctgggagctaatttattgtattatttaaaatataaattggctagcaaatctatttattgaggtgtaacagctaggcaacaatgcgtaactttctcttggctgttgagtggtttttaatcatctattgaaatttaacagagagccatttcattaaatggaaagcggataaatactgagaagtctgtattttagagtattatccacacatggagaatgatatttgaggaacaaagagggaattgggatttgctttttaaacagtaactttttgttgttttaatttgaagtgtaaccaaagactcacaaaatcacttgtggggatgttaaagcaagaaataaacaaataaggcggaagatggagtagggtagagaaatagagggatggaaaaaattgtgttaagatagtgctacgttgtcaattagactaatgcagtatttttgccttgtacagttttggggttggcagtaaaaactatgtaccttgttttattttaacaaatggcatcatgaaaatatcatgctatttggcatgctgtgtattctaaattcccagaacctagagacatttaggggttctggggttcccttaggctgtgagtcgagaacatagttgaatccagataccctgtctggtctggagttctcctaaacagatcagctctggagcatctcatggtgctctgatataaatggaagagtcttgtagatgttgtagcatggtcttgaaattctatcattgtcacttatactagaactcagtgagggtaggcagaaatggaaacaagtgacaggcctttctcacctttctgcaaatttttgaaatctggctcatctgatttccagagccaaggtttcaaagaagttttagaccttgtaatttacgataggtggatgttccttaggttcacgttggaaagaagaagaatgtcagaatgtttcctctttccccaggacagccaaaatgtcagagaaccaggagtctgagggtaaactgaagagagagggcactgatttgtagtatttgccaatttccatagtgtaaatactctcacgcagccaatttcaagctaccaacttcacaccactaaacatggagttgggcagcaatgtgcacagttggttctcctgatccagtgagggagtgcccacacactgctgctagaactatcttgcattgctgtctgcaggaagtaaatctttagcaggttagccggctcttgtttccatggtacacagaagatgatgagtcaggatctaagtccttgcagcactcctggcgagagcatttactgctataacctctgccccttcctcccccactcttattctctcttccaaaagaaaactacaaatgtagatcataaagctagatattgagtaagtataaataatgaactggattgtgctttttatcctcttgtggatatcaagtataaaaactggacagggaaggagagaatgaggatgggaaatcttgattactttttaattgcatcaaatcctgtgtggtaaagtgggttcctaaaaggggaaaaagacagaacagttttttgccattctgctacgacaagtaaggtcttgctttcctggcatttctgtgtaggaagaagagggtgtttggcattgcatgtggtgaaaggggagagagggtggagggaaagaatgggatggatcatggcattggcagggataaaactcactgtagacacagttaagttgagtgcagtgacagaggtttgctttcacattcaagctgcttcattaatttcattggggccttttccaaggaaagggaggattcataaaagggaaatcaaagaagcctggaaaagaaacatttccccttctgaagactgtattataatccttttttctgaaaaagtacagtaatgaatgttatatttattagtgctgtaatttctcctcttcacttcttaaagggaaaaaaatactctgtttgtcataaaaactctgataatagtagtagagtgaccactggttgagtccttattatctttgtaatacactggcacatggtacaaacttaagacatatttgtaatgggccaggcgcgatgggtaatctcagcactttgggaggccgaggcaggaggatcccttgagcccaggagtttgagaccagcctggggaacatggtgaaaccccgtttctactgaaaatacaaaaaaaattagctggatgtagtggcgtatgcctgtaatcccagctactcgggaggctgaggcacaagaatcacttgaactcagcaagcagaggctgcagggagccaagatcacatcactgcactccagcctgggccacagagtgagaccctgtctcaaaaaaaaaaaaagaaaagaaaagaaaaaaaagacatatttgtagaccagatgccctgcatcattttataagtattgtctctgaatccttacaaaaagttattcatagagtaagcaaccattccagattgcctgacccttttttacttttagcactcaaagccctatgttcccagaaacctcttggtcctaggcacagtgcgacaagtggtcacccaaattcttatccccattttacaaatgagaaaatagagatagttaagtcattatgccaaagtcacagaactgggctcaaaccctgagctaccaaatcccaagtccgtgctcttaaccatcaggccaaatggcttcctattatcctgtactctgcttttagattttcattaactatactgcccaaggaaagagaaagcttcaacttcaataattcatcccaaccttgctgaaattaacacagaaattcttggagttaatacagagaaaattttccatgactttttttttaatagacacgttcaaagcacgtctaactgatctggtgaaataaggctaacagatttgagtgaaaggtcttcacctctcaggtgttcattctgtatctctctaaaaggaaggaacgtatcacaagatttcttctcatttgagtaccctgattgtcagaagtctgtttctcatatcatgcagtaatgccccttccacttactctcatccttttattcaatctattatttggaagtcatactgccctccccttttaccactattaacttgataatagtagattattttatctatcctttatgtttatgtagagattatgacatatatcctgatttaagaaagtcagctttgagtcaatacatttaccattgtccagatataaaagtgtaccttacaacaggggcccccaatccctcggtcacagactggtaactgtttgtggccttttaggaagcaggccacacagcaggaggtgagtgcaggcaagcattactgtctgagctccgcctcctgtcaaatcagtggcagcattagattcttataggaggggaaccctattgtgaactgcgcatgtgagggatctagttgtgcgcttctcatgagaatctaatgcctgatgatctgaagtggaacagtgtcatccccaaaccatcccccactccccaacactgtgcatggaaaaaccgtctcccacaaaattggtccctggtgtcagaaaggttggggactgctgccttacaacactttaactcactttacaccctttttgtgcaagtactgtcttttgaaaaacttctacatagttcaaactccagaagacattattaattattgatactccttacttgtgttcacatatgtaaattctcagttgctgttcattctcctctgcattttcatgtttccattacggataattttccttctgtctgaagaacagtctttcgtacttcttttagtgcaggtctgctgacaaaaaccttttccaattttttgtttccttgaaaatatctttacaaggtcttcatttctatacaagttctaggttggcagatattttatttaagaactttcaagatattatttcactgtcttccagctttcattgtttctgttgaatagtcagttttcttgctgctttgaaggtactgaaagtactgtttccttttctgtgacagcttttaagattttctctgtctttgcttttagcaaaatgctattctctctgtgtgtgtgtgtgtgtgtgtgtgtgtgtgtgtgcacatacacatactatagtgtaatatactattactaatatactattatgtgtactatgatgtggttagtttgctttgtgtttattctatctaggattcatagagcttctcaaaactggcttggtaacttttgtcaattttggaaatttttcagtcagtatctctgaatgttactatttgtttcttttgccccatttcttccttatcattttatcctaaaattccagttaaacataagttaaatgtttcatggtttcatgtcgtcttcttcttctttttttttttttttgaattttcatacctttattctccatgcttcagtctggatatcctctactgacctaacttccagttcatatagtgtttcttctgcatgcctaatctattgctaaaacatcgatcgagttcttaatttcaatgaacaatgaatgagtaactttttagttaaagaacttccatttgattctcttgtatatattctaattatcttgtgaattatttttctatacctaacatcttgcatattttgttcacagttactttaaaatgtgtacctggtaactctaatatctggatcatttgtggtctctgttatctttttttctccttaaattttagtcattgctcttgttctttgtcatgcctattaaattttattaaatgttgaagaaaatgtacaaaaaatatatgaagaaaaatgttatttttcttccaaaaggatttaattttttctagaaggcatacagagtagaggcatatcaccttgataaagtttgggattgagacgattgattcagtgcttgatttcaaggtttgtgaggactaacctgtttctggtttgttattacttccaaggcattgtctttcacggatctgatggaaagcctgcagtgcttaccagtgctgtccctccttgacaggcttgcaactccaattttttgttgttgttgtttcttttggggttttctttgtttttgttttttggtctctcaagcaccgtaagactaatgagcttttcattctcttatctgccgcttttgacttggcgtcttggtcagagaagcttaaaaattggtgaatttcccaaagggaaaagcagcacaaaatgttaggctcacttccttgtgattcccttttctctacgatcatggcctctctcaaggcttggttgctctctgatacctttaaacagcttattttctttttaattcagcttctattgttttctgtggaataagattggtttggtacatgctattgtgtcacagtcagaagtggaagtcctaaattacaaacagtattgttgcaactgattaagatggtgtacataggaagccccttggggatgttgtaaaggagcaccaaaagttttaactgctctagaatatgtaccattatgatggctacatgtgggtctcctgaagcacctagaagctccttttctcattctattcaataaccatttgctacaccagaggtgtacagtgggagacctcaagaatcgtttccctgagtgctgttgaagcaactcccagccttgatctctgctgttttttcattctttcgaaagtgacttattgcattctttgttaggaagtgtctgctaacaagctttggcttgttttttattctcgttcccaagtaccttttcaatatatttttatttgaatgctatttacgttatttctacttatagttagtgtctgaaagcaaactaaaagataagatttagtaaatgtggaatggcataaaagtagtgaaaagaacttacatgtaaatcagaaaccacaacaagcagattctcttgagggtatgcatctttctatactggaaagcagaaacaataaaccaatcctacatattctgcctgccaaacttgcagctcccatgtggctcttttctctttcctggtgagtagcaccacctttgcaaatctgcccatgtcagaagcctgaaatggggaggatttgttggggatgggtagagaaagtctcacttaacctttatatcggattaatcactgattttattggttccatcttactaccctcttctccttccccttctccccatccctcccatgagcccacatcctctcacctgatttggtagtctcctaatttgcctccttgtaccagtatgactcccttccaaattagccttctccttagacattaagtcatttttctacaatgcaaacataagcatgtcactaccctacttaaaaccctctaggatatatccagaataggcaagtctatagagacagaaagcagattaatggttgccaggggctgatggggagagggggagtgggaagtgattactgatgtatatgggtttcctttggggtggtgaaaatattctgcaacaagatagtgtgaatatactaaatgctaccgaatcatacgctttaaaatggttaaagtggtaattttatgttaagtgtattttaccacagttgcttttaaagcccgataggataaggtccactcatcccagcatgtcacttggggcatcacatggtcccactagcgtttgcctgttcatctgtctcttctgtttccctctattcctgcatgaatgggacgacatacagactgctgcatgcgcccgcagcttcacggcccctgcatttccacacagaatttcctttgtctgcggtgatccctctgctgcacgtgcccacagtttcatgcccctgcatttccatgcctcctgcatttccacgcctcctgcatttccacacacagtttcctctgtctgcggtgatccctccacccccaccccacccacccaccactctccgtccagtgatctccttcaagacccccaagaagcttcccctgacacatctacatcaacaagcccactgtgaacttccctctcttttctgtcatctaaacaggcacaactgggaaacctttactggcaggtctttgtttctagttccagggagaattatattgttttctaaaaaataaagtggaatgaagctttaaagtgtttgtataaccctgttgaaattcatgacacatttgtgaccccctggaacatctggaacacatgaagttagaggctgcagctctagtgtggaggcacccaaaatagacagttctattcagggagccctgagattaatcttgtcacatttaaatacgtaattacagccctgacacattttgttactcacctctaaaagctgccaagtggtctgttatctttgatttgtgaatggtgagtggcttgattataaacaagagtggtttatttttccacactcaaaaatatatttatttttgaagttaatatcagtaaaatgctgtatgtgaatctgcttaagaatatgctttgtttgggaattttgtggcatgtgaaacaactttgtcccttaacaagctttctcttcccgcaatattaatgacacaatttacaatgccattagggtcattacacatggcagctcagaaattggaccagggtcaggtaggtcactgagaggaagagtaagaacacctgaccccttcagcgctccttcaccacttgataattatggacagggaaggtgtgtgtgagtgcgcatgtgcgtgagtaaataccaggagagcggatacccaggaagtgaggtcatgagtaggtattaaacaaatttctacttgtattcatgagtaggtgttaatcaaattaccttgatgtaaacagagaattccctggtttgccacagagtagaaaaaaattctcaacagtggttttcaacaagcagttggaacaacagcatcagcattgctttggaattgttagatatgcaaattcttaggccctgccctggaccttccgaattggaaagtctggaggtgatgcctcacgatctcttttaatgagctcaggtatttttatgcacgttcaaatttgagagtcattgttttgttttttcgtttgtttttgaggtggagtctcactctgtcgcccaggctggagtgcagtggtgccatcttagctcactgcaacctctgcctcccaggttcaagtgattctcctgcttcagcctccccagtagttgggattacaggcgtgcatcaccatgcctggctaatttttttttttttaatttttagtagaggcagggtttcgccatgttggccaggctctttcaaactcctgacctcaaatgatccgcccacctcagcctcccaaagtgttgggattacaggcatgagccactgcacccagccaagaaccattgttttaaacaaccttttttaaaccaaggttttttatgaaaatgtaggaacaccatgttctagggtagtttaggttcatataagcatatatacatatatattcatttatatttagagatgggatctccttttgttgcccaggctggagtgcagtggctcagtcagctcactgtagcctcaaactcctgcaatcaagcgatcctcctgatctcaagcaatcctcctgatataggcattttgattctggattttcaatatagataagtgttggaagaataacgaaaatgaccaatatttactgaagaccatcccaggccgggcactgtgctaagtattccagggacattattccactaactcctcacacaactcacaactaccccacgaggtaatacttgtttcccaattgtaaagtaaaggaacctgaggctcagtgaggttaagttccttgcccaaggtcacacaacgtgcagtgcctcttgccatgctcctgcactctgacttctgagcccaggctgtgaagcactgtgctatcctttgtttgtcctttgttagctgcagcactggttctgggggcaggatgacctttagctcttgggtattcaatttattggtgttccggaccattgctgccaaactcatttccagttgaagcagctgggttttgtcacacatatgactttgttaagtgtcctcagctgagctgaagctgcacagaaatcactcatcagattcctgccagggccaaagatatggatgaaaatctgcaggaagaaatgtctagcctgggattgcggggcggcaaaagtttccctctggagtcctatgaaggggttcttgtcacacatgtttactttcagtttgtgataaaaagtgggcatgctgggtttccagttgcaagcttctacacaaatggtagatgggtttcacagtcattgcagtgaggcacaaatgaccattgatcaactgcagcaaaacacaaaagtttcacatttgcatgtgaaagagttaagtctggtcccacatctgcaaatgggatctgcctccctcactgtaatatgtgaaatacaaaaatacagaaaattatgtttatatctgcaataatttttttcatcaataaacctcagagggctattgagaggtgaataaaaatgttccgtgactaagctgcaaatttcagttcaaagataaggaatgattgagtagagggtttgattaaaaacaccacaccggtgtttgcatcagaagtcagggttctagctagttatctcacttaacaattgtgtgacttggacaagccactgaataattccaaatagctcagtcatctcaatgctcatgtgaatggtgctccctgtagttgttcagtgcacagcttgtatagctgcgagctgcaaggtaaatatcatctaatagtgttaatcctacttactgcacgtcaaggttgtaagatggagggtgttgggatataggggcagattttcaatataataaatgtactatccaaatatactttttctgcctcagtttccttcagattgagacaagtagaagaacccagcctcttaggaagggccttgtgaaggtgacctttaattgctgatcgtgattctgcagctagaagttactgaaagtgatgtcctttttgcatacgtttattcacttcatgcactggtacctcctgtccattctccacatatggaacgagacatctcaggccaggctgacctctcctgcatgtaacttcccaacactcagtgtcagtccttgcccttttggcactgaatcatgacagcttcccgtttccctctacttggaatattagctgacgctagaggcttaacgagtacagagaccagtccccttttgctgattggatctcatcattagagatgttttaggaatgctttatatcacccaacaagggcttgggttacgtaggtgtctgcatttgtcagaactggtgagtctacacttaagactagcgcatttcattgtatcttaacttgactcaaaagaagaaaatctgtaaacacatgttgaactctgatgaataatgtgatgctgaagtgtgtgggggaagtgcactacatgcccattttactttgagatgtgtcaaaaatataagatggattagtggatgggtgtgggatgcatagaggcacccttatgtgagaaagcaagtaaagtgaaagttcatgactcaaaatcgtcactatatgaattgttttcgaatttgctatgttttaaaatgttttataatccaatattaggaaaaaaaagaatgttctaactctatgctgctcaaaacatgactttctttcatttcaagtgtcaagttttttgttttgttttttttttttttgttttttggtttttttttttaagagatggggtctcattctgtcaaccaggctggagtgcagtgcatgtggcatgatcctagctcactgcagactccaactcctgggttcaggtcatcttcctgcctcagtcaaggatgtttttggttttttaatttttttgagaaccaaaatttccccttcattactgataaaggctaaaatggaaggtatggctcagtgtgagggacaattgggctggctaactgaagggtggaattaagtagacaatccaccccattgcaggcagacctcacataccccagaagtagtgggctggaggtgggcctcaccaggtaacacttgcagtgcagcactctcaactgcggggagcaggcagatcactctctagaggaaaaagacaaggaataattttcaatttttacactcatgagaaaagaatacaactttgcgtaaaattaactaagctttaaaacttttctaccttaacatgcctgttacaaaagccatcaacagccagataggcagcttgtccaaaggcatgcattgggagaatatgttctgtatttcatgtgagttttaaatagagtttaatctgaatgaaaactttccatgtatttagaatatccatttctctaaatttccgttcttttactttttctttttaaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttg

SEQ ID NO: 2 - RNA, 2213 nt, Homo sapiens
  source 1..2213: mol_type = genomic RNA
gctcctttcgcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggctgcaatacagtgacacgatcacagctcactgctacagcctctacctcctgggcttaatccatcctccctcctcagcctcccaagcagctgggactacaggtgattttgatatgctgctaaggtttgataagagaaatgagattcagagaacagattatattgttgaagagcttgactggaaatgatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttg

SEQ ID NO: 3 - RNA, 2087 nt, Homo sapiens
  source 1..2087: mol_type = genomic RNA
gtgactgggtggggctgcctcacttctgcctgatttgggaagcgctgcaaggacaaccggctggggtccttgcgcgccgcggctcagggaggagcaccgactgcgccgcaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaa

SEQ ID NO: 4 - RNA, 1739 nt, Homo sapiens
  source 1..1739: mol_type = genomic RNA
agcgctgcaaggacaaccggctggggtccttgcgcgccgcggctcagggaggagcaccgactgcgccgcgtaagtgccgcctgccctgcgtgggtcgtgccagctcagcgggacaggtcctcgcctcggtccctcggacttagggagcgcggggcagaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaa

SEQ ID NO: 5 - RNA, 3219 nt, Homo sapiens
  source 1..3219: mol_type = genomic RNA
gtgcacagacggcccaggatgggttaatgagcaggcagctcagacgccgcaggtagcggccctgcctctgaacaaagaggagtccacacctggggtgcagacaaagggaggccgcctctgctacccaattcccagttcacatcgcctgctgctggggacccctagtggcggcacacgacgcaggcacaggtgattttgatatgctgctaaggtttgataagagaaatgagattcagagaacagattatattgttgaagagcttgactggaaatgatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaagaatcttgctctgttgccaggctggagtgcagtggtgcagcctcggctcactgcgctctgcacctcctgcgttcaagcgattctcctgcctcagcctcctgagtagctggaactacaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 6 - RNA, 2889 nt, Homo sapiens
  source 1..2889: mol_type = genomic RNA
gtgcacagacggcccaggatgggttaatgagcaggcagctcagacgccgcaggtagcggccctgcctctgaacaaagaggagtccacacctggggtgcagacaaagggaggccgcctctgctacccaattcccagttcacatcgcctgctgctggggacccctagtggcggcacacgacgcaggcacaggcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 7 - RNA, 3380 nt, Homo sapiens
  source 1..3380: mol_type = genomic RNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggctgcaatacagtgacacgatcacagctcactgctacagcctctacctcctgggcttaatccatcctccctcctcagcctcccaagcagctgggactacaggtgattttgatatgctgctaaggtttgataagagaaatgagattcagagaacagattatattgttgaagagcttgactggaaatgatccaaacggaaacattggctcttcctggggttccagcctgctgtcttgacaggaactataccatcagctctcctgggtctcttacatgccaagtcatcctgcagatctgggactcactccgtaatcaagaatgccaccaattcagcacttaaaaaattctgggccaggtgtggtggctcacacctgtaatcctagtactttgggaggccaagaccggcagaccacttgaggccagaagtttgagaccagcctggccaacatggtgaaaccctgtctctactaaaaatacaaaagttagccaggcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 8 - RNA, 3401 nt, Homo sapiens
  source 1..3401: mol_type = genomic RNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggctgcaatacagtgacacgatcacagctcactgctacagcctctacctcctgggcttaatccatcctccctcctcagcctcccaagcagctgggactacaggtgattttgatatgctgctaaggtttgataagagaaatgagattcagagaacagattatattgttgaagagcttgactggaaatgatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaatatggagttttgctcttttttgcccaggctggagtgcgatggcacaatcttggctcactgcaacctccacctcttgggttcaagcaatgcttctgcctcagcctcccaagtagctgggattacagagacagggtttcaccatgttggacaggccgggcacctgacctcaagtgatctgcccgcctcagcctgccaaaaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 9 - RNA, 3205 nt, Homo sapiens
  source 1..3205: mol_type = genomic RNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggctgcaatacagtgacacgatcacagctcactgctacagcctctacctcctgggcttaatccatcctccctcctcagcctcccaagcagctgggactacaggtgattttgatatgctgctaaggtttgataagagaaatgagattcagagaacagattatattgttgaagagcttgactggaaatgatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 10 - RNA, 3162 nt, Homo sapiens
  source 1..3162: mol_type = genomic RNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggctgcaatacagtgacacgatcacagctcactgctacagcctctacctcctgggcttaatccatcctccctcctcagcctcccaagcagctgggactacagatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagcatccatttgtgattggccatttcaacatctgtccttctcagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 11 - RNA, 2869 nt, Homo sapiens
  source 1..2869: mol_type = genomic RNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggctgcaatacagtgacacgatcacagctcactgctacagcctctacctcctgggcttaatccatcctccctcctcagcctcccaagcagctgggactacagaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 12 - RNA, 3380 nt, Homo sapiens
  source 1..3380: mol_type = genomic RNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggtgattttgatatgctgctaaggtttgataagagaaatgagattcagagaacagattatattgttgaagagcttgactggaaatgatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaagggtctcgctgtgttatccaggctggagtgcagtggcatgatcatggctcactgcagccttgacctcctgggctcaagtgatcctcccacctcagcctcccgagttgctgggactacaagcactgccaccatgctcagctaagaggcatgccactacttcccttcccactccttgccacctgcaccttctgactcactctagttgaaagccctgggggaaggaacaaactctgcagcctgactgctgtgtttaatcccagctctcctctactagctggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 13 - RNA, 3248 nt, Homo sapiens
  source 1..3248: mol_type = genomic RNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggtgattttgatatgctgctaaggtttgataagagaaatgagattcagagaacagattatattgttgaagagcttgactggaaatgatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaagggtctcgctgtgttatccaggctggagtgcagtggcatgatcatggctcactgcagccttgacctcctgggctcaagtgatcctcccacctcagcctcccgagttgctgggactacaagcactgccaccatgctcagctaagaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 14 - RNA, 3215 nt, Homo sapiens
  source 1..3215: mol_type = genomic RNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacagatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaatatggagttttgctcttttttgcccaggctggagtgcgatggcacaatcttggctcactgcaacctccacctcttgggttcaagcaatgcttctgcctcagcctcccaagtagctgggattacagagacagggtttcaccatgttggacaggccgggcacctgacctcaagtgatctgcccgcctcagcctgccaaaaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 15 - RNA, 3087 nt, Homo sapiens
  source 1..3087: mol_type = genomic RNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggcttgattgctgtaagttgaacacgtcagtggcatgggcaccagctgcacaaccctaggagccaccaaacacagtatattctatataaatgttgctgccaggagccccagttcaaactacaatatggagttttgctcttttttgcccaggctggagtgcgatggcacaatcttggctcactgcaacctccacctcttgggttcaagcaatgcttctgcctcagcctcccaagtagctgggattacagagacagggtttcaccatgttggacaggccgggcacctgacctcaagtgatctgcccgcctcagcctgccaaaaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 16 - RNA, 2418 nt, Homo sapiens
  source 1..2418: mol_type = genomic RNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggaggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 17 - RNA, 2179 nt, Homo sapiens
  source 1..2179: mol_type = genomic RNA
gtgactgggtggggctgcctcacttctgcctgatttgggaagcgctgcaaggacaaccggctggggtccttgcgcgccgcggctcagggaggagcaccgactgcgccgcgtaagtgccgcctgccctgcgtgggtcgtgccagctcagcgggacaggtcctcgcctcggtccctcggacttagggagcgcggggcagaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaa

SEQ ID NO: 18 - RNA, 2608 nt, Homo sapiens
  source 1..2608: mol_type = genomic RNA
aaacaaagaaaatcaagtctgcgaggagcgagggtttgctcccctctgctccggatttgggtgtccagggtagcgggcgcggcacggcaaacaggtcggggcgctctgcttcaccccaaccttctccgccccgccagcccccgctccagctgcgccagaaagtgcgagcctgcccgctcctttcgcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacagatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaa

SEQ ID NO: 19 - RNA, 2480 nt, Homo sapiens
  source 1..2480: mol_type = genomic RNA
aaacaaagaaaatcaagtctgcgaggagcgagggtttgctcccctctgctccggatttgggtgtccagggtagcgggcgcggcacggcaaacaggtcggggcgctctgcttcaccccaaccttctccgccccgccagcccccgctccagctgcgccagaaagtgcgagcctgcccgctcctttcgcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaa

SEQ ID NO: 20 - RNA, 2021 nt, Homo sapiens
  source 1..2021: mol_type = genomic RNA
gcggctcagggaggagcaccgactgcgccgcaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaaaaaaaaaa

SEQ ID NO: 21 - RNA, 1581 nt, Homo sapiens
  source 1..1581: mol_type = genomic RNA
gagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagattctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatcccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagtgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaaaaaaa

SEQ ID NO: 22 - RNA, 1638 nt, Homo sapiens
  source 1..1638: mol_type = genomic RNA
ggacaaccggctggggtccttgcgcgccgcggctcagggaggagcaccgactgcgccgcaccctgagagatggttggtgccatgtggaaggtgattgtttcgctggtcctgttgatgcctggcccctgtgatgggctgtttcactccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggcttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcactccttgatgggcatggactggaaaggatcccaggaatacaagaaggcagaaaaaaaagtttggaagatccttaaatctgacagtgaagtggctggttacatccggcaagcgggtgactcccatcaggtaattattcgaggtggaggacatattttaccctatgaccagcctctgagagcttttgacatgattaatcgattcatttatggaaaaggatgggatccttatgttggataaactaccttcccgaaagagaacatcagaggttttcattgctgaaaagaaaatcgtaaaaacagaaaatgtcataggaataaaaaaattatcttttcatatctgcaagatttttttcatcaataaaaattatccttgaaa

SEQ ID NO: 23 - RNA, 1672 nt, Homo sapiens
  source 1..1672: mol_type = genomic RNA
aggacaaccggctggggtccttgcgcgccgcggctcagggaggagcaccgactgcgccgcaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcactccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaaaaaaaaaaaaaaaaaaaaaaaaaaaaaaaaaaa

SEQ ID NO: 24 - RNA, 1772 nt, Homo sapiens
  source 1..1772: mol_type = genomic RNA
agcgctgcaaggacaaccggctggggtccttgcgcgccgcggctcagggaggagcaccgactgcgccgcgtaagtgccgcctgccctgcgtgggtcgtgccagctcagcgggacaggtcctcgcctcggtccctcggacttagggagcgcggggcagaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagtgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaaaaaaaaaaaaaaaaaaaaaaaaaaaaaaaaaaaaaa

SEQ ID NO: 25 - RNA, 1605 nt, Homo sapiens
  source 1..1605: mol_type = genomic RNA
ggctcagggaggagcaccgactgcgccgcaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggcttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcactccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttg

SEQ ID NO: 26 - RNA, 1686 nt, Homo sapiens
  source 1..1686: mol_type = genomic RNA
ggtgactgggtggggctgcctcacttctgcctgatttgggaagcgctgcaaggacaaccggctggggtccttgcgcgccgcggctcagggaggagcaccgactgcgccgcaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcccttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagtgggtgacttccatcaggtaattattcgaggtggaggacatattttaccctatgaccagcctctgagagcttttgacatgattaatcgattcatttatggaaaaggatgggatccttatgttggataaactaccttcctaaaagagaacatcagaggttttcattgctgaaaagaaaatcgtaaaaacagaaaatgtcataggaataaaaaaattatcttttcatatctgcaagatttttttcatcaataaaaattatccttg

SEQ ID NO: 27 - DNA, 2213 bp, Homo sapiens
  source 1..2213: mol_type = genomic DNA
gctcctttcgcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggctgcaatacagtgacacgatcacagctcactgctacagcctctacctcctgggcttaatccatcctccctcctcagcctcccaagcagctgggactacaggtgattttgatatgctgctaaggtttgataagagaaatgagattcagagaacagattatattgttgaagagcttgactggaaatgatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttg

SEQ ID NO: 28 - DNA, 2087 bp, Homo sapiens
  source 1..2087: mol_type = genomic DNA
gtgactgggtggggctgcctcacttctgcctgatttgggaagcgctgcaaggacaaccggctggggtccttgcgcgccgcggctcagggaggagcaccgactgcgccgcaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaa

SEQ ID NO: 29 - DNA, 1739 bp, Homo sapiens
  source 1..1739: mol_type = genomic DNA
agcgctgcaaggacaaccggctggggtccttgcgcgccgcggctcagggaggagcaccgactgcgccgcgtaagtgccgcctgccctgcgtgggtcgtgccagctcagcgggacaggtcctcgcctcggtccctcggacttagggagcgcggggcagaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaa

SEQ ID NO: 30 - DNA, 3219 bp, Homo sapiens
  source 1..3219: mol_type = genomic DNA
gtgcacagacggcccaggatgggttaatgagcaggcagctcagacgccgcaggtagcggccctgcctctgaacaaagaggagtccacacctggggtgcagacaaagggaggccgcctctgctacccaattcccagttcacatcgcctgctgctggggacccctagtggcggcacacgacgcaggcacaggtgattttgatatgctgctaaggtttgataagagaaatgagattcagagaacagattatattgttgaagagcttgactggaaatgatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaagaatcttgctctgttgccaggctggagtgcagtggtgcagcctcggctcactgcgctctgcacctcctgcgttcaagcgattctcctgcctcagcctcctgagtagctggaactacaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 31 - DNA, 2889 bp, Homo sapiens
  source 1..2889: mol_type = genomic DNA
gtgcacagacggcccaggatgggttaatgagcaggcagctcagacgccgcaggtagcggccctgcctctgaacaaagaggagtccacacctggggtgcagacaaagggaggccgcctctgctacccaattcccagttcacatcgcctgctgctggggacccctagtggcggcacacgacgcaggcacaggcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 32 - DNA, 3380 bp, Homo sapiens
  source 1..3380: mol_type = genomic DNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggctgcaatacagtgacacgatcacagctcactgctacagcctctacctcctgggcttaatccatcctccctcctcagcctcccaagcagctgggactacaggtgattttgatatgctgctaaggtttgataagagaaatgagattcagagaacagattatattgttgaagagcttgactggaaatgatccaaacggaaacattggctcttcctggggttccagcctgctgtcttgacaggaactataccatcagctctcctgggtctcttacatgccaagtcatcctgcagatctgggactcactccgtaatcaagaatgccaccaattcagcacttaaaaaattctgggccaggtgtggtggctcacacctgtaatcctagtactttgggaggccaagaccggcagaccacttgaggccagaagtttgagaccagcctggccaacatggtgaaaccctgtctctactaaaaatacaaaagttagccaggcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 33 - DNA, 3401 bp, Homo sapiens
  source 1..3401: mol_type = genomic DNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggctgcaatacagtgacacgatcacagctcactgctacagcctctacctcctgggcttaatccatcctccctcctcagcctcccaagcagctgggactacaggtgattttgatatgctgctaaggtttgataagagaaatgagattcagagaacagattatattgttgaagagcttgactggaaatgatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaatatggagttttgctcttttttgcccaggctggagtgcgatggcacaatcttggctcactgcaacctccacctcttgggttcaagcaatgcttctgcctcagcctcccaagtagctgggattacagagacagggtttcaccatgttggacaggccgggcacctgacctcaagtgatctgcccgcctcagcctgccaaaaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 34 - DNA, 3205 bp, Homo sapiens
  source 1..3205: mol_type = genomic DNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggctgcaatacagtgacacgatcacagctcactgctacagcctctacctcctgggcttaatccatcctccctcctcagcctcccaagcagctgggactacaggtgattttgatatgctgctaaggtttgataagagaaatgagattcagagaacagattatattgttgaagagcttgactggaaatgatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 35 - DNA, 3162 bp, Homo sapiens
  source 1..3162: mol_type = genomic DNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggctgcaatacagtgacacgatcacagctcactgctacagcctctacctcctgggcttaatccatcctccctcctcagcctcccaagcagctgggactacagatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagcatccatttgtgattggccatttcaacatctgtccttctcagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 36 - DNA, 2869 bp, Homo sapiens
  source 1..2869: mol_type = genomic DNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggctgcaatacagtgacacgatcacagctcactgctacagcctctacctcctgggcttaatccatcctccctcctcagcctcccaagcagctgggactacagaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 37 - DNA, 3380 bp, Homo sapiens
  source 1..3380: mol_type = genomic DNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggtgattttgatatgctgctaaggtttgataagagaaatgagattcagagaacagattatattgttgaagagcttgactggaaatgatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaagggtctcgctgtgttatccaggctggagtgcagtggcatgatcatggctcactgcagccttgacctcctgggctcaagtgatcctcccacctcagcctcccgagttgctgggactacaagcactgccaccatgctcagctaagaggcatgccactacttcccttcccactccttgccacctgcaccttctgactcactctagttgaaagccctgggggaaggaacaaactctgcagcctgactgctgtgtttaatcccagctctcctctactagctggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 38 - DNA, 3248 bp, Homo sapiens
  source 1..3248: mol_type = genomic DNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggtgattttgatatgctgctaaggtttgataagagaaatgagattcagagaacagattatattgttgaagagcttgactggaaatgatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaagggtctcgctgtgttatccaggctggagtgcagtggcatgatcatggctcactgcagccttgacctcctgggctcaagtgatcctcccacctcagcctcccgagttgctgggactacaagcactgccaccatgctcagctaagaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 39 - DNA, 3215 bp, Homo sapiens
  source 1..3215: mol_type = genomic DNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacagatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaatatggagttttgctcttttttgcccaggctggagtgcgatggcacaatcttggctcactgcaacctccacctcttgggttcaagcaatgcttctgcctcagcctcccaagtagctgggattacagagacagggtttcaccatgttggacaggccgggcacctgacctcaagtgatctgcccgcctcagcctgccaaaaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 40 - DNA, 3087 bp, Homo sapiens
  source 1..3087: mol_type = genomic DNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggcttgattgctgtaagttgaacacgtcagtggcatgggcaccagctgcacaaccctaggagccaccaaacacagtatattctatataaatgttgctgccaggagccccagttcaaactacaatatggagttttgctcttttttgcccaggctggagtgcgatggcacaatcttggctcactgcaacctccacctcttgggttcaagcaatgcttctgcctcagcctcccaagtagctgggattacagagacagggtttcaccatgttggacaggccgggcacctgacctcaagtgatctgcccgcctcagcctgccaaaaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 41 - DNA, 2418 bp, Homo sapiens
  source 1..2418: mol_type = genomic DNA
gcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggaggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaatgatgagtcaattcttgataagcagatgctcttatgcagagaacaaacttaatctttgccttttcattttctttttccttctttgagtttgaggtgtacacacttctgaaagagcctgcaggctacattagttataagagccattttaatttgggcttcaaattctctacttcttttccccaaataaagaacaacctaattttgtatcattgttagaatatcaaaaaaaattaagataagctggcatcaatatatacatttataaatatacattcattagcagttttctgactaaaatgtcacatcctggcacatcttttcgatttatgcatcatgtgctcacatctctgaaattctacaagacgtgtggatttttccacatcacttccttctcatattacccatctatgaactggctcactggagaattaaatttaaaaagtcaaagcctgttcttgcggcaaatagtttatggagtttattcttttaatttctcatgttgtgcctgattacgttcaggtttgtgatcttcctttttaaattgttcattgtacccatgtcctagaagtcattaaatcaaatattctgatcaaaa

SEQ ID NO: 42 - DNA, 2179 bp, Homo sapiens
  source 1..2179: mol_type = genomic DNA
gtgactgggtggggctgcctcacttctgcctgatttgggaagcgctgcaaggacaaccggctggggtccttgcgcgccgcggctcagggaggagcaccgactgcgccgcgtaagtgccgcctgccctgcgtgggtcgtgccagctcagcgggacaggtcctcgcctcggtccctcggacttagggagcgcggggcagaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaa

SEQ ID NO: 43 - DNA, 2608 bp, Homo sapiens
  source 1..2608: mol_type = genomic DNA
aaacaaagaaaatcaagtctgcgaggagcgagggtttgctcccctctgctccggatttgggtgtccagggtagcgggcgcggcacggcaaacaggtcggggcgctctgcttcaccccaaccttctccgccccgccagcccccgctccagctgcgccagaaagtgcgagcctgcccgctcctttcgcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacagatccaaacggaaacattggctcttcctggggttccagcctgctgtcttgacaggaactataccatcagctctcctgggtctcttacatgccaagtcatcctgcagatctgggactcactccgtaatcagcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaa

SEQ ID NO: 44 - DNA, 2480 bp, Homo sapiens
  source 1..2480: mol_type = genomic DNA
aaacaaagaaaatcaagtctgcgaggagcgagggtttgctcccctctgctccggatttgggtgtccagggtagcgggcgcggcacggcaaacaggtcggggcgctctgcttcaccccaaccttctccgccccgccagcccccgctccagctgcgccagaaagtgcgagcctgcccgctcctttcgcttttcgctcgctgcactccaagccccggaacacccgcgtccgcacactaagggcaccaccgctctcccacctctgcgctgtcagatgtcaggcgcggaggtgctctgggcaccgaggtgctggcgaaccaaacaagtatcaccccggacgcctgcccccattccgagagagcgggcgggatggccacaggcttgattgctgtaagttgaacacgtcagtggcatgggcaccagctgcacaaccctaggagccaccaaacacagtatattctatataaatgttgctgccaggagccccagttcaaactacaataccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaa

SEQ ID NO: 45 - DNA, 2021 bp, Homo sapiens
  source 1..2021: mol_type = genomic DNA
gcggctcagggaggagcaccgactgcgccgcaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaagtgagcttttgtttttggggggagatgtttactacaaaattaacatgagtacatgagtaagaattacattatttaacttaaaggatgaaaggtatggatgatgtgacactgagacaagatgtataaatgaaattttagggtcttgaataggaagttttaatttcttctaagagtaagtgaaaagtgcagttgtaacaaacaaagctgtaacatctttttctgccaataacagaagtttggcatgccgtgaaggtgtttggaaatattattggataagaatagctcaattatcccaaataaatggatgaagctataatagttttggggaaaagattctcaaatgtataaagtcttagaacaaaagaattctttgaaataaaaatattatatataaaagtaaaaaaaaaa

SEQ ID NO: 46 - DNA, 1581 bp, Homo sapiens
  source 1..1581: mol_type = genomic DNA
gagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagattctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatcccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagtgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaaaaaaa

SEQ ID NO: 47 - DNA, 1638 bp, Homo sapiens
  source 1..1638: mol_type = genomic DNA
ggacaaccggctggggtccttgcgcgccgcggctcagggaggagcaccgactgcgccgcaccctgagagatggttggtgccatgtggaaggtgattgtttcgctggtcctgttgatgcctggcccctgtgatgggctgtttcactccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggcttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcactccttgatgggcatggactggaaaggatcccaggaatacaagaaggcagaaaaaaaagtttggaagatccttaaatctgacagtgaagtggctggttacatccggcaagcgggtgactcccatcaggtaattattcgaggtggaggacatattttaccctatgaccagcctctgagagcttttgacatgattaatcgattcatttatggaaaaggatgggatccttatgttggataaactaccttcccgaaagagaacatcagaggttttcattgctgaaaagaaaatcgtaaaaacagaaaatgtcataggaataaaaaaattatcttttcatatctgcaagatttttttcatcaataaaaattatccttgaaa

SEQ ID NO: 48 - DNA, 1672 bp, Homo sapiens
  source 1..1672: mol_type = genomic DNA
aggacaaccggctggggtccttgcgcgccgcggctcagggaggagcaccgactgcgccgcaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcactccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaaaaaaaaaaaaaaaaaaaaaaaaaaaaaaaaaaa

SEQ ID NO: 49 - DNA, 1772 bp, Homo sapiens
  source 1..1772: mol_type = genomic DNA
agcgctgcaaggacaaccggctggggtccttgcgcgccgcggctcagggaggagcaccgactgcgccgcgtaagtgccgcctgccctgcgtgggtcgtgccagctcagcgggacaggtcctcgcctcggtccctcggacttagggagcgcggggcagaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagtgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaaaaaaaaaaaaaaaaaaaaaaaaaaaaaaaaaaaaaa

SEQ ID NO: 50 - DNA, 1605 bp, Homo sapiens
  source 1..1605: mol_type = genomic DNA
ggctcagggaggagcaccgactgcgccgcaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggcttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcactccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttg

SEQ ID NO: 51 - DNA, 1686 bp, Homo sapiens
  source 1..1686: mol_type = genomic DNA
ggtgactgggtggggctgcctcacttctgcctgatttgggaagcgctgcaaggacaaccggctggggtccttgcgcgccgcggctcagggaggagcaccgactgcgccgcaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcccttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagtgggtgacttccatcaggtaattattcgaggtggaggacatattttaccctatgaccagcctctgagagcttttgacatgattaatcgattcatttatggaaaaggatgggatccttatgttggataaactaccttcctaaaagagaacatcagaggttttcattgctgaaaagaaaatcgtaaaaacagaaaatgtcataggaataaaaaaattatcttttcatatctgcaagatttttttcatcaataaaaattatccttg

SEQ ID NO: 52 - protein, 476 aa, Homo sapiens
MVGAMWKVIVSLVLLMPGPCDGLFRSLYRSVSMPPKGDSGQPLFLTPYIEAGKIQKGRELSLVGPFPGLNMKSYAGFLTVNKTYNSNLFFWFFPAQIQPEDAPVVLWLQGGPGGSSMFGLFVEHGPYVVTSNMTLRDRDFPWTTTLSMLYIDNPVGTGFSFTDDTHGYAVNEDDVARDLYSALIQFFQIFPEYKNNDFYVTGESYAGKYVPAIAHLIHSLNPVREVKINLNGIAIGDGYSDPESIIGGYAEFLYQIGLLDEKQKKYFQKQCHECIEHIRKQNWFEAFEILDKLLDGDLTSDPSYFQNVTGCSNYYNFLRCTEPEDQLYYVKFLSLPEVRQAIHVGNQTFNDGTIVEKYLREDTVQSVKPWLTEIMNNYKVLIYNGQLDIIVAAALTERSLMGMDWKGSQEYKKAEKKVWKIFKSDSEVAGYIRQAGDFHQVIIRGGGHILPYDQPLRAFDMINRFIYGKGWDPYVG

SEQ ID NO: 53 - protein, 406 aa, Homo sapiens
MKSYAGFLTVNKTYNSNLFFWFFPAQIQPEDAPVVLWLQGGPGGSSMFGLFVEHGPYVVTSNMTLRDRDFPWTTTLSMLYIDNPVGTGFSFTDDTHGYAVNEDDVARDLYSALIQFFQIFPEYKNNDFYVTGESYAGKYVPAIAHLIHSLNPVREVKINLNGIAIGDGYSDPESIIGGYAEFLYQIGLLDEKQKKYFQKQCHECIEHIRKQNWFEAFEILDKLLDGDLTSDPSYFQNVTGCSNYYNFLRCTEPEDQLYYVKFLSLPEVRQAIHVGNQTFNDGTIVEKYLREDTVQSVKPWLTEIMNNYKVLIYNGQLDIIVAAALTERSLMGMDWKGSQEYKKAEKKVWKIFKSDSEVAGYIRQAGDFHQVIIRGGGHILPYDQPLRAFDMINRFIYGKGWDPYVG

SEQ ID NO: 54 - protein, 490 aa, Homo sapiens
MVGAMWKVIVSLVLLMPGPCDGLFRSLYRSVSMPPKGDSGQPLFLTPYIEAGKIQKGRELSLVGPFPGLNMKSYAGFLTVNKTYNSNLFFWFFPAQIQPEDAPVVLWLQGGPGGSSMFGLFVEHGPYVVTSNMTLRDRDFPWTTTLSMLYIDNPVGTGFSFTDDTHGYAVNEDDVARDLYSALIQFFQIFPEYKNNDFYVTGEHPFVIGHFNICPSQSYAGKYVPAIAHLIHSLNPVREVKINLNGIAIGDGYSDPESIIGGYAEFLYQIGLLDEKQKKYFQKQCHECIEHIRKQNWFEAFEILDKLLDGDLTSDPSYFQNVTGCSNYYNFLRCTEPEDQLYYVKFLSLPEVRQAIHVGNQTFNDGTIVEKYLREDTVQSVKPWLTEIMNNYKVLIYNGQLDIIVAAALTERSLMGMDWKGSQEYKKAEKKVWKIFKSDSEVAGYIRQAGDFHQVIIRGGGHILPYDQPLRAFDMINRFIYGKGWDPYVG

SEQ ID NO: 55 - protein, 298 aa, Homo sapiens
MGCFAPYTEVFPCHLRETQDSHYFSPLTLKLGRSKKIQPEDAPVVLWLQGGPGGSSMFGLFVEHGPYVVTSNMTLRDRDFPWTTTLSMLYIDNPVGTGFSFTDDTHGYAVNEDDVARDLYSALIQFFQIFPEYKNNDFYVTGEEPEDQLYYVKFLSLPEVRQAIHVGNQTFNDGTIVEKYLREDTVQSVKPWLTEIMNNYKVLIYNGQLDIIVAAALTERSLMGMDWKGSQEYKKAEKKVWKIFKSDSEVAGYIRQAGDFHQVIIRGGGHILPYDQPLRAFDMINRFIYGKGWDPYVG

SEQ ID NO: 56 - protein, 233 aa, Homo sapiens
MFGLFVEHGPYVVTSNMTLRDRDFPWTTTLSMLYIDNPVGTGFSFTDDTHGYAVNEDDVARDLYSALIQFFQIFPEYKNNDFYVTGESYAGKYVPAIAHLIHSLNPVREVKINLNGIAIGDGYSDPESIIGGYAEFLYQIGLLDEKQKKYFQKQCHECIEHIRKQNWFEAFEILDKLLDGDLTSDPSYFQNVTGCSNYYNFLRCTEPEDQLYYVKFLSLPEVRQAIPRGESDF

SEQ ID NO: 57 - DNA, 23 bp, synthetic construct
  source 1..23: mol_type = other DNA
  misc_feature 1..23: note = Synthetic sequence; gRNA Recognition Sequence
gtaaagcatggagagcgttgtgg

SEQ ID NO: 58 - DNA, 23 bp, synthetic construct
  source 1..23: mol_type = other DNA
  misc_feature 1..23: note = Synthetic sequence; gRNA Recognition Sequence
ctcattgactgcatatccgtggg

SEQ ID NO: 59 - DNA, 23 bp, synthetic construct
  source 1..23: mol_type = other DNA
  misc_feature 1..23: note = Synthetic sequence; gRNA Recognition Sequence
ctgtagccagagaactactgggg

SEQ ID NO: 60 - DNA, 23 bp, synthetic construct
  source 1..23: mol_type = other DNA
  misc_feature 1..23: note = Synthetic sequence; gRNA Recognition Sequence
ccacggatatgcagtcaatgagg

SEQ ID NO: 61 - DNA, 23 bp, synthetic construct
  source 1..23: mol_type = other DNA
  misc_feature 1..23: note = Synthetic sequence; gRNA Recognition Sequence
tcaatgaggacgatgtagcacgg

SEQ ID NO: 62 - DNA, 23 bp, synthetic construct
  source 1..23: mol_type = other DNA
  misc_feature 1..23: note = Synthetic sequence; gRNA Recognition Sequence
tcaccccttacattgaagctggg

SEQ ID NO: 63 - DNA, 23 bp, synthetic construct
  source 1..23: mol_type = other DNA
  misc_feature 1..23: note = Synthetic sequence; gRNA Recognition Sequence
gcatagccccctataatctgagg

SEQ ID NO: 64 - DNA, 23 bp, synthetic construct
  source 1..23: mol_type = other DNA
  misc_feature 1..23: note = Synthetic sequence; gRNA Recognition Sequence
agtgtttccatgccacctaaggg

SEQ ID NO: 65 - DNA, 23 bp, synthetic construct
  source 1..23: mol_type = other DNA
  misc_feature 1..23: note = Synthetic sequence; gRNA Recognition Sequence
cttcccagcttcaatgtaagggg

SEQ ID NO: 66 - DNA, 23 bp, synthetic construct
  source 1..23: mol_type = other DNA
  misc_feature 1..23: note = Synthetic sequence; gRNA Recognition Sequence
ttggactctttgtggaacatggg

SEQ ID NO: 67 - DNA, 23 bp, synthetic construct
  source 1..23: mol_type = other DNA
  misc_feature 1..23: note = Synthetic sequence; gRNA Recognition Sequence
atgccacctaagggagactcagg

SEQ ID NO: 68 - DNA, 23 bp, synthetic construct
  source 1..23: mol_type = other DNA
  misc_feature 1..23: note = Synthetic sequence; gRNA Recognition Sequence
gtccttctcagtcttatgcaggg

SEQ ID NO: 69 - DNA, 23 bp, synthetic construct
  source 1..23: mol_type = other DNA
  misc_feature 1..23: note = Synthetic sequence; gRNA Recognition Sequence
ccaacaagccaatttggtacagg

SEQ ID NO: 70 - DNA, 23 bp, synthetic construct
  source 1..23: mol_type = other DNA
  misc_feature 1..23: note = Synthetic sequence; gRNA Recognition Sequence
gaggtgaagatcaacctgaacgg

SEQ ID NO: 71 - DNA, 23 bp, synthetic construct
  source 1..23: mol_type = other DNA
  misc_feature 1..23: note = Synthetic sequence; gRNA Recognition Sequence
ttccacaaagagtccaaacatgg

SEQ ID NO: 72 - DNA, 23 bp, synthetic construct
  source 1..23: mol_type = other DNA
  misc_feature 1..23: note = Synthetic sequence; gRNA Recognition Sequence
tgtaagtcttattcacggtgagg

SEQ ID NO: 73 - DNA, 23 bp, synthetic construct
  source 1..23: mol_type = other DNA
  misc_feature 1..23: note = Synthetic sequence; gRNA Recognition Sequence
tgtcctgagtctcccttaggtgg

SEQ ID NO: 74 - DNA, 23 bp, synthetic construct
  source 1..23: mol_type = other DNA
  misc_feature 1..23: note = Synthetic sequence; gRNA Recognition Sequence
caatgaggacgatgtagcacggg

SEQ ID NO: 75 - DNA, 23 bp, synthetic construct
  source 1..23: mol_type = other DNA
  misc_feature 1..23: note = Synthetic sequence; gRNA Recognition Sequence
atactggataaactactagatgg

SEQ ID NO: 76 - DNA, 23 bp, synthetic construct
  misc_feature 1..23: note = Synthetic sequence; gRNA Recognition Sequence
  source 1..23: mol_type = other DNA
tgcattcatggcactgcttctgg